(19) United States
(12) Patent Application Publication
(10) Pub. No.: US 2014/0287471 A1
(43) Pub. Date: Sep. 25, 2014

(54) VARIANT CBH I POLYPEPTIDES WITH REDUCED PRODUCT INHIBITION

(71) Applicant: BP CORPORATION NORTN AMERICA INC., Houston, TX (US)

(72) Inventors: Sarah Richardson Hanson, San Marcos, CA (US); Justin T. Stege, San Diego, CA (US); Cecilia Cheng, San Diego, CA (US); Peter Luginbuhl, San Diego, CA (US)

(73) Assignee: Bp Corporation North America Inc., Houston, TX (US)

(21) Appl. No.: 14/349,253

(22) PCT Filed: Oct. 5, 2012
(86) PCT No.: PCT/US12/59005

§ 371 Date: Apr. 2, 2014

Related U.S. Application Data
(60) Provisional application No. 61/622,971, filed on Apr. 11, 2012.
(60) Provisional application No. 61/544,256, filed on Oct. 6, 2011.

(51) Int. Cl. C12N 9/24

(52) U.S. Cl. CPC C12N 9/2402
USPC 435/100; 536/23.2; 536/55.3; 435/209; 435/105; 435/161; 435/320.1; 435/254.3; 435/254.5; 435/254.9; 435/254.11; 435/254.6; 435/254.7; 435/254.21; 435/254.22; 435/254.23; 435/254.2; 435/252.33; 435/252.31; 435/252.34; 435/252.35; 435/252.3

(57) ABSTRACT

The present disclosure relates to variant CBH I polypeptides that have reduced product inhibition, and compositions, e.g., cellulase compositions, comprising variant CBH I polypeptides. The variant CBH I polypeptides and related compositions can be used in variety of agricultural and industrial applications. The present disclosure further relates to nucleic acids encoding variant CBH I polypeptides and host cells that recombinantly express the variant CBH I polypeptides.

### BACKGROUND

Cellulose is an unbranched polymer of glucose linked by β(1→4)-glycosidic bonds. Cellulose chains can interact with each other via hydrogen bonding to form a crystalline solid of high mechanical strength and chemical stability. The cellulose chains are depolymerized into glucose and short oligosaccharides before organisms, such as the fermenting microbes used in ethanol production, can use them as metabolic fuel. Cellulase enzymes catalyze the hydrolysis of the cellulose (hydrolysis of β-1,4-D-glucan linkages) in the biomass into products such as glucose, cellobiose, and other cellooligosaccharides. Cellulase is a generic term denoting a multienzyme mixture comprising exo-acting cellobiohydrolases (CBHs), endoglucanases (EGs) and β-glucosidases (BGs) that can be produced by a number of plants and microorganisms. Enzymes in the cellulase of Trichoderma reesei include CBH I (more generally, Cel7A), CBH2 (Cel6A), EG1 (Cel7B), EG2 (Cel5), EG3 (Cel12), EG4 (Cel61A), EG5 (Cel45A), EG6 (Cel74A), Cip1, Cip2, β-glucosidases (including, e.g., Cel3A), acetyl xylan esterase, β-mannanase, and swollenin.

Cellulase enzymes work synergistically to hydrolyze cellulose to glucose. CBH I and CBH II act on opposing ends of cellulose chains (Barr et al., 1996, Biochemistry 35:586-92), while the endoglucanases act at internal locations in the cellulose. The primary product of these enzymes is cellobiose, which is further hydrolyzed to glucose by one or more β-glucosidases.

The cellobiohydrolases are subject to inhibition by their direct product, cellobiose, which results in a slowing down of saccharification reactions as product accumulates. There is a need for new and improved cellobiohyrolases with improved productivity that maintain their reaction rates during the course of a saccharification reaction, for use in the conversion of cellulose into fermentable sugars and for related fields of cellulosic material processing such as pulp and paper, textiles and animal feeds.

### SUMMARY

The present disclosure relates to variant CBH I polypeptides. Most naturally occurring CBH I polypeptides have arginines at positions corresponding to R268 and R411 of T. reesei CBH I (SEQ ID NO:2). The variant CBH I polypeptides of the present disclosure include a substitution at either or both positions resulting in a reduction or decrease in product (e.g., cellobiose) inhibition. Such variants are sometimes referred to herein as “product tolerant.” In some instances, the variants have an increased specific activity towards a CBH I substrate.

Accordingly, the present invention provides polypeptides (variant CBH I polypeptides) in which the CBH I catalytic domain has been engineered to incorporate an amino acid substitution that results in increased tolerance to cellobiose, increased specific activity, or both. The variant CBH I polypeptides of the disclosure minimally contain at least a CBH I catalytic domain, comprising (a) a substitution at the amino acid position corresponding to R268 of T. reesei CBH I (“R268 substitution”); (b) a substitution at the amino acid position corresponding to R411 of T. reesei CBH I (“R411 substitution”); or (c) both an R268 substitution and an R411 substitution. The amino acid positions of exemplary CBH I polypeptides into which R268 and/or R411 substitutions can be introduced are shown in Table 1, and the amino acid positions corresponding to R268 and/or R411 in these exemplary CBH I polypeptides are shown in Table 2.

The polypeptides of the disclosure show at least 2-fold, at least 5-fold, at least 10-fold, at least 15-fold, at least 20-fold, at least 25-fold, at least 50-fold, at least 100-fold, at least 150-fold, at least 200-fold, at least 250-fold, at least 500-fold or at least 700-fold greater tolerance to cellobiose, and in some cases up to 750-fold or up to 1,000-fold greater tolerance to cellobiose, a wild type CBH I which does not have a substitution at the amino acid corresponding to R268 or the amino acid position corresponding to R411. Product tolerance can suitably be determined by assaying the IC₅₀, the half maximal inhibitory concentration, of cellobiose towards the polypeptide.

In certain aspects, the polypeptides of the disclosure are characterized by an IC₅₀ of cellobiose is at least 0.1 mM, at least 0.5 mM, at least 1 mM, at least 2 mM, at least 3 mM, at least 5 mM, at least 7 mM, at least 10 mM, at least 12 mM, at least 15 mM, at least 20 mM, at least 25 mM or at least 30 mM.

In certain embodiments, a polypeptide of the disclosure comprises an R268 substitution. The R268 substitution preferably results in an IC₅₀ of cellobiose that is at least 2-fold, at least 5-fold, at least 7.5-fold or at least 10-fold the IC₅₀ of cellobiose on the reference CBH I (e.g., a CBH I without an R268 or R411 substitution). In certain embodiments, the R411 substitution results in an IC₅₀ of cellobiose of at least 0.1 mM, at least 0.25 mM, or at least 0.5 mM. Exemplary R268 substituents are (a) histidine or lysine; (b) isoleucine, leucine, valine, phenylalanine, tyrosine, asparagine, serine, threonine, cysteine, or glycine; (c) alanine, tryptophan, aspartate, glutamate, or proline; or (d) glutamine or methionine. R268 substitutions were generally found to increase the specific activity of CBH I, in some cases up to 4.4-fold (see Table 13).

In certain embodiments, a polypeptide of the disclosure comprises an R411 substitution. The R411 substitution preferably results in an IC₅₀ of cellobiose that is at least 10-fold, at least 15-fold, at least 20-fold, at least 25-fold, at least 50-fold, at least 100-fold or at least 140-fold the IC50 of cellobiose on the reference CBH I (e.g., a CBH I without an R268 or R411 substitution). In certain embodiments, the R411 substitution results in an IC50 of cellobiose of at least 1 mM, at least 2 mM, at least 3 mM, at least 4 mM, at least 5 mM, at least 6 mM, at least 7 mM or at least 8 mM. Exemplary R411 substituents are (a) alanine, aspartate, serine, cysteine, or proline; (b) valine, glutamate, histidine, lysine, threonine, glycine, methionine, or, optionally, glutamine; (c) leucine, phenylalanine, tryptophan, tyrosine, or asparagine; or (d) isoleucine. R411 substitutions were generally found to not impact or slightly decrease the specific activity of CBH I.

It was surprisingly discovered that introducing both R268 and R411 substitutions resulted in synergistic effects on CBH I product tolerance (see Table 12), without meaningfully affecting, and in several cases increasing, specific activity of the enzyme (see Table 13). Accordingly, introducing both R268 and R411 substitutions into a CBH I molecule is particularly beneficial.

The CBH I polypeptides the disclosure with both R268 and R411 substitutions preferably show a 100-fold to 1,000-fold improvement in tolerance to cellobiose, and a specific activity of 0.7-fold to 3-fold the specific activity, of a wild type CBH I which does not have either R268 or R411 substitutions. In some embodiments of the foregoing ranges, the improvement in cellobiose tolerance is at least 200- or 300-fold, and the specific activity is at least 1-fold or at least 1.5-fold the specific activity of said wild type CBH I.

In certain aspects, a CBH I polypeptide of the disclosure is any variant having the amino acid substitutions enumerated in Table 14, which shows 399 possible R268 and/or R411 amino acid substitutions (with a dash “-” indicating a wild type “R” residue). Thus, the variant can be characterized by a single R268 or R411 substitution or a double R268/R411 substitution. Variants with single R268 substitutions can be selected from variant nos. 281-299 in Table 14, and variants with single R411 substitutions can be selected from variant nos. 15, 35, 55, 75, 95, 115, 135, 155, 175, 215, 235, 255, 275, 314, 334, 354, 374, and 396 in Table 14. Variants with a double R268/R411 substitution can be selected from variant nos. 1-14, 16-34, 36-54, 56-74, 76-94, 96-114, 116-134, 136-154, 156-174, 176-194, 196-214, 216-234, 236-254, 256-74, 276-280, 300-313, 315-333, 335-353, 355-373, 375-393, and 395-399. In specific embodiments, the variant does not have the same substitutions as one or more of variants 1, 9, 15, 161, 169, 175, 281 and/or 289 of Table 14.

In certain embodiments, R268 and/or R411 substituents can include lysines and/or alanines. Accordingly, the present disclosure provides a variant CBH I polypeptide comprising a CBH I catalytic domain with one of the following amino acid substitutions or pairs of R268 and/or R411 substitutions: (a) R268K and R411K; (b) R268K and R411A; (c) R268A and R411K; (d) R268A and R411A; (e) R268A; (f) R268K; (g) R411A; and (h) R411K. In some embodiments, however, the amino acid sequence of the variant CBH I polypeptide does not comprise or consist of SEQ ID NO:299, SEQ ID NO:300, SEQ ID NO:301, or SEQ ID NO:302.

The variant CBHI polypeptides of the disclosure typically include a CD comprising an amino acid sequence having at least 50% sequence identity to a CD of a reference CBH I exemplified in Table 1. The CD portions of the CBH I polypeptides exemplified in Table 1 are delineated in Table 3. The variant CBH I polypeptides can have a cellulose binding domain (“CBD”) sequence in addition to the catalytic domain (“CD”) sequence. The CBD can be N- or C-terminal to the CD, and the CBD and CD are optionally connected via a linker sequence.

The variant CBH I polypeptides can be mature polypeptides or they may further comprise a signal sequence.

Additional embodiments of the variant CBH I polypeptides are provided in Section 1.1.

The variant CBH I polypeptides of the disclosure typically exhibit reduced product inhibition by cellobiose. In certain embodiments, the IC₅₀ of cellobiose towards a variant CBH I polypeptide of the disclosure is at least 1.2-fold, at least 1.5-fold, or at least 2-fold the IC₅₀ of cellobiose towards a reference CBH I lacking the R268 substitution and/or R411 substitution present in the variant. Additional embodiments of the product inhibition characteristics of the variant CBH I polypeptides are provided in Section 1.1.

The variant CBH I polypeptides of the disclosure typically retain some cellobiohydrolase activity. In certain embodiments, a variant CBH I polypeptide retains at least 50% the CBH I activity of a reference CBH I lacking the R268 substitution and/or R411 substitution present in the variant. Additional embodiments of cellobiohydrolase activity of the variant CBH I polypeptides are provided in Section 1.1.

The present disclosure further provides compositions (including cellulase compositions, e.g., whole cellulase compositions, and fermentation broths) comprising variant CBH I polypeptides. Additional embodiments of compositions comprising variant CBH I polypeptides are provided in Section 1.3. The variant CBH I polypeptides and compositions comprising them can be used, inter alia, in processes for saccharifying biomass. Additional details of saccharification reactions, and additional applications of the variant CBH I polypeptides, are provided in Section 1.4.

The present disclosure further provides nucleic acids (e.g., vectors) comprising nucleotide sequences encoding variant CBH I polypeptides as described herein, and recombinant cells engineered to express the variant CBH I polypeptides. The recombinant cell can be a prokaryotic (e.g., bacterial) or eukaryotic (e.g., yeast or filamentous fungal) cell. Further provided are methods of producing and optionally recovering the variant CBH I polypeptides. Additional embodiments of the recombinant expression system suitable for expression and production of the variant CBH I polypeptides are provided in Section 1.2.

### BRIEF DESCRIPTION OF THE FIGURES AND TABLES

FIG. 1A-1B: Cellobiose dose-response curves using a 4-MUL assay for a wild-type CBH I (BD29555; FIG. 1A) and a R268K/R411K variant CBH I (BD29555 with the substitutions R273K/R422K; FIG. 1B).

FIG. 2A-2B: The effect of cellobiose accumulation on the activity of wild-type CBH I and a R268K/R411K variant CBH I, based on percent conversion of glucan after 72 hours in the bagasse assay. FIG. 2A shows relative activity in the presence (+) and absence (−) of β-glucosidase (BG), where relative activity is normalized to wild type activity with BG (WT+=1). FIG. 2B shows tolerance to cellobiose as a function of the ratio of activity in the absence vs. presence of β-glucosidase (activity ratio=Activity−BG/Activity+BG).

FIG. 3: Cellobiose dose-response curves using PASC assay for a R268K/R411K variant CBH I polypeptide as compared to two wild type CBH I polypeptides.

FIG. 4: The effect of cellobiose accumulation on the activity of a wild-type CBH I and a R268K/R411K variant CBH I based on percent conversion of glucan after 72 hours in the bagasse assay in the presence (+) and absence (−) of β-glucosidase (BG). Activity is normalized to wild type activity with BG (WT+=1).

FIG. 5: Characterization of cellobiose product tolerance of variant CBH I polypeptides, based on percent conversion of glucan after 72 hours in the absence and presence of β-glucosidase (BG) in the bagasse assay; tolerance is evaluated as a function of the ratio of activity in the absence vs. presence of β-glucosidase.

FIG. 6: Scheme 1. Primary Screening flow sheet.

FIG. 7: Scheme 2. Secondary Screening flow sheet.

FIG. 8: Saccharification assay demonstrating that variant library retains enzymatic activity.

FIG. 9: Representative IC₅₀ curves for the serine mutation with IC₅₀ values of 0.45, 0.89, 6.8, and 9.12 for 268S, 411 S, 268A/411S, and 268S/411A, respectively. Curves show the clear synergistic shift in IC₅₀ value resulting from the double mutants. Specific activity effects can be clearly seen with higher relative fluorescence units for variants having the 268 mutation.

FIG. 10: Three dimensional plot of IC₅₀ values: x-axis indicates amino acid mutations; bars on the z-axis represents experimentally determined IC₅₀ values; y-axis shows the sequence context of the mutations.

FIG. 11: Three dimensional plot for specific activity increases by 4MUL: x-axis indicates amino acid mutations; bars on the z-axis represents experimentally determined SA values; y-axis shows the sequence context of the mutations.

TABLE 1: Amino acid sequences of exemplary “reference” CBH I polypeptides that can be modified at positions corresponding to R268 and/or R411 in T. reesei CBH I (SEQ ID NO:2). The database accession numbers are indicated in the second column. Unless indicated otherwise, the accession numbers refer to the Genbank database. “#” indicates that the CBH I has no signal peptide; “&” indicate that the sequence is from the PDB database and represents the catalytic domain only without signal sequence; * indicates a nonpublic database. These amino acid sequences are mostly wild type, with the exception of some sequences from the PDB database which contain mutations to facilitate protein crystallization.

TABLE 2: Amino acid positions in the exemplary reference CBH I polypeptides that correspond to R268 and R411 in T. reesei CBH I. Database descriptors are as for Table 1.

TABLE 3: Approximate amino acid positions of CBH I polypeptide domains. Abbreviations used: SS is signal sequence; CD is catalytic domain; and CBD is cellulose binding domain. Database descriptors are as for Table 1.

TABLE 4: Table 4 shows a segment within the catalytic domain of each exemplary reference CBH I polypeptide containing the active site loop (shown in bold, underlined text) and the catalytic residues (glutamates in most CBH I polypeptides) (shown in bold, double underlined text). Database descriptors are as for Table 1.

TABLE 5: MUL and bagasse assay results for variants of BD29555. ND means not determined. ±% Activity (+/− cellobiose)=[(Activity with cellobiose)/(Activity without cellobiose)]*100. ¥ % Activity (−/+BG)=[(Activity without BG)/(Activity with BG)]*100]

TABLE 6: MUL and bagasse assay results for variants of T. reesei CBH I. ND means not determined. +% Activity (+/− cellobiose)=[(Activity with cellobiose)/(Activity without cellobiose)]*100. ¥% Activity (−/+BG)=[(Activity without BG)/(Activity with BG)]*100.

TABLE 7: Informal sequence listing. SEQ ID NO:1-149 correspond to the exemplary reference CBH I polypeptides. SEQ ID NO:299 corresponds to mature T. reesei CBH I (amino acids 26-529 of SEQ ID NO:2) with an R268A substitution. SEQ ID NO:300 corresponds to mature T. reesei CBH I (amino acids 26-529 of SEQ ID NO:2) with an R411A substitution. SEQ ID NO:301 corresponds to full length BD29555 with both an R268K substitution and an R411K substitution. SEQ ID NO:302 corresponds to mature BD29555 with both an R268K substitution and an R411K substitution.

TABLE 8: Primary Screening Results (10 μL enzyme; cellobiose range: 0.0001-100 mM; n=1)

TABLE 9: Secondary Screening IC₅₀s (CBH I levels normalized to 5 μg/μL; cellobiose range: 0.0001-100 mM)

TABLE 10: Secondary Screening IC₅₀, (CBH I levels normalized to 5 μg/μL, cellobiose range: 0.00085-100 mM)

TABLE 11: Secondary Screening IC₅₀s (304 harvested supernatant; cellobiose range: 0.00085-100 mM)

TABLE 12: Merged IC₅₀ values (from Tables 8-11) showing increased tolerance by single mutations and synergistic increase by double mutation. ND=not determined; ^{¥}=data with fewer than 3 replicates and/or curve fitting with R²<0.95; * Improvement of variant IC₅₀ value over wild type=variant/WT (where WT IC50=0.046); ̂ expected=additive IC₅₀ value based on single measurements; ** synergistic increase=measured/expected.

TABLE 13: Specific Activity (SA, μmol 4 MU/min/mg CBH I) values. *Δ SA: change in specific activity; ratio of variant: WT; ^{¥} data derived from variants with low protein quantification, with fewer than 3 replicates and/or curve fitting with R²<0.95; WT Specific Activity=0.76.

TABLE 14: Table of possible single and double R268 and/or R411 substitutions that can be introduced into a CBH I polypeptide.

### DETAILED DESCRIPTION

The present disclosure relates to variant CBH I polypeptides. Most naturally occurring CBH I polypeptides have arginines at positions corresponding to R268 and R411 of T. reesei CBH I (SEQ ID NO:2). The variant CBH I polypeptides of the present disclosure include a substitution at either or both positions resulting in a reduction of product (e.g., cellobiose) inhibition, and/or an improved specific activity. The following subsections describe in greater detail the variant CBH I polypeptides and exemplary methods of their production, exemplary cellulase compositions comprising them, and some industrial applications of the polypeptides and cellulase compositions.

1.1. Variant CBH I Polypeptides

The present disclosure provides variant CBH I polypeptides comprising at least one amino acid substitution that results in reduced product inhibition. “Variant” means a polypeptide which differs in sequence from a reference polypeptide by substitution of one or more amino acids at one or a number of different sites in the amino acid sequence. Exemplary reference CBH I polypeptides are shown in Table 1.

The variant CBH I polypeptides of the disclosure have an amino acid substitution at the amino acid position corresponding to R268 of T. reesei CBH I (SEQ ID NO:2) (an “R268 substitution”), (b) a substitution at the amino acid position corresponding to R411 of T. reesei CBH I (“R411 substitution”); or (c) both an R268 substitution and an R411 substitution, as compared to a reference CBH I polypeptide. It is noted that the R268 and R411 numbering is made by reference to the full length T. reesei CBH I, which includes a signal sequence that is generally absent from the mature enzyme. The corresponding numbering in the mature T. reesei CBH I (see, e.g., SEQ ID NO:4) is R251 and R394, respectively.

Accordingly, the present disclosure provides variant CBH I polypeptides in which at least one of the amino acid positions corresponding to R268 and R411 of T. reesei CBH I, and optionally both the amino acid positions corresponding to 8268 and R411 of T. reesei CBH I, is not an arginine.

The amino acid positions in the reference polypeptides of Table 1 that correspond to R268 and R411 in T. reesei CBH I are shown in Table 2. Amino acid positions in other CBH 1 polypeptides that correspond to R268 and R411 can be identified through alignment of their sequences with T. reesei CBH I using a sequence comparison algorithm. Optimal alignment of sequences for comparison can be conducted, e.g., by the local homology algorithm of Smith & Waterman, 1981, Adv. Appl. Math. 2:482-89; by the homology alignment algorithm of Needleman & Wunsch, 1970, J. Mol. Biol. 48:443-53; by the search for similarity method of Pearson & Lipman, 1988, Proc. Nat'l Acad. Sci. USA 85:2444-48, by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

The R268 and/or R411 substitutions can be selected from Table 14, which includes all possible 399 possible single and double R268 and R411 substitutions. In certain embodiments, the variants (a) R268K and R411K; (b) R268K and R411A; (c) R268A and R411K; (d) R268A and R411A; (e) R268A; (f) R268K; (g) R411A; or (h) R411K. In other embodiments, the variants are any variants in Table 14 except one or more of the variants (a) R268K and R411K; (b) R268K and R411A; (c) R268A and R411K; (d) R268A and R411A; (e) R268A; (f) R268K; (g) R411A; and (h) R411K.

CBH I polypeptides belong to the glycosyl hydrolase family 7 (“GH7”). The glycosyl hydrolases of this family include endoglucanases and cellobiohydrolases (exoglucanases). The cellobiohydrolases act processively from the reducing ends of cellulose chains to generate cellobiose. Cellulases of bacterial and fungal origin characteristically have a small cellulose-binding domain (“CBD”) connected to either the N or the C terminus of the catalytic domain (“CD”) via a linker peptide (see Suumakki et al., 2000, Cellulose 7: 189-209). The CD contains the active site whereas the CBD interacts with cellulose by binding the enzyme to it (van Tilbeurgh et al., 1986, FEBS Lett. 204(2): 223-227; Tomme et al., 1988, Eur. J. Biochem. 170:575-581). The three-dimensional structure of the catalytic domain of T. reesei CBH I has been solved (Divne et al., 1994, Science 265:524-528). The CD consists of two β-sheets that pack face-to-face to form a β-sandwich. Most of the remaining amino acids in the CD are loops connecting the β-sheets. Some loops are elongated and bend around the active site, forming cellulose-binding tunnel of (˜50 Å). In contrast, endoglucanases have an open substrate binding cleft/groove rather than a tunnel. Typically, the catalytic residues are glutamic acids corresponding to E229 and E234 of T. reesei CBH I.

The loops characteristic of the active sites (“the active site loops”) of reference CBH I polypeptides, which are absent from GH7 family endoglucanases, as well as catalytic glutamate residues of the reference CBH I polypeptides, are shown in Table 4. The variant CBH I polypeptides of the disclosure preferably retain the catalytic glutamate residues or may include a glutamine instead at the position corresponding to E234, as for SEQ ID NO:4. In some embodiments, the variant CBH I polypeptides contain no substitutions or only conservative substitutions in the active site loops relative to the reference CBH I polypeptides from which the variants are derived.

Many CBH I polypeptides do not have a CBD, and most studies concerning the activity of cellulase domains on different substrates have been carried out with only the catalytic domains of CBH I polypeptides. Because CDs with cellobiohydrolase activity can be generated by limited proteolysis of mature CBH I by papain (see, e.g., Chen et al., 1993, Biochem. Mol. Biol. Int. 30(5):901-10), they are often referred to as “core” domains. Accordingly, a variant CBH I can include only the CD “core” of CBH I. Exemplary reference CDs comprise amino acid sequences corresponding to positions 26 to 455 of SEQ ID NO:1, positions 18 to 444 of SEQ ID NO:2, positions 26 to 455 of SEQ ID NO:3, positions 1 to 427 of SEQ ID NO:4, positions 24 to 457 of SEQ ID NO:5, positions 18 to 448 of SEQ ID NO:6, positions 27 to 460 of SEQ ID NO:7, positions 27 to 460 of SEQ ID NO:8, positions 20 to 449 of SEQ ID NO:9, positions 1 to 424 of SEQ ID NO:10, positions 18 to 447 of SEQ ID NO:11, positions 18 to 434 of SEQ ID NO:12, positions 18 to 445 of SEQ ID NO:13, positions 19 to 454 of SEQ ID NO:14, positions 19 to 443 of SEQ ID NO:15, positions 2 to 426 of SEQ ID NO:16, positions 23 to 446 of SEQ ID NO:17, positions 19 to 449 of SEQ ID NO:18, positions 23 to 446 of SEQ ID NO:19, positions 19 to 449 of SEQ ID NO:20, positions 2 to 416 of SEQ ID NO:21, positions 19 to 454 of SEQ ID NO:22, positions 19 to 447 of SEQ ID NO:23, positions 19 to 447 of SEQ ID NO:24, positions 20 to 443 of SEQ ID NO:25, positions 18 to 447 of SEQ ID NO:26, positions 19 to 442 of SEQ ID NO:27, positions 18 to 451 of SEQ ID NO:28, positions 23 to 446 of SEQ ID NO:29, positions 18 to 444 of SEQ ID NO:30, positions 18 to 451 of SEQ ID NO:31, positions 18 to 447 of SEQ ID NO:32, positions 19 to 449 of SEQ ID NO:33, positions 18 to 447 of SEQ ID NO:34, positions 26 to 459 of SEQ ID NO:35, positions 19 to 450 of SEQ ID NO:36, positions 19 to 453 of SEQ ID NO:37, positions 18 to 448 of SEQ ID NO:38, positions 19 to 443 of SEQ ID NO:39, positions 19 to 442 of SEQ ID NO:40, positions 18 to 444 of SEQ ID NO:41, positions 24 to 457 of SEQ ID NO:42, positions 18 to 449 of SEQ ID NO:43, positions 19 to 453 of SEQ ID NO:44, positions 26 to 456 of SEQ ID NO:45, positions 19 to 451 of SEQ ID NO:46, positions 18 to 443 of SEQ ID NO:47, positions 18 to 448 of SEQ ID NO:48, positions 19 to 451 of SEQ ID NO:49, positions 18 to 444 of SEQ ID NO:50, positions 2 to 419 of SEQ ID NO:51, positions 27 to 461 of SEQ ID NO:52, positions 21 to 445 of SEQ ID NO:53, positions 19 to 449 of SEQ ID NO:54, positions 19 to 448 of SEQ ID NO:55, positions 18 to 443 of SEQ ID NO:56, positions 20 to 443 of SEQ ID NO:57, positions 18 to 448 of SEQ ID NO:58, positions 18 to 447 of SEQ ID NO:59, positions 26 to 455 of SEQ ID NO:60, positions 19 to 449 of SEQ ID NO:61, positions 19 to 449 of SEQ ID NO:62, positions 26 to 460 of SEQ ID NO:63, positions 18 to 448 of SEQ ID NO:64, positions 19 to 451 of SEQ ID NO:65, positions 19 to 447 of SEQ ID NO:66, positions 1 to 424 of SEQ ID NO:67, positions 19 to 448 of SEQ ID NO:68, positions 19 to 443 of SEQ ID NO:69, positions 23 to 447 of SEQ ID NO:70, positions 17 to 448 of SEQ ID NO:71, positions 19 to 449 of SEQ ID NO:72, positions 18 to 444 of SEQ ID NO:73, positions 23 to 458 of SEQ ID NO:74, positions 20 to 452 of SEQ ID NO:75, positions 18 to 435 of SEQ ID NO:76, positions 18 to 446 of SEQ ID NO:77, positions 22 to 457 of SEQ ID NO:78, positions 18 to 448 of SEQ ID NO:79, positions 1 to 431 of SEQ ID NO:80, positions 19 to 453 of SEQ ID NO:81, positions 21 to 440 of SEQ ID NO:82, positions 19 to 442 of SEQ ID NO:83, positions 18 to 448 of SEQ ID NO:84, positions 17 to 446 of SEQ ID NO:85, positions 18 to 447 of SEQ ID NO:86, positions 18 to 443 of SEQ ID NO:87, positions 23 to 448 of SEQ ID NO:88, positions 18 to 451 of SEQ ID NO:89, positions 21 to 447 of SEQ ID NO:90, positions 18 to 444 of SEQ ID NO:91, positions 19 to 442 of SEQ ID NO:92, positions 20 to 436 of SEQ ID NO:93, positions 18 to 450 of SEQ ID NO:94, positions 22 to 453 of SEQ ID NO:95, positions 16 to 472 of SEQ ID NO:96, positions 21 to 445 of SEQ ID NO:97, positions 19 to 447 of SEQ ID NO:98, positions 19 to 450 of SEQ ID NO:99, positions 19 to 451 of SEQ ID NO:100, positions 18 to 448 of SEQ ID NO:101, positions 19 to 442 of SEQ ID NO:102, positions 20 to 457 of SEQ ID NO:103, positions 19 to 454 of SEQ ID NO:104, positions 18 to 440 of SEQ ID NO:105, positions 18 to 439 of SEQ ID NO:106, positions 27 to 460 of SEQ ID NO:107, positions 23 to 446 of SEQ ID NO:108, positions 17 to 446 of SEQ ID NO:109, positions 21 to 447 of SEQ ID NO:110, positions 19 to 447 of SEQ ID NO:111, positions 18 to 449 of SEQ ID NO:112, positions 22 to 457 of SEQ ID NO:113, positions 18 to 445 of SEQ ID NO:114, positions 18 to 448 of SEQ ID NO:115, positions 18 to 448 of SEQ ID NO:116, positions 23 to 435 of SEQ ID NO:117, positions 21 to 442 of SEQ ID NO:118, positions 23 to 435 of SEQ ID NO:119, positions 20 to 445 of SEQ ID NO:120, positions 21 to 443 of SEQ ID NO:121, positions 20 to 445 of SEQ ID NO:122, positions 23 to 443 of SEQ ID NO:123, positions 20 to 445 of SEQ ID NO:124, positions 21 to 435 of SEQ ID NO:125, positions 20 to 437 of SEQ ID NO:126, positions 21 to 442 of SEQ ID NO:127, positions 23 to 434 of SEQ ID NO:128, positions 20 to 444 of SEQ ID NO:129, positions 21 to 435 of SEQ ID NO:130, positions 20 to 445 of SEQ ID NO:131, positions 21 to 446 of SEQ ID NO:132, positions 21 to 435 of SEQ ID NO:133, positions 22 to 448 of SEQ ID NO:134, positions 23 to 433 of SEQ ID NO:135, positions 23 to 434 of SEQ ID NO:136, positions 23 to 435 of SEQ ID NO:137, positions 23 to 435 of SEQ ID NO:138, positions 20 to 445 of SEQ ID NO:139, positions 20 to 437 of SEQ ID NO:140, positions 21 to 435 of SEQ ID NO:141, positions 20 to 437 of SEQ ID NO:142, positions 21 to 435 of SEQ ID NO:143, positions 26 to 435 of SEQ ID NO:144, positions 23 to 435 of SEQ ID NO:145, positions 24 to 443 of SEQ ID NO:146, positions 20 to 445 of SEQ ID NO:147, positions 21 to 441 of SEQ ID NO:148, and positions 20 to 437 of SEQ ID NO:149.

The CBDs are particularly involved in the hydrolysis of crystalline cellulose. It has been shown that the ability of cellobiohydrolases to degrade crystalline cellulose decreases when the CBD is absent (Linder and Teed, 1997, Journal of Biotechnol. 57:15-28). The variant CBH I polypeptides of the disclosure can further include a CBD. Exemplary CBDs comprise amino acid sequences corresponding to positions 494 to 529 of SEQ ID NO:1, positions 480 to 514 of SEQ ID NO:2, positions 494 to 529 of SEQ ID NO:3, positions 491 to 526 of SEQ ID NO:5, positions 477 to 512 of SEQ ID NO:6, positions 497 to 532 of SEQ ID NO:7, positions 504 to 539 of SEQ ID NO:8, positions 486 to 521 of SEQ ID NO:13, positions 556 to 596 of SEQ ID NO:15, positions 490 to 525 of SEQ ID NO:18, positions 495 to 530 of SEQ ID NO:20, positions 471 to 506 of SEQ ID NO:23, positions 481 to 516 of SEQ ID NO:27, positions 480 to 514 of SEQ ID NO:30, positions 495 to 529 of SEQ ID NO:35, positions 493 to 528 of SEQ ID NO:36, positions 477 to 512 of SEQ ID NO:38, positions 547 to 586 of SEQ ID NO:39, positions 475 to 510 of SEQ ID NO:40, positions 479 to 513 of SEQ ID NO:41, positions 506 to 541 of SEQ ID NO:42, positions 481 to 516 of SEQ ID NO:43, positions 503 to 537 of SEQ ID NO:45, positions 488 to 523 of SEQ ID NO:46, positions 476 to 511 of SEQ ID NO:48, positions 488 to 523 of SEQ ID NO:49, positions 479 to 513 of SEQ ID NO:50, positions 500 to 535 of SEQ ID NO:52, positions 493 to 528 of SEQ ID NO:55, positions 479 to 514 of SEQ ID NO:58, positions 494 to 529 of SEQ ID NO:60, positions 490 to 525 of SEQ ID NO:61, positions 497 to 532 of SEQ ID NO:62, positions 475 to 510 of SEQ ID NO:64, positions 477 to 512 of SEQ ID NO:65, positions 486 to 521 of SEQ ID NO:66, positions 470 to 505 of SEQ ID NO:67, positions 491 to 526 of SEQ ID NO:68, positions 476 to 511 of SEQ ID NO:69, positions 480 to 514 of SEQ ID NO:73, positions 506 to 540 of SEQ ID NO:74, positions 471 to 504 of SEQ ID NO:76, positions 501 to 536 of SEQ ID NO:78, positions 473 to 508 of SEQ ID NO:79, positions 481 to 516 of SEQ ID NO:83, positions 488 to 523 of SEQ ID NO:86, positions 475 to 510 of SEQ ID NO:92, positions 468 to 504 of SEQ ID NO:93, positions 501 to 536 of SEQ ID NO:96, positions 482 to 517 of SEQ ID NO:98, positions 481 to 516 of SEQ ID NO:99, positions 488 to 523 of SEQ ID NO:100, positions 472 to 507 of SEQ ID NO:101, positions 481 to 516 of SEQ ID NO:102, positions 471 to 505 of SEQ ID NO:105, positions 481 to 516 of SEQ ID NO:106, positions 495 to 530 of SEQ ID NO:107, positions 488 to 523 of SEQ ID NO:111, positions 478 to 513 of SEQ ID NO:112, positions 501 to 536 of SEQ ID NO:113, positions 491 to 526 of SEQ ID NO:115, and positions 503 to 538 of SEQ ID NO:116.

The CD and CBD are often connected via a linker. Exemplary linker sequences correspond to positions 456 to 493 of SEQ ID NO:1, positions 445 to 479 of SEQ ID NO:2, positions 456 to 493 of SEQ ID NO:3, positions 458 to 490 of SEQ ID NO:5, positions 449 to 476 of SEQ ID NO:6, positions 461 to 496 of SEQ ID NO:7, positions 461 to 503 of SEQ ID NO:8, positions 446 to 485 of SEQ ID NO:13, positions 444 to 555 of SEQ ID NO:15, positions 450 to 489 of SEQ ID NO:18, positions 450 to 494 of SEQ ID NO:20, positions 448 to 470 of SEQ ID NO:23, positions 443 to 480 of SEQ ID NO:27, positions 445 to 479 of SEQ ID NO:30, positions 460 to 494 of SEQ ID NO:35, positions 451 to 492 of SEQ ID NO:36, positions 449 to 476 of SEQ ID NO:38, positions 444 to 546 of SEQ ID NO:39, positions 443 to 474 of SEQ ID NO:40, positions 445 to 478 of SEQ ID NO:41, positions 458 to 505 of SEQ ID NO:42, positions 450 to 480 of SEQ ID NO:43, positions 457 to 502 of SEQ ID NO:45, positions 452 to 487 of SEQ ID NO:46, positions 449 to 475 of SEQ ID NO:48, positions 452 to 487 of SEQ ID NO:49, positions 445 to 478 of SEQ ID NO:50, positions 462 to 499 of SEQ ID NO:52, positions 449 to 492 of SEQ ID NO:55, positions 449 to 478 of SEQ ID NO:58, positions 456 to 493 of SEQ ID NO:60, positions 450 to 489 of SEQ ID NO:61, positions 450 to 496 of SEQ ID NO:62, positions 449 to 474 of SEQ ID NO:64, positions 452 to 476 of SEQ ID NO:65, positions 448 to 485 of SEQ ID NO:66, positions 425 to 469 of SEQ ID NO:67, positions 449 to 490 of SEQ ID NO:68, positions 444 to 475 of SEQ ID NO:69, positions 445 to 479 of SEQ ID NO:73, positions 459 to 505 of SEQ ID NO:74, positions 436 to 470 of SEQ ID NO:76, positions 458 to 500 of SEQ ID NO:78, positions 449 to 472 of SEQ ID NO:79, positions 443 to 480 of SEQ ID NO:83, positions 448 to 487 of SEQ ID NO:86, positions 443 to 474 of SEQ ID NO:92, positions 437 to 467 of SEQ ID NO:93, positions 473 to 500 of SEQ ID NO:96, positions 448 to 481 of SEQ ID NO:98, positions 451 to 480 of SEQ ID NO:99, positions 452 to 487 of SEQ ID NO:100, positions 449 to 471 of SEQ ID NO:101, positions 443 to 480 of SEQ ID NO:102, positions 441 to 470 of SEQ ID NO:105, positions 440 to 480 of SEQ ID NO:106, positions 461 to 494 of SEQ ID NO:107, positions 448 to 487 of SEQ ID NO:111, positions 450 to 478 of SEQ ID NO:112, positions 458 to 500 of SEQ ID NO:113, positions 449 to 490 of SEQ ID NO:115, and positions 449 to 502 of SEQ ID NO:116.

Because CBH I polypeptides are modular, the CBDs, CDs and linkers of different CBH I polypeptides, such as the exemplary CBH I polypeptides of Table 1, can be used interchangeably. However, in a preferred embodiment, the CBDs, CDs and linkers of a variant CBH I of the disclosure originate from the same polypeptide.

The variant CBH I polypeptides of the disclosure preferably have at least a two-fold reduction of product inhibition, such that cellobiose has an IC₅₀ towards the variant CBH I that is at least 2-fold the IC₅₀ of the corresponding reference CBH I, e.g., CBH I lacking the R268 substitution and/or R411 substitution. More preferably the IC₅₀ of cellobiose towards the variant CBH I is at least 3-fold, at least 5-fold, at least 8-fold, at least 10-fold, at least 12-fold, at least 15-fold, at least 20-fold, at least 25-fold, at least 50-fold, at least 100-fold, at least 150-fold, at least 200-fold, at least 250-fold, at least 500-fold or at least 700-fold greater tolerance to cellobiose, and in some cases up to 750-fold or up to 1,000-fold, the IC₅₀ of the corresponding reference CBH I. In specific embodiments the IC₅₀ of cellobiose towards the variant CBH I is ranges from 2-fold to 15-fold, from 2-fold to 10-fold, from 3-fold to 10-fold, from 5-fold to 12-fold, from 4-fold to 12-fold, from 5-fold to 10-fold, from 5-fold to 12-fold, from 2-fold to 8-fold, from 8-fold to 20-fold, from 20-fold to 100-fold, from 50-fold to 150-fold, from 150-fold to 500-fold, from 200-fold to 750-fold, from 50-fold to 700-fold, or from 100-fold to 1,000-fold the IC₅₀ of the corresponding reference CBH I.

The IC₅₀ can be determined in a phosphoric acid swollen cellulose (“PASC”) assay (Du et al., 2010, Applied Biochemistry and Biotechnology 161:313-317) or a methylumbelliferyl lactoside (“MUL”) assay (van Tilbeurgh and Claeyssens, 1985, FEBS Letts. 187(2):283-288), as exemplified in the Examples below.

The variant CBH I polypeptides of the disclosure preferably have a cellobiohydrolase activity that is at least 30% the cellobiohydrolase activity of the corresponding reference CBH I, e.g., CBH I lacking the R268 substitution and/or R411 substitution. More preferably, the cellobiohydrolase activity of the variant CBH I is at least 40%, at least 50%, at least 60%, at least 70%, at least 80%, at least 90%, or 100% the cellobiohydrolase activity of the corresponding reference CBH I, and in some cases 150%, 200%, 250%, 300%, 350%, 400% or 450% the cellobiohydrolase activity of the corresponding reference CBH I. In specific embodiments the cellobiohydrolase activity of the variant CBH I is ranges from 30% to 80%, from 40% to 70%, 30% to 60%, from 50% to 80%, from 60% to 80%, from 70% to 450%, from 80% to 350%, from 100% to 450%, from 150% to 450%, from 100% to 400%, from 150% to 400%, or from 90% to 450% of the cellobiohydrolase activity of the corresponding reference CBH I. Assays for cellobiohydrolase activity are described, for example, in Becker et al., 2011, Biochem J. 356:19-30 and Mitsuishi et al., 1990, FEBS Letts. 275:135-138, each of which is expressly incorporated by reference herein. The ability of CBH I to hydrolyze isolated soluble and insoluble substrates can also be measured using assays described in Srisodsuk et al., 1997, J. Biotech. 57:4957 and Nidetzky and Claeyssens, 1994, Biotech. Bioeng. 44:961-966. Substrates useful for assaying cellobiohydrolase activity include crystalline cellulose, filter paper, phosphoric acid swollen cellulose, cellooligosaccharides, methylumbelliferyl lactoside, methylumbelliferyl cellobioside, orthonitrophenyl lactoside, paranitrophenyl lactoside, orthonitrophenyl cellobioside, paranitrophenyl cellobioside. Cellobiohydrolase activity can be measured in an assay utilizing PASC as the substrate and a calcofluor white detection method (Du et al., 2010, Applied Biochemistry and Biotechnology 161:313-317). PASC can be prepared as described by Walseth, 1952, TAPPI 35:228-235 and Wood, 1971, Biochem. J. 121:353-362.

Other than said R268 and/or R411 substitution, the variant CBH I polypeptides of the disclosure preferably:
comprise an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to a CD of a reference CBH I exemplified in Table 1 (i.e., a CD comprising an amino acid sequence corresponding to positions 26 to 455 of SEQ ID NO:1, positions 18 to 444 of SEQ ID NO:2, positions 26 to 455 of SEQ ID NO:3, positions 1 to 427 of SEQ ID NO:4, positions 24 to 457 of SEQ ID NO:5, positions 18 to 448 of SEQ ID NO:6, positions 27 to 460 of SEQ ID NO:7, positions 27 to 460 of SEQ ID NO:8, positions 20 to 449 of SEQ ID NO:9, positions 1 to 424 of SEQ ID NO:10, positions 18 to 447 of SEQ ID NO:11, positions 18 to 434 of SEQ ID NO:12, positions 18 to 445 of SEQ ID NO:13, positions 19 to 454 of SEQ ID NO:14, positions 19 to 443 of SEQ ID NO:15, positions 2 to 426 of SEQ ID NO:16, positions 23 to 446 of SEQ ID NO:17, positions 19 to 449 of SEQ ID NO:18, positions 23 to 446 of SEQ ID NO:19, positions 19 to 449 of SEQ ID NO:20, positions 2 to 416 of SEQ ID NO:21, positions 19 to 454 of SEQ ID NO:22, positions 19 to 447 of SEQ ID NO:23, positions 19 to 447 of SEQ ID NO:24, positions 20 to 443 of SEQ ID NO:25, positions 18 to 447 of SEQ ID NO:26, positions 19 to 442 of SEQ ID NO:27, positions 18 to 451 of SEQ ID NO:28, positions 23 to 446 of SEQ ID NO:29, positions 18 to 444 of SEQ ID NO:30, positions 18 to 451 of SEQ ID NO:31, positions 18 to 447 of SEQ ID NO:32, positions 19 to 449 of SEQ ID NO:33, positions 18 to 447 of SEQ ID NO:34, positions 26 to 459 of SEQ ID NO:35, positions 19 to 450 of SEQ ID NO:36, positions 19 to 453 of SEQ ID NO:37, positions 18 to 448 of SEQ ID NO:38, positions 19 to 443 of SEQ ID NO:39, positions 19 to 442 of SEQ ID NO:40, positions 18 to 444 of SEQ ID NO:41, positions 24 to 457 of SEQ ID NO:42, positions 18 to 449 of SEQ ID NO:43, positions 19 to 453 of SEQ ID NO:44, positions 26 to 456 of SEQ ID NO:45, positions 19 to 451 of SEQ ID NO:46, positions 18 to 443 of SEQ ID NO:47, positions 18 to 448 of SEQ ID NO:48, positions 19 to 451 of SEQ ID NO:49, positions 18 to 444 of SEQ ID NO:50, positions 2 to 419 of SEQ ID NO:51, positions 27 to 461 of SEQ ID NO:52, positions 21 to 445 of SEQ ID NO:53, positions 19 to 449 of SEQ ID NO:54, positions 19 to 448 of SEQ ID NO:55, positions 18 to 443 of SEQ ID NO:56, positions 20 to 443 of SEQ ID NO:57, positions 18 to 448 of SEQ ID NO:58, positions 18 to 447 of SEQ ID NO:59, positions 26 to 455 of SEQ ID NO:60, positions 19 to 449 of SEQ ID NO:61, positions 19 to 449 of SEQ ID NO:62, positions 26 to 460 of SEQ ID NO:63, positions 18 to 448 of SEQ ID NO:64, positions 19 to 451 of SEQ ID NO:65, positions 19 to 447 of SEQ ID NO:66, positions 1 to 424 of SEQ ID NO:67, positions 19 to 448 of SEQ ID NO:68, positions 19 to 443 of SEQ ID NO:69, positions 23 to 447 of SEQ ID NO:70, positions 17 to 448 of SEQ ID NO:71, positions 19 to 449 of SEQ ID NO:72, positions 18 to 444 of SEQ ID NO:73, positions 23 to 458 of SEQ ID NO:74, positions 20 to 452 of SEQ ID NO:75, positions 18 to 435 of SEQ ID NO:76, positions 18 to 446 of SEQ ID NO:77, positions 22 to 457 of SEQ ID NO:78, positions 18 to 448 of SEQ ID NO:79, positions 1 to 431 of SEQ ID NO:80, positions 19 to 453 of SEQ ID NO:81, positions 21 to 440 of SEQ ID NO:82, positions 19 to 442 of SEQ ID NO:83, positions 18 to 448 of SEQ ID NO:84, positions 17 to 446 of SEQ ID NO:85, positions 18 to 447 of SEQ ID NO:86, positions 18 to 443 of SEQ ID NO:87, positions 23 to 448 of SEQ ID NO:88, positions 18 to 451 of SEQ ID NO:89, positions 21 to 447 of SEQ ID NO:90, positions 18 to 444 of SEQ ID NO:91, positions 19 to 442 of SEQ ID NO:92, positions 20 to 436 of SEQ ID NO:93, positions 18 to 450 of SEQ ID NO:94, positions 22 to 453 of SEQ ID NO:95, positions 16 to 472 of SEQ ID NO:96, positions 21 to 445 of SEQ ID NO:97, positions 19 to 447 of SEQ ID NO:98, positions 19 to 450 of SEQ ID NO:99, positions 19 to 451 of SEQ ID NO:100, positions 18 to 448 of SEQ ID NO:101, positions 19 to 442 of SEQ ID NO:102, positions 20 to 457 of SEQ ID NO:103, positions 19 to 454 of SEQ ID NO:104, positions 18 to 440 of SEQ ID NO:105, positions 18 to 439 of SEQ ID NO:106, positions 27 to 460 of SEQ ID NO:107, positions 23 to 446 of SEQ ID NO:108, positions 17 to 446 of SEQ ID NO:109, positions 21 to 447 of SEQ ID NO:110, positions 19 to 447 of SEQ ID NO:111, positions 18 to 449 of SEQ ID NO:112, positions 22 to 457 of SEQ ID NO:113, positions 18 to 445 of SEQ ID NO:114, positions 18 to 448 of SEQ ID NO:115, positions 18 to 448 of SEQ ID NO:116, positions 23 to 435 of SEQ ID NO:117, positions 21 to 442 of SEQ ID NO:118, positions 23 to 435 of SEQ ID NO:119, positions 20 to 445 of SEQ ID NO:120, positions 21 to 443 of SEQ ID NO:121, positions 20 to 445 of SEQ ID NO:122, positions 23 to 443 of SEQ ID NO:123, positions 20 to 445 of SEQ ID NO:124, positions 21 to 435 of SEQ ID NO:125, positions 20 to 437 of SEQ ID NO:126, positions 21 to 442 of SEQ ID NO:127, positions 23 to 434 of SEQ ID NO:128, positions 20 to 444 of SEQ ID NO:129, positions 21 to 435 of SEQ ID NO:130, positions 20 to 445 of SEQ ID NO:131, positions 21 to 446 of SEQ ID NO:132, positions 21 to 435 of SEQ ID NO:133, positions 22 to 448 of SEQ ID NO:134, positions 23 to 433 of SEQ ID NO:135, positions 23 to 434 of SEQ ID NO:136, positions 23 to 435 of SEQ ID NO:137, positions 23 to 435 of SEQ ID NO:138, positions 20 to 445 of SEQ ID NO:139, positions 20 to 437 of SEQ ID NO:140, positions 21 to 435 of SEQ ID NO:141, positions 20 to 437 of SEQ ID NO:142, positions 21 to 435 of SEQ ID NO:143, positions 26 to 435 of SEQ ID NO:144, positions 23 to 435 of SEQ ID NO:145, positions 24 to 443 of SEQ ID NO:146, positions 20 to 445 of SEQ ID NO:147, positions 21 to 441 of SEQ ID NO:148, and positions 20 to 437 of SEQ ID NO:149 (preferably the CD corresponding to positions 26-455 of SEQ ID NO:1 or 18-444 of SEQ ID NO:2); and/or
comprise an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to a mature polypeptide of a reference CBH I exemplified in Table 1 (i.e., a mature protein comprising an amino acid sequence corresponding to positions 26 to 529 of SEQ ID NO:1, positions 18 to 514 of SEQ ID NO:2, positions 26 to 529 of SEQ ID NO:3, positions 1 to 427 of SEQ ID NO:4, positions 24 to 526 of SEQ ID NO:5, positions 18 to 512 of SEQ ID NO:6, positions 27 to 532 of SEQ ID NO:7, positions 27 to 539 of SEQ ID NO:8, positions 20 to 449 of SEQ ID NO:9, positions 1 to 424 of SEQ ID NO:10, positions 18 to 447 of SEQ ID NO:11, positions 18 to 434 of SEQ ID NO:12, positions 18 to 521 of SEQ ID NO:13, positions 19 to 454 of SEQ ID NO:14, positions 19 to 596 of SEQ ID NO:15, positions 2 to 426 of SEQ ID NO:16, positions 23 to 446 of SEQ ID NO:17, positions 19 to 525 of SEQ ID NO:18, positions 23 to 446 of SEQ ID NO:19, positions 19 to 530 of SEQ ID NO:20, positions 2 to 416 of SEQ ID NO:21, positions 19 to 454 of SEQ ID NO:22, positions 19 to 506 of SEQ ID NO:23, positions 19 to 447 of SEQ ID NO:24, positions 20 to 443 of SEQ ID NO:25, positions 18 to 447 of SEQ ID NO:26, positions 19 to 516 of SEQ ID NO:27, positions 18 to 451 of SEQ ID NO:28, positions 23 to 446 of SEQ ID NO:29, positions 18 to 514 of SEQ ID NO:30, positions 18 to 451 of SEQ ID NO:31, positions 18 to 447 of SEQ ID NO:32, positions 19 to 449 of SEQ ID NO:33, positions 18 to 447 of SEQ ID NO:34, positions 26 to 529 of SEQ ID NO:35, positions 19 to 528 of SEQ ID NO:36, positions 19 to 453 of SEQ ID NO:37, positions 18 to 512 of SEQ ID NO:38, positions 19 to 586 of SEQ ID NO:39, positions 19 to 510 of SEQ ID NO:40, positions 18 to 513 of SEQ ID NO:41, positions 24 to 541 of SEQ ID NO:42, positions 18 to 516 of SEQ ID NO:43, positions 19 to 453 of SEQ ID NO:44, positions 26 to 537 of SEQ ID NO:45, positions 19 to 523 of SEQ ID NO:46, positions 18 to 443 of SEQ ID NO:47, positions 18 to 511 of SEQ ID NO:48, positions 19 to 523 of SEQ ID NO:49, positions 18 to 513 of SEQ ID NO:50, positions 2 to 419 of SEQ ID NO:51, positions 27 to 535 of SEQ ID NO:52, positions 21 to 445 of SEQ ID NO:53, positions 19 to 449 of SEQ ID NO:54, positions 19 to 528 of SEQ ID NO:55, positions 18 to 443 of SEQ ID NO:56, positions 20 to 443 of SEQ ID NO:57, positions 18 to 514 of SEQ ID NO:58, positions 18 to 447 of SEQ ID NO:59, positions 26 to 529 of SEQ ID NO:60, positions 19 to 525 of SEQ ID NO:61, positions 19 to 532 of SEQ ID NO:62, positions 26 to 460 of SEQ ID NO:63, positions 18 to 510 of SEQ ID NO:64, positions 19 to 512 of SEQ ID NO:65, positions 19 to 521 of SEQ ID NO:66, positions 1 to 505 of SEQ ID NO:67, positions 19 to 526 of SEQ ID NO:68, positions 19 to 511 of SEQ ID NO:69, positions 23 to 447 of SEQ ID NO:70, positions 17 to 448 of SEQ ID NO:71, positions 19 to 449 of SEQ ID NO:72, positions 18 to 514 of SEQ ID NO:73, positions 23 to 540 of SEQ ID NO:74, positions 20 to 452 of SEQ ID NO:75, positions 18 to 504 of SEQ ID NO:76, positions 18 to 446 of SEQ ID NO:77, positions 22 to 536 of SEQ ID NO:78, positions 18 to 508 of SEQ ID NO:79, positions 1 to 431 of SEQ ID NO:80, positions 19 to 453 of SEQ ID NO:81, positions 21 to 440 of SEQ ID NO:82, positions 19 to 516 of SEQ ID NO:83, positions 18 to 448 of SEQ ID NO:84, positions 17 to 446 of SEQ ID NO:85, positions 18 to 523 of SEQ ID NO:86, positions 18 to 443 of SEQ ID NO:87, positions 23 to 448 of SEQ ID NO:88, positions 18 to 451 of SEQ ID NO:89, positions 21 to 447 of SEQ ID NO:90, positions 18 to 444 of SEQ ID NO:91, positions 19 to 510 of SEQ ID NO:92, positions 20 to 504 of SEQ ID NO:93, positions 18 to 450 of SEQ ID NO:94, positions 22 to 453 of SEQ ID NO:95, positions 16 to 536 of SEQ ID NO:96, positions 21 to 445 of SEQ ID NO:97, positions 19 to 517 of SEQ ID NO:98, positions 19 to 516 of SEQ ID NO:99, positions 19 to 523 of SEQ ID NO:100, positions 18 to 507 of SEQ ID NO:101, positions 19 to 516 of SEQ ID NO:102, positions 20 to 457 of SEQ ID NO:103, positions 19 to 454 of SEQ ID NO:104, positions 18 to 505 of SEQ ID NO:105, positions 18 to 516 of SEQ ID NO:106, positions 27 to 530 of SEQ ID NO:107, positions 23 to 446 of SEQ ID NO:108, positions 17 to 446 of SEQ ID NO:109, positions 21 to 447 of SEQ ID NO:110, positions 19 to 523 of SEQ ID NO:111, positions 18 to 513 of SEQ ID NO:112, positions 22 to 536 of SEQ ID NO:113, positions 18 to 445 of SEQ ID NO:114, positions 18 to 526 of SEQ ID NO:115, positions 18 to 538 of SEQ ID NO:116, positions 23 to 435 of SEQ ID NO:117, positions 21 to 442 of SEQ ID NO:118, positions 23 to 435 of SEQ ID NO:119, positions 20 to 445 of SEQ ID NO:120, positions 21 to 443 of SEQ ID NO:121, positions 20 to 445 of SEQ ID NO:122, positions 23 to 443 of SEQ ID NO:123, positions 20 to 445 of SEQ ID NO:124, positions 21 to 435 of SEQ ID NO:125, positions 20 to 437 of SEQ ID NO:126, positions 21 to 442 of SEQ ID NO:127, positions 23 to 434 of SEQ ID NO:128, positions 20 to 444 of SEQ ID NO:129, positions 21 to 435 of SEQ ID NO:130, positions 20 to 445 of SEQ ID NO:131, positions 21 to 446 of SEQ ID NO:132, positions 21 to 435 of SEQ ID NO:133, positions 22 to 448 of SEQ ID NO:134, positions 23 to 433 of SEQ ID NO:135, positions 23 to 434 of SEQ ID NO:136, positions 23 to 435 of SEQ ID NO:137, positions 23 to 435 of SEQ ID NO:138, positions 20 to 445, of SEQ ID NO:139, positions 20 to 437 of SEQ ID NO:140, positions 21 to 435 of SEQ ID NO:141, positions 20 to 437 of SEQ ID NO:142, positions 21 to 435 of SEQ ID NO:143, positions 26 to 435 of SEQ ID NO:144, positions 23 to 435 of SEQ ID NO:145, positions 24 to 443 of SEQ ID NO:146, positions 20 to 445 of SEQ ID NO:147, positions 21 to 441 of SEQ ID NO:148, and positions 20 to 437 of SEQ ID NO:149, preferably the mature polypeptide corresponding to positions 26-529 of SEQ ID NO:1 or 18-514 of SEQ ID NO:2).

An example of an algorithm that is suitable for determining sequence similarity is the BLAST algorithm, which is described in Altschul et al., 1990, J. Mol. Biol. 215:403-410. Software for performing BLAST analyses is publicly available through the National Center for Biotechnology Information. This algorithm involves first identifying high scoring sequence pairs (HSPs) by identifying short words of length W in the query sequence that either match or satisfy some positive-valued threshold score T when aligned with a word of the same length in a database sequence. These initial neighborhood word hits act as starting points to find longer HSPs containing them. The word hits are expanded in both directions along each of the two sequences being compared for as far as the cumulative alignment score can be increased. Extension of the word hits is stopped when: the cumulative alignment score falls off by the quantity X from a maximum achieved value; the cumulative score goes to zero or below; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff & Henikoff, 1992, Proc. Nat'l. Acad. Sci. USA 89:10915-10919) alignments (B) of 50, expectation (E) of 10, M′5, N′-4, and a comparison of both strands.

Most CBH I polypeptides are secreted and are therefore expressed with a signal sequence that is cleaved upon secretion of the polypeptide from the cell. Accordingly, in certain aspects, the variant CBH I polypeptides of the disclosure further include a signal sequence. Exemplary signal sequences comprise amino acid sequences corresponding to positions 1 to 25 of SEQ ID NO:1, positions 1 to 17 of SEQ ID NO:2, positions 1 to 25 of SEQ ID NO:3, positions 1 to 23 of SEQ ID NO:5, positions 1 to 17 of SEQ ID NO:6, positions 1 to 26 of SEQ ID NO:7, positions 1 to 27 of SEQ ID NO:8, positions 1 to 19 of SEQ ID NO:9, positions 1 to 17 of SEQ ID NO:11, positions 1 to 17 of SEQ ID NO:12, positions 1 to 17 of SEQ ID NO:13, positions 1 to 18 of SEQ ID NO:14, positions 1 to 18 of SEQ ID NO:15, positions 1 to 22 of SEQ ID NO:17, positions 1 to 18 of SEQ ID NO:18, positions 1 to 22 of SEQ ID NO:19, positions 1 to 18 of SEQ ID NO:20, positions 1 to 18 of SEQ ID NO:22, positions 1 to 18 of SEQ ID NO:23, positions 1 to 18 of SEQ ID NO:24, positions 1 to 19 of SEQ ID NO:25, positions 1 to 17 of SEQ ID NO:26, positions 1 to 18 of SEQ ID NO:27, positions 1 to 17 of SEQ ID NO:28, positions 1 to 22 of SEQ ID NO:29, positions 1 to 18 of SEQ ID NO:30, positions 1 to 17 of SEQ ID NO:31, positions 1 to 17 of SEQ ID NO:32, positions 1 to 18 of SEQ ID NO:33, positions 1 to 17 of SEQ ID NO:34, positions 1 to 25 of SEQ ID NO:35, positions 1 to 18 of SEQ ID NO:36, positions 1 to 18 of SEQ ID NO:37, positions 1 to 17 of SEQ ID NO:38, positions 1 to 18 of SEQ ID NO:39, positions 1 to 18 of SEQ ID NO:40, positions 1 to 17 of SEQ ID NO:41, positions 1 to 23 of SEQ ID NO:42, positions 1 to 17 of SEQ ID NO:43, positions 1 to 18 of SEQ ID NO:44, positions 1 to 25 of SEQ ID NO:45, positions 1 to 18 of SEQ ID NO:46, positions 1 to 17 of SEQ ID NO:47, positions 1 to 17 of SEQ ID NO:48, positions 1 to 18 of SEQ ID NO:49, positions 1 to 17 of SEQ ID NO:50, positions 1 to 26 of SEQ ID NO:52, positions 1 to 20 of SEQ ID NO:53, positions 1 to 18 of SEQ ID NO:54, positions 1 to 18 of SEQ ID NO:55, positions 1 to 17 of SEQ ID NO:56, positions 1 to 19 of SEQ ID NO:57, positions 1 to 17 of SEQ ID NO:58, positions 1 to 17 of SEQ ID NO:59, positions 1 to 25 of SEQ ID NO:60, positions 1 to 18 of SEQ ID NO:61, positions 1 to 18 of SEQ ID NO:62, positions 1 to 25 of SEQ ID NO:63, positions 1 to 17 of SEQ ID NO:64, positions 1 to 18 of SEQ ID NO:65, positions 1 to 18 of SEQ ID NO:66, positions 1 to 18 of SEQ ID NO:68, positions 1 to 18 of SEQ ID NO:69, positions 1 to 23 of SEQ ID NO:70, positions 1 to 17 of SEQ ID NO:71, positions 1 to 18 of SEQ ID NO:72, positions 1 to 17 of SEQ ID NO:73, positions 1 to 22 of SEQ ID NO:74, positions 1 to 19 of SEQ ID NO:75, positions 1 to 17 of SEQ ID NO:76, positions 1 to 17 of SEQ ID NO:77, positions 1 to 21 of SEQ ID NO:78, positions 1 to 18 of SEQ ID NO:79, positions 1 to 18 of SEQ ID NO:81, positions 1 to 20 of SEQ ID NO:82, positions 1 to 18 of SEQ ID NO:83, positions 1 to 17 of SEQ ID NO:84, positions 1 to 16 of SEQ ID NO:85, positions 1 to 17 of SEQ ID NO:86, positions 1 to 17 of SEQ ID NO:87, positions 1 to 22 of SEQ ID NO:88, positions 1 to 17 of SEQ ID NO:89, positions 1 to 20 of SEQ ID NO:90, positions 1 to 17 of SEQ ID NO:91, positions 1 to 18 of SEQ ID NO:92, positions 1 to 19 of SEQ ID NO:93, positions 1 to 17 of SEQ ID NO:94, positions 1 to 21 of SEQ ID NO:95, positions 1 to 15 of SEQ ID NO:96, positions 1 to 20 of SEQ ID NO:97, positions 1 to 18 of SEQ ID NO:98, positions 1 to 18 of SEQ ID NO:99, positions 1 to 18 of SEQ ID NO:100, positions 1 to 17 of SEQ ID NO:101, positions 1 to 18 of SEQ ID NO:102, positions 1 to 19 of SEQ ID NO:103, positions 1 to 18 of SEQ ID NO:104, positions 1 to 17 of SEQ ID NO:105, positions 1 to 17 of SEQ ID NO:106, positions 1 to 26 of SEQ ID NO:107, positions 1 to 22 of SEQ ID NO:108, positions 1 to 16 of SEQ ID NO:109, positions 1 to 20 of SEQ ID NO:110, positions 1 to 18 of SEQ ID NO:111, positions 1 to 17 of SEQ ID NO:112, positions 1 to 21 of SEQ ID NO:113, positions 1 to 17 of SEQ ID NO:114, positions 1 to 17 of SEQ ID NO:115, positions 1 to 18 of SEQ ID NO:116, positions 1 to 22 of SEQ ID NO:117, positions 1 to 20 of SEQ ID NO:118, positions 1 to 22 of SEQ ID NO:119, positions 1 to 19 of SEQ ID NO:120, positions 1 to 20 of SEQ ID NO:121, positions 1 to 19 of SEQ ID NO:122, positions 1 to 22 of SEQ ID NO:123, positions 1 to 19 of SEQ ID NO:124, positions 1 to 20 of SEQ ID NO:125, positions 1 to 19 of SEQ ID NO:126, positions 1 to 21 of SEQ ID NO:127, positions 1 to 22 of SEQ ID NO:128, positions 1 to 19 of SEQ ID NO:129, positions 1 to 20 of SEQ ID NO:130, positions 1 to 19 of SEQ ID NO:131, positions 1 to 20 of SEQ ID NO:132, positions 1 to 20 of SEQ ID NO:133, positions 1 to 21 of SEQ ID NO:134, positions 1 to 22 of SEQ ID NO:135, positions 1 to 22 of SEQ ID NO:136, positions 1 to 22 of SEQ ID NO:137, positions 1 to 22 of SEQ ID NO:138, positions 1 to 19 of SEQ ID NO:139, positions 1 to 19 of SEQ ID NO:140, positions 1 to 20 of SEQ ID NO:141, positions 1 to 19 of SEQ ID NO:142, positions 1 to 20 of SEQ ID NO:143, positions 1 to 25 of SEQ ID NO:144, positions 1 to 22 of SEQ ID NO:145, positions 1 to 23 of SEQ ID NO:146, positions 1 to 19 of SEQ ID NO:147, positions 1 to 20 of SEQ ID NO:148, and positions 1 to 19 of SEQ ID NO:149.

1.2. Recombinant Expression of Variant CBH I Polypeptides

1.2.1. Cell Culture Systems

The disclosure also provides recombinant cells engineered to express variant CBH I polypeptides. Suitably, the variant CBH I polypeptide is encoded by a nucleic acid operably linked to a promoter. The promoters can be homologous or heterologous, and constitutive or inducible.

Suitable host cells include cells of any microorganism (e.g., cells of a bacterium, a protist, an alga, a fungus (e.g., a yeast or filamentous fungus), or other microbe), and are preferably cells of a bacterium, a yeast, or a filamentous fungus.

Where recombinant expression in a filamentous fungal host is desired, the promoter can be a fungal promoter (including but not limited to a filamentous fungal promoter), a promoter operable in plant cells, a promoter operable in mammalian cells.

As described in U.S. provisional application No. 61/553,901, filed Oct. 31, 2011, the contents of which are hereby incorporated in their entireties, promoters that are constitutively active in mammalian cells (which can derived from a mammalian genome or the genome of a mammalian virus) are capable of eliciting high expression levels in filamentous fungi such as Trichoderma reesei. An exemplary promoter is the cytomegalovirus (“CMV”) promoter.

As described in U.S. provisional application No. 61/553,897, filed Oct. 31, 2011, the contents of which are hereby incorporated in their entireties, promoters that are constitutively active in plant cells (which can derived from a plant genome or the genome of a plant virus) are capable of eliciting high expression levels in filamentous fungi such as Trichoderma reesei. Exemplary promoters are the cauliflower mosaic virus (“CaMV”) 35S promoter or the Commelina yellow mottle virus (“CoYMV”) promoter.

Mammalian, mammalian viral, plant and plant viral promoters can drive particularly high expression when the associated 5′ UTR sequence (i.e., the sequence which begins at the transcription start site and ends one nucleotide (nt) before the start codon) normally associated with the mammalian or mammalian viral promoter is replaced by a fungal 5′ UTR sequence.

The source of the 5′ UTR can vary provided it is operable in the filamentous fungal cell. In various embodiments, the 5′ UTR can be derived from a yeast gene or a filamentous fungal gene. The 5′ UTR can be from the same species one other component in the expression cassette (e.g. the promoter or the CBH I coding sequence), or from a different species. The 5′ UTR can be from the same species as the filamentous fungal cell that the expression construct is intended to operate in. In an exemplary embodiment, the 5′ UTR comprises a sequence corresponding to a fragment of a 5′ UTR from a T. reesei glyceraldehyde-3-phosphate dehydrogenase (gpd). In a specific embodiment, the 5′ UTR is not naturally associated with the CMV promoter

Examples of other promoters that can be used include, but are not limited to, a cellulase promoter, a xylanase promoter, the 1818 promoter (previously identified as a highly expressed protein by EST mapping Trichoderma). For example, the promoter can suitably be a cellobiohydrolase, endoglucanase, or β-glucosidase promoter. A particularly suitable promoter can be, for example, a T. reesei cellobiohydrolase, endoglucanase, or β-glucosidase promoter. Non-limiting examples of promoters include a cbh1, cbh2, egl1, egl2, egl3, egl4, egl5, pki1, gpd1, xyn1, or xyn2 promoter.

Suitable host cells of the bacterial genera include, but are not limited to, cells of Escherichia, Bacillus, Lactobacillus, Pseudomonas, and Streptomyces. Suitable cells of bacterial species include, but are not limited to; cells of Escherichia coli, Bacillus subtilis, Bacillus licheniformis, Lactobacillus brevis, Pseudomonas aeruginosa, and Streptomyces lividans.

Suitable host cells of the genera of yeast include, but are not limited to, cells of Saccharomyces, Schizosaccharomyces, Candida, Hansenula, Pichia, Kluyveromyces, and Phaffia. Suitable cells of yeast species include, but are not limited to, cells of Saccharomyces cerevisiae, Schizosaccharomyces pombe, Candida albicans, Hansenula polymorpha, Pichia pastoris, P. canadensis, Kluyveromyces marxianus, and Phaffia rhodozyma.

Suitable host cells of filamentous fungi include all filamentous forms of the subdivision Eumycotina. Suitable cells of filamentous fungal genera include, but are not limited to, cells of Acremonium, Aspergillus, Aureobasidium, Bjerkandera, Ceriporiopsis, Chrysoporium, Coprinus, Coriolus, Corynascus, Chaetomium, Cryptococcus, Filobasidium, Fusarium, Gibberella, Humicola, Hypocrea, Magnaporthe, Mucor, Myceliophthora, Mucor, Neocallimastix, Neurospora, Paecilomyces, Penicillium, Phanerochaete, Phlebia, Piromyces, Pleurotus, Scytaldium, Schizophyllum, Sporotrichum, Talaromyces, Thermoascus, Thielavia, Tolypocladium, Trametes, and Trichoderma. More preferably, the recombinant cell is a Trichoderma sp. (e.g., Trichoderma reesei), Penicillium sp., Humicola sp. (e.g., Humicola insolens); Aspergillus sp. (e.g., Aspergillus niger), Chrysosporium sp., Fusarium sp., or Hypocrea sp. Suitable cells can also include cells of various anamorph and teleomorph forms of these filamentous fungal genera.

Suitable cells of filamentous fungal species include, but are not limited to, cells of Aspergillus awamori, Aspergillus fumigatus, Aspergillus foetidus, Aspergillus japonicus, Aspergillus nidulans, Aspergillus niger, Aspergillus oryzae, Chrysosporium lucknowense, Fusarium bactridioides, Fusarium cerealis, Fusarium crookwellense, Fusarium culmorum, Fusarium graminearum, Fusarium graminurn, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Coprinus cinereus, Coriolus hirsutus, Humicola insolens, Humicola lanuginosa, Mucor miehei, Myceliophthora thermophila, Neurospora crassa, Neurospora intermedia, Penicillium purpurogenum, Penicillium canescens, Penicillium solitum, Penicillium funiculosum, Phanerochaete chrysosporium, Phlebia radiate, Pleurotus eryngii, Talaromyces flavus, Thielavia terrestris, Trametes villosa, Trametes versicolor, Trichoderma harzianum, Trichoderma koningii, Trichoderma longibrachiatum, Trichoderma reesei, and Trichoderma viride.

The engineered host cells can be cultured in conventional nutrient media modified as appropriate for activating promoters, selecting transformants, or amplifying the nucleic acid sequence encoding the variant CBH I polypeptide. Culture conditions, such as temperature, pH and the like, are those previously used with the host cell selected for expression, and will be apparent to those skilled in the art. As noted, many references are available for the culture and production of many cells, including cells of bacterial and fungal origin. Cell culture media in general are set forth in Atlas and Parks (eds.), 1993, The Handbook of Microbiological Media, CRC Press, Boca Raton, Fla., which is incorporated herein by reference. For recombinant expression in filamentous fungal cells, the cells are cultured in a standard medium containing physiological salts and nutrients, such as described in Pourquie et al., 1988, Biochemistry and Genetics of Cellulose Degradation, eds. Aubert, et al., Academic Press, pp. 71-86; and Ilmen et al., 1997, Appl. Environ. Microbiol. 63:1298-1306. Culture conditions are also standard, e.g., cultures are incubated at 28° C. in shaker cultures or fermenters until desired levels of variant CBH I expression are achieved. Preferred culture conditions for a given filamentous fungus may be found in the scientific literature and/or from the source of the fungi such as the American Type Culture Collection (ATCC). After fungal growth has been established, the cells are exposed to conditions effective to cause or permit the expression of a variant CBH I.

In cases where a variant CBH I coding sequence is under the control of an inducible promoter, the inducing agent, e.g., a sugar, metal salt or antibiotics, is added to the medium at a concentration effective to induce variant CBH I expression.

In one embodiment, the recombinant cell is an Aspergillus niger, which is a useful strain for obtaining overexpressed polypeptide. For example A. niger var. awamori dgr246 is known to product elevated amounts of secreted cellulases (Goedegebuur et al., 2002, Curr. Genet. 41:89-98). Other strains of Aspergillus niger var awamori such as GCDAP3, GCDAP4 and GAPS-4 are known (Ward et al., 1993, Appl. Microbiol. Biotechnol. 39:738-743).

In another embodiment, the recombinant cell is a Trichoderma reesei, which is a useful strain for obtaining overexpressed polypeptide. For example, RL-P37, described by Sheir-Neiss et al., 1984, Appl. Microbiol. Biotechnol. 20:46-53, is known to secrete elevated amounts of cellulase enzymes. Functional equivalents of RL-P37 include Trichoderma reesei strain RUT-C30 (ATCC No. 56765) and strain QM9414 (ATCC No. 26921). It is contemplated that these strains would also be useful in overexpressing variant CBH I polypeptides.

Cells expressing the variant CBH I polypeptides of the disclosure can be grown under batch, fed-batch or continuous fermentations conditions. Classical batch fermentation is a closed system, wherein the compositions of the medium is set at the beginning of the fermentation and is not subject to artificial alternations during the fermentation. A variation of the batch system is a fed-batch fermentation in which the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art. Continuous fermentation is an open system where a defined fermentation medium is added continuously to a bioreactor and an equal amount of conditioned medium is removed simultaneously for processing. Continuous fermentation generally maintains the cultures at a constant high density where cells are primarily in log phase growth. Continuous fermentation systems strive to maintain steady state growth conditions. Methods for modulating nutrients and growth factors for continuous fermentation processes as well as techniques for maximizing the rate of product formation are well known in the art of industrial microbiology.

1.2.2. Recombinant Expression in Plants

The disclosure provides transgenic plants and seeds that recombinantly express a variant CBH I polypeptide. The disclosure also provides plant products, e.g., oils, seeds, leaves, extracts and the like, comprising a variant CBH I polypeptide.

The transgenic plant can be dicotyledonous (a dicot) or monocotyledonous (a monocot). The disclosure also provides methods of making and using these transgenic plants and seeds. The transgenic plant or plant cell expressing a variant CBH I can be constructed in accordance with any method known in the art. See, for example, U.S. Pat. No. 6,309,872. T. reesei CBH I has been successfully expressed in transgenic tobacco (Nicotiana tabaccum) and potato (Solanum tuberosum). See Hooker et al., 2000, in Glycosyl Hydrolases for Biomass Conversion, ACS Symposium Series, Vol. 769, Chapter 4, pp. 55-90.

In a particular aspect, the present disclosure provides for the expression of CBH I variants in transgenic plants or plant organs and methods for the production thereof. DNA expression constructs are provided for the transformation of plants with a nucleic acid encoding the variant CBH I polypeptide, preferably under the control of regulatory sequences which are capable of directing expression of the variant CBH I polypeptide. These regulatory sequences include sequences capable of directing transcription in plants, either constitutively, or in stage and/or tissue specific manners.

The expression of variant CBH I polypeptides in plants can be achieved by a variety of means. Specifically, for example, technologies are available for transforming a large number of plant species, including dicotyledonous species (e.g., tobacco, potato, tomato, Petunia, Brassica) and monocot species. Additionally, for example, strategies for the expression of foreign genes in plants are available. Additionally still, regulatory sequences from plant genes have been identified that are serviceable for the construction of chimeric genes that can be functionally expressed in plants and in plant cells (e.g., Klee, 1987, Ann. Rev. of Plant Phys. 38:467-486; Clark et al., 1990, Virology 179(2):640-7; Smith et al., 1990, Mol. Gen. Genet. 224(3):477-81.

The introduction of nucleic acids into plants can be achieved using several technologies including transformation with Agrobacterium tumefaciens or Agrobacterium rhizogenes. Non-limiting examples of plant tissues that can be transformed include protoplasts, microspores or pollen, and explants such as leaves, stems, roots, hypocotyls, and cotyls. Furthermore, DNA encoding a variant CBH I can be introduced directly into protoplasts and plant cells or tissues by microinjection, electroporation, particle bombardment, and direct DNA uptake.

Variant CBH I polypeptides can be produced in plants by a variety of expression systems. For instance, the use of a constitutive promoter such as the 35S promoter of Cauliflower Mosaic Virus (Guilley et al., 1982, Cell 30:763-73) is serviceable for the accumulation of the expressed protein in virtually all organs of the transgenic plant. Alternatively, promoters that are tissue-specific and/or stage-specific can be used (Higgins, 1984, Annu. Rev. Plant Physiol. 35:191-221; Shotwell and Larkins, 1989, In: The Biochemistry of Plants Vol. 15 (Academic Press, San Diego: Stumpf and Conn, eds.), p. 297), permit expression of variant CBH I polypeptides in a target tissue and/or during a desired stage of development.

1.3. Compositions Of Variant CBH I Polypeptides

In general, a variant CBH I polypeptide produced in cell culture is secreted into the medium and may be purified or isolated, e.g., by removing unwanted components from the cell culture medium. However, in some cases, a variant CBH I polypeptide may be produced in a cellular form necessitating recovery from a cell lysate. In such cases the variant CBH I polypeptide is purified from the cells in which it was produced using techniques routinely employed by those skilled in the art. Examples include, but are not limited to, affinity chromatography (Van Tilbeurgh et al., 1984, FEBS Lett. 169(2):215-218), ion-exchange chromatographic methods (Goyal et al., 1991, Bioresource Technology, 36:37-50; Fliess et al., 1983, Eur. J. Appl. Microbiol. Biotechnol. 17:314-318; Bhikhabhai et al., 1984, J. Appl. Biochem. 6:336-345; Ellouz et al., 1987, Journal of Chromatography, 396:307-317), including ion-exchange using materials with high resolution power (Medve et al., 1998, J. Chromatography A, 808:153-165), hydrophobic interaction chromatography (Tomaz and Queiroz, 1999, J. Chromatography A, 865:123-128), and two-phase partitioning (Brumbauer et al., 1999, Bioseparation 7:287-295).

The variant CBH I polypeptides of the disclosure are suitably used in cellulase compositions. Cellulases are known in the art as enzymes that hydrolyze cellulose (beta-1,4-glucan or beta D-glucosidic linkages) resulting in the formation of glucose, cellobiose, cellooligosaccharides, and the like. Cellulase enzymes have been traditionally divided into three major classes: endoglucanases (“EG”), exoglucanases or cellobiohydrolases (EC 3.2.1.91) (“CBH”) and beta-glucosidases (EC 3.2.1.21) (“BG”) (Knowles et al., 1987, TIBTECH 5:255-261; Schulein, 1988, Methods in Enzymology 160(25):234-243).

Certain fungi produce complete cellulase systems which include exo-cellobiohydrolases or CBH-type cellulases, endoglucanases or EG-type cellulases and β-glucosidases or BG-type cellulases (Schulein, 1988, Methods in Enzymology 160(25):234-243). Such cellulase compositions are referred to herein as “whole” cellulases. However, sometimes these systems lack CBH-type cellulases and bacterial cellulases also typically include little or no CBH-type cellulases. In addition, it has been shown that the EG components and CBH components synergistically interact to more efficiently degrade cellulose. See, e.g., Wood, 1985, Biochemical Society Transactions 13(2):407-410.

The cellulase compositions of the disclosure typically include, in addition to a variant CBH I polypeptide, one or more cellobiohydrolases, endoglucanases and/or β-glucosidases. In their crudest form, cellulase compositions contain the microorganism culture that produced the enzyme components. “Cellulase compositions” also refers to a crude fermentation product of the microorganisms. A crude fermentation is preferably a fermentation broth that has been separated from the microorganism cells and/or cellular debris (e.g., by centrifugation and/or filtration). In some cases, the enzymes in the broth can be optionally diluted, concentrated, partially purified or purified and/or dried. The variant CBH I polypeptide can be co-expressed with one or more of the other components of the cellulase composition or it can be expressed separately, optionally purified and combined with a composition comprising one or more of the other cellulase components.

When employed in cellulase compositions, the variant CBH I is generally present in an amount sufficient to allow release of soluble sugars from the biomass. The amount of variant CBH I enzymes added depends upon the type of biomass to be saccharified which can be readily determined by the skilled artisan. In certain embodiments, the weight percent of variant CBH I polypeptide is suitably at least 1, at least 5, at least 10, or at least 20 weight percent of the total polypeptides in a cellulase composition. Exemplary cellulase compositions include a variant CBH I of the disclosure in an amount ranging from about 1 to about 20 weight percent, from about 1 to about 25 weight percent, from about 5 to about 20 weight percent, from about 5 to about 25 weight percent, from about 5 to about 30 weight percent, from about 5 to about 35 weight percent, from about 5 to about 40 weight percent, from about 5 to about 45 weight percent, from about 5 to about 50 weight percent, from about 10 to about 20 weight percent, from about 10 to about 25 weight percent, from about 10 to about 30 weight percent, from about 10 to about 35 weight percent, from about 10 to about 40 weight percent, from about 10 to about 45 weight percent, from about 10 to about 50 weight percent, from about 15 to about 20 weight percent, from about 15 to about 25 weight percent, from about 15 to about 30 weight percent, from about 15 to about 35 weight percent, from about 15 to about 30 weight percent, from about 15 to about 45 weight percent, or from about 15 to about 50 weight percent of the total polypeptides in the composition.

1.4. Utility of Variant CBH I Polypeptides

It can be appreciated that the variant CBH I polypeptides of the disclosure and compositions comprising the variant CBH I polypeptides find utility in a wide variety applications, for example detergent compositions that exhibit enhanced cleaning ability, function as a softening agent and/or improve the feel of cotton fabrics (e.g., “stone washing” or “biopolishing”), or in cellulase compositions for degrading wood pulp into sugars (e.g., for bio-ethanol production). Other applications include the treatment of mechanical pulp (Pere et al., 1996, Tappi Pulping Conference, pp. 693-696 (Nashville, Tenn., Oct. 27-31, 1996)), for use as a feed additive (see, e.g., WO 91/04673) and in grain wet milling.

1.4.1. Saccharification Reactions

Ethanol can be produced via saccharification and fermentation processes from cellulosic biomass such as trees, herbaceous plants, municipal solid waste and agricultural and forestry residues. However, the ratio of individual cellulase enzymes within a naturally occurring cellulase mixture produced by a microbe may not be the most efficient for rapid conversion of cellulose in biomass to glucose. It is known that endoglucanases act to produce new cellulose chain ends which themselves are substrates for the action of cellobiohydrolases and thereby improve the efficiency of hydrolysis of the entire cellulase system. The use of optimized cellobiohydrolase activity may greatly enhance the production of ethanol.

Cellulase compositions comprising one or more of the variant CBH I polypeptides of the disclosure can be used in saccharification reaction to produce simple sugars for fermentation. Accordingly, the present disclosure provides methods for saccharification comprising contacting biomass with a cellulase composition comprising a variant CBH I polypeptide of the disclosure and, optionally, subjecting the resulting sugars to fermentation by a microorganism.

The term “biomass,” as used herein, refers to any composition comprising cellulose (optionally also hemicellulose and/or lignin). As used herein, biomass includes, without limitation, seeds, grains, tubers, plant waste or byproducts of food processing or industrial processing (e.g., stalks), corn (including, e.g., cobs, stover, and the like), grasses (including, e.g., Indian grass, such as Sorghastrum nutans; or, switchgrass, e.g., Panicum species, such as Panicum virgatum), wood (including, e.g., wood chips, processing waste), paper, pulp, and recycled paper (including, e.g., newspaper, printer paper, and the like). Other biomass materials include, without limitation, potatoes, soybean (e.g., rapeseed), barley, rye, oats, wheat, beets, and sugar cane bagasse.

The saccharified biomass (e.g., lignocellulosic material processed by enzymes of the disclosure) can be made into a number of bio-based products, via processes such as, e.g., microbial fermentation and/or chemical synthesis. As used herein, “microbial fermentation” refers to a process of growing and harvesting fermenting microorganisms under suitable conditions. The fermenting microorganism can be any microorganism suitable for use in a desired fermentation process for the production of bio-based products. Suitable fermenting microorganisms include, without limitation, filamentous fungi, yeast, and bacteria. The saccharified biomass can, for example, be made into a fuel (e.g., a biofuel such as a bioethanol, biobutanol, biomethanol, a biopropanol, a biodiesel, a jet fuel, or the like) via fermentation and/or chemical synthesis. The saccharified biomass can, for example, also be made into a commodity chemical (e.g., ascorbic acid, isoprene, 1,3-propanediol), lipids, amino acids, polypeptides, and enzymes, via fermentation and/or chemical synthesis.

Thus, in certain aspects, the variant CBH I polypeptides of the disclosure find utility in the generation of ethanol from biomass in either separate or simultaneous saccharification and fermentation processes. Separate saccharification and fermentation is a process whereby cellulose present in biomass is saccharified into simple sugars (e.g., glucose) and the simple sugars subsequently fermented by microorganisms (e.g., yeast) into ethanol. Simultaneous saccharification and fermentation is a process whereby cellulose present in biomass is saccharified into simple sugars (e.g., glucose) and, at the same time and in the same reactor, microorganisms (e.g., yeast) ferment the simple sugars into ethanol.

Prior to saccharification, biomass is preferably subject to one or more pretreatment step(s) in order to render cellulose material more accessible or susceptible to enzymes and thus more amenable to hydrolysis by the variant CBH I polypeptides of the disclosure.

In an exemplary embodiment, the pretreatment entails subjecting biomass material to a catalyst comprising a dilute solution of a strong acid and a metal salt in a reactor. The biomass material can, e.g., be a raw material or a dried material. This pretreatment can lower the activation energy, or the temperature, of cellulose hydrolysis, ultimately allowing higher yields of fermentable sugars. See, e.g., U.S. Pat. Nos. 6,660,506; 6,423,145.

Another exemplary pretreatment method entails hydrolyzing biomass by subjecting the biomass material to a first hydrolysis step in an aqueous medium at a temperature and a pressure chosen to effectuate primarily depolymerization of hemicellulose without achieving significant depolymerization of cellulose into glucose. This step yields a slurry in which the liquid aqueous phase contains dissolved monosaccharides resulting from depolymerization of hemicellulose, and a solid phase containing cellulose and lignin. The slurry is then subject to a second hydrolysis step under conditions that allow a major portion of the cellulose to be depolymerized, yielding a liquid aqueous phase containing dissolved/soluble depolymerization products of cellulose. See, e.g., U.S. Pat. No. 5,536,325.

A further exemplary method involves processing a biomass material by one or more stages of dilute acid hydrolysis using about 0.4% to about 2% of a strong acid; followed by treating the unreacted solid lignocellulosic component of the acid hydrolyzed material with alkaline delignification. See, e.g., U.S. Pat. No. 6,409,841. Another exemplary pretreatment method comprises prehydrolyzing biomass (e.g., lignocellulosic materials) in a prehydrolysis reactor; adding an acidic liquid to the solid lignocellulosic material to make a mixture; heating the mixture to reaction temperature; maintaining reaction temperature for a period of time sufficient to fractionate the lignocellulosic material into a solubilized portion containing at least about 20% of the lignin from the lignocellulosic material, and a solid fraction containing cellulose; separating the solubilized portion from the solid fraction, and removing the solubilized portion while at or near reaction temperature; and recovering the solubilized portion. The cellulose in the solid fraction is rendered more amenable to enzymatic digestion. See, e.g., U.S. Pat. No. 5,705,369. Further pretreatment methods can involve the use of hydrogen peroxide H₂O₂. See Gould, 1984, Biotech, and Bioengr. 26:46-52.

Pretreatment can also comprise contacting a biomass material with stoichiometric amounts of sodium hydroxide and ammonium hydroxide at a very low concentration. See Teixeira et al., 1999, Appl. Biochem. and Biotech. 77-79:19-34. Pretreatment can also comprise contacting a lignocellulose with a chemical (e.g., a base, such as sodium carbonate or potassium hydroxide) at a pH of about 9 to about 14 at moderate temperature, pressure, and pH. See PCT Publication WO2004/081185.

Ammonia pretreatment can also be used. Such a pretreatment method comprises subjecting a biomass material to low ammonia concentration under conditions of high solids. See, e.g., U.S. Patent Publication No. 20070031918 and PCT publication WO 06/110901.

1.4.2. Detergent Compositions Comprising Variant CBH I Proteins

The present disclosure also provides detergent compositions comprising a variant CBH I polypeptide of the disclosure. The detergent compositions may employ besides the variant CBH I polypeptide one or more of a surfactant, including anionic, non-ionic and ampholytic surfactants; a hydrolase; a bleaching agents; a bluing agent; a caking inhibitors; a solubilizer; and a cationic surfactant. All of these components are known in the detergent art.

The variant CBH I polypeptide is preferably provided as part of cellulase composition. The cellulase composition can be employed from about 0.00005 weight percent to about 5 weight percent or from about 0.0002 weight percent to about 2 weight percent of the total detergent composition. The cellulase composition can be in the form of a liquid diluent, granule, emulsion, gel, paste, and the like. Such forms are known to the skilled artisan. When a solid detergent composition is employed, the cellulase composition is preferably formulated as granules. CL 2. Example 1

### Identification and Characterization of Product Tolerant Variants of CBH I

2.1. Materials and Methods

2.1.1. Preparation of CBH I Polypeptides for Biochemical Characterization

Protein expression was carried out in an Aspergillus niger host strain that had been transformed using PEG-mediated transformation with expression constructs for CBH I that included the hygromycin resistance gene as a selectable marker, in which the full length CBH I sequences (signal sequence, catalytic domain, linker and cellulose binding domain) were under the control of the glyceraldehyde-3-phosphate dehydrogenase (gpd) promoter. Transformants were selected on the regeneration medium based on resistance to hygromycin. The selected transformants were cultured in Aspergillus salts medium, pH 6.2 supplemented with the antibiotics penicillin, streptomycin, and hygromycin, and 80 g/L glycerol, 20 g/L soytone, 10 mM uridine, 20 g/L MES) in baffled shake flasks at 30° C., 170 rpm. After five days of incubation, the total secreted protein supernatant was recovered, and then subjected to hollow fiber filtration to concentrate and exchange the sample into acetate buffer (50 mM NaAc, pH 5). CBH I protein represented over 90% of the total protein in these samples. Protein purity was analyzed by SDS-PAGE. Protein concentration was determined by gel densitometry and/or HPLC analysis. All CBH I protein concentrations were normalized before assay and concentrated to 1-2.5 mg/ml.

2.1.2. CBH I Activity Assays

Methylumbelliferyl Lactoside (4-MUL) Assay:

This assay measures the activity of CBH I on the fluorogenic substrate 4-MUL (also known as MUL). Assays were run in a costar 96-well black bottom plate, where reactions were initiated by the addition of 4-MUL to enzyme in buffer (2 mM 4-MUL in 200 mM MES pH 6). Enzymatic rates were monitored by fluorescent readouts over five minutes on a SPECTRAMAX™ plate reader (ex/em 365/450 nm). Data in the linear range was used to calculate initial rates (Vo).

Phosphoric Acid Swollen Cellulose (PASC) Assay:

This assay measures the activity of CBH I using PASC as the substrate. During the assay, the concentration of PASC is monitored by a fluorescent signal derived from calcofluor binding to PASC (ex/em 365/440 nm). The assay is initiated by mixing enzyme (15 μl) and reaction buffer (85 μl of 0.2% PASC, 200 mM MES, pH 6), and then incubating at 35° C. while shaking at 225 RPM. After 2 hours, one reaction volume of calcofluor stop solution (100 μg/ml in 500 mM glycine pH 10) is added and fluorescence read-outs obtained (ex/em 365/440 nm).

Saccharification Assay (Bagasse Assay):

This assay measures the activity of CBH I on bagasse, a lignocellulosic substrate. Reactions were run in 10 ml vials with 5% dilute acid pretreated bagasse (250 mg solids per 5 ml reaction). Each reaction contained 4 mg CBH I enzyme/g solids, 200 mM MES pH 6, kanamycin, and chloramphenicol. Reactions were incubated at 35° C. in hybridization incubators (Robbins Scientific), rotating at 20 RPM. Time points were taken by transferring a sample of homogenous slurry (150 μl) into a 96-well deep well plate and quenching the reaction with stop buffer (450 μl of 500 mM sodium carbonate, pH 10). Time point measurements were taken every 24 hours for 72 hours.

Cellobiose Tolerance Assays (or Cellobiose Inhibition Assays):

Tolerance to cellobiose (or inhibition caused by cellobiose) was tested in two ways in the CBH I assays. A direct-dose tolerance method can be applied to all of the CBH I assays (i.e., 4-MUL, PASC, and/or bagasse assays), and entails the exogenous addition of a known amount of cellobiose into assay mixtures. A different indirect method entails the addition of an excess amount of β-glucosidase (BG) to PASC and bagasse assays (typically, 1 mg β-glucosidase/g solids loaded). BG will enzymatically hydrolyze the cellobiose generated during these assays; therefore, CBH I activity in the presence of BG can be taken as a measure of activity in the absence of cellobiose. Furthermore, when activity in the presence and absence of BG are similar, this indicates tolerance to cellobiose. Notably, in cases where BG activity is undesired, but may be present in crude CBH I enzyme preparations, the BG inhibitor gluconolactone can be added into CBH I assays to prevent cellobiose breakdown.

2.2. Library Screening Assays

The wild type CBH I polypeptide BD29555 was mutagenized to identify variants with improved product tolerance. A small (60-member) library of BD29555 variants was designed to identify variant CBH I polypeptides with reduced product inhibition. This product-release-site library was designed based on residues directly interacting with the cellobiose product in an attempt to identify variants with weakened interactions with cellobiose from which the product would be released more readily than the wild type enzyme. The 60-member evolution library contained wild-type residues and mutations at positions R273, W405, and R422 of BD29555 (SEQ ID NO:1), and included the following substitutions: R273 (WT), R273Q, R273K, R273A, W405 (WT), W405Q, W405H, R422 (WT), R422Q, R422K, R422L, and R422E (4 variants at position 273×3 variants at position 405×5 variants at position 422 equals 60 variants in total). All members of the library were screened using the 4-MUL assay in the presence and absence of 250 mg/L cellobiose and using gluconolactone to inhibit any BG activity. The R273A, R273Q, and R273K/R422K variants showed enhanced product tolerance. The R273K/R422K variant showed greatest activity, expression, and cellobiose tolerance at 250 mg/L (730 mM). Due to low expression, other variants were not tested further.

2.3. Characterization of Product Tolerant Variants of BD29555

The R273K/R422K substitutions were characterized in both a wild type BD29555 background and also in combination with the substitutions Y274Q, D281K, Y410H, P411G, which were identified in a screen of an expanded product release site evolution library.

The wild type, the R273K/R422K variant and the R273K/Y274Q/D281K/Y410H/P411G/R422K variants were tested for activity on 4-MUL in the presence and absence of 250 mg/L cellobiose, and the R273K/R422K variant was also tested in the bagasse assay in the presence and absence of BG. The results are summarized in Table 5.

The results from these activity assays were converted into the percentage of activity remaining with and without cellobiose present, where values close to 100% indicated cellobiose tolerance. The percent of activity remaining in the MUL assay in the presence cellobiose versus in the absence of cellobiose shows that the R273K/R422K variant was the most tolerant, followed by the R273K/Y274Q/D281K/Y410H/P411G/R422K variant, and then wild-type, at 95%, 78%, and 25% activity, respectively.

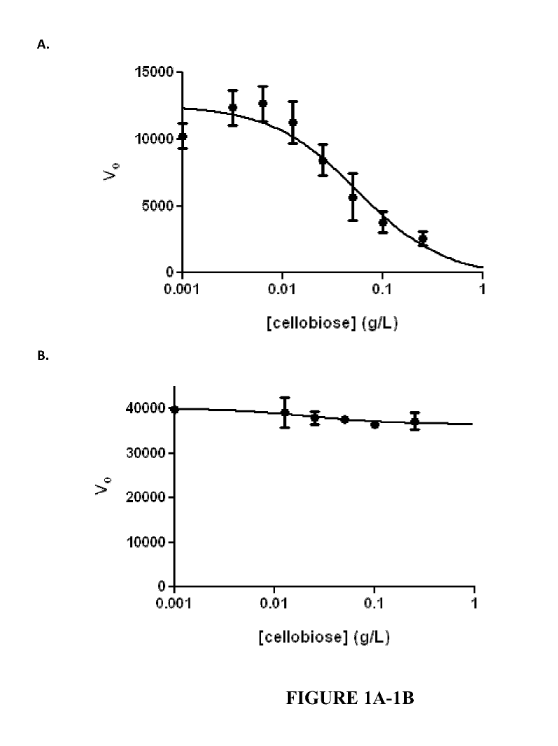

Cellobiose dose response curves of the wild-type and R273K/R422K variant of BD29555 were obtained during the 4-MUL assay. Enzyme rates (Vo) were measured in the presence of different concentrations of cellobiose (200 mM MES pH 6, 25° C.). Rates were measured in quadruplicate. The results are shown in FIG. 1A-1B. FIG. 1A shows that wild type BD2955 is inhibited by cellobiose, with a half maximal inhibitory concentration (IC₅₀ value) of 60 mg/L. FIG. 1B shows that the R273K/R422K variant is tolerant to cellobiose up to 250 mg/L.

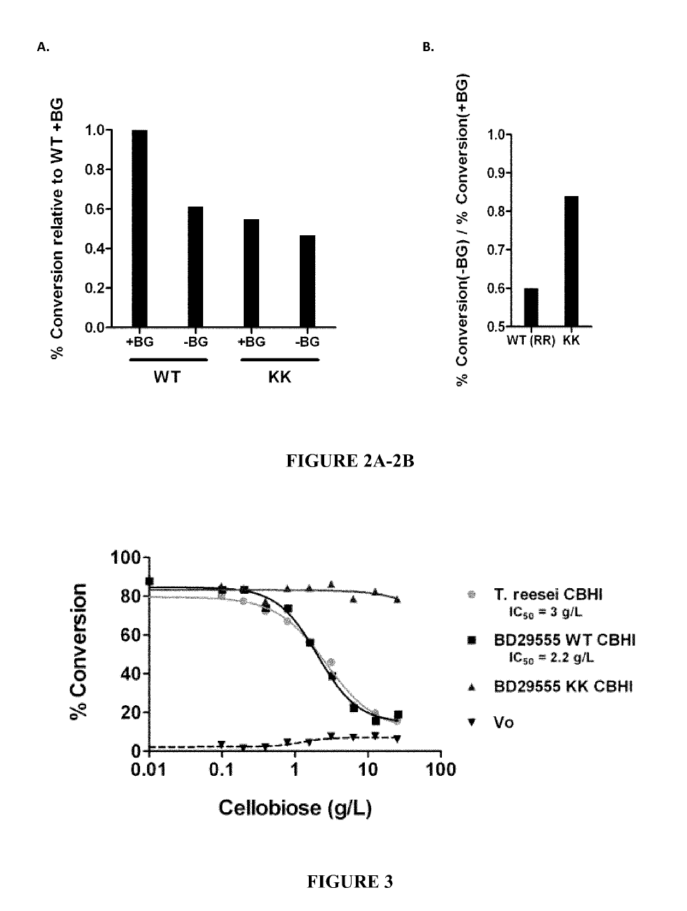

The bagasse assay results shown in Table 5, which lists the percentage of activity remaining in the absence vs. presence of BG, also demonstrate that the percentage activity of the wild type BD29555 is lower than the percentage activity of the R273K/R422K variant, indicating that the R273K/R422K variant is less sensitive to the presence of cellobiose than the wild type. FIG. 2A-2B shows bar graph data for the bagasse assay of BD29555 vs. the R273K/R422K variant. In FIG. 2A, bars represent relative activity, which has been normalized to wild type activity in the absence of cellobiose (WT+BG=uninhibited activity=1). In FIG. 2B, bars indicate tolerance to cellobiose, as represented by the ratio of activity in the presence of cellobiose (−BG) to that of activity in the absence of cellobiose (+BG); ratios close to 1 indicate greater tolerance to cellobiose. These data again demonstrate that the R273K/R422K variant of BD29555 is more tolerant to cellobiose than the wild type BD29555.

The wild type and R273K/R422K variant were also characterized in the PASC assay. Results are shown in FIG. 3. The activities of both wild type BD29555 (SEQ ID NO:1) and wild type T. reesei CBH I (SEQ ID NO:2) were inhibited by cellobiose concentrations starting around 1 g/L (with IC₅₀ values of 2.2 and 3 g/L, respectively), whereas the R273K/R422K variant showed little inhibition in the presence of 10 g/L cellobiose.

2.4. Characterization of Product Tolerant Variants of T. reesei CBH I

Cellobiose product tolerant substitutions were introduced into T. reesei CBH I (SEQ ID NO:2). A panel of variants with single and double alanine and lysine substitutions at R268 and R411 were expressed and analyzed. The variants were tested for activity on 4-MUL in the presence and absence of 250 mg/L cellobiose and also in the bagasse assay in the absence and presence of BG. The results from these assays were converted into the percentage activity remaining in the presence and absence of cellobiose and BG, respectively. Values are summarized in Table 6.

The 4-MUL assay results shown in Table 6 demonstrate that the activity of the wild type T. reesei CBH I was reduced to 23% in the presence of cellobiose, whereas the double mutants at R268 and R411 retained more than 90% of their activity under the same conditions.

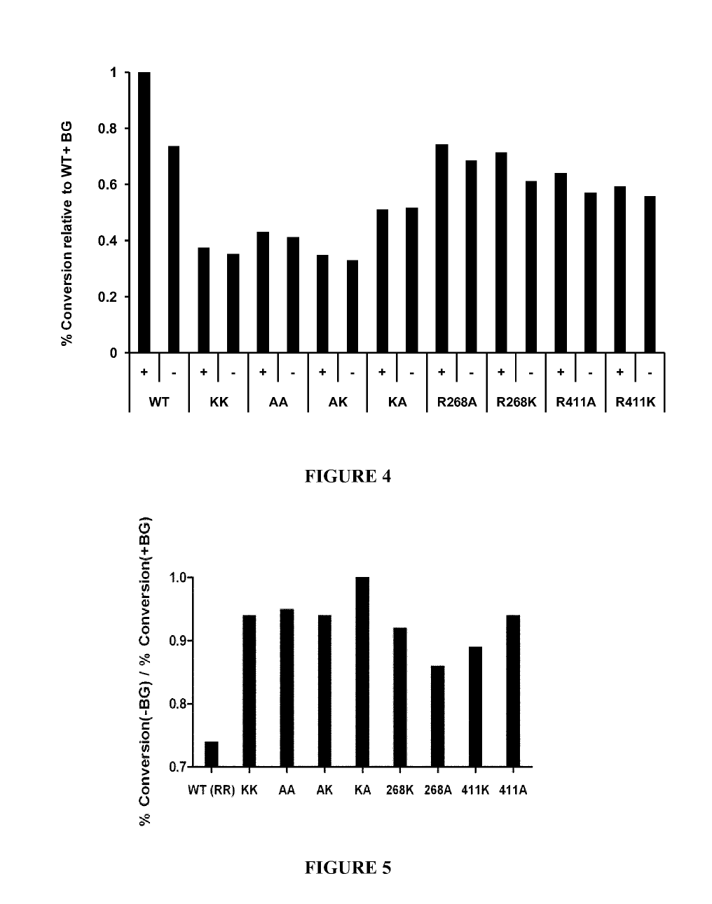

The bagasse assay results shown in Table 6 demonstrate that the activity of the wild type T. reesei CBH I is more significantly impacted by the presence of BG than is the activity of the single or double substitution variants, indicating that the variants are less sensitive to the accumulation of cellobiose than the wild type. FIGS. 4 and 5 show bar graph data for the bagasse assay of wild type T. reesei CBH I vs. the variants. In FIG. 4, bars represent relative activity, normalized to wild type activity in the absence of cellobiose (WT+BG=1). In FIG. 5, bars represent tolerance to cellobiose, as represented by the ratio of activity in the presence of accumulating cellobiose (−BG) to that of activity in the absence of cellobiose (+BG); ratios close to 1 indicate greater tolerance to cellobiose.

### 3. Example 2

### Identification and Characterization of Additional Product Tolerant Variants of CBH I

3.1. Materials and Methods

3.1.1. Preparation of CBH I Polypeptides for Biochemical Characterization:

Protein Expression:

Protein expression was carried out in a strain of Trichoderma reesei in which the native CBH I gene had been knocked out. The strain was transformed with a library of CBH I variant expression constructs that included the hygromycin resistance gene as a selectable marker. Expression constructs contained full-length CBH I wild-type or variant sequences (signal sequence, catalytic domain, linker and carbohydrate binding domain) under the control of a constitutive promoter. Transformants were selected on potato dextrose agar containing hygromycin (50 μg/mL). The selected isolates were subsequently cultured on 96-well plates containing potato dextrose agar without hygromycin. After sporulation, the transformants were stocked in 20% glycerol at −80° C. For screening, transformants were grown in 96-deep-well format for 6 days at 26° C., shaking at 850 rpm in a Multitron II shaker (3 mm throw), in 0.4 mL of liquid medium (2.5 g/L sodium citrate; 5 g/L KH₂PO₄; 2 g/L NH₄NO₃; 0.2 g/L MgSO4.7H₂O; 0.1 g/L CaCl₂; 9.1 g/L soytone; 80 g/L glycerol; 10 g/L MES buffer pH 6; 5 mg/L citric acid; 5 mg/L ZnSO₄.7H₂O; 1 mg/L Fe(NH₄)₂(SO₄)₂; 0.25 mg/L CuSO₄.₅H₂O; 0.05 mg/L MnSO₄; 0.05 mg/L H₃BO₃; 0.05 mg/L Na₂MoO₄.2H₂O; 5 μg/L biotin). Total secreted protein supernatants were harvested by filtration. The knock-out strain alone produced no CBH I protein. Protein concentration was determined by gel densitometry and/or RP-HPLC analysis.

Protein Quantification by Reverse-Phase (RP) High Performance Liquid chromatography (HPLC):

CBH I protein concentrations in supernatants were quantified using RP-HPLC. The system used was an Agilent 1100 series model, equipped with quaternary pump (connected to reservoirs A and B, where reservoir A contained water with 0.1% trifluoroacetic acid and reservoir B contained acetonitrile with 0.1% trifluoroacetic acid), a diode array detector (monitored at 225 nm and 280 nm), and a fluorescence detector (monitored at ex/em 280/340 nm). An Agilent Zorbax 300SB-C3 (5 μM, 4.6×150 mm) was used to separate samples using a 20 minute method (30-50% B over 10 minutes; 100% B for 5 minutes; 30% B for 5 min; at 60° C. at a flow rate of 1 mL/min). CBH I was identified by a retention time at 7.8-8.2 minutes and quantitated by area. Concentrations were determined by reference to a standard curve generated with a commercial CBH I (E-CBH I from Megazymes).

3.1.2. Biochemical Characterization:

Methylumbelliferyl Lactoside (4-MUL) Assay:

CBH I activity on was measured using the 4-MUL assay using gluconolactone to inhibit any BG activity. The fluorogenic 4-MUL substrate (SIGMA) was prepared at 100 mM concentration in DMSO. Assays were run in black 96-well-flat-bottomed plates (Costar) and 4-MU fluorescence was read on a BioTek H4 plate reader (ex/em 365/450 nm). Assay plates were filled with buffer (final concentrations of 100 mM MES, pH 6, 25 mM gluconolactone, with or without cellobiose; cellobiose concentrations are listed with appropriate data sets), to which enzyme mixture was added (10-30 μl, 5 μg/mL final) and then assays were initiated by addition of 4-MUL (0.5 mM final concentration in 100 μl total volume). Enzyme mixtures were either CBH I variants from harvested supernatants or standards. Standards included: a negative control, consisting of harvested supernatant from the CBH I knock-out strain; a positive control, consisting of wild-type CBH I from harvested supernatants; and, a commercial CBH I standard (E-CBHI from Megazymes). Activity standards were run by serial dilution of commercial CBH I from 40 to 0.02 μg/mL and 4-MU (SIGMA, prepared at 20 mM in DMSO) (in dilution increments of 2-fold; all dilutions were made using harvested supernatant from the knock-out control). Kinetic rates were monitored over the first 15 mins following 4-MUL addition; initial rates were calculated based on data in the linear range. After 1 hr, a final endpoint read was taken, both before and after reaction quenching (100 μL of 200 mM Sodium Carbonate, pH 10.0). Activity was calculated for kinetic and endpoint reads; background resulting from the CBH I knock-out supernatant remained negligible. 4MU standard curves and HPLC quantification values were used to calculate specific activity.

Saccharification Assay:

CBH I activity on a native lignocellulosic substrate was measured using the saccharification assay. Reactions were run in 96-well plates with the following composition in each well: 22 μL of variant/enzyme sample, 0.7% solids (dilute acid pretreated bagasse at 0.4% cellulose), β-glucosidase (50 ug/mL), and buffer (50 mM Sodium Citrate pH 5.5.), in a final volume of 227 μL. Time points were taken by transferring the reaction solution (15 into another 384-well plate and quenching the reaction with stop buffer (45 μl of 200 mM sodium carbonate, pH 10). Stop plates were sealed and stored at 4° C. for 14 hours before running a secondary BG digest: 15 ul of the stopped reaction into 35 ul of BG mix (50 ug/ml BG, 250 mM Sodium Citrate pH 5.5) and incubated at 37° C. for 14 hr. After the incubation, glucose was quantified by a glucose oxidase detection assay (GO assay), and percent cellulose conversion was calculated (based on 100% conversion at 25 mM) using a standard curve of known glucose concentrations (0.01-3.0 mM).

Cellobiose Tolerance/Inhibition Assays:

Tolerance/inhibition values represent activity ratios and/or percent activity remaining/percent activity decreased in the presence versus the absence of cellobiose. Tolerant variants show less inhibition in the presence of cellobiose as compared to wild type, where an activity ratio of 1 (with vs. without a given concentration of cellobiose) is equivalent to 0% inhibition by cellobiose, or 100% tolerance. The effect of cellobiose on CBH I variant performance was monitored by dose-response in the 4MUL assay. Dose-response curves were generated by assaying variant activity in the presence of 6-8 different cellobiose concentrations ranging up to 100 mM cellobiose. CBH I samples were diluted to 5 μg/mL final concentration or were used directly in the case of protein quantification levels below 5 μg/mL. Half maximal inhibitory concentration (IC₅₀) values were determined by plotting 4MUL activity versus cellobiose concentration and fitting with a four parameter dose-response fitting algorithm, with zero activity (or 100% inhibition) constrained to background activity (as established by CBH I knockout values) and with automatic outlier elimination (on GraphPad Prism 5).

Remazolbrilliant Blue R Stained Carboxymethyl-Cellulose (Azo-CMC) Assay:

Endoglycosidase activity was measured using the Azo-CMC assay. The colorimetric substrate Azo-CMC was obtained from Megazymes. The substrate was used as provided in solution (4M partially depolymerized and dyed CM-cellulose containing approximately one Remazolbrilliant Blue R dye molecule per 20 sugar residues). Assays were run in clear 96-well-flat-bottomed plates (Costar) and released Remazolbrilliant Blue R was monitored at 590 nm on a BioTek H4 reader. Assay plates were charged with equal volumes (40 uL) of supernatant/standard and Azo-CM-cellulose, incubated 14 h at 35° C., and stopped (200 μL; 80% EtOH, 0.3 M NaOAc, 0.03 M ZnOAc, pH 5.0). After stopping, the reaction plates were centrifuged (4000 rpm, 5 mins), and the clarified supernatant was transferred to a second clear flat bottom plate for absorbance reading. Activity was calibrated using an endoglycosidase standard (20 μg/mL); in all cases, harvested supernatants had activity values below the standard.

3.1.3. Library Design, Screening, and Characterization:

Library Design:

Example 1 describes CBH I variants that retain activity in the presence of cellobiose levels which are inhibitory to the wild-type enzyme. These cellobiose-tolerant variants were garnered when two arginines found at positions 268 and 411 in the enzyme's product release site were mutagenized to any combination of lysine and alanine. To further characterize single amino acid mutations that contribute to CBH I variants with cellobiose tolerance, a 40-member library was designed to individually mutate position 268 and 411 to each of the 20 naturally occurring amino acids. Additionally, the contribution of double amino acid mutations to CBH I variants with cellobiose tolerance was scanned with a 40-member library introducing each of the 20 amino acids to positions 268 and 411, while the other position was held constant at alanine. The final 80-member library contained: 20 variants with site 268 mutagenized to all possible amino acids (R268aa); 20 variants with site 268 mutagenized to all possible amino acids, and site 411 mutated to alanine (R268aa/R411A); 20 variants with site 411 mutagenized to all possible amino acids (R411 aa); 20 variants s with site 411 mutagenized to all possible amino acids, and site 268 mutated to alanine (R268A/R411aa).

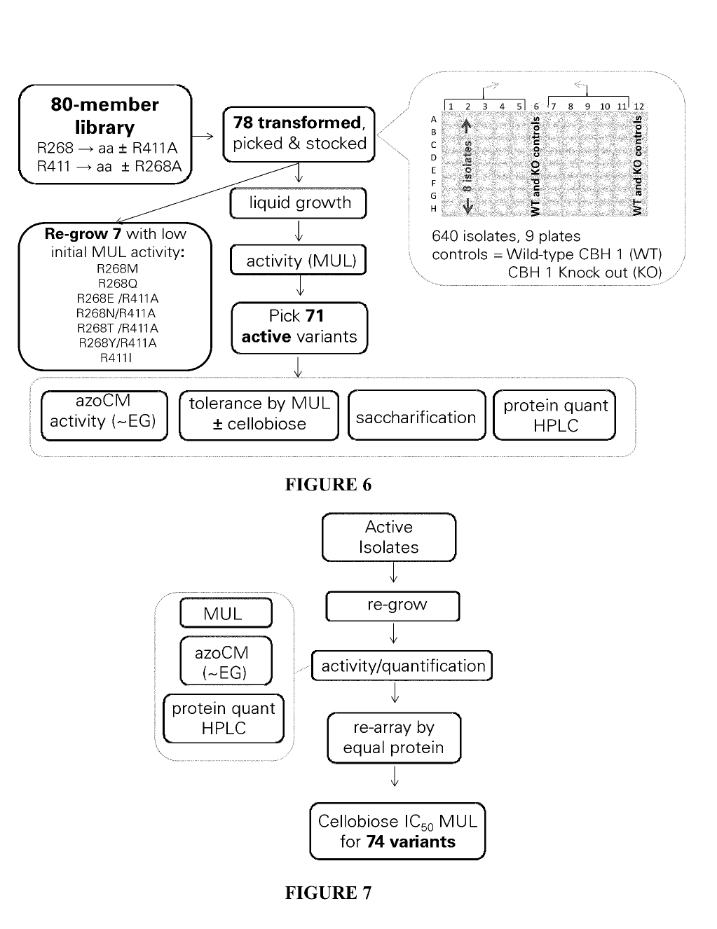

Transformation and Primary Screening for Active Isolates (Scheme 1 (FIG. 6)):

The variant library was successfully transformed with the exception of R268A/R411N and R268A/R411Y variants. For the 78 transformed variants, 8 isolates of each were picked, stocked, and grown. Supernatants were harvested for the primary screening by 4-MUL assay (see FIG. 6). Active isolates were identified for 71 out of 78; for R268M, R268Q, R268E/R411A, R268N/R411A, R268T/R411A, R268Y/R411A, and R4111, no active isolate was identified. For these variants, an additional 16 isolates were screened, yielding active isolates for R268N/R411A, R268E/R411A, and R268Y/R411A. Notably, all 20 amino acids at each position were covered either individually or in combination with alanine at the other site.

Active Variants:

The harvested protein samples from active isolates were evaluated for CBH I activity, by 4-MUL assay, and CBH I concentration, by HPLC. EG activity was assessed by Azo-CMC assay to verify no background interference. Protein samples were then directly tested in a primary screen for cellobiose tolerance in the 4-MUL assay and for activity on native substrate in the saccharification assay, as shown in FIG. 6. A master re-growth plate was prepared for the 71 active isolates. The plate was used to prepare additional supernatants for secondary screening, wherein dose-response curves were generated and IC₅₀ values were determined using normalized CBH I concentrations wherever possible (FIG. 7).

Screening by 4-MUL:

Harvested supernatants from active variant isolates were evaluated for cellobiose tolerance at 1 mM cellobiose in the 4-MUL activity assay. Table 8 lists the tolerance of variants at 1 mM. All non-WT variants demonstrated enhanced tolerance compared with the wild-type enzyme, which is significantly inhibited (% tolerance=6%, or 94% inhibited). Notably, the library contained a wild-type sequence member; this isolate showed consistent behavior with 3% tolerance at 1 mM. Additional cellobiose concentrations at 0.25, 5, 10, 50, and 100 mM were tested leading to full dose-response curves for which half maximal inhibitory concentration (IC₅₀) values were generated (Table 8). The IC₅₀ values support that the variant library has decreased product inhibition, or increased tolerance to cellobiose, when compared to the wild-type enzyme (WT IC₅₀=0.03 mM; see first entry, Table 8).

Primary Screening by Saccharification:

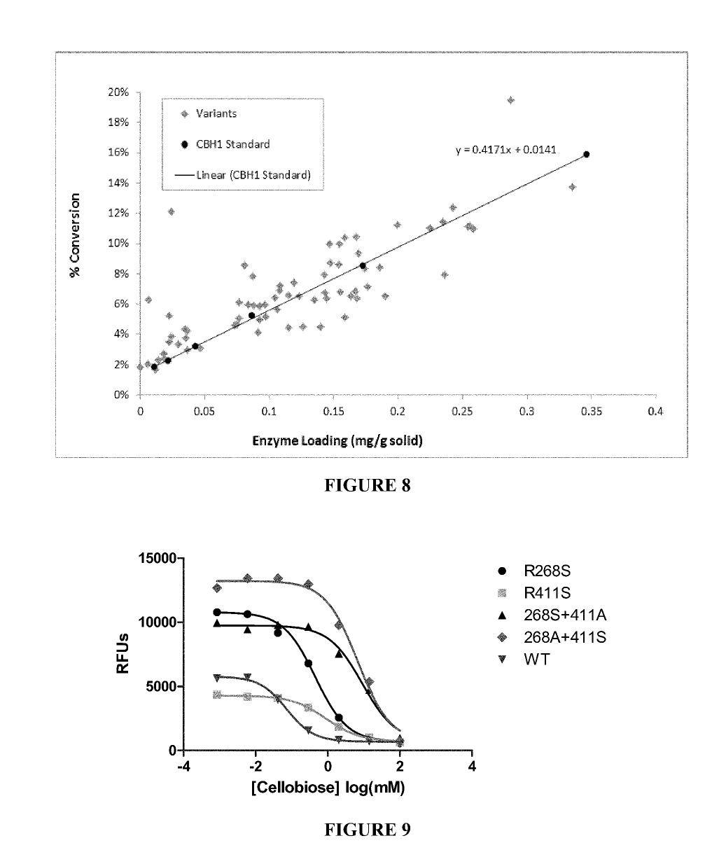

In one example, picked mutants were tested using the saccharification assay, which measures the extent to which CBH I converts polymeric cellulose into cellobiose. Saccharification was carried out for 48 hours and the percent of cellulose converted was calculated for each variant. FIG. 8 shows the plot of variant enzyme loading (mg CBH l/g solids) versus percent conversion; the commercial CBH I standard was plotted in serial dilution to generate a standard curve of enzyme loading versus percent conversion. Importantly, this graph shows that the mutant library retains activity on the native substrate and its activity distribution remains near to that of the commercial CBH I standard. Table 8 lists the measured saccharification activity of each variant and also lists expected conversion values based on variant loading as calculated using the commercial CBH I standard curve (% conversion estimated).

Secondary Screening: IC₅₀ Values:

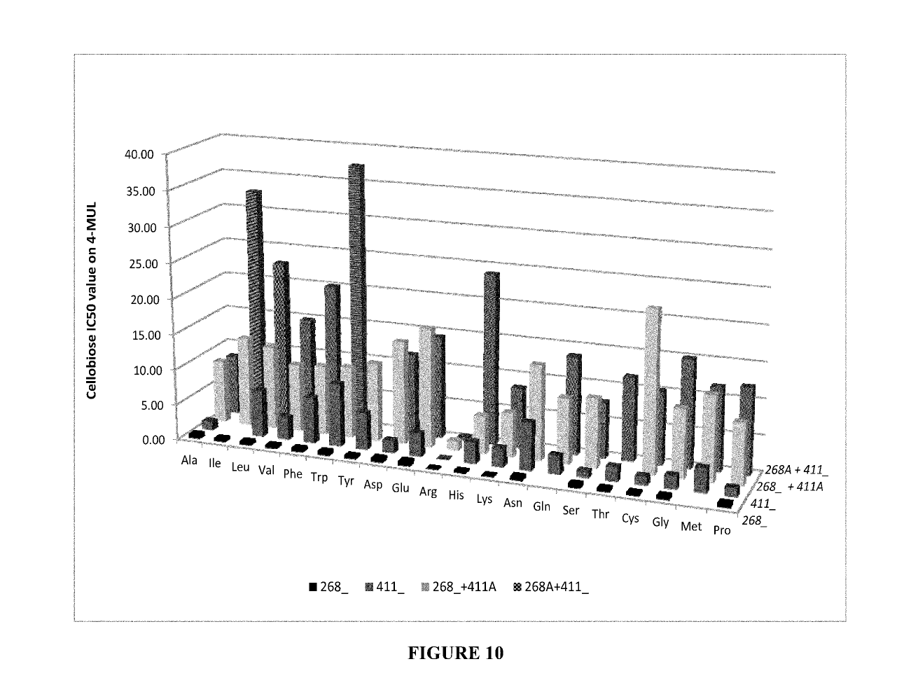

In one example, the cellobiose tolerance of the library was explored in more detail by generating dose-response curves and determining half maximal inhibitory concentration (IC₅₀) values, the point at which the enzyme is 50% inhibited. In two instances, IC₅₀ values were generated using samples with CBH I variant protein levels normalized to 5 μg/mL and using cellobiose concentrations in the range of 0.0001-100 mM (Table 9) or in the range of 0.00085-100 mM (Table 10). In another instance, IC₅₀ curves were generated using 30 μl of variant supernatant characterized by CBH I levels lower than 5 μg/mL and using cellobiose concentrations in the range of 0.00085-100 mM (Table 11). FIG. 9 shows representative IC₅₀ data and fitting using Prism (GraphPad). Averaged IC₅₀ values from Tables 8-11 are merged into Table 12 and are graphically presented in FIG. 10.

3.2. Results

Table 5 and FIG. 10 show important trends in the cellobiose IC₅₀ values of the variant library. These data show that both single mutant sites can increase tolerance relative to wild type (average WT IC₅₀=0.05 mM), with mutations at position 411 having a larger impact on increasing tolerance: on average, mutations at position 411 yield an IC₅₀ of 3.2 mM cellobiose, improving tolerance by 70-fold; whereas, mutations at position 268 yield an IC₅₀ of 0.4 mM cellobiose, improving tolerance by 9-fold. The double mutants show even larger increases over the wild type: with 268aa/411A mutants having an averaged IC₅₀ value of 11 mM cellobiose, or 230-fold improved tolerance; and 268A/411aa mutants having an averaged IC₅₀ value of 15 mM cellobiose, or 335-fold improved tolerance. Moreover, the average cellobiose tolerance increase for the double mutant is 4- to 7-fold higher than what would be expected from the additive effect of each single mutation measurement, demonstrating the apparent synergy of double mutations; see columns in Table 12 for measured IC₅₀, expected IC₅₀ (additive values), and synergy (fold-increase of measured over expected). As an example, a single mutations of 268N and 411A were respectively measured to be 0.49 and 1.17 each, giving an expected additive increase of 1.66 for the double mutant 268N/411A; the measured IC₅₀ value 268N/411A is 8-fold higher at 13.28. FIG. 9 shows the IC₅₀ curve shifts of single and synergistic double mutations for serine variants.

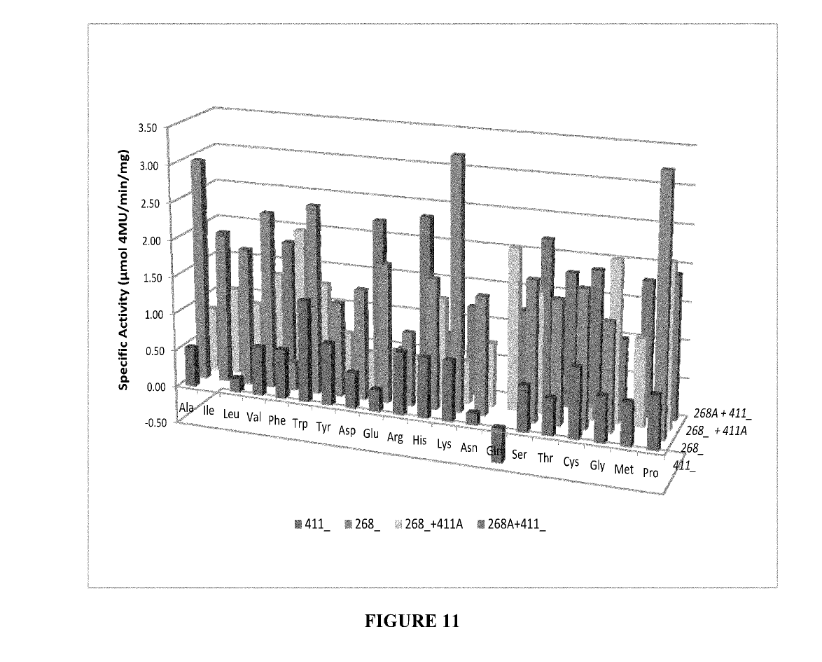

The specific activity (SA) of the variant library was evaluated in a secondary 4-MUL assay. Table 13 lists the specific activity for the variant library and FIG. 11 shows a graphical representation. These data show that the specific activity of variants is increased when mutations are introduced at position 268. On average, a mutation at position 268 increases the specific activity by 2.5 fold over that of wild type. A mutation at 268 in combination with 411 is around 1.5-1.6 fold higher than wild-type, on average. FIG. 9 shows these trends in specific activity for the serine variants, as represented by the higher relative fluorescence units for variants having the 268 mutation in the uninhibited zone of the IC₅₀ curves (low cellobiose concentrations, far left of curve).

### 4. Specific Embodiments and Incorporation by Reference

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

TABLE 1

| Sequence | Database | | |
|---|---|---|---|
| Identifier | Accession | | |
| (SEQ ID NO:) | Number | Species of Origin | Amino acid sequence |
| SEQ ID NO: 1 | BD29555* | Unknown | MSALNSFNMY KSALILGSLL ATAGAQQIGT YTAETHPSLS WSTCKSGGSC TTNSGAITLD ANWRWVHGVN TSTNCYTGNT |
| | | | WNTAICDTDA SCAQDCALDG ADYSGTYGIT TSGNSLRLNF VTGSNVGSRT YLMADNTHYQ IFDLLNQEFT FTVDVSHLPC |
| | | | GLNGALYFVT MDADGGVSKY PNNKAGAQYG VGYCDSQCPR DLKFIAGQAN VEGWTPSSNN ANTGLGNHGA CCAELDIWEA |
| | | | NSISEALTPH PCDTPGLSVC TTDACGGTYS SDRYAGTCDP DGCDFNPYRL GVTDFYGSGK TVDTTKPITV VTQFVTDDGT |
| | | | STGTLSEIRR YYVQNGVVIP QPSSKISGVS GNVINSDFCD AEISTFGETA SFSKHGGLAK MGAGMEAGMV LVMSLWDDYS |
| | | | VNMLWLDSTY PTNATGTPGA ARGSCPTTSG DPKTVESQSG SSYVTFSDIR VGPFNSTFSG GSSTGGSSTT TASGTTTTKA |
| | | | SSTSTSSTST GTGVAAHWGQ CGGQGWTGPT TCASGTTCTV VNPYYSQCL |
| SEQ ID NO: 2 | 340514556 | Trichoderma | MYRKLAVISA FLATARAQSA CTLQSETHPP LTWQKCSSGG TCTQQTGSVV IDANWRWTHA TNSSTNCYDG NTWSSTLCPD |
| | | reesei | NETCAKNCCL DGAAYASTYG VTTSGNSLSI GFVTQSAQKN VGARLYLMAS DTTYQEFTLL GNEFSFDVDV SQLPCGLNGA |
| | | | LYFVSMDADG GVSKYPTNTA GAKYGTGYCD SQCPRDLKFI NGQANVEGWE PSSNNANTGI GGHGSCCSEM DIWEANSISE |
| | | | ALTPHPCTTV GQEICEGDGC GGTYSDNRYG GTCDPDGCDW NPYRLGNTSF YGPGSSFTLD TTKKLTVVTQ FETSGAINRY |
| | | | YVQNGVTFQQ PNAELGSYSG NELNDDYCTA EEAEFGGSSF SDKGGLTQFK KATSGGMVLV MSLWDDYYAN MLWLDSTYPT |
| | | | NETSSTPGAV RGSCSTSSGV PAQVESQSPN AKVTFSNIKF GPIGSTGNPS GGNPPGGNPP GTTTTRRPAT TTGSSPGPTQ |
| | | | SHYGQCGGIG YSGPTVCASG TTCQVLNPYY SQCL |
| SEQ ID NO: 3 | 51243029 | Penicillium | MSALNSFNMY KSALILGSLL ATAGAQQIGT YTAETHPSLS WSTCKSGGSC TTNSGAITLD ANWRWVHGVN TSTNCYTGNT |
| | | occitanis | WNSAICDTDA SCAQDCALDG ADYSGTYGIT TSGNSLRLNF VTGSNVGSRT YLMADNTHYQ IFDLLNQEFT FTVDVSHLPC |
| | | | GLNGALYFVT MDADGGVSKY PNNKAGAQYG VGYCDSQCPR DLKFIAGQAN VEGWTPSANN ANTGIGNHGA CCAELDIWEA |
| | | | NSISEALTPH PCDTPGLSVC TTDACGGTYS SDRYAGTCDP DGCDFNPYRL GVTDFYGSGK TVDTTKPFTV VTQFVTNDGT |
| | | | STGSLSEIRR YYVQNGVVIP QPSSKISGIS GNVINSDYCA AEISTFGGTA SFNKHGGLTN MAAGMEAGMV LVMSLWDDYA |
| | | | VNMLWLDSTY PTNATGTPGA ARGTCATTSG DPKTVESQSG SSYVTFSDIR VGPFNSTFSG GSSTGGSTTT TASRTTTTSA |
| | | | SSTSTSSTST GTGVAGHWGQ CGGQGWTGPT TCVSGTTCTV VNPYYSQCL |
| SEQ ID NO: 4 | 7cel (PDB) & | Trichoderma | ESACTLQSET HPPLTWQKCS SGGTCTQQTG SVVIDANWRW THATNSSTNC YDGNTWSSTL CPDNETCAKN CCLDGAAYAS |
| | | reesei | TYGVTTSGNS LSIDFVTQSA QKNVGARLYL MASDTTYQEF TLLGNEFSFD VDVSQLPCGL NGALYFVSMD ADGGVSKYPT |
| | | | NTAGAKYGTG YCDSQCPRDL KFINGQANVE GWEPSSNNAN TGIGGHGSCC SEMDIWQANS ISEALTPHPC TTVGQEICEG |
| | | | DGCGGTYSDN RYGGTCDPDG CDWNPYRLGN TSFYGPGSSF TLDTTKKLTV VTQFETSGAI NRYYVQNGVT FQQPNAELGS |
| | | | YSGNELNDDY CTAEEAEFGG SSFSDKGGLT QFKKATSGGM VLVMSLWDDY YANMLWLDST YPTNETSSTP GAVRGSCSTS |
| | | | SGVPAQVESQ SPNAKVTFSN IKFGPIGSTG NPSG |
| SEQ ID NO: 5 | 67516425 | Aspergillus | MASSFQLYKA LLFFSSLLSA VQAQKVGTQQ AEVHPGLTWQ TCTSSGSCTT VNGEVTIDAN WRWLHTVNGY TNCYTGNEWD |
| | | nidulans FGSC A4 | TSICTSNEVC AEQCAVDGAN YASTYGITTS GSSLRLNFVT QSQQKNIGSR VYLMDDEDTY TMFYLLNKEF TFDVDVSELP |
| | | | CGLNGAVYFV SMDADGGKSR YATNEAGAKY GTGYCDSQCP RDLKFINGVA NVEGWESSDT NPNGGVGNHG SCCAEMDIWE |
| | | | ANSISTAFTP HPCDTPGQTL CTGDSCGGTY SNDRYGGTCD PDGCDFNSYR QGNKTFYGPG LTVDTNSPVT VVTQFLTDDN |
| | | | TDTGTLSEIK RFYVQNGVVI PNSESTYPAN PGNSITTEFC ESQKELFGDV DVFSAHGGMA GMGAALEQGM VLVLSLWDDN |
| | | | YSNMLWLDSN YPTDADPTQP GIARGTCPTD SGVPSEVEAQ YPNAYVVYSN IKFGPIGSTF GNGGGSGPTT TVTTSTATST |
| | | | TSSATSTATG QAQHWEQCGG NGWTGPTVCA SPWACTVVNS WYSQCL |
| SEQ ID NO: 6 | 46107376 | Gibberella zeae | MYRAIATASA LIAAVRAQQV CSLTQESKPS LNWSKCTSSG CSNVKGSVTI DANWRWTHQV SGSTNCYTGN KWDTSVCTSG |
| | | PH-1 | KVCAEKCCLD GADYASTYGI TSSGDQLSLS FVTKGPYSTN IGSRTYLMED ENTYQMFQLL GNEFTFDVDV SNIGCGLNGA |
| | | | LYFVSMDADG GKAKYPGNKA GAKYGTGYCD AQCPRDVKFI NGQANSDGWQ PSDSDVNGGI GNLGTCCPEM DIWEANSIST |
| | | | AYTPHPCTKL TQHSCTGDSC GGTYSNDRYG GTCDADGCDF NSYRQGNKTF YGPGSGFNVD TTKKVTVVTQ FHKGSNGRLS |
| | | | EITRLYVQNG KVIANSESKI AGVPGNSLTA DFCTKQKKVF NDPDDFTKKG AWSGMSDALE APMVLVMSLW HDHHSNMLWL |
| | | | DSTYPTDSTK LGSQRGSCST SSGVPADLEK NVPNSKVAFS NIKFGPIGST YKSDGTTPTN PTNPSEPSNT ANPNPGTVDQ |
| | | | WGQCGGSNYS GPTACKSGFT CKKINDFYSQ CQ |
| SEQ ID NO: 7 | 70992391 | Aspergillus | MLASTFSYRM YKTALILAAL LGSGQAQQVG TSQAEVHPSM TWQSCTAGGS CTTNNGKVVI DANWRWVHKV GDYTNCYTGN |
| | | fumigatus Af293 | TWDTTICPDD ATCASNCALE GANYESTYGV TASGNSLRLN FVTTSQQKNI GSRLYMMKDD STYEMFKLLN QEFTFDVDVS |
| | | | NLPCGLNGAL YFVAMDADGG MSKYPTNKAG AKYGTGYCDS QCPRDLKFIN GQANVEGWQP SSNDANAGTG NHGSCCAEMD |
| | | | IWEANSISTA FTPHPCDTPG QVMCTGDACG GTYSSDRYGG TCDPDGCDFN SFRQGNKTFY GPGMTVDTKS KFTVVTQFIT |
| | | | DDGTSSGTLK EIKRFYVQNG KVIPNSESTW TGVSGNSITT EYCTAQKSLF QDQNVFEKHG GLEGMGAALA QGMVLVMSLW |
| | | | DDHSANMLWL DSNYPTTASS TTPGVARGTC DISSGVPADV EANHPDAYVV YSNIKVGPIG STFNSGGSNP GGGTTTTTTT |
| | | | QPTTTTTTAG NPGGTGVAQH YGQCGGIGWT GPTTCASPYT CQKLNDYYSQ CL |
| SEQ ID NO: 8 | 121699984 | Aspergillus | MLPSTISYRI YKNALFFAAL FGAVQAQKVG TSKAEVHPSM AWQTCAADGT CTTKNGKVVI DANWRWVHDV KGYTNCYTGN |
| | | clavatus NRRL 1 | TWNAELCPDN ESCAENCALE GADYAATYGA TTSGNALSLK FVTQSQQKNI GSRLYMMKDD NTYETFKLLN QEFTFDVDVS |
| | | | NLPCGLNGAL YFVSMDADGG LSRYTGNEAG AKYGTGYCDS QCPRDLKFIN GLANVEGWTP SSSDANAGNG GHGSCCAEMD |
| | | | IWEANSISTA YTPHPCDTPG QAMCNGDSCG GTYSSDRYGG TCDPDGCDFN SYRQGNKSFY GPGMTVDTKK KMTVVTQFLT |
| | | | NDGTATGTLS EIKRFYVQDG KVIANSESTW PNLGGNSLTN DFCKAQKTVF GDMDTFSKHG GMEGMGAALA EGMVLVMSLW |
| | | | DDHNSNMLWL DSNSPTTGTS TTPGVARGSC DISSGDPKDL EANHPDASVV YSNIKVGPIG STFNSGGSNP GGSTTTTKPA |
| | | | TSTTTTKATT TATTNTTGPT GTGVAQPWAQ CGGIGYSGPT QCAAPYTCTK QNDYYSQCL |
| SEQ ID NO: 9 | 1906845 | Claviceps | MHPSLQTILL SALFTTAHAQ QACSSKPETH PPLSWSRCSR SGCRSVQGAV TVDANWLWTT VDGSQNCYTG NRWDTSICSS |
| | | purpurea | EKTCSESCCI DGADYAGTYG VTTTGDALSL KFVQQGPYSK NVGSRLYLMK DESRYEMFTL LGNEFTFDVD VSKLGCGLNG |
| | | | ALYFVSMDED GGMKRFPMNK AGAKFGTGYC DSQCPRDVKF INGMANSKDW IPSKSDANAG IGSLGACCRE MDIWEANNIA |
| | | | SAFTPHPCKN SAYHSCTGDG CGGTYSKNRY SGDCDPDGCD FNSYRLGNTT FYGPGPKFTI DTTRKISVVT QFLKGRDGSL |
| | | | REIKRFYVQN GKVIPNSVSR VRGVPGNSIT QGFCNAQKKM FGAHESFNAK GGMKGMSAAV SKPMVLVMSL WDDHNSNMLW |
| | | | LDSTYPTNSR QRGSKRGSCP ASSGRPTDVE SSAPDSTVVF SNIKFGPIGS TFSRGK |
| SEQ ID NO: 10 | 1gpi (PDB) & | Phanerochaete | EQAGTNTAEN HPQLQSQQCT TSGGCKPLST KVVLDSNWRW VHSTSGYTNC YTGNEWDTSL CPDGKTCAAN CALDGADYSG |
| | | chrysosporium | TYGITSTGTA LTLKFVTGSN VGSRVYLMAD DTHYQLLKLL NQEFTFDVDM SNLPCGLNGA LYLSAMDADG GMSKYPGNKA |
| | | | GAKYGTGYCD SQCPKDIKFI NGEANVGNWT ETGSNTGTGS YGTCCSEMDI WEANNDAAAF TPHPCTTTGQ TRCSGDDCAR |
| | | | NTGLCDGDGC DFNSFRMGDK TFLGKGMTVD TSKPFTVVTQ FLTNDNTSTG TLSEIRRIYI QNGKVIQNSV ANIPGVDPVN |
| | | | SITDNFCAQQ KTAFGDTNWF AQKGGLKQMG EALGNGMVLA LSIWDDHAAN MLWLDSDYPT DKDPSAPGVA RGTCATTSGV |
| | | | PSDVESQVPN SQVVFSNIKF GDIGSTFSGT S |
| SEQ ID NO: 11 | 119468034 | Neosartorya | MHQRALLFSA LAVAANAQQV GTQKPETHPP LTWQKCTAAG SCSQQSGSVV IDANWRWLHS TKDTTNCYTG NTWNTELCPD |
| | | fischeri NRRL 181 | NESCAQNCAV DGADYAGTYG VTTSGSELKL SFVTGANVGS RLYLMQDDET YQHFNLLNNE FTFDVDVSNL PCGLNGALYF |
| | | | VAMDADGGMS KYPSNKAGAK YGTGYCDSQC PRDLKFINGM ANVEGWKPSS NDKNAGVGGH GSCCPEMDIW EANSISTAVT |
| | | | PHPCDDVSQT MCSGDACGGT YSATRYAGTC DPDGCDFNPF RMGNESFYGP GKIVDTKSEM TVVTQFITAD GTDTGALSEI |
| | | | KRLYVQNGKV IANSVSNVAD VSGNSISSDF CTAQKKAFGD EDIFAKHGGL SGMGKALSEM VLIMSIWDDH HSSMMWLDST |
| | | | YPTDADPSKP GVARGTCEHG AGDPEKVESQ HPDASVTFSN IKFGPIGSTY KA |
| SEQ ID NO: 12 | 7804883 | Leptosphaeria | MYRSLIFATS LLSLAKGQLV GNLYCKGSCT AKNGKVVIDA NWRWLHVKGG YTNCYTGNEW NATACPDNKS CATNCAIDGA |
| | | maculans | DYRRLRHYCE RQLLGTEVHH QGLYSTNIGS RTYLMQDDST YQLFKFTGSQ EFTFDVDLSN LPCGLNGALY FVSMDADGGL |
| | | | KKYPTNKAGA KYGTGYCDAQ CPRDLKFING EGNVEGWQPS KNDQNAGVGG HGSCCAEMDI WEANSVSTAV TPHSCSTIEQ |
| | | | SRCDGDGCGG TYSADRYAGV CDPDGCDFNS YRMGVKDFYG KGKTVDTSKK FTVVTQFIGS GDAMEIKRFY VQNGKTIPQP |
| | | | DSTIPGVTGN SITTFFCDAQ KKAFGDKYTF KDKGGMANMP STCNGMVLVM SLWDDHYSNM LWLDSTYPTD KNPDTDAGSG |
| | | | RGECAITSGV PADVESQHPD ASVIYSNIKF GPINTTFG |
| SEQ ID NO: 13 | 85108032 | Neurospora crassa | MLAKFAALAA LVASANAQAV CSLTAETHPS LNWSKCTSSG CTNVAGSITV DANWRWTHIT SGSTNCYSGN EWDTSLCSTN |
| | | N150(OR74A) | TDCATKCCVD GAEYSSTYGI QTSGNSLSLQ FVTKGSYSTN IGSRTYLMNG ADAYQGFELL GNEFTFDVDV SGTGCGLNGA |
| | | | LYFVSMDLDG GKAKYTNNKA GAKYGTGYCD AQCPRDLKYI NGIANVEGWT PSTNDANAGI GDHGTCCSEM DIWEANKVST |
| | | | AFTPHPCTTI EQHMCEGDSC GGTYSDDRYG GTCDADGCDF NSYRMGNTTF YGEGKTVDTS SKFTVVTQFI KDSAGDLAEI |
| | | | KRFYVQNGKV IENSQSNVDG VSGNSITQSF CNAQKTAFGD IDDFNKKGGL KQMGKALAKP MVLVMSIWDD HAANMLWLDS |
| | | | TYPVEGGPGA YRGECPTTSG VPAEVEANAP NSKVIFSNIK FGPIGSTFSG GSSGTPPSNP SSSVKPVTST AKPSSTSTAS |
| | | | NPSGTGAAHW AQCGGIGFSG PTTCQSPYTC QKINDYYSQC V |
| SEQ ID NO: 14 | 169859458 | Coprinopsis | MFKKVALTAL CFLAVAQAQQ VGREVAENHP RLPWQRCTRN GGCQTVSNGQ VVLDANWRWL HVTDGYTNCY TGNSWNSTVC |
| | | cinerea okayama | SDPTTCAQRC ALEGANYQQT YGITTNGDAL TIKFLTRSQQ TNVGARVYLM ENENRYQMFN LLNKEFTFDV DVSKVPCGIN |
| | | | GALYFIQMDA DGGMSKQPNN RAGAKYGTGY CDSQCPRDIK FIDGVANSAD WTPSETDPNA GRGRYGICCA EMDIWEANSI |
| | | | SNAYTPHPCR TQNDGGYQRC EGRDCNQPRY EGLCDPDGCD YNPFRMGNKD FYGPGKTVDT NRKMTVVTQF ITHDNTDTGT |
| | | | LVDIRRLYVQ DGRVIANPPT NFPGLMPAHD SITEQFCTDQ KNLFGDYSSF ARDGGLAHMG RSLAKGHVLA LSIWNDHGAH |
| | | | MLWLDSNYPT DADPNKPGIA RGTCPTTGGT PRETEQNHPD AQVIFSNIKF GDIGSTFSGY |
| SEQ ID NO: 15 | 154292161 | Botryotinia | MYSAAVLATF SFLLGAGAQQ VGTSTAETHP ALTVQKCAAG GTCTDESDSI VLDANWRWLH STSGSTNCYT GNTWDTTLCP |
| | | fuckeliana B05-10 | DAATCTTNCA LDGADYEGTY GITTSGDSLK LSFVTGSNVG SRTYLMDSET TYKEFALLGN EFTFTVDVSK LPCGLNGALY |
| | | | FVPMDADGGM SKYPTNKAGA KYGTGYCDAQ CPQDMKFVNG TANVEGWVPD SNSANSGTGN IGSCCSEFDV WEANSMSQAL |
| | | | TPHVCTVDSQ TACTGDDCAS NTGVCDGDGC DFNPYRMGNT TFYGSGMTID TSKPFSVVTQ FITDDGTETG TLTEIKRFYV |
| | | | QDDVVYEQPS SDISGVSGNS ITDDFCAAQK TAFGDTDYFT QNGGMAAMGK KMADGMVLVL SIWDDYNVNM LWLDSDYPTT |
| | | | KDASTPGVSR GSCATDSGVP ATVEAASGSA YVTFSSIKYG PIGSTFNAPA DSSSSVSASS SPAPIASSSS SASIAPVSSV |
| | | | VAAIVSSSAQ AISSAAPVVS SSAQAISSAA PVVSSVVSSA APVATSSTKS KCSKVSSTLK TSVAAPATSA TSAAVVATSS |
| | | | AASSTGSVPL YGNCTGGKTC SEGTCVVQND YYSQCVASS |
| SEQ ID NO: 16 | 169615761 # | Phaeosphaeria | MTWQRCTGTG GSSCTNVNGE IVIDANWRWI HATGGYTNCF DGNEWNKTAC PSNAACTKNC AIEGSDYRGT YGITTSGNSL |
| | | nodorum SN15 | TLKFITKGQY STNVGSRTYL MKDTNNYEMF NLIGNEFTFD VDLSQLPCGL NGALYFVSMP EKGQGTPGAK YGTGKLSQCS |
| | | | VHISKTLTDA CARDLKFVGG EANADGWQAS TSDPNAGVGK KGACCAEMDV WEANSMSTAL TPHSCQPEGY AVCEESNCGG |
| | | | TYSLDRYAGT CDANGCDFNP YRVGNKDFYG KGKTVDTSKK MTVVTQFLGT GSDLTELKRF YVQDGKVISN PEPTIPGMTG |
| | | | NSITQKWCDT QKEVFKEEVY PFNQWGGMAS MGKGMAQGMV LVMSLWDDHY SNMLWLDSTY PTDRDPESPG AARGECAITS |
| | | | GAPAEVEANN PDASVMFSNI KFGPIGSTFQ QPA |
| SEQ ID NO: 17 | 4883502 | Humicola grisea | MQIKSYIQYL AAALPLLSSV AAQQAGTITA ENHPRMTWKR CSGPGNCQTV QGEVVIDANW RWLHNNGQNC YEGNKWTSQC |
| | | | SSATDCAQRC ALDGANYQST YGASTSGDSL TLKFVTKHEY GTNIGSRFYL MANQNKYQMF TLMNNEFAFD VDLSKVECGI |
| | | | NSALYFVAME EDGGMASYPS NRAGAKYGTG YCDAQCARDL KFIGGKANIE GWRPSTNDPN AGVGPMGACC AEIDVWESNA |
| | | | YAYAFTPHAC GSKNRYHICE TNNCGGTYSD DRFAGYCDAN GCDYNPYRMG NKDFYGKGKT VDTNRKFTVV SRFERNRLSQ |
| | | | FFVQDGRKIE VPPPTWPGLP NSADITPELC DAQFRVFDDR NRFAETGGFD ALNEALTIPM VLVMSIWDDH HSNMLWLDSS |
| | | | YPPEKAGLPG GDRGPCPTTS GVPAEVEAQY PNAQVVWSNI RFGPIGSTVN V |
| SEQ ID NO: 18 | 950686 | Humicola grisea | MRTAKFATLA ALVASAAAQQ ACSLTTERHP SLSWKKCTAG GQCQTVQASI TLDSNWRWTH QVSGSTNCYT GNKWDTSICT |
| | | | DAKSCAQNCC VDGADYTSTY GITTNGDSLS LKFVTKGQYS TNVGSRTYLM DGEDKYQTFE LLGNEFTFDV DVSNIGCGLN |
| | | | GALYFVSMDA DGGLSRYPGN KAGAKYGTGY CDAQCPRDIK FINGEANIEG WTGSTNDPNA GAGRYGTCCS EMDIWEANNM |
| | | | ATAFTPHPCT IIGQSRCEGD SCGGTYSNER YAGVCDPDGC DFNSYRQGNK TFYGKGMTVD TTKKITVVTQ FLKDANGDLG |
| | | | EIKRFYVQDG KIIPNSESTI PGVEGNSITQ DWCDRQKVAF GDIDDFNRKG GMKQMGKALA GPMVLVMSIW DDHASNMLWL |
| | | | DSTFPVDAAG KPGAERGACP TTSGVPAEVE AEAPNSNVVF SNIRFGPIGS TVAGLPGAGN GGNNGGNPPP PTTTTSSAPA |
| | | | TTTTASAGPK AGRWQQCGGI GFTGPTQCEE PYTCTKLNDW YSQCL |
| SEQ ID NO: 19 | 124491660 | Chaetomium | MQIKQYLQYL AAALPLVNMA AAQRAGTQQT ETHPRLSWKR CSSGGNCQTV NAEIVIDANW RWLHDSNYQN CYDGNRWTSA |
| | | thermophilum | CSSATDCAQK CYLEGANYGS TYGVSTSGDA LTLKFVTKHE YGTNIGSRVY LMNGSDKYQM FTLMNNEFAF DVDLSKVECG |
| | | | LNSALYFVAM EEDGGMRSYS SNKAGAKYGT GYCDAQCARD LKFVGGKANI EGWRPSTNDA NAGVGPYGAC CAEIDVWESN |
| | | | AYAFAFTPHG CLNNNYHVCE TSNCGGTYSE DRFGGLCDAN GCDYNPYRMG NKDFYGKGKT VDTSRKFTVV TRFEENKLTQ |
| | | | FFIQDGRKID IPPPTWPGLP NSSAITPELC TNLSKVFDDR DRYEETGGFR TINEALRIPM VLVMSIWDGH YANMLWLDSV |
| | | | YPPEKAGQPG AERGPCAPTS GVPAEVEAQF PNAQVIWSNI RFGPIGSTYQ V |
| SEQ ID NO: 20 | 58045187 | Chaetomium | MMYKKFAALA ALVAGAAAQQ ACSLTTETHP RLTWKRCTSG GNCSTVNGAV TIDANWRWTH TVSGSTNCYT GNEWDTSICS |
| | | thermophilum | DGKSCAQTCC VDGADYSSTY GITTSGDSLN LKFVTKHQHG TNVGSRVYLM ENDTKYQMFE LLGNEFTFDV DVSNLGCGLN |
| | | | GALYFVSMDA DGGMSKYSGN KAGAKYGTGY CDAQCPRDLK FINGEANIEN WTPSTNDANA GFGRYGSCCS EMDIWDANNM |
| | | | ATAFTPHPCT IIGQSRCEGN SCGGTYSSER YAGVCDPDGC DFNAYRQGDK TFYGKGMTVD TTKKMTVVTQ FHKNSAGVLS |
| | | | EIKRFYVQDG KIIANAESKI PGNPGNSITQ EWCDAQKVAF GDIDDFNRKG GMAQMSKALE GPMVLVMSVW DDHYANMLWL |
| | | | DSTYPIDKAG TPGAERGACP TTSGVPAEIE AQVPNSNVIF SNIRFGPIGS TVPGLDGSTP SNPTATVAPP TSTTTSVRSS |
| | | | TTQISTPTSQ PGGCTTQKWG QCGGIGYTGC TNCVAGTTCT ELNPWYSQCL |
| SEQ ID NO: 21 | 169601100 # | Phaeosphaeria | MYRNFLYAAS LLSVARSQLV GTQTTETHPG MTWQSCTAKG SCTTCSDNKA CASNCAVDGA DYKGTYGITA SGNSLQLKFI |
| | | nodorum SN15 | TKGSYSTNIG SRTYLMASDT AYQMFKFDGN KEFTFDVDLS GLPCGFNGAL YFVSMDEDGG LKKYSGNKAG AKYGTGYCDA |
| | | | QCPRDLKFIN GEGNVEGWKP SDNDANAGVG GHGSCCAEMD IWEANSISTA VTPHACSTIE QTRCDGDGCG GTYSADRYAG |
| | | | VCDPDGCDFN AYRMGVKNFY GKGMTVDTSK KFTVVTQFIG TGDAMEIKRF YVQGGKTIEQ PASTIPGVEG NSITTKFCDQ |
| | | | QKQVFGDRYT YKEKGGTANM AKALAQGMVL VMSLWDDHYS NMLWLDSTYP TDKNPDTDLG SGRGSCDVKS GAPADVESKS |
| | | | PDATVIYSNI KFGPLNSTY |
| SEQ ID NO: 22 | 169870197 | Coprinopsis | MLGKIAIASL SFLAIAKGQQ VGREVAENHP RLPWQRCTRN GGCQTVSNGQ VVLDANWRWL HVTDGYTNCY TGNSWNSSVC |
| | | cinerea Okayama | SDGTTCAQRC ALEGANYQQT YGITTSGNSL TMKFLTRSQG TNVGGRVYLM ENENRYQMFN LLNKEFTFDV DVSKVPCGIN |
| | | | GALYFIQMDA DGGMSSQPNN RAGAKYGTGY CDSQCPRDIK FIDGVANSVG WEPSETDSNA GRGRYGICCA EMDIWEANSI |
| | | | SNAYTPHPCR TQNDGGYQRC EGRDCNQPRY EGLCDPDGCD YNPFRMGNKD FYGPGKTIDT NRKMTVVTQF ITHDNTDTGT |
| | | | LVDIRRLYVQ DGRVIANPPT NFPGLMPAHD SITEQFCTDQ KNLFGDYSSF ARDGGLAHMG RSLAKGHVLA LSIWNDHGAH |
| | | | MLWLDSNYPT DADPNKPGIA RGTCPTTGGT PRETEQNHPD AQVIFSNIKF GDIGSTFSGY |
| SEQ ID NO: 23 | 3913806 | Agaricus bisporus | MFPRSILLAL SLTAVALGQQ VGTNMAENHP SLTWQRCTSS GCQNVNGKVT LDANWRWTHR INDFTNCYTG NEWDTSICPD |
| | | | GVTCAENCAL DGADYAGTYG VTSSGTALTL KFVTESQQKN IGSRLYLMAD DSNYEIFNLL NKEFTFDVDV SKLPCGLNGA |
| | | | LYFSEMAADG GMSSTNTAGA KYGTGYCDSQ CPRDIKFIDG EANSEGWEGS PNDVNAGTGN FGACCGEMDI WEANSISSAY |
| | | | TPHPCREPGL QRCEGNTCSV NDRYATECDP DGCDFNSFRM GDKSFYGPGM TVDTNQPITV VTQFITDNGS DNGNLQEIRR |
| | | | IYVQNGQVIQ NSNVNIPGID SGNSISAEFC DQAKEAFGDE RSFQDRGGLS GMGSALDRGM VLVLSIWDDH AVNMLWLDSD |
| | | | YPLDASPSQP GISRGTCSRD SGKPEDVEAN AGGVQVVYSN IKFGDINSTF NNNGGGGGNP SPTTTRPNSP AQTMWGQCGG |
| | | | QGWTGPTACQ SPSTCHVIND FYSQCF |
| SEQ ID NO: 24 | 169611094 | Phaeosphaeria | MYRNLALASL SLFGAARAQQ AGTVTTETHP SLSWKTCTGT GGTSCTTKAG KITLDANWRW THVTTGYTNC YDGNSWNTTA |
| | | nodorum SN15 | CPDGATCTKN CAVDGADYSG TYGITTSSNS LSIKFVTKGS NSANIGSRTY LMESDTKYQM FNLIGQEFTF DVDVSKLPCG |
| | | | LNGALYFVEM AADGGIGKGN NKAGAKYGTG YCDSQCPHDI KFINGKANVE GWNPSDADPN AGSGKIGACC PEMDIWEANS |
| | | | ISTAYTPHPC KGTGLQECTD DVSCGDGSNR YSGLCDKDGC DFNSYRMGVK DFYGPGATLD TTKKMTVVTQ FLGSGSTLSE |
| | | | IKRFYVQNGK VFKNSDSAIE GVTGNSITES FCAAQKTAFG DTNSFKTLGG LNEMGASLAR GHVLVMSLWD DHAVNMLWLD |
| | | | STYPTNSTKL GAQRGTCAID SGKPEDVEKN HPDATVVFSD IKFGPIGSTF QQPS |
| SEQ ID NO: 25 | 3131 | Phanerochaete | MVDIQIATFL LLGVVGVAAQ QVGTYIPENH PLLATQSCTA SGGCTTSSSK IVLDANRRWI HSTLGTTSCL TANGWDPTLC |
| | | chrysosporium | PDGITCANYC ALDGVSYSST YGITTSGSAL RLQFVTGTNI GSRVFLMADD THYRTFQLLN QELAFDVDVS KLPCGLNGAL |
| | | | YFVAMDADGG KSKYPGNRAG AKYGTGYCDS QCPRDVQFIN GQANVQGWNA TSATTGTGSY GSCCTELDIW EANSNAAALT |
| | | | PHTCTNNAQT RCSGSNCTSN TGFCDADGCD FNSFRLGNTT FLGAGMSVDT TKTFTVVTQF ITSDNTSTGN LTEIRRFYVQ |
| | | | NGNVIPNSVV NVTGIGAVNS ITDPFCSQQK KAFIETNYFA QHGGLAQLGQ ALRTGMVLAF SISDDPANHM LWLDSNFPPS |
| | | | ANPAVPGVAR GMCSITSGNP ADVGILNPSP YVSFLNIKFG SIGTTFRPA |
| SEQ ID NO: 26 | 70991503 | Aspergillus | MHQRALLFSA LAVAANAQQV GTQTPETHPP LTWQKCTAAG SCSQQSGSVV IDANWRWLHS TKDTTNCYTG NTWNTELCPD |
| | | fumigatus Af293 | NESCAQNCAL DGADYAGTYG VTTSGSELKL SFVTGANVGS RLYLMQDDET YQHFNLLNHE FTFDVDVSNL PCGLNGALYF |
| | | | VAMDADGGMS KYPSNKAGAK YGTGYCDSQC PRDLKFINGM ANVEGWEPSS SDKNAGVGGH GSCCPEMDIW EANSISTAVT |
| | | | PHPCDDVSQT MCSGDACGGT YSESRYAGTC DPDGCDFNPF RMGNESFYGP GKIVDTKSKM TVVTQFITAD GTDSGALSEI |
| | | | KRLYVQNGKV IANSVSNVAG VSGNSITSDF CTAQKKAFGD EDIFAKHGGL SGMGKALSEM VLIMSIWDDH HSSMMWLDST |
| | | | YPTDADPSKP GVARGTCEHG AGDPENVESQ HPDASVTFSN IKFGPIGSTY EG |
| SEQ ID NO: 27 | 294196 | Phanerochaete | MFRTATLLAF TMAAMVFGQQ VGTNTAENHR TLTSQKCTKS GGCSNLNTKI VLDANWRWLH STSGYTNCYT GNQWDATLCP |
| | | chrysosporium | DGKTCAANCA LDGADYTGTY GITASGSSLK LQFVTGSNVG SRVYLMADDT HYQMFQLLNQ EFTFDVDMSN LPCGLNGALY |
| | | | LSAMDADGGM AKYPTNKAGA KYGTGYCDSQ CPRDIKFING EANVEGWNAT SANAGTGNYG TCCTEMDIWE ANNDAAAYTP |
| | | | HPCTTNAQTR CSGSDCTRDT GLCDADGCDF NSFRMGDQTF LGKGLTVDTS KPFTVVTQFI TNDGTSAGTL TEIRRLYVQN |
| | | | GKVIQNSSVK IPGIDPVNSI TDNFCSQQKT AFGDTNYFAQ HGGLKQVGEA LRTGMVLALS IWDDYAANML WLDSNYPTNK |
| | | | DPSTPGVARG TCATTSGVPA QIEAQSPNAY VVFSNIKFGD LNTTYTGTVS SSSVSSSHSS TSTSSSHSSS STPPTQPTGV |
| | | | TVPQWGQCGG IGYTGSTTCA SPYTCHVLNP YYSQCY |
| SEQ ID NO: 28 | 18997123 | Thermoascus | MYQRALLFSF FLAAARAHEA GTVTAENHPS LTWQQCSSGG SCTTQNGKVV IDANWRWVHT TSGYTNCYTG NTWDTSICPD |
| | | aurantiacus | DVTCAQNCAL DGADYSGTYG VTTSGNALRL NFVTQSSGKN IGSRLYLLQD DTTYQIFKLL GQEFTFDVDV SNLPCGLNGA |
| | | | LYFVAMDADG NLSKYPGNKA GAKYGTGYCD SQCPRDLKFI NGQANVEGWQ PSANDPNAGV GNHGSSCAEM DVWEANSIST |
| | | | AVTPHPCDTP GQTMCQGDDC GGTYSSTRYA GTCDPDGCDF NPYQPGNHSF YGPGKIVDTS SKFTVVTQFI TDDGTPSGTL |
| | | | TEIKRFYVQN GKVIPQSEST ISGVTGNSIT TEYCTAQKAA FGDNTGFFTH GGLQKISQAL AQGMVLVMSL WDDHAANMLW |
| | | | LDSTYPTDAD PDTPGVARGT CPTTSGVPAD VESQNPNSYV IYSNIKVGPI NSTFTAN |
| SEQ ID NO: 29 | 4204214 | Humicola grisea | MQIKSYIQYL AAALPLLSSV AAQQAGTITA ENHPRMTWKR CSGPGNCQTV QGEVVIDANW RWLHNNGQNC YEGNKWTSQC |
| | | var thermoidea | SSATDCAQRC ALDGANYQST YGASTSGDSL TLKFVTKHEY GTNIGSRFYL MANQNKYQMF TLMNNEFAFD VDLSKVECGI |
| | | | NSALYFVAME EDGGMASYPS NRAGAKYGTG YCDAQCARDL KFIGGKANIE GWRPSTNDPN AGVGPMGACC AEIDVWESNA |
| | | | YAYAFTPHAC GSKNRYHICE TNNCGGTYSD DRFAGYCDAN GCDYNPYRMG NKDFYGKGKT VDTNRKFTVV SRFERNRLSQ |
| | | | FFVQDGRKIE VPPPTWPGLP NSADITPELC DAQFRVFDDR NRFAETGGFD ALNEALTIPM VLVMSIWDDH HSNMLWLDSS |
| | | | YPPEKAGLPG GDRGPCPTTS GVPAEVEAQY PDAQVVWSNI RFGPIGSTVN V |
| SEQ ID NO: 30 | 34582632 | Trichoderma | MYRKLAVISA FLATARAQSA CTLQSETHPP LTWQKCSSGG TCTQQTGSVV IDANWRWTHA TNSSTNCYDG NTWSSTLCPD |
| | | viride (also known | NETCAKNCCL DGAAYASTYG VTTSGNSLSI GFVTQSAQKN VGARLYLMAS DTTYQEFTLL GNEFSFDVDV SQLPCGLNGA |
| | | as Hypochrea rufa) | LYFVSMDADG GVSKYPTNTA GAKYGTGYCD SQCPRDLKFI NGQANVEGWE PSSNNANTGI GGHGSCCSEM DIWEANSISE |
| | | | ALTPHPCTTV GQEICEGDGC GGTYSDNRYG GTCDPDGCDW DPYRLGNTSF YGPGSSFTLD TTKKLTVVTQ FETSGAINRY |
| | | | YVQNGVTFQQ PNAELGSYSG NGLNDDYCTA EEAEFGGSSF SDKGGLTQFK KATSGGMVLV MSLWDDYYAN MLWLDSTYPT |
| | | | NETSSTPGAV RGSCSTSSGV PAQVESQSPN AKVTFSNIKF GPIGSTGDPS GGNPPGGNPP GTTTTRRPAT TTGSSPGPTQ |
| | | | SHYGQCGGIG YSGPTVCASG TTCQVLNPYY SQCL |
| SEQ ID NO: 31 | 156712284 | Thermoascus | MYQRALLFSF FLAAARAQQA GTVTAENHPS LTWQQCSSGG SCTTQNGKVV IDANWRWVHT TSGYTNCYTG NTWDTSICPD |
| | | aurantiacus | DVTCAQNCAL DGADYSGTYG VTTSGNALRL NFVTQSSGKN IGSRLYLLQD DTTYQIFKLL GQEFTFDVDV SNLPCGLNGA |
| | | | LYFVAMDADG GLSKYPGNKA GAKYGTGYCD SQCPRDLKFI NGQANVEGWQ PSANDPNAGV GNHGSCCAEM DVWEANSIST |
| | | | AVTPHPCDTP GQTMCQGDDC GGTYSSTRYA GTCDPDGCDF NPYRQGNHSF YGPGQIVDTS SKFTVVTQFI TDDGTPSGTL |
| | | | TEIKRFYVQN GKVIPQSEST ISGVTGNSIT TEYCTAQKAA FGDNTGFFTH GGLQKISQAL AQGMVLVMSL WDDHAANMLW |
| | | | LDSTYPTDAD PDTPGVARGT CPTTSGVPAD VESQYPNSYV IYSNIKVGPI NSTFTAN |
| SEQ ID NO: 32 | 39977899 | Magnaporthe | MIRKITTLAA LVGVVRGQAA CSLTAETHPS LTWQKCSSGG SCTNVAGSVT IDANWRWTHT TSGYTNCYTG NKWDTSICST |
| | | grisea (oryzae) 70- | NADCASKCCV DGANYQQTYG ASTSGNALSL QYVTQSSGKN VGSRLYLLES ENKYQMFNLL GNEFTFDVDA SKLGCGLNGA |
| | | 15 | VYFVSMDADG GQSKYSGNKA GAKYGTGYCD SQCPRDLKYI NGAANVEGWQ PSSGDANSGV GNMGSCCAEM DIWEANSIST |
| | | | AYTPHPCSNN AQHSCKGDDC GGTYSSVRYA GDCDPDGCDF NSYRQGNRTF YGPGSNFNVD SSKKVTVVTQ FISSGGQLTD |
| | | | IKRFYVQNGK VIPNSQSTIT GVTGNSVTQD YCDKQKTAFG DQNVFNQRGG LRQMGDALAK GMVLVMSVWD DHHSQMLWLD |
| | | | STYPTTSTAP GAARGSCSTS SGKPSDVQSQ TPGATVVYSN IKFGPIGSTF KSS |
| SEQ ID NO: 33 | 20986705 | Talaromyces | MLRRALLLSS SAILAVKAQQ AGTATAENHP PLTWQECTAP GSCTTQNGAV VLDANWRWVH DVNGYTNCYT GNTWDPTYCP |
| | | emersonii | DDETCAQNCA LDGADYEGTY GVTSSGSSLK LNFVTGSNVG SRLYLLQDDS TYQIFKLLNR EFSFDVDVSN LPCGLNGALY |
| | | | FVAMDADGGV SKYPNNKAGA KYGTGYCDSQ CPRDLKFIDG EANVEGWQPS SNNANTGIGD HGSCCAEMDV WEANSISNAV |
| | | | TPHPCDTPGQ TMCSGDDCGG TYSNDRYAGT CDPDGCDFNP YRMGNTSFYG PGKIIDTTKP FTVVTQFLTD DGTDTGTLSE |
| | | | IKRFYIQNSN VIPQPNSDIS GVTGNSITTE FCTAQKQAFG DTDDFSQHGG LAKMGAAMQQ GMVLVMSLWD DYAAQMLWLD |
| | | | SDYPTDADPT TPGIARGTCP TDSGVPSDVE SQSPNSYVTY SNIKFGPINS TFTAS |
| SEQ ID NO: 34 | 22138843 | Aspergillus oryzae | MHQRALLFSA FWTAVQAQQA GTLTAETHPS LTWQKCAAGG TCTEQKGSVV LDSNWRWLHS VDGSTNCYTG NTWDATLCPD |
| | | | NESCASNCAL DGADYEGTYG VTTSGDALTL QFVTGANIGS RLYLMADDDE SYQTFNLLNN EFTFDVDASK LPCGLNGAVY |
| | | | FVSMDADGGV AKYSTNKAGA KYGTGYCDSQ CPRDLKFING QVRKGWEPSD SDKNAGVGGH GSCCPQMDIW EANSISTAYT |
| | | | PHPCDDTAQT MCEGDTCGGT YSSERYAGTC DPDGCDFNAY RMGNESFYGP SKLVDSSSPV TVVTQFITAD GTDSGALSEI |
| | | | KRFYVQGGKV IANAASNVDG VTGNSITADF CTAQKKAFGD DDIFAQHGGL QGMGNALSSM VLTLSIWDDH HSSMMWLDSS |
| | | | YPEDADATAP GVARGTCEPH AGDPEKVESQ SGSATVTYSN IKYGPIGSTF DAPA |
| SEQ ID NO: 35 | 55775695 | Penicillium | MASTLSFKIY KNALLLAAFL GAAQAQQVGT STAEVHPSLT WQKCTAGGSC TSQSGKVVID SNWRWVHNTG GYTNCYTGND |
| | | chrysogenum | WDRTLCPDDV TCATNCALDG ADYKGTYGVT ASGSSLRLNF VTQASQKNIG SRLYLMADDS KYEMFQLLNQ EFTFDVDVSN |
| | | | LPCGLNGALY FVAMDEDGGM ARYPTNKAGA KYGTGYCDAQ CPRDLKFING QANVEGWEPS SSDVNGGTGN YGSCCAEMDI |
| | | | WEANSISTAF TPHPCDDPAQ TRCTGDSCGG TYSSDRYGGT CDPDGCDFNP YRMGNQSFYG PSKIVDTESP FTVVTQFITN |
| | | | DGTSTGTLSE IKRFYVQNGK VIPQSVSTIS AVTGNSITDS FCSAQKTAFK DTDVFAKHGG MAGMGAGLAE GMVLVMSLWD |
| | | | DHAANMLWLD STYPTSASST TPGAARGSCD ISSGEPSDVE ANHSNAYVVY SNIKVGPLGS TFGSTDSGSG TTTTKVTTTT |
| | | | ATKTTTTTGP STTGAAHYAQ CGGQNWTGPT TCASPYTCQR QGDYYSQCL |
| SEQ ID NO: 36 | 171676762 | Podospora | MVSAKFAALA ALVASASAQQ VCSLTPESHP PLTWQRCSAG GSCTNVAGSV TLDSNWRWTH TLQGSTNCYS GNEWDTSICT |
| | | anserina | TGTKCAQNCC VEGAEYAATY GITTSGNQLN LKFVTEGKYS TNVGSRTYLM ENATKYQGFN LLGNEFTFDV DVSNIGCGLN |
| | | | GALYFVSMDL DGGLAKYSGN KAGAKYGTGY CDAQCPRDIK FINGEANIEG WNPSTNDVNA GAGRYGTCCS EMDIWEANNM |
| | | | ATAYTPHSCT ILDQSRCEGE SCGGTYSSDR YGGVCDPDGC DFNSYRMGNK EFYGKGKTVD TTKKMTVVTQ FLKNAAGELS |
| | | | EIKRFYVQNG VVIPNSVSSI PGVPNQNSIT QDWCDAQKIA FGDPDDNTAK GGLRQMGLAL DKPMVLVMSI WNDHAAHMLW |
| | | | LDSTYPVDAA GRPGAERGAC PTTSGVPSEV EAEAPNSNVA FSNIKFGPIG STFNSGSTNP NPISSSTATT PTSTRVSSTS |
| | | | TAAQTPTSAP GGTVPRWGQC GGQGYTGPTQ CVAPYTCVVS NQWYSQCL |
| SEQ ID NO: 37 | 146350520 | Pleurotus sp | MFPYIALVSF SFLSVVLAQQ VGTLTAETHP QLTVQQCTRG GSCTTQQRSV VLDGNWRWLH STSGSNNCYT GNTWDTSLCP |
| | | Florida | DAATCSRNCA LDGADYSGTY GITSSGNALT LKFVTHGPYS TNIGSRVYLL ADDSHYQMFN LKNKEFTFDV DVSQLPCGLN |
| | | | GALYFSQMDA DGGTGRFPNN KAGAKYGTGY CDSQCPHDIK FINGEANVQG WQPSPNDSNA GKGQYGSCCA EMDIWEANSM |
| | | | ASAYTPHPCT VTTPTRCQGN DCGDGDNRYG GVCDKDGCDF NSFRMGDKNF LGPGKTVNTN SKFTVVTQFL TSDNTTSGTL |
| | | | SEIRRLYVQN GRVIQNSKVN IPGMASTLDS ITESFCSTQK TVFGDTNSFA SKGGLRAMGN AFDKGMVLVL SIWDDHEAKM |
| | | | LWLDSNYPLD KSASAPGVAR GTCATTSGEP KDVESQSPNA QVIFSNIKYG DIGSTYSN |
| SEQ ID NO: 38 | 37732123 | Gibberella zeae | MYRAIATASA LIAAVRAQQV CSLTQESKPS LNWSKCTSSG CSNVKGSVTI DANWRWTHQV SGSTNCYTGN KWDTSVCTSG |
| | | | KVCAERCCLD GADYASTYGI TSSGDQLSLS FVTKGPYSTN IGSRTYLMED ENTYQMFQLL GNEFTFDVDV SNIGCGLNGA |
| | | | LYFVSMDADG GKAKYPGNKA GAKYGTGYCD AQCPRDVKFI NGQANSDGWQ PSDSDVNGGI GNLGTCCPEM DIWEANSIST |
| | | | AYTPHPCTKL TQHSCTGDSC GGTYSNDRYG GTCDADGCDF NSYRQGNKTF YGPGSGFNVD TTKKVTVVTQ FHKGSNGRLS |
| | | | EITRLYVQNG KVIANSESKI AGVPGNSLTA DFCTKQKKVF NDPDDFTKKG AWSGMSDALE APMVLVMSLW HDHHSNMLWL |
| | | | DSTYPTDSTK LGSQRGSCST SSGVPADLEK NVPNSKVAFS NIKFGPIGST YKSDGTTPTN PTNPSEPSNT ANPNPGTVDQ |
| | | | WGQCGGSNYS GPTACKSGFT CKKINDFYSQ CQ |
| SEQ ID NO: 39 | 156055188 | Sclerotinia | MYSAAVLATF SFLLGAGAQQ VGTLKTESHP PLTIQKCAAG GTCTDEADSV VLDANWRWLH STSGSTNCYT GNTWDTTLCP |
| | | sclerotiorum 1980 | DAATCTANCA FDGADYEGTY GITSSGDSLK LSFVTGSNVG SRTYLMDSET TYKEFALLGN EFTFTVDVSK LPCGLNGALY |
| | | | FVPMDADGGM SKYPTNKAGA KYGTGYCDAQ CPQDMKFVSG GANNEGWVPD SNSANSGTGN IGSCCSEFDV WEANSMSQAL |
| | | | TPHTCTVDGQ TACTGDDCAG NTGVCDADGC DFNPYRMGNT TFYGSGKTID TTKPFSVVTQ FITDDGTETG TLTEIKRFYV |
| | | | QDDVVYEQPN SDISGVSGNS ITDDFCTAQK TAFGDTDYFS QKGGMAAMGK KMADGMVLVL SIWDDYNVNM LWLDSDYPTT |
| | | | KDASTPGVSR GSCATTSGVP ATVEAASGSA YVTFSSIKYG PIGSTFKAPA DSSSPVVASS SPAAVAAVVS TSSAQAVPSH |
| | | | PAVSSSQAAV STPEAVSSAP EVPASSSAAQ SVAPTSTKPK CSKVSQSSTL ATSVAAPATT ATSAAVAATS AASSSGSVPL |
| | | | YGNCTGGKTC SEGTCVVQNP WYSQCVASS |
| SEQ ID NO: 40 | 453224 | Phanerochaete | MFRAAALLAF TCLAMVSGQQ AGTNTAENHP QLQSQQCTTS GGCKPLSTKV VLDSNWRWVH STSGYTNCYT GNEWDTSLCP |
| | | chrysosporium | DGKTCAANCA LDGADYSGTY GITSTGTALT LKFVTGSNVG SRVYLMADDT HYQLLKLLNQ EFTFDVDMSN LPCGLNGALY |
| | | | LSAMDADGGM SKYPGNKAGA KYGTGYCDSQ CPKDIKFING EANVGNWTET GSNTGTGSYG TCCSEMDIWE ANNDAAAFTP |
| | | | HPCTTTGQTR CSGDDCARNT GLCDGDGCDF NSFRMGDKTF LGKGMTVDTS KPFTVVTQFL TNDNTSTGTL SEIRRIYIQN |
| | | | GKVIQNSVAN IPGVDPVNSI TDNFCAQQKT AFGDTNWFAQ KGGLKQMGEA LGNGMVLALS IWDDHAANML WLDSDYPTDK |
| | | | DPSAPGVARG TCATTSGVPS DVESQVPNSQ VVFSNIKFGD IGSTFSGTSS PNPPGGSTTS SPVTTSPTPP PTGPTVPQWG |
| | | | QCGGIGYSGS TTCASPYTCH VLNPYYSQCY |
| SEQ ID NO: 41 | 50402144 | Trichoderma | MYRKLAVISA FLATARAQSA CTLQSETHPP LTWQKCSSGG TCTQQTGSVV IDANWRWTHA TNSSTNCYDG NTWSSTLCPD |
| | | reesei | NETCAKNCCL DGAAYASTYG VTTSGNSLSI GFVTQSAQKN VGARLYLMAS DTTYQEFTLL GNEFSFDVDV SQLPCGLNGA |
| | | | LYFVSMDADG GVSKYPTNTA GAKYGTGYCD SQCPRDLKFI NGQANVEGWE PSSNNANTGI GGHGSCCSEM DIWEANSISE |
| | | | ALTPHPCTTV GQEICEGDGC GGTYSDNRYG GTCDPDGCDW NPYRLGNTSF YGPGSSFTLD TTKKLTVVTQ FETSGAINRY |
| | | | YVQNGVTFQQ PNAELGSYSG NELNDDYCTA EEAEFGGSSF SDKGGLTQFK KATSGGMVLV MSLWDDYYAN MLWLDSTYPT |
| | | | NETSSTPGAV RGSCSTSSGV PAQVESQSPN AKVTFSNIKF GPIGSTGNPS GGNPPGGNRG TTTTRRPATT TGSSPGPTQS |
| | | | HYGQCGGIGY SGPTVCASGT TCQVLNPYYS QCL |
| SEQ ID NO: 42 | 115397177 | Aspergillus terreus | MPSTYDIYKK LLLLASFLSA SQAQQVGTSK AEVHPSLTWQ TCTSGGSCTT VNGKVVVDAN WRWVHNVDGY NNCYTGNTWD |
| | | NIH2624 | TTLCPDDETC ASNCALEGAD YSGTYGVTTS GNSLRLNFVT QASQKNIGSR LYLMEDDSTY KMFKLLNQEF TFDVDVSNLP |
| | | | CGLNGAVYFV SMDADGGMAK YPANKAGAKY GTGYCDSQCP RDLKFINGMA NVEGWEPSAN DANAGTGNHG SCCAEMDIWE |
| | | | ANSISTAYTP HPCDTPGQVM CTGDSCGGTY SSDRYGGTCD PDGCDFNSYR QGNKTFYGPG MTVDTKSKIT VVTQFLTNDG |
| | | | TASGTLSEIK RFYVQNGKVI PNSESTWSGV SGNSITTAYC NAQKTLFGDT DVFTKHGGME GMGAALAEGM VLVLSLWDDH |
| | | | NSNMLWLDSN YPTDKPSTTP GVARGSCDIS SGDPKDVEAN DANAYVVYSN IKVGPIGSTF SGSTGGGSSS STTATSKTTT |
| | | | TSATKTTTTT TKTTTTTSAS STSTGGAQHW AQCGGIGWTG PTTCVAPYTC QKQNDYYSQC L |
| SEQ ID NO: 43 | 154312003 | Botryotinia | MISKVLAFTS LLAAARAQQA GTLTTETHPP LSVSQCTASG CTTSAQSIVV DANWRWLHST TGSTNCYTGN TWDKTLCPDG |
| | | fuckeliana B05-10 | ATCAANCALD GADYSGVYGI TTSGNSIKLN FVTKGANTNV GSRTYLMAAG STTQYQMLKL LNQEFTFDVD VSNLPCGLNG |
| | | | ALYFAAMDAD GGLSRFPTNK AGAKYGTGYC DAQCPQDIKF INGVANSVGW TPSSNDVNAG AGQYGSCCSE MDIWEANKIS |
| | | | AAYTPHPCSV DTQTRCTGTD CGIGARYSSL CDADGCDFNS YRQGNTSFYG AGLTVNTNKV FTVVTQFITN DGTASGTLKE |
| | | | IRRFYVQNGV VIPNSQSTIA GVPGNSITDS FCAAQKTAFG DTNEFATKGG LATMSKALAK GMVLVMSIWD DHTANMLWLD |
| | | | APYPATKSPS APGVTRGSCS ATSGNPVDVE ANSPGSSVTF SNIKWGPINS TYTGSGAAPS VPGTTTVSSA PASTATSGAG |
| | | | GVAKYAQCGG SGYSGATACV SGSTCVALNP YYSQCQ |
| SEQ ID NO: 44 | 49333365 | Volvariella | MFPAATLFAF SLFAAVYGQQ VGTQLAETHP RLTWQKCTRS GGCQTQSNGA IVLDANWRWV HNVGGYTNCY TGNTWNTSLC |
| | | volvacea | PDGATCAKNC ALDGANYQST YGITTSGNAL TLKFVTQSEQ KNIGSRVYLL ESDTKYQLFN PLNQEFTFDV DVSQLPCGLN |
| | | | GAVYFSAMDA DGGMSKFPNN AAGAKYGTGY CDSQCPRDIK FINGEANVQG WQPSPNDTNA GTGNYGACCN EMDVWEANSI |
| | | | STAYTPHPCT QQGLVRCSGT ACGGGSNRYG SICDPDGCDF NSFRMGDKSF YGPGLTVNTQ QKFTVVTQFL TNNNSSSGTL |
| | | | REIRRLYVQN GRVIQNSKVN IPGMPSTMDS VTTEFCNAQK TAFNDTFSFQ QKGGMANMSE ALRRGMVLVL SIWDDHAANM |
| | | | LWLDSNYPTD RPASQPGVAR GTCPTSSGKP SDVENSTANS QVIYSNIKFG DIGSTYSA |
| SEQ ID NO: 45 | 729650 | Penicillium | MKGSISYQIY KGALLLSALL NSVSAQQVGT LTAETHPALT WSKCTAGXCS QVSGSVVIDA NWPXVHSTSG STNCYTGNTW |
| | | janthinellum | DATLCPDDVT CAANCAVDGA RRQHLRVTTS GNSLRINFVT TASQKNIGSR LYLLENDTTY QKFNLLNQEF TFDVDVSNLP |
| | | | CGLNGALYFV DMDADGGMAK YPTNKAGAKY GTGYCDSQCP RDLKFINGQA NVDGWTPSKN DVNSGIGNHG SCCAEMDIWE |
| | | | ANSISNAVTP HPCDTPSQTM CTGQRCGGTY STDRYGGTCD PDGCDFNPYR MGVTNFYGPG ETIDTKSPFT VVTQFLTNDG |
| | | | TSTGTLSEIK RFYVQGGKVI GNPQSTIVGV SGNSITDSWC NAQKSAFGDT NEFSKHGGMA GMGAGLADGM VLVMSLWDDH |
| | | | ASDMLWLDST YPTNATSTTP GAKRGTCDIS RRPNTVESTY PNAYVIYSNI KTGPLNSTFT GGTTSSSSTT TTTSKSTSTS |
| | | | SSSKTTTTVT TTTTSSGSSG TGARDWAQCG GNGWTGPTTC VSPYTCTKQN DWYSQCL |
| SEQ ID NO: 46 | 146424871 | Pleurotus sp | MFRTAALTAF TLAAVVLGQQ VGTLTAENHP ALSIQQCTAS GCTTQQKSVV LDSNWRWTHS LPVHTNCYTG NAWDASLCPD |
| | | Florida | PTTCATNCAI DGADYSGTYG ITTSGNALTL RFVTNGPYSK NIGSRVYLLD DADHYKMFDL KNQEFTFDVD MSGLPCGLNG |
| | | | ALYFSEMPAD GGKAAHTSNK AGAKYGTGYC DAQCPHDIKW INGEANILDW SASATDANAG NGRYGACCAE MDIWEANSEA |
| | | | TAYTPHVCRD EGLYRCSGTE CGDGDNRYGG VCDKDGCDFN SYRMGDKNFL GRGKTIDTTK KITVVTQFIT DDNTSSGNLV |
| | | | EIRRVYVQDG VTYQNSFSTF PSLSQYNSIS DDFCVAQKTL FGDNQYYNTH GGTEKMGDAM ANGMVLIMSL WSDHAAHMLW |
| | | | LDSDYPLDKS PSEPGVSRGA CATTTGDPDD VVANHPNASV TFSNIKYGPI GSTYGGSTPP VSSGNTSAPP VTSTTSSGPT |
| | | | TPTGPTGTVP KWGQCGGNGY SGPTTCVAGS TCTYSNDWYS QCL |
| SEQ ID NO: 47 | 67538012 | Aspergillus | MYQRALLFSA LLSVSRAQQA GTAQEEVHPS LTWQRCEASG SCTEVAGSVV LDSNWRWTHS VDGYTNCYTG NEWDATLCPD |
| | | nidulans FGSC A4 | NESCAQNCAV DGADYEATYG ITSNGDSLTL KFVTGSNVGS RVYLMEDDET YQMFDLLNNE FTFDVDVSNL PCGLNGALYF |
| | | | TSMDADGGLS KYEGNTAGAK YGTGYCDSQC PRDIKFINGL GNVEGWEPSD SDANAGVGGM GTCCPEMDIW EANSISTAYT |
| | | | PHPCDSVEQT MCEGDSCGGT YSDDRYGGTC DPDGCDFNSY RMGNTSFYGP GAIIDTSSKF TVVTQFIADG GSLSEIKRFY |
| | | | VQNGEVIPNS ESNISGVEGN SITSEFCTAQ KTAFGDEDIF AQHGGLSAMG DAASAMVLIL SIWDDHHSSM MWLDSSYPTD |
| | | | ADPSQPGVAR GTCEQGAGDP DVVESEHADA SVTFSNIKFG PIGSTF |
| SEQ ID NO: 48 | 62006162 | Fusarium poae | MYRAIATASA LIAAVRAQQV CSLTTETKPA LTWSKCTSSG CSNVQGSVTI DANWRWTHQV SGSTNCHTGN KWDTSVCTSG |
| | | | KVCAEKCCVD GADYASTYGI TSSGNQLSLS FVTKGSYGTN IGSRTYLMED ENTYQMFQLL GNEFTFDVDV SNIGCGLNGA |
| | | | LYFVSMDADG GKAKYPGNKA GAKYGTGYCD AQCPRDVKFI NGQANSDGWE PSKSDVNGGI GNLGTCCPEM DIWEANSIST |
| | | | AYTPHPCTKL TQHACTGDSC GGTYSNDRYG GTCDADGCDF NAYRQGNKTF YGPGSGFNVD TTKKVTVVTQ FHKGSNGRLS |
| | | | EITRLYVQNG KVIANSESKI AGNPGSSLTS DFCTTQKKVF GDIDDFAKKG AWNGMSDALE APMVLVMSLW HDHHSNMLWL |
| | | | DSTYPTDSTA LGSQRGSCST SSGVPADLEK NVPNSKVAFS NIKFGPIGST YNKEGTQPQP TNPTNPNPTN PTNPGTVDQW |
| | | | GQCGGTNYSG PTACKSPFTC KKINDFYSQC Q |
| SEQ ID NO: 49 | 146424873 | Pleurotus sp | MFRTAALTAF TLAAVVLGQQ VGTLAAENHP ALSIQQCTAS GCTTQQKSVV LDSNWRWTHS TAGATNCYTG NAWDSSLCPN |
| | | Florida | PTTCATNCAI DGADYSGTYG ITTSGNSLTL RFVTNGQYSE NIGSRVYLLD DADHYKLFNL KNQEFTFDVD MSGLPCGLNG |
| | | | ALYFSEMAAD GGKAAHTGNN AGAKYGTGYC DAQCPHDIKW INGEANILDW SGSATDPNAG NGRYGACCAE MDIWEANSEA |
| | | | TAYTPHVCRD EGLYRCSGTE CGDGDNRYGG VCDKDGCDFN SYRMGDKNFL GRGKTIDTTK KITVVTQFIT DDNTPTGNLV |
| | | | EIRRVYVQDG VTYQNSFSTF PSLSQYNSIS DDFCVAQKTL FGDNQYYNTH GGTEKMGDSL ANGMVLIMSL WSDHAAHMLW |
| | | | LDSDYPLDKS PSEPGVSRGA CATTTGDPDD VVANHPNASV TFSNIKYGPI GSTYGGSTPP VSSGNTSVPP VTSTTSSGPT |
| | | | TPTGPTGTVP KWGQCGGIGY SGPTSCVAGS TCTYSNEWYS QCL |
| SEQ ID NO: 50 | 295937 | Trichoderma | MYQKLALISA FLATARAQSA CTLQAETHPP LTWQKCSSGG TCTQQTGSVV IDANWRWTHA TNSSTNCYDG NTWSSTLCPD |
| | | viride | NETCAKNCCL DGAAYASTYG VTTSADSLSI GFVTQSAQKN VGARLYLMAS DTTYQEFTLL GNEFSFDVDV SQLPCGLNGA |
| | | | LYFVSMDADG GVTKYPTNTA GAKYGTGYCD SQCPRDLKFI NGQANVEGWE PSSNNANTGI GGHGSCCSEM DIWEANSISE |
| | | | ALTPHPCTTV GQEICEGDSC GGTYSGDRYG GTCDPDGCDW NPYRLGNTSF YGPGSSFTLD TTKKLTVVTQ FETSGAINRY |
| | | | YVQNGVTFQQ PNAELGDYSG NSLDDDYCAA EEAEFGGSSF SDKGGLTQFK KATSGGMVLV MSLWDDYYAN MLWLDSTYPT |
| | | | DETSSTPGAV RGSSSTSSGV PAQLESNSPN AKVVYSNIKF GPIGSTGNPS GGNPPGGNPP GTTTPRPATS TGSSPGPTQT |
| | | | HYGQCGGIGY IGPTVCASGS TCQVLNPYYS QCL |
| SEQ ID NO: 51 | 6179889 # | Alternaria | MTWQSCTAKG SCTNKNGKIV IDANWRWLHK KEGYDNCYTG NEWDATACPD NKACAANCAV DGADYSGTYG ITAGSNSLKL |
| | | alternata | KFITKGSYST NIGSRTYLMK DDTTYEMFKF TGNQEFTFDV DVSNLPCGFN GALYFVSMDA DGGLKKYSTN KAGAKYGTGY |
| | | | CDAQCPRDLK FINGEGNVEG WKPSSNDANA GVGGHGSCCA EMDIWEANSV STAVTPHSCS TIEQSRCDGD GCGGTYSADR |
| | | | YAGVCDPDGC DFNSYRMGVK DFYGKGKTVD TSKKFTVVTQ FIGTGDAMEI KRFYVQNGKT IAQPASAVPG VEGNSITTKF |
| | | | CDQQKAVFGD TYTFKDKGGM ANMAKALANG MVLVMSLWDD HYSNMLWLDS TYPTDKNPDT DLGTGRGECE TSSGVPADVE |
| | | | SQHADATVVY SNIKFGPLNS TFG |
| SEQ ID NO: 52 | 119483864 | Neosartorya | MASAISFQVY RSALILSAFL PSITQAQQIG TYTTETHPSM TWETCTSGGS CATNQGSVVM DANWRWVHQV GSTTNCYTGN |
| | | fischeri NRRL 181 | TWDTSICDTD ETCATECAVD GADYESTYGV TTSGSQIRLN FVTQNSNGAN VGSRLYMMAD NTHYQMFKLL NQEFTFDVDV |
| | | | SNLPCGLNGA LYFVTMDEDG GVSKYPNNKA GAQYGVGYCD SQCPRDLKFI QGQANVEGWT PSSNNENTGL GNYGSCCAEL |
| | | | DIWESNSISQ ALTPHPCDTA TNTMCTGDAC GGTYSSDRYA GTCDPDGCDF NPYRMGNTTF YGPGKTIDTN SPFTVVTQFI |
| | | | TDDGTDTGTL SEIRRYYVQN GVTYAQPDSD ISGITGNAIN ADYCTAENTV FDGPGTFAKH GGFSAMSEAM STGMVLVMSL |
| | | | WDDYYADMLW LDSTYPTNAS SSTPGAVRGS CSTDSGVPAT IESESPDSYV TYSNIKVGPI GSTFSSGSGS GSSGSGSSGS |
| | | | ASTSTTSTKT TAATSTSTAV AQHYSQCGGQ DWTGPTTCVS PYTCQVQNAY YSQCL |
| SEQ ID NO: 53 | 85083281 | Neurospora crassa | MKAYFEYLVA ALPLLGLATA QQVGKQTTET HPKLSWKKCT GKANCNTVNA EVVIDSNWRW LHDSSGKNCY DGNKWTSACS |
| | | OR74A | SATDCASKCQ LDGANYGTTY GASTSGDALT LKFVTKHEYG TNIGSRFYLM NGASKYQMFT LMNNEFAFDV DLSTVECGLN |
| | | | AALYFVAMEE DGGMASYSSN KAGAKYGTGY CDAQCARDLK FVGGKANIEG WTPSTNDANA GVGPYGGCCA EIDVWESNAH |
| | | | SFAFTPHACK TNKYHVCERD NCGGTYSEDR FAGLCDANGC DYNPYRMGNT DFYGKGKTVD TSKKFTVVSR FEENKLTQFF |
| | | | VQNGQKIEIP GPKWDGIPSD NANITPEFCS AQFQAFGDRD RFAEVGGFAQ LNSALRMPMV LVMSIWDDHY ANMLWLDSVY |
| | | | PPEKEGQPGA ARGDCPQSSG VPAEVESQYA NSKVVYSNIR FGPVGSTVNV |
| SEQ ID NO: 54 | 3913803 | Cryphonectria | MFSKFALTGS LLAGAVNAQG VGTQQTETHP QMTWQSCTSP SSCTTNQGEV VIDSNWRWVH DKDGYVNCYT GNTWNTTLCP |
| | | parasitica | DDKTCAANCV LDGADYSSTY GITTSGNALS LQFVTQSSGK NIGSRTYLME SSTKYHLFDL IGNEFAFDVD LSKLPCGLNG |
| | | | ALYFVTMDAD GGMAKYSTNT AGAEYGTGYC DSQCPRDLKF INGQGNVEGW TPSTNDANAG VGGLGSCCSE MDVWEANSMD |
| | | | MAYTPHPCET AAQHSCNADE CGGTYSSSRY AGDCDPDGCD WNPFRMGNKD FYGSGDTVDT SQKFTVVTQF HGSGSSLTEI |
| | | | SQYYIQGGTK IQQPNSTWPT LTGYNSITDD FCKAQKVEFN DTDVFSEKGG LAQMGAGMAD GMVLVMSLWD DHYANMLWLD |
| | | | STYPVDADAS SPGKQRGTCA TTSGVPADVE SSDASATVIY SNIKFGPIGA TY |
| SEQ ID NO: 55 | 60729633 | Corticium rolfsii | MFPAAALLSF TLLAVASAQQ IGTNTAEVHP SLTVSQCTTS GGCTSSTQSI VLDANWRWLH STSGYTNCYT GNQWNSDLCP |
| | | | DPDTCATNCA LDGASYESTY GISTDGNAVT LNFVTQGSQT NVGSRVYLLS DDTHYQTFSL LNKEFSFDVD ASNIGCGING |
| | | | AVYFVQMDAD GGLSKYSSNK AGAQYGTGYC DSQCPQDIKF INGEANLLDW NATSANSGTG SYGSCCPEMD IWEANKYAAA |
| | | | YTPHPCSVSG QTRCTGTSCG AGSERYDGYC DKDGCDFNSW RMGNETFLGP GMTIDTNKKF TIVTQFITDD NTANGTLSEI |
| | | | RRLYVQGGTV IQNSVANQPN IPKVNSITDS FCTAQKTEFG DQDYFGTIGG LSQMGKAMSD MVLVMSIWDD YDAEMLWLDS |
| | | | NYPTSGSAST PGISRGPCSA TSGLPATVES QQASASVTYS NIKWGDIGST YSGSGSSGSS SSSSSSAASA STSTHTSAAA |
| | | | TATSSAAAAT GSPVPAYGQC GGQSYTGSTT CASPYVCKVS NAYYSQCLPA |
| SEQ ID NO: 56 | 39971383 | Magnaporthe | MKRALCASLS LLAAAVAQQV GTNEPEVHPK MTWKKCSSGG SCSTVNGEVV IDGNWRWIHN IGGYENCYSG NKWTSVCSTN |
| | | grisea 70-15 | ADCATKCAME GAKYQETYGV STSGDALTLK FVQQNSSGKN VGSRMYLMNG ANKYQMFTLK NNEFAFDVDL SSVECGMNSA |
| | | | LYFVPMKEDG GMSTEPNNKA GAKYGTGYCD AQCARDLKFI GGKGNIEGWQ PSSTDSSAGI GAQGACCAEI DIWESNKNAF |
| | | | AFTPHPCENN EYHVCTEPNC GGTYADDRYG GGCDANGCDY NPYRMGNPDF YGPGKTIDTN RKFTVISRFE NNRNYQILMQ |
| | | | DGVAHRIPGP KFDGLEGETG ELNEQFCTDQ FTVFDERNRF NEVGGWSKLN AAYEIPMVLV MSIWSDHFAN MLWLDSTYPP |
| | | | EKAGQPGSAR GPCPADGGDP NGVVNQYPNA KVIWSNVRFG PIGSTYQVD |
| SEQ ID NO: 57 | 39973029 | Magnaporthe | MQLTKAGVFL GALMGGAAAQ QVGTQTAENH PKMTWKKCTG KASCTTVNGE VVIDANWRWL HDASSKNCYD GNRWTDSCRT |
| | | grisea 70-15 | ASDCAAKCSL EGADYAKTYG ASTSGDALSL KFVTRHDYGT NIGSRFYLMN GASKYQMFSL LGNEFAFDVD LSTIECGLNS |
| | | | ALYFVAMEED GGMKSYSSNK AGAKYGTGYC DAQCARDLKF VGGKANIEGW KPSSNDANAG VGPYGACCAE IDVWESNAHA |
| | | | FAFTPHPCTD NKYHVCQDSN CGGTYSDDRF AGKCDANGCD INPYRLGNTD FYGKGKTVDT SKKFTVVTRF ERDALTQFFV |
| | | | QNNKRIDMPS PALEGLPATG AITAEYCTNV FNVFGDRNRF DEVGGWSQLQ QALSLPMVLV MSIWDDHYSN MLWLDSVYPP |
| | | | DKEGSPGAAR GDCPQDSGVP SEVESQIPGA TVVWSNIRFG PVGSTVNV |
| SEQ ID NO: 58 | 1170141 | Fusarium | MYRIVATASA LIAAARAQQV CSLNTETKPA LTWSKCTSSG CSDVKGSVVI DANWRWTHQT SGSTNCYTGN KWDTSICTDG |
| | | oxysporum | KTCAEKCCLD GADYSGTYGI TSSGNQLSLG FVTNGPYSKN IGSRTYLMEN ENTYQMFQLL GNEFTFDVDV SGIGCGLNGA |
| | | | PHFVSMDEDG GKAKYSGNKA GAKYGTGYCD AQCPRDVKFI NGVANSEGWK PSDSDVNAGV GNLGTCCPEM DIWEANSIST |
| | | | AFTPHPCTKL TQHSCTGDSC GGTYSSDRYG GTCDADGCDF NAYRQGNKTF YGPGSNFNID TTKKMTVVTQ FHKGSNGRLS |
| | | | EITRLYVQNG KVIANSESKI AGNPGSSLTS DFCSKQKSVF GDIDDFSKKG GWNGMSDALS APMVLVMSLW HDHHSNMLWL |
| | | | DSTYPTDSTK VGSQRGSCAT TSGKPSDLER DVPNSKVSFS NIKFGPIGST YKSDGTTPNP PASSSTTGSS TPTNPPAGSV |
| | | | DQWGQCGGQN YSGPTTCKSP FTCKKINDFY SQCQ |
| SEQ ID NO: 59 | 121710012 | Aspergillus | MYQRALLFSA LATAVSAQQV GTQKAEVHPA LTWQKCTAAG SCTDQKGSVV IDANWRWLHS TEDTTNCYTG NEWNAELCPD |
| | | clavatus NRRL 1 | NEACAKNCAL DGADYSGTYG VTADGSSLKL NFVTSANVGS RLYLMEDDET YQMFNLLNNE FTFDVDVSNL PCGLNGALYF |
| | | | VSMDADGGLS KYPGNKAGAK YGTGYCDSQC PRDLKFINGE ANVEGWKPSD NDKNAGVGGY GSCCPEMDIW EANSISTAYT |
| | | | PHPCDGMEQT RCDGNDCGGT YSSTRYAGTC DPDGCDFNSF RMGNESFYGP GGLVDTKSPI TVVTQFVTAG GTDSGALKEI |
| | | | RRVYVQGGKV IGNSASNVAG VEGDSITSDF CTAQKKAFGD EDIFSKHGGL EGMGKALNKM ALIVSIWDDH ASSMMWLDST |
| | | | YPVDADASTP GVARGTCEHG LGDPETVESQ HPDASVTFSN IKFGPIGSTY KSV |
| SEQ ID NO: 60 | 17902580 | Penicillium | MSALNSFNMY KSALILGSLL ATAGAQQIGT YTAETHPSLS WSTCKSGGSC TTNSGAITLD ANWRWVHGVN TSTNCYTGNT |
| | | funiculosum | WNTAICDTDA SCAQDCALDG ADYSGTYGIT TSGNSLRLNF VTGSNVGSRT YLMADNTHYQ IFDLLNQEFT FTVDVSNLPC |
| | | | GLNGALYFVT MDADGGVSKY PNNKAGAQYG VGYCDSQCPR DLKFIAGQAN VEGWTPSTNN SNTGIGNHGS CCAELDIWEA |
| | | | NSISEALTPH PCDTPGLTVC TADDCGGTYS SNRYAGTCDP DGCDFNPYRL GVTDFYGSGK TVDTTKPFTV VTQFVTDDGT |
| | | | SSGSLSEIRR YYVQNGVVIP QPSSKISGIS GNVINSDFCA AELSAFGETA SFTNHGGLKN MGSALEAGMV LVMSLWDDYS |
| | | | VNMLWLDSTY PANETGTPGA ARGSCPTTSG NPKTVESQSG SSYVVFSDIK VGPFNSTFSG GTSTGGSTTT TASGTTSTKA |
| | | | STTSTSSTST GTGVAAHWGQ CGGQGWTGPT TCASGTTCTV VNPYYSQCL |
| SEQ ID NO: 61 | 1346226 | Humicola grisea | MRTAKFATLA ALVASAAAQQ ACSLTTERHP SLSWNKCTAG GQCQTVQASI TLDSNWRWTH QVSGSTNCYT GNKWDTSICT |
| | | var thermoidea | DAKSCAQNCC VDGADYTSTY GITTNGDSLS LKFVTKGQHS TNVGSRTYLM DGEDKYQTFE LLGNEFTFDV DVSNIGCGLN |
| | | | GALYFVSMDA DGGLSRYPGN KAGAKYGTGY CDAQCPRDIK FINGEANIEG WTGSTNDPNA GAGRYGTCCS EMDIWEANNM |
| | | | ATAFTPHPCT IIGQSRCEGD SCGGTYSNER YAGVCDPDGC DFNSYRQGNK TFYGKGMTVD TTKKITVVTQ FLKDANGDLG |
| | | | EIKRFYVQDG KIIPNSESTI PGVEGNSITQ DWCDRQKVAF GDIDDFNRKG GMKQMGKALA GPMVLVMSIW DDHASNMLWL |
| | | | DSTFPVDAAG KPGAERGACP TTSGVPAEVE AEAPNSNVVF SNIRFGPIGS TVAGLPGAGN GGNNGGNPPP PTTTTSSAPA |
| | | | TTTTASAGPK AGRWQQCGGI GFTGPTQCEE PYICTKLNDW YSQCL |
| SEQ ID NO: 62 | 156712282 | Chaetomium | MMYKKFAALA ALVAGASAQQ ACSLTAENHP SLTWKRCTSG GSCSTVNGAV TIDANWRWTH TVSGSTNCYT GNQWDTSLCT |
| | | thermophilum | DGKSCAQTCC VDGADYSSTY GITTSGDSLN LKFVTKHQYG TNVGSRVYLM ENDTKYQMFE LLGNEFTFDV DVSNLGCGLN |
| | | | GALYFVSMDA DGGMSKYSGN KAGAKYGTGY CDAQCPRDLK FINGEANVGN WTPSTNDANA GFGRYGSCCS EMDVWEANNM |
| | | | ATAFTPHPCT TVGQSRCEAD TCGGTYSSDR YAGVCDPDGC DFNAYRQGDK TFYGKGMTVD TNKKMTVVTQ FHKNSAGVLS |
| | | | EIKRFYVQDG KIIANAESKI PGNPGNSITQ EYCDAQKVAF SNTDDFNRKG GMAQMSKALA GPMVLVMSVW DDHYANMLWL |
| | | | DSTYPIDQAG APGAERGACP TTSGVPAEIE AQVPNSNVIF SNIRFGPIGS TVPGLDGSNP GNPTTTVVPP ASTSTSRPTS |
| | | | STSSPVSTPT GQPGGCTTQK WGQCGGIGYT GCTNCVAGTT CTQLNPWYSQ CL |
| SEQ ID NO: 63 | 169768818 | Aspergillus oryzae | MASLSLSKIC RNALILSSVL STAQGQQVGT YQTETHPSMT WQTCGNGGSC STNQGSVVLD ANWRWVHQTG SSSNCYTGNK |
| | | RIB40 | WDTSYCSTND ACAQKCALDG ADYSNTYGIT TSGSEVRLNF VTSNSNGKNV GSRVYMMADD THYEVYKLLN QEFTFDVDVS |
| | | | KLPCGLNGAL YFVVMDADGG VSKYPNNKAG AKYGTGYCDS QCPRDLKFIQ GQANVEGWVS STNNANTGTG NHGSCCAELD |
| | | | IWESNSISQA LTPHPCDTPT NTLCTGDACG GTYSSDRYSG TCDPDGCDFN PYRVGNTTFY GPGKTIDTNK PITVVTQFIT |
| | | | DDGTSSGTLS EIKRFYVQDG VTYPQPSADV SGLSGNTINS EYCTAENTLF EGSGSFAKHG GLAGMGEAMS TGMVLVMSLW |
| | | | DDYYANMLWL DSNYPTNEST SKPGVARGTC STSSGVPSEV EASNPSAYVA YSNIKVGPIG STFKS |
| SEQ ID NO: 64 | 46241270 | Gibberella | MYRAIATASA LIAAVRAQQV CSLTPETKPA LSWSKCTSSG CSNVQGSVTI DANWRWTHQL SGSTNCYTGN KWDTSICTSG |
| | | pulicaris | KVCAEKCCID GAEYASTYGI TSSGNQLSLS FVTKGAYGTN IGSRTYLMED ENTYQMFQLL GNEFTFDVDV SNIGCGLNGA |
| | | | LYFVSMDADG GKAKYPGNKA GAKYGTGYCD AQCPRDVKFI NGQANSDGWQ PSKSDVNAGI GNMGTCCPEM DIWEANSIST |
| | | | AYTPHPCTKL TQHSCTGDSC GGTYSNDRYG GTCDADGCDF NAYRQGNKTF YGPGSGFNVD TTKKVTVVTQ FHKGSNGRLS |
| | | | EITRLYVQNG KVIANSESKI AGVPGSSLTP EFCTAQKKVF GDTDDFAKKG AWSGMSDALE APMVLVMSLW HDHHSNMLWL |
| | | | DSTYPTDSTK LGAQRGSCST SSGVPADLEK NVPNSKVAFS NIKFGPIGST YKEGVPEPTN PTNPTNPTNP TNPGTVDQWA |
| | | | QCGGTNYSGP TACKSPFTCK KINDFYSQCQ |
| SEQ ID NO: 65 | 49333363 | Volvariella | MFPKSSLLVL SFLATAYAQQ VGTQTAEVHP SLNWARCTSS GCTNVAGSVT LDANWRWLHT TSGYTNCYTG NSWNTTLCPD |
| | | volvacea | GATCAQNCAL DGANYQSTCG ITTSGNALTL KFVTQGEQKN IGSRVYLMAS ESRYEMFGLL NKEFTFDVDV SNLPCGLNGA |
| | | | LYFSSMDADG GMAKNPGNKA GAKYGTGYCD SQCPRDIKFI NGEANVAGWN GSPNDTNAGT GNWGACCNEM DIWEANSISA |
| | | | AYTPHPCTVQ GLSRCSGTAC GTNDRYGTVC DPDGCDFNSY RMGDKTYYGP GGTGVDTRSK FTVVTQFLTN NNSSSGTLSE |
| | | | IRRLYVQNGR VVQNSKVNIP GMSNTLDSIT TGFCDSQKTA FGDTRSFQNK GGMSAMGQAL GAGMVLVLSV WDDHAANMLW |
| | | | LDSNYPVDAD PSKPGIARGT CSTTSGKPTD VEQSAANSSV TFSNIKFGDI GTTYTGGSVT TTPGNPGTTT STAPGAVQTK |
| | | | WGQCGGQGWT GPTRCESGST CTVVNQWYSQ CI |
| SEQ ID NO: 66 | 46395332 | Irpex lacteus | MFRKAALLAF SFLAIAHGQQ VGTNQAENHP SLPSQHCTAS GCTTSSTSVV LDANWRWVHT TTGYTNCYTG QTWDASICPD |
| | | | GVTCAKACAL DGADYSGTYG ITTSGNALTL QFVKGTNVGS RVYLLQDASN YQLFKLINQE FTFDVDMSNL PCGLNGAVYL |
| | | | SQMDQDGGVS RFPTNTAGAK YGTGYCDSQC PRDIKFINGE ANVAGWTGSS SDPNSGTGNY GTCCSEMDIW EANSVAAAYT |
| | | | PHPCSVNQQT RCTGADCGQD ANRYKGVCDP DGCDFNSFRM GDQTFLGKGL TVDTSRKFTI VTQFISDDGT SSGNLAEIRR |
| | | | FYVQDGKVIP NSKVNIAGCD AVNSITDKFC TQQKTAFGDT NRFADQGGLK QMGAALKSGM VLALSLWDDH AANMLWLDSD |
| | | | YPTTADASKP GVARGTCPNT SGVPKDVESQ SGSATVTYSN IKWGDLNSTF SGTASNPTGP SSSPSGPSSS SSSTAGSQPT |
| | | | QPSSGSVAQW GQCGGIGYSG ATGCVSPYTC HVVNPYYSQC Y |
| SEQ ID NO: 67 | 50844407 # | Chaetomium | TETHPRLTWK RCTSGGNCST VNGAVTIDAN WRWTHTVSGS TNCYTGNEWD TSICSDGKSC AQTCCVDGAD YSSTYGITTS |
| | | thermophilum var | GDSLNLKFVT KHQHGTNVGS RVYLMENDTK YQMFELLGNE FTFDVDVSNL GCGLNGALYF VSMDADGGMS KYSGNKAGAK |
| | | thermophilum | YGTGYCDAQC PRDLKFINGE ANIENWTPST NDANAGFGRY GSCCSEMDIW EANNMATAFT PHPCTIIGQS RCEGNSCGGT |
| | | | YSSERYAGVC DPDGCDFNAY RQGDKTFYGK GMTVDTTKKM TVVTQFHKNS AGVLSEIKRF YVQDGKIIAN AESKIPGNPG |
| | | | NSITQEWCDA QKVAFGDIDD FNRKGGMAQM SKALEGPMVL VMSVWDDHYA NMLWLDSTYP IDKAGTPGAE RGACPTTSGV |
| | | | PAEIEAQVPN SNVIFSNIRF GPIGSTVPGL DGSTPSNPTA TVAPPTSTTT SVRSSTTQIS TPTSQPGGCT TQKWGQCGGI |
| | | | GYTGCTNCVA GTTCTELNPW YSQCL |
| SEQ ID NO: 68 | 4586347 | Irpex lacteus | MFHKAVLVAF SLVTIVHGQQ AGTQTAENHP QLSSQKCTAG GSCTSASTSV VLDSNWRWVH TTSGYTNCYT GNTWDASICS |
| | | | DPVSCAQNCA LDGADYAGTY GITTSGDALT LKFVTGSNVG SRVYLMEDET NYQMFKLMNQ EFTFDVDVSN LPCGLNGAVY |
| | | | FVQMDQDGGT SKFPNNKAGA KFGTGYCDSQ CPQDIKFING EANIVDWTAS AGDANSGTGS FGTCCQEMDI WEANSISAAY |
| | | | TPHPCTVTEQ TRCSGSDCGQ GSDRFNGICD PDGCDFNSFR MGNTEFYGKG LTVDTSQKFT IVTQFISDDG TADGNLAEIR |
| | | | RFYVQNGKVI PNSVVQITGI DPVNSITEDF CTQQKTVFGD TNNFAAKGGL KQMGEAVKNG MVLALSLWDD YAAQMLWLDS |
| | | | DYPTTADPSQ PGVARGTCPT TSGVPSQVEG QEGSSSVIYS NIKFGDLNST FTGTLTNPSS PAGPPVTSSP SEPSQSTQPS |
| | | | QPAQPTQPAG TAAQWAQCGG MGFTGPTVCA SPFTCHVLNP YYSQCY |
| SEQ ID NO: 69 | 3980202 | Phanerochaete | MFRAAALLAF TCLAMVSGQQ AGTNTAENHP QLQSQQCTTS GGCKPLSTKV VLDSNWRWVH STSGYTNCYT GNEWNTSLCP |
| | | chrysosporium | DGKTCAANCA LDGADYSGTY GITSTGTALT LKFVTGSNVG SRVYLMADDT HYQLLKLLNQ EFTFDVDMSN LPCGLNGALY |
| | | | LSAMDADGGM SKYPGNKAGA KYGTGYCDSQ CPKDIKFING EANVGNWTET GSNTGTGSYG TCCSEMDIWE ANNDAAAFTP |
| | | | HPCTTTGQTR CSGDDCARNT GLCDHGDGCD FNSFRMGDKT FLGKGMTVDT SKPFTDVTQF LTNDNTSTGT LSEIRRIYIQ |
| | | | NGKVIQNSVA NIPGVDPVNS ITDNFCAQQK TAFGDTNWFA QKGGLKQMGE ALGNGMVLAL SIWDDHAANM LWLDSDYPTD |
| | | | KDPSAPGVAR GTCATTSGVP SDVESQVPNS QVVFSNIKFG DIGSTFSGTS SPNPPGGSTT SSPVTTSPTP PPTGPTVPQW |
| | | | GQCGGIGYSG STTCASPYTC HVLNPYYSQC Y |
| SEQ ID NO: 70 | 27125837 | Melanocarpus | MMMKQYLQYL AAALPLVGLA AGQRAGNETP ENHPPLTWQR CTAPGNCQTV NAEVVIDANW RWLHDDNMQN CYDGNQWTNA |
| | | albomyces | CSTATDCAEK CMIEGAGDYL GTYGASTSGD ALTLKFVTKH EYGTNVGSRF YLMNGPDKYQ MFNLMGNELA FDVDLSTVEC |
| | | | GINSALYFVA MEEDGGMASY PSNQAGARYG TGYCDAQCAR DLKFVGGKAN IEGWKSSTSD PNAGVGPYGS CCAEIDVWES |
| | | | NAYAFAFTPH ACTTNEYHVC ETTNCGGTYS EDRFAGKCDA NGCDYNPYRM GNPDFYGKGK TLDTSRKFTV VSRFEENKLS |
| | | | QYFIQDGRKI EIPPPTWEGM PNSSEITPEL CSTMFDVFND RNRFEEVGGF EQLNNALRVP MVLVMSIWDD HYANMLWLDS |
| | | | IYPPEKEGQP GAARGDCPTD SGVPAEVEAQ FPDAQVVWSN IRFGPIGSTY DF |
| SEQ ID NO: 71 | 171696102 | Podospora | MYRSATFLTF ASLVLGQQVG TYTAERHPSM PIQVCTAPGQ CTRESTEVVL DANWRWTHIT NGYTNCYTGN EWNATACPDG |
| | | anserina | ATCAKNCAVD GADYSGTYGI TTPSSGALRL QFVKKNDNGQ NVGSRVYLMA SSDKYKLFNL LNKEFTFDVD VSKLPCGLNG |
| | | | AVYFSEMLED GGLKSFSGNK AGAKYGTGYC DSQCPQDIKF INGEANVEGW GGADGNSGTG KYGICCAEMD IWEANSDATA |
| | | | YTPHVCSVNE QTRCEGVDCG AGSDRYNSIC DKDGCDFNSY RLGNREFYGP GKTVDTTRPF TIVTQFVTDD GTDSGNLKSI |
| | | | HRYYVQDGNV IPNSVTEVAG VDQTNFISEG FCEQQKSAFG DNNYFGQLGG MRAMGESLKK MVLVLSIWDD HAVNMNWLDS |
| | | | IFPNDADPEQ PGVARGRCDP ADGVPATIEA AHPDAYVIYS NIKFGAINST FTAN |
| SEQ ID NO: 72 | 3913802 | Cochliobolus | MYRTLAFASL SLYGAARAQQ VGTSTAENHP KLTWQTCTGT GGTNCSNKSG SVVLDSNWRW AHNVGGYTNC YTGNSWSTQY |
| | | carbonum | CPDGDSCTKN CAIDGADYSG TYGITTSNNA LSLKFVTKGS FSSNIGSRTY LMETDTKYQM FNLINKEFTF DVDVSKLPCG |
| | | | LNGALYFVEM AADGGIGKGN NKAGAKYGTG YCDSQCPHDI KFINGKANVE GWNPSDADPN GGAGKIGACC PEMDIWEANS |
| | | | ISTAYTPHPC RGVGLQECSD AASCGDGSNR YDGQCDKDGC DFNSYRMGVK DFYGPGATLD TTKKMTVITQ FLGSGSSLSE |
| | | | IKRFYVQNGK VYKNSQSAVA GVTGNSITES FCTAQKKAFG DTSSFAALGG LNEMGASLAR GHVLIMSLWG DHAVNMLWLD |
| | | | STYPTDADPS KPGAARGTCP TTSGKPEDVE KNSPDATVVF SNIKFGPIGS TFAQPA |
| SEQ ID NO: 73 | 50403723 | Trichoderma | MYQKLALISA FLATARAQSA CTLQAETHPP LTWQKCSSGG TCTQQTGSVV IDANWRWTHA TNSSTNCYDG NTWSSTLCPD |
| | | viride | NETCAKNCCL DGAAYASTYG VTTSADSLSI GFVTQSAQKN VGARLYLMAS DTTYQEFTLL GNEFSFDVDV SQLPCGLNGA |
| | | | LYFVSMDADG GVSKYPTNTA GAKYGTGYCD SQCPRDLKFI NGQANVEGWE PSSNNANTGI GGHGSCCSEM DIWEANSISE |
| | | | ALTPHPCTTV GQEICDGDSC GGTYSGDRYG GTCDPDGCDW NPYRLGNTSF YGPGSSFTLD TTKKLTVVTQ FETSGAINRY |
| | | | YVQNGVTFQQ PNAELGDYSG NSLDDDYCAA EEAEFGGSSF SDKGGLTQFK KATSGGMVLV MSLWDDYYAN MLWLDSTYPT |
| | | | NETSSTPGAV RGSCSTSSGV PAQLESNSPN AKVVYSNIKF GPIGSTGNSS GGNPPGGNPP GTTTTRRPAT STGSSPGPTQ |
| | | | THYGQCGGIG YSGPTVCASG STCQVLNPYY SQCL |
| SEQ ID NO: 74 | 3913798 | Aspergillus | MVDSFSIYKT ALLLSMLATS NAQQVGTYTA ETHPSLTWQT CSGSGSCTTT SGSVVIDANW RWVHEVGGYT NCYSGNTWDS |
| | | aculeatus | SICSTDTTCA SECALEGATY ESTYGVTTSG SSLRLNFVTT ASQKNIGSRL YLLADDSTYE TFKLFNREFT FDVDVSNLPC |
| | | | GLNGALYFVS MDADGGVSRF PTNKAGAKYG TGYCDSQCPR DLKFIDGQAN IEGWEPSSTD VNAGTGNHGS CCPEMDIWEA |
| | | | NSISSAFTAH PCDSVQQTMC TGDTCGGTYS DTTDRYSGTC DPDGCDFNPY RFGNTNFYGP GKTVDNSKPF TVVTQFITHD |
| | | | GTDTGTLTEI RRLYVQNGVV IGNGPSTYTA ASGNSITESF CKAEKTLFGD TNVFETHGGL SAMGDALGDG MVLVLSLWDD |
| | | | HAADMLWLDS DYPTTSCASS PGVARGTCPT TTGNATYVEA NYPNSYVTYS NIKFGTLNST YSGTSSGGSS SSSTTLTTKA |
| | | | STSTTSSKTT TTTSKTSTTS SSSTNVAQLY GQCGGQGWTG PTTCASGTCTKQNDYYSQCL |
| SEQ ID NO: 75 | 66828465 | Dictyostelium | MYRILKSFIL LSLVNMSLSQ KIGKLTPEVH PPMTFQKCSE GGSCETIQGE VVVDANWRWV HSAQGQNCYT GNTWNPTICP |
| | | discoideum | DDETCAENCY LDGANYESVY GVTTSEDSVR LNFVTQSQGK NIGSRLFLMS NESNYQLFHV LGQEFTFDVD VSNLDCGLNG |
| | | | ALYLVSMDSD GGSARFPTNE AGAKYGTGYC DAQCPRDLKF ISGSANVDGW IPSTNNPNTG YGNLGSCCAE MDLWEANNMA |
| | | | TAVTPHPCDT SSQSVCKSDS CGGAASSNRY GGICDPDGCD YNPYRMGNTS FFGPNKMIDT NSVITVVTQF ITDDGSSDGK |
| | | | LTSIKRLYVQ DGNVISQSVS TIDGVEGNEV NEEFCTNQKK VFGDEDSFTK HGGLAKMGEA LKDGMVLVLS LWDDYQANML |
| | | | WLDSSYPTTS SPTDPGVARG SCPTTSGVPS KVEQNYPNAY VVYSNIKVGP IDSTYKK |
| SEQ ID NO: 76 | 156060391 | Sclerotinia | MISRVLAISS LLAAARAQQI GTNTAEVHPA LTSIVIDANW RWLHTTSGYT NCYTGNSWDA TLCPDAVTCA ANCALDGADY |
| | | sclerotiorum 1980 | SGTYGITTSG NSLKLNFVTK GANTNVGSRT YLMAAGSKTQ YQLLKLLGQE FTFDVDVSNL PCGLNGALYF AEMDADGGVS |
| | | | RFPTNKAGAQ YGTGYCDAQC PQDIKFINGQ ANSVGWTPSS NDVNTGTGQY GSCCSEMDIW EANKISAAYT PHPCSVDGQT |
| | | | RCTGTDCGIG ARYSSLCDAD GCDFNSYRMG DTGFYGAGLT VDTSKVFTVV TQFITNDGTT SGTLSEIRRF YVQNGKVIPN |
| | | | SQSKVTGVSG NSITDSFCAA QKTAFGDTNE FATKGGLATM SKALAKGMVL VMSIWDDHSA NMLWLDAPYP ASKSPSAAGV |
| | | | SRGSCSASSG VPADVEANSP GASVTYSNIK WGPINSTYSA GTGSNTGSGS GSTTTLVSSV PSSTPTSTTG VPKYGQCGGS |
| | | | GYTGPTNCIG STCVSMGQYY SQCQ |
| SEQ ID NO: 77 | 116181754 | Chaetomium | MYRQVATALS FASLVLGQQV GTLTAETHPS LPIEVCTAPG SCTKEDTTVV LDANWRWTHV TDGYTNCYTG NAWNETACPD |
| | | globosum CBS | GKTCAANCAI DGAEYEKTYG ITTPEEGALR LNFVTESNVG SRVYLMAGED KYRLFNLLNK EFTMDVDVSN LPCGLNGAVY |
| | | 148-51 | FSEMDEDGGM SRFEGNKAGA KYGTGYCDSQ CPRDIKFING EANSEGWGGE DGNSGTGKYG TCCAEMDIWE ANLDATAYTP |
| | | | HPCKVTEQTR CEDDTECGAG DARYEGLCDR DGCDFNSFRL GNKEFYGPEK TVDTSKPFTL VTQFVTADGT DTGALQSIRR |
| | | | FYVQDGTVIP NSETVVEGVD PTNEITDDFC AQQKTAFGDN NHFKTIGGLP AMGKSLEKMV LVLSIWDDHA VYMNWLDSNY |
| | | | PTDADPTKPG VARGRCDPEA GVPETVEAAH PDAYVIYSNI KIGALNSTFA AA |
| SEQ ID NO: 78 | 145230535 | Aspergillus niger | MSSFQVYRAA LLLSILATAN AQQVGTYTTE THPSLTWQTC TSDGSCTTND GEVVIDANWR WVHSTSSATN CYTGNEWDTS |
| | | | ICTDDVTCAA NCALDGATYE ATYGVTTSGS ELRLNFVTQG SSKNIGSRLY LMSDDSNYEL FKLLGQEFTF DVDVSNLPCG |
| | | | LNGALYFVAM DADGGTSEYS GNKAGAKYGT GYCDSQCPRD LKFINGEANC DGWEPSSNNV NTGVGDHGSC CAEMDVWEAN |
| | | | SISNAFTAHP CDSVSQTMCD GDSCGGTYSA SGDRYSGTCD PDGCDYNPYR LGNTDFYGPG LTVDTNSPFT VVTQFITDDG |
| | | | TSSGTLTEIK RLYVQNGEVI ANGASTYSSV NGSSITSAFC ESEKTLFGDE NVFDKHGGLE GMGEAMAKGM VLVLSLWDDY |
| | | | AADMLWLDSD YPVNSSASTP GVARGTCSTD SGVPATVEAE SPNAYVTYSN IKFGPIGSTY SSGSSSGSGS SSSSSSTTTK |
| | | | ATSTTLKTTS TTSSGSSSTS AAQAYGQCGG QGWTGPTTCV SGYTCTYENA YYSQCL |
| SEQ ID NO: 79 | 46241266 | Nectria | MYRAIATASA LLATARAQQV CTLNTENKPA LTWAKCTSSG CSNVRGSVVV DANWRWAHST SSSTNCYTGN TWDKTLCPDG |
| | | haematococca | KTCADKCCLD GADYSGTYGV TSSGNQLNLK FVTVGPYSTN VGSRLYLMED ENNYQMFDLL GNEFTFDVDV NNIGCGLNGA |
| | | mpVI | LYFVSMDKDG GKSRFSTNKA GAKYGTGYCD AQCPRDVKFI NGVANSDEWK PSDSDKNAGV GKYGTCCPEM DIWEANKIST |
| | | | AYTPHPCKSL TQQSCEGDAC GGTYSATRYA GTCDPDGCDF NPYRQGNKTF YGPGSGFNVD TTKKVTVVTQ FIKGSDGKLS |
| | | | EIKRLYVQNG KVIGNPQSEI ANNPGSSVTD SFCKAQKVAF NDPDDFNKKG GWSGMSDALA KPMVLVMSLW HDHYANMLWL |
| | | | DSTYPKGSKT PGSARGSCPE DSGDPDTLEK EVPNSGVSFS NIKFGPIGST YTGTGGSNPD PEEPEEPEEP VGTVPQYGQC |
| | | | GGINYSGPTA CVSPYKCNKI NDFYSQCQ |
| SEQ ID NO: 80 | 1q9h (PDB) # | Talaromyces | EQAGTATAEN HPPLTWQECT APGSCTTQNG AVVLDANWRW VHDVNGYTNC YTGNTWDPTY CPDDETCAQN CALDGADYEG |
| | | emersonii | TYGVTSSGSS LKLNFVTGSN VGSRLYLLQD DSTYQIFKLL NREFSFDVDV SNLPCGLNGA LYFVAMDADG GVSKYPNNKA |
| | | | GAKYGTGYCD SQCPRDLKFI DGEANVEGWQ PSSNNANTGI GDHGSCCAEM DVWEANSISN AVTPHPCDTP GQTMCSGDDC |
| | | | GGTYSNDRYA GTCDPDGCDF NPYRMGNTSF YGPGKIIDTT KPFTVVTQFL TDDGTDTGTL SEIKRFYIQN SNVIPQPNSD |
| | | | ISGVTGNSIT TEFCTAQKQA FGDTDDFSQH GGLAKMGAAM QQGMVLVMSL WDDYAAQMLW LDSDYPTDAD PTTPGIARGT |
| | | | CPTDSGVPSD VESQSPNSYV TYSNIKFGPI NSTFTAS |
| SEQ ID NO: 81 | 157362170 | Polyporus | MFPTLALVSL SFLAIAYGQQ VGTLTAETHP KLSVSQCTAG GSCTTVQRSV VLDSNWRWLH DVGGSTNCYT GNTWDDSLCP |
| | | arcularius | DPTTCAANCA LDGADYSGTY GITTSGNALS LKFVTQGPYS TNIGSRVYLL SEDDSTYEMF NLKNQEFTFD VDMSALPCGL |
| | | | NGALYFVEMD KDGGSGRFPT NKAGSKYGTG YCDTQCPHDI KFINGEANVL DWAGSSNDPN AGTGHYGTCC NEMDIWEANS |
| | | | MGAAVTPHVC TVQGQTRCEG TDCGDGDERY DGICDKDGCD FNSWRMGDQT FLGPGKTVDT SSKFTVVTQF ITADNTTSGD |
| | | | LSEIRRLYVQ NGKVIANSKT QIAGMDAYDS ITDDFCNAQK TTFGDTNTFE QMGGLATMGD AFETGMVLVM SIWDDHEAKM |
| | | | LWLDSDYPTD ADASAPGVSR GPCPTTSGDP TDVESQSPGA TVIFSNIKTG PIGSTFTS |
| SEQ ID NO: 82 | 7804885 | Leptosphaeria | MLSASKAAAI LAFCAHTASA WVVGDQQTET HPKLNWQRCT GKGRSSCTNV NGEVVIDANW RWLAHRSGYT NCYTGSEWNQ |
| | | maculans | SACPNNEACT KNCAIEGSDY AGTYGITTSG NQMNIKFITK RPYSTNIGAR TYLMKDEQNY EMFQLIGNEF TFDVDLSQRC |
| | | | GMNGALYFVS MPQKGQGAPG AKYGTGYCDA QCARDLKFVR GSANAEGWTK SASDPNSGVG KKGACCAQMD VWEANSAATA |
| | | | LTPHSCQPAG YSVCEDTNCG GTYSEDRYAG TCDANGCDFN PFRVGVKDFY GKGKTVDTTK KMTVVTQFVG SGNQLSEIKR |
| | | | FYVQDGKVIA NPEPTIPGME WCNTQKKVFQ EEAYPFNEFG GMASMSEGMS QGMVLVMSLW DDHYANMLWL DSNWPREADP |
| | | | AKPGVARRDC PTSGGKPSEV EAANPNAQVM FSNIKFGPIG STFAHAA |
| SEQ ID NO: 83 | 121852 | Phanerochaete | MFRTATLLAF TMAAMVFGQQ VGTNTARSHP ALTSQKCTKS GGCSNLNTKI VLDANWRWLH STSGYTNCYT GNQWDATLCP |
| | | chrysosporium | DGKTCAANCA LDGADYTGTY GITASGSSLK LQFVTGSNVG SRVYLMADDT HYQMFQLLNQ EFTFDVDMSN LPCGLNGALY |
| | | | LSAMDADGGM AKYPTNKAGA KYGTGYCDSQ CPRDIKFING EANVEGWNAT SANAGTGNYG TCCTEMDIWE ANNDAAAYTP |
| | | | HPCTTNAQTR CSGSDCTRDT GLCDADGCDF NSFRMGDQTF LGKGLTVDTS KPFTVVTQFI TNDGTSAGTL TEIRRLYVQN |
| | | | GKVIQNSSVK IPGIDPVNSI TDNFCSQQKT AFGDTNYFAQ HGGLKQVGEA LRTGMVLALS IWDDYAANML WLDSNYPTNK |
| | | | DPSTPGVARG TCATTSGVPA QIEAQSPNAY VVFSNIKFGD LNTTYTGTVS SSSVSSSHSS TSTSSSHSSS STPPTQPTGV |
| | | | TVPQWGQCGG IGYTGSTTCA SPYTCHVLNP YYSQCY |
| SEQ ID NO: 84 | 126013214 | Penicillium | MYQRALLFSA LMAGVSAQQV GTQKPETHPP LAWKECTSSG CTSKDGSVVI DANWRWVHSV DGYKNCYTGN EWDSTLCPDD |
| | | decumbens | ATCATNCAVD GADYAGTYGA TTEGDSLSIN FVTGSNIGSR FYLMEDENKY QMFKLLNKEF TFDVDVSTLP CGLNGALYFV |
| | | | SMDADGGMSK YETNKAGAKY GTGYCDSQCP RDLKFINGKG NVEGWKPSAN DKNAGVGPHG SCCAEMDIWE ANSISTALTP |
| | | | HPCDTNGQTI CEGDSCGGTY STTRYAGTCD PDGCDFNPFR MGNESFYGPG KMVDTKSKMT VVTQFITSDG TDTGSLKEIK |
| | | | RVYVQNGKVI ANSASDVSGI TGNSITSDFC TAQKKTFGDE DVFNKHGGLS GMGDALGEGM VLVMSLWDDH NSNMLWLDGE |
| | | | KYPTDAAASK AGVSRGTCST DSGKPSTVES ESGSAKVVFS NIKVGSIGST FSA |
| SEQ ID NO: 85 | 156048578 | Sclerotinia | MTSKIALASL FAAAYGQQIG TYTTETHPSL TWQSCTAKGS CTTQSGSIVL DGNWRWTHST TSSTNCYTGN TWDATLCPDD |
| | | sclerotiorum 1980 | ATCAQNCALD GADYSGTYGI TTSGDSLRLN FVTQTANKNV GSRVYLLADN THYKTFNLLN QEFTFDVDVS NLPCGLNGAV |
| | | | YFANLPADGG ISSTNKAGAQ YGTGYCDSQC PRDGKFINGK ANVDGWVPSS NNPNTGVGNY GSCCAEMDIW EANSISTAVT |
| | | | PHSCDTVTQT VCTGDNCGGT YSTTRYAGTC DPDGCDFNPY RQGNESFYGP GKTVDTNSVF TIVTQFLTTD GTSSGTLNEI |
| | | | KRFYVQNGKV IPNSESTISG VTGNSITTPF CTAQKTAFGD PTSFSDHGGL ASMSAAFEAG MVLVLSLWDD YYANMLWLDS |
| | | | TYPTTKTGAG GPRGTCSTSS GVPASVEASS PNAYVVYSNI KVGAINSTFG |
| SEQ ID NO: 86 | 156712278 | Acremonium | MYTKFAALAA LVATVRGQAA CSLTAETHPS LQWQKCTAPG SCTTVSGQVT IDANWRWLHQ TNSSTNCYTG NEWDTSICSS |
| | | thermophilum | DTDCATKCCL DGADYTGTYG VTASGNSLNL KFVTQGPYSK NIGSRMYLME SESKYQGFTL LGQEFTFDVD VSNLGCGLNG |
| | | | ALYFVSMDLD GGVSKYTTNK AGAKYGTGYC DSQCPRDLKF INGQANIDGW QPSSNDANAG LGNHGSCCSE MDIWEANKVS |
| | | | AAYTPHPCTT IGQTMCTGDD CGGTYSSDRY AGICDPDGCD FNSYRMGDTS FYGPGKTVDT GSKFTVVTQF LTGSDGNLSE |
| | | | IKRFYVQNGK VIPNSESKIA GVSGNSITTD FCTAQKTAFG DTNVFEERGG LAQMGKALAE PMVLVLSVWD DHAVNMLWLD |
| | | | STYPTDSTKP GAARGDCPIT SGVPADVESQ APNSNVIYSN IRFGPINSTY TGTPSGGNPP GGGTTTTTTT TTSKPSGPTT |
| | | | TTNPSGPQQT HWGQCGGQGW TGPTVCQSPY TCKYSNDWYS QCL |
| SEQ ID NO: 87 | 21449327 | Aspergillus | MYQRALLFSA LLSVSRAQQA GTAQEEVHPS LTWQRCEASG SCTEVAGSVV LDSNWRWTHS VDGYTNCYTG NEWDATLCPD |
| | | nidulans (also | NESCAQNCAV DGADYEATYG ITSNGDSLTL KFVTGSNVGS RVYLMEDDET YQMFDLLNNE FTFDVDVSNF PCGLNGALYF |
| | | known as | TSMDADGGLS KYEGNTAGAK YGTGYCDSQC PRDIKFINGL GNVEGWEPSD SDANAGVGGM GTCCPEMDIW EANSISTAYT |
| | | Emericella | PHPCDSVEQT MCEGDSCGGT YSDDRYGGTC DPDGCDFNSY RMGNTRFYGP GAIIDTSSKF TVVTQFIADG GSLSEIKRFY |
| | | nidulans) | VQNGEVIPNS ESNISGVEGN SITSEFCTAQ KTAFGDEDIF AQHGGLSAMG DAASAMVLIL SIWDDHHSSM MWLDSSYPTD |
| | | | ADPSQPGVAR GTCEQGAGDP DVVESEHADA SVTFSNIKFG PIGSTF |
| SEQ ID NO: 88 | 171683762 | Podospora | MMMKQYLQYL AAGSLMTGLV AGQGVGTQQT ETHPRITWKR CTGKANCTTV QAEVVIDSNW RWIHTSGGTN CYDGNAWNTA |
| | | anserine (S mat+) | ACSTATDCAS KCLMEGAGNY QQTYGASTSG DSLTLKFVTK HEYGTNVGSR FYLMNGASKY QMFTLMNNEF TFDVDLSTVE |
| | | | CGLNSALYFV AMEEDGGMRS YPTNKAGAKY GTGYCDAQCA RDLKFVGGKA NIEGWRESSN DENAGVGPYG GCCAEIDVWE |
| | | | SNAHAYAFTP HACENNNYHV CERDTCGGTY SEDRFAGGCD ANGCDYNPYR MGNPDFYGKG KTVDTTKKFT VVTRFQDDNL |
| | | | EQFFVQNGQK ILAPAPTFDG IPASPNLTPE FCSTQFDVFT DRNRFREVGD FPQLNAALRI PMVLVMSIWA DHYANMLWLD |
| | | | SVYPPEKEGE PGAARGPCAQ DSGVPSEVKA NYPNAKVVWS NIRFGPIGST VNV |
| SEQ ID NO: 89 | 56718412 | Thermoascus | MYQRALLFSF FLAAARAQQA GTVTAENHPS LTWQQCSSGG SCTTQNGKVV IDANWRWVHT TSGYTNCYTG NTWDTSICPD |
| | | aurantiacus var | DVTCAQNCAL DGADYSGTYG VTTSGNALRL NFVTQSSGKN IGSRLYLLQD DTTYQIFKLL GQEFTFDVDV SNLPCGLNGA |
| | | levisporus | LYFVAMDADG GLSKYPGNKA GAKYGTGYCD SQCPRDLKFI NGQANVEGWQ PSANDPNAGV GNHGSCCAEM DVWEANSIST |
| | | | AVTPHPCDTP GQTMCQGDDC GGTYSSTRYA GTCDPDGCDF NPYRQGNHSF YGPGKIVDTS SKFTVVTQFI TDDGTPSGTL |
| | | | TEIKRFYVQN GKVIPQSEST ISGVTGNSIT TEYCTAQKAA FGDNTGFFTH GGLQKISQAL AQGMVLVMSL WDDHAANMLW |
| | | | LDSTYPTDAD PDTPGVARGT CPTTSGVPAD VESQNPNSYV IYSNIKVGPI NSTFTAN |
| SEQ ID NO: 90 | 15824273 | Pseudotrichonympha | MFAIVLLGLT RSLGTGTNQA ENHPSLSWQN CRSGGSCTQT SGSVVLDSNW RWTHDSSLTN CYDGNEWSSS LCPDPKTCSD |
| | | grassii | NCLIDGADYS GTYGITSSGN SLKLVFVTNG PYSTNIGSRV YLLKDESHYQ IFDLKNKEFT FTVDDSNLDC GLNGALYFVS |
| | | | MDEDGGTSRF SSNKAGAKYG TGYCDAQCPH DIKFINGEAN VENWKPQTND ENAGNGRYGA CCTEMDIWEA NKYATAYTPH |
| | | | ICTVNGEYRC DGSECGDTDS GNRYGGVCDK DGCDFNSYRM GNTSFWGPGL IIDTGKPVTV VTQFVTKDGT DNGQLSEIRR |
| | | | KYVQGGKVIE NTVVNIAGMS SGNSITDDFC NEQKSAFGDT NDFEKKGGLS GLGKAFDYGM VLVLSLWDDH QVNMLWLDSI |
| | | | YPTDQPASQP GVKRGPCATS SGAPSDVESQ HPDSSVTFSD IRFGPIDSTY |
| SEQ ID NO: 91 | 115390801 | Aspergillus terreus | MHQRALLFSA LVGAVRAQQA GTLTEEVHPP LTWQKCTADG SCTEQSGSVV IDSNWRWLHS TNGSTNCYTG NTWDESLCPD |
| | | NIH2624 | NEACAANCAL DGADYESTYG ITTSGDALTL TFVTGENVGS RVYLMAEDDE SYQTFDLVGN EFTFDVDVSN LPCGLNGALY |
| | | | FTSMDADGGV SKYPANKAGA KYGTGYCDSQ CPRDLKFING MANVEGWTPS DNDKNAGVGG HGSCCPELDI WEANSISSAF |
| | | | TPHPCDDLGQ TMCSGDDCGG TYSETRYAGT CDPDGCDFNA YRMGNTSYYG PDKIVDTNSV MTVVTQFIGD GGSLSEIKRL |
| | | | YVQNGKVIAN AQSNVDGVTG NSITSDFCTA QKTAFGDQDI FSKHGGLSGM GDAMSAMVLI LSIWDDHNSS MMWLDSTYPE |
| | | | DADASEPGVA RGTCEHGVGD PETVESQHPG ATVTFSKIKF GPIGSTYSSN STA |
| SEQ ID NO: 92 | 453223 | Phanerochaete | MFRAAALLAF TCLAMVSGQQ AGTNTAENHP QLQSQQCTTS GGCKPLSTKV VLDSNWRWVH STSGYTNCYT GNEWDTSLCP |
| | | chrysosporium | DGKTCAANCA LDGADYSGTY GITSTGTALT LKFVTGSNVG SRVYLMADDT HYQLLKLLNQ EFTFDVDMSN LPCGLNGALY |
| | | | LSAMDADGGM SKYPGNKAGA KYGTGYCDSQ CPKDIKFING EANVGNWTET GSNTGTGSYG TCCSEMDIWE ANNDAAAFTP |
| | | | HPCTTTGQTR CSGDDCARNT GLCDGDGCDF NSFRMGDKTF LGKGMTVDTS KPFTVVTQFL TNDNTSTGTL SEIRRIYIQN |
| | | | GKVIQNSVAN IPGVDPVNSI TDNFCAQQKT AFGDTNWFAQ KGGLKQMGEA LGNGMVLALS IWDDHAANML WLDSDYPTDK |
| | | | DPSAPGVARG TCATTSGVPS DVESQVPNSQ VVFSNIKFGD IGSTFSGTSS PNPPGGSTTS SPVTTSPTPP PTGPTVPQWG |
| | | | QCGGIGYSGS TTCASPYTCH VLNPCESILS LQRSSNADQY LQTTRSATKR RLDTALQPRK |
| SEQ ID NO: 93 | 3132 | Phanerochaete | MRTALALILA LAAFSAVSAQ QAGTITAETH PTLTIQQCTQ SGGCAPLTTK VVLDVNWRWI HSTTGYTNCY SGNTWDAILC |
| | | chrysosporium | PDPVTCAANC ALDGADYTGT FGILPSGTSV TLRPVDGLGL RLFLLADDSH YQMFQLLNKE FTFDVEMPNM RCGSSGAIHL |
| | | | TAMDADGGLA KYPGNQAGAK YGTGFCSAQC PKGVKFINGQ ANVEGWLGTT ATTGTGFFGS CCTDIALWEA NDNSASFAPH |
| | | | PCTTNSQTRC SGSDCTADSG LCDADGCNFN SFRMGNTTFF GAGMSVDTTK LFTVVTQFIT SDNTSMGALV EIHRLYIQNG |
| | | | QVIQNSVVNI PGINPATSIT DDLCAQENAA FGGTSSFAQH GGLAQVGEAL RSGMVLALSI VNSAADTLWL DSNYPADADP |
| | | | SAPGVARGTC PQDSASIPEA PTPSVVFSNI KLGDIGTTFG AGSALFSGRS PPGPVPGSAP ASSATATAPP FGSQCGGLGY |
| | | | AGPTGVCPSP YTCQALNIYY SQCI |
| SEQ ID NO: 94 | 16304152 | Thermoascus | MYQRALLFSF FLAAARAHEA GTVTAENHPS LTWQQCSSGG SCTTQNGKVV IDANWRWVHT TSGYTNCYTG NTWDTSICPD |
| | | aurantiacus | DVTCAQNCAL DGADYSGTYG VTTSGNALRL NFVTQSSGKN IGSRLYLLQD DTTYQIFKLL GQEFTFDVDV SNLPCGLNGA |
| | | | LYFVAMDADG NLSKYPGNKA GAKYGTGYCD SQCPRDLKFI NGQANVEGWQ PSANDPNAGV GNHGSSCAEM DVWEANSIST |
| | | | AVTPHPCDTP GQTMCQGDDC GGTYSSTRYA GTCDTDGCDF NPYQPGNHSF YGPGKIVDTS SKFTVVTQFI TDDGTPSGTL |
| | | | TEIKRFYVQN GKVIPQSEST ISGVTGNSIT TEYCTAQKAA FDNTGFFTHG GLQKISQALA QGMVLVMSLW DDHAANMLWL |
| | | | DSTYPTDADP DTPGVARGTC PTTSGVPADV ESQNPNSYVI YSNIKVGPIN STFTAN |
| SEQ ID NO: 95 | 156712280 | Acremonium | MHKRAATLSA LVVAAAGFAR GQGVGTQQTE THPKLTFQKC SAAGSCTTQN GEVVIDANWR WVHDKNGYTN CYTGNEWNTT |
| | | thermophilum | ICADAASCAS NCVVDGADYQ GTYGASTSGN ALTLKFVTKG SYATNIGSRM YLMASPTKYA MFTLLGHEFA FDVDLSKLPC |
| | | | GLNGAVYFVS MDEDGGTSKY PSNKAGAKYG TGYCDSQCPR DLKFIDGKAN SASWQPSSND QNAGVGGMGS CCAEMDIWEA |
| | | | NSVSAAYTPH PCQNYQQHSC SGDDCGGTYS ATRFAGDCDP DGCDWNAYRM GVHDFYGNGK TVDTGKKFSI VTQFKGSGST |
| | | | LTEIKQFYVQ DGRKIENPNA TWPGLEPFNS ITPDFCKAQK QVFGDPDRFN DMGGFTNMAK ALANPMVLVL SLWDDHYSNM |
| | | | LWLDSTYPTD ADPSAPGKGR GTCDTSSGVP SDVESKNGDA TVIYSNIKFG PLDSTYTAS |
| SEQ ID NO: 96 | 5231154 | Volvariella | MRASLLAFSL NSAAGQQAGT LQTKNHPSLT SQKCRQGGCP QVNTTIVLDA NWRWTHSTSG STNCYTGNTW QATLCPDGKT |
| | | volvacea | CAANCALDGA DYTGTYGVTT SGNSLTLQFV TQSNVGARLG YLMADDTTYQ MFNLLNQEFW FDVDMSNLPC GLNGALYFSA |
| | | | MARTAAWMPM VVCASTPLIS TRRSTARLLR LPVPPRSRYG RGICDSQCPR DIKFINGEAN VQGWQPSPND TNAGTGNYGA |
| | | | CCNKMDVWEA NSISTAYTPH PCTQRGLVRC SGTACGGGSN RYGSICDHDG LGFQNLFGMG RTRVRARVGR VKQFNRSSRV |
| | | | VEPISWTKQT TLHLGNLPWK SADCNVQNGR VIQNSKVNIP GMPSTMDSVT TEFCNAQKTA FNDTFSFQQK GGMANMSEAL |
| | | | RRGMVLVLSI WDDHAANMLW LDSITSAAAC RSTPSEVHAT PLRESQIRSS HSRQTRYVTF TNIKFGPFNS TGTTYTTGSV |
| | | | PTTSTSTGTT GSSTPPQPTG VTVPQGQCGG IGYTGPTTCA SPTTCHVLNP YYSQCY |
| SEQ ID NO: 97 | 116200349 | Chaetomium | MKQYLQYLAA ALPLMSLVSA QGVGTSTSET HPKITWKKCS SGGSCSTVNA EVVIDANWRW LHNADSKNCY DGNEWTDACT |
| | | globosum CBS | SSDDCTSKCV LEGAEYGKTY GASTSGDSLS LKFLTKHEYG TNIGSRFYLM NGASKYQMFT LMNNEFAFDV DLSTVECGLN |
| | | 148-51 | SALYFVAMEE DGGMASYSTN KAGAKYGTGY CDAQCARDLK FVGGKANYDG WTPSSNDANA GVGALGGCCA EIDVWESNAH |
| | | | AFAFTPHACE NNNYHVCEDT TCGGTYSEDR FAGDCDANGC DYNPYRVGNT DFYGKGMTVD TSKKFTVVSQ FQENKLTQFF |
| | | | VQNGKKIEIP GPKHEGLPTE SSDITPELCS AMPEVFGDRD RFAEVGGFDA LNKALAVPMV LVMSIWDDHY ANMLWLDSSY |
| | | | PPEKAGTPGG DRGPCAQDSG VPSEVESQYP DATVVWSNIR FGPIGSTVQV |
| SEQ ID NO: 98 | 4586343 | Irpex lacteus | MFPKASLIAL SFIAAVYGQQ VGTQMAEVHP KLPSQLCTKS GCTNQNTAVV LDANWRWLHT TSGYTNCYTG NSWDATLCPD |
| | | | ATTCAQNCAV DGADYSGTYG ITTSGNALTL KFKTGTNVGS RVYLMQTDTA YQMFQLLNQE FTFDVDMSNL PCGLNGALYL |
| | | | SQMDQDGGLS KFPTNKAGAK YGTGYCDSQC PHDIKFINGM ANVAGWAGSA SDPNAGSGTL GTCCSEMDIW EANNDAAAFT |
| | | | PHPCSVDGQT QCSGTQCGDD DERYSGLCDK DGCDFNSFRM GDKSFLGKGM TVDTSRKFTV VTQFVTTDGT TNGDLHEIRR |
| | | | LYVQDGKVIQ NSVVSIPGID AVDSITDNFC AQQKSVFGDT NYFATLGGLK KMGAALKSGM VLAMSVWDDH AASMQWLDSN |
| | | | YPADGDATKP GVARGTCSAD SGLPTNVESQ SASASVTFSN IKWGDINTTF TGTGSTSPSS PAGPVSSSTS VASQPTQPAQ |
| | | | GTVAQWGQCG GTGFTGPTVC ASPFTCHVVNPYYSQCY |
| SEQ ID NO: 99 | 15321718 | Lentinula edodes | MFRTAALLSF AYLAVVYGQQ AGTSTAETHP PLTWEQCTSG GSCTTQSSSV VLDSNWRWTH VVGGYTNCYT GNEWNTTVCP |
| | | | DGTTCAANCA LDGADYEGTY GISTSGNALT LKFVTASAQT NVGSRVYLMA PGSETEYQMF NPLNQEFTFD VDVSALPCGL |
| | | | NGALYFSEMD ADGGLSEYPT NKAGAKYGTG YCDSQCPRDI KFIEGKANVE GWTPSSTSPN AGTGGTGICC NEMDIWEANS |
| | | | ISEALTPHPC TAQGGTACTG DSCSSPNSTA GICDQAGCDF NSFRMGDTSF YGPGLTVDTT SKITVVTQFI TSDNTTTGDL |
| | | | TAIRRIYVQN GQVIQNSMSN IAGVTPTNEI TTDFCDQQKT AFGDTNTFSE KGGLTGMGAA FSRGMVLVLS IWDDDAAEML |
| | | | WLDSTYPVGK TGPGAARGTC ATTSGQPDQV ETQSPNAQVV FSNIKFGAIG STFSSTGTGT GTGTGTGTGT GTTTSSAPAA |
| | | | TQTKYGQCGG QGWTGATVCA SGSTCTSSGP YYSQCL |
| SEQ ID NO: 100 | 146424875 | Pleurotus sp | MFRTAALTAF TFAAVVLGQQ VGTLTTENHP ALSIQQCTAT GCTTQQKSVV LDSNWRWTHS TAGATNCYTG NAWDPALCPD |
| | | Florida | PATCATNCAI DGADYSGTYG ITTSGNALTL RFVTNGQYSQ NIGSRVYLLD DADHYKLFDL KNQEFTFDVD MSGLPCGLNG |
| | | | ALYFSEMAAD GGKAAHAGNN AGAKYGTGYC DAQCPHDIKW INGEANVLDW SASATDDNAG NGRYGACCAE MDIWEANSEA |
| | | | TAYTPHVCRD EGLYRCSGTE CGDGNNRYGG VCDKDGCDFN SYRMGDKNFL GRGKTIDTTK KVTVVTQFIT DNNTPTGNLV |
| | | | EIRRVYVQNG VVYQNSFSTF PSLSQYNSIS DEFCVAQKTL FGDNQYYNTH GGTTKMGDAF DNGMVLIMSL WSDHAAHMLW |
| | | | LDSDYPLDKS PSEPGVSRGA CPTSSGDPDD VVANHPNASV TFSNIKYGPI GSTFGGSTPP VSSGGSSVPP VTSTTSSGTT |
| | | | TPTGPTGTVP KWGQCGGIGY SGPTACVAGS TCTYSNDWYS QCL |
| SEQ ID NO: 101 | 62006158 | Fusarium | MYRAIATASA LIAAVRAQQV CSLTPETKPA LSWSKCTSSG CSNVQGSVTI DANWRWTHQL SGSTNCYTGN KWDTSICTSG |
| | | venenatum | KVCAEKCCID GAEYASTYGI TSSGNQLSLS FVTKGTYGTN IGSRTYLMED ENTYQMFQLL GNEFTFDVDV SNIGCGLNGA |
| | | | LYFVSMDADG GKAKYPGNKA GAKYGTGYCD AQCPRDVKFI NGQANSDGWQ PSKSDVNGGI GNLGTCCPEM DIWEANSIST |
| | | | AHTPHPCTKL TQHSCTGDSC GGTYSEDRYG GTCDADGCDF NAYRQGNKTF YGPGSGFNVD TTKKVTVVTQ FHKGSNGRLS |
| | | | EITRLYVQNG KVIANSESKI AGVPGSSLTP EFCTAQKKVF GDIDDFEKKG AWGGMSDALE APMVLVMSLW HDHHSNMLWL |
| | | | DSTYPTDSTK LGAQRGSCST SSGVPADLEK NVPNSKVAFS NIKFGPIGST YKEGQPEPTN PTNPNPTTPG GTVDQWGQCG |
| | | | GTNYSGPTAC KSPFTCKKIN DFYSQCQ |
| SEQ ID NO: 102 | 296027 | Phanerochaete | MFRTATLLAF TMAAMVFGQQ VGTNTAENHR TLTSQKCTKS GGCSNLNTKI VLDANWRWLH STSGYTNCYT GNQWDATLCP |
| | | chrysosporium | DGKTCAANCA LDGADYTGTY GITASGSSLK LQFVTGSNVG SRVYLMADDT HYQMFQLLNQ EFTFDVDMSN LPCGLNGALY |
| | | | LSAMDADGGM AKYPTNKAGA KYGTGYCDSQ CPRDIKFING EANVEGWNAT SANAGTGNYG TCCTEMDIWE ANNDAAAYTP |
| | | | HPCTTNAQTR CSGSDCTRDT GLCDADGCDF NSFRMGDQTF LGKGLTVDTS KPFTVVTQFI TNDGTSAGTL TEIRRLYVQN |
| | | | GKVIQNSSVK IPGIDLVNSI TDNFCSQQKT AFGDTNYFAQ HGGLKQVGEA LRTGMVLALS IWDDYAANML WLDSNYPTNK |
| | | | DPSTPGVARG TCATTSGVPA QIEAQSPNAY VVFSNIKFGD LNTTYTGTVS SSSVSSSHSS TSTSSSHSSS STPPTQPTGV |
| | | | TVPQWGQCGG IGYTGSTTCA SPYTCHVLNP YYSQCY |
| SEQ ID NO: 103 | 154449709 | Fusicoccum sp | MYQTSLLASL SFLLATSQAQ QVGTQTAETH PKLTTQKCTT AGGCTDQSTS IVLDANWRWL HTVDGYTNCY TGQEWDTSIC |
| | | BCC4124 | TDGKTCAEKC ALDGADYEST YGISTSGNAL TMNFVTKSSQ TNIGGRVYLL AADSDDTYEL FKLKNQEFTF DVDVSNLPCG |
| | | | LNGALYFSEM DSDGGLSKYT TNKAGAKYGT GYCDTQCPHD IKFINGEANV QNWTASSTDK NAGTGHYGSC CNEMDIWEAN |
| | | | SQATAFTPHV CEAKVEGQYR CEGTECGDGD NRYGGVCDKD GCDFNSYRMG NETFYGSNGS TIDTTKKFTV VTQFITADNT |
| | | | ATGALTEIRR KYVQNDVVIE NSYADYETLS KFNSITDDFC AAQKTLSGDT NDFKTKGGIA RMGESFERGM VLVMSVWDDH |
| | | | AANALWLDSS YPTDADASKP GVKRGPCSTS SGVPSDVEAN DADSSVIYSN IRYGDIGSTF NKTA |
| SEQ ID NO: 104 | 169859460 | Coprinopsis | MFSKVALTAL CFLAVAQAQQ VGREVAENHP RLPWQRCTRN GGCQTVSNGQ VVLDANWRWL HVTDGYTNCY TGNAWNSSVC |
| | | cinerea okayama | SDGATCAQRC ALEGANYQQT YGITTSGDAL TIKFLTRSEQ TNIGARVYLM ENEDRYQMFN LLNKEFTFDV DVSKVPCGIN |
| | | | GALYFIQMDA DGGLSSQPNN RAGAKYGTGY CDSQCPRDIK FINGEANSVG WEPSETDPNA GKGQYGICCA EMDIWEANSI |
| | | | SNAYTPHPCQ TVNDGGYQRC QGRDCNQPRY EGLCDPDGCD YNPFRMGNKD FYGPGKTVDT NRKMTVVTQF ITHDNTDTGT |
| | | | LVDIRRLYVQ DGRVIANPPT NFPGLMPAHD SITQEFCDDA KRAFEDNDSF GRNGGLAHMG RSLAKGHVLA LSIWNDHTAH |
| | | | MLWLDSNYPT DADPNKPGIA RGTCPTTGGS PRDTEQNHPD AQVIFSNIKF GDIGSTFSGN |
| SEQ ID NO: 105 | 50400675 | Trichoderma | MYRKLAVISA FLAAARAQQV CTQQAETHPP LTWQKCTASG CTPQQGSVVL DANWRWTHDT KSTTNCYDGN TWSSTLCPDD |
| | | harzianum | ATCAKNCCLD GANYSGTYGV TTSGDALTLQ FVTASNVGSR LYLMANDSTY QEFTLSGNEF SFDVDVSQLP CGLNGALYFV |
| | | (anamorph of | SMDADGGQSK YPGNAAGAKY GTGYCDSQCP RDLKFINGQA NVEGWEPSSN NANTGVGGHG SCCSEMDIWE ANSISEALTP |
| | | Hypocrea lixii) | HPCETVGQTM CSGDSCGGTY SNDRYGGTCD PDGCDWNPYR LGNTSFYGPG SSFALDTTKK LTVVTQFATD GSISRYYVQN |
| | | | GVKFQQPNAQ VGSYSGNTIN TDYCAAEQTA FGGTSFTDKG GLAQINKAFQ GGMVLVMSLW DDYAVNMLWL DSTYPTNATA |
| | | | STPGAKRGSC STSSGVPAQV EAQSPNSKVI YSNIRFGPIG STGGNTGSNP PGTSTTRAPP SSTGSSPTAT QTHYGQCGGT |
| | | | GWTGPTRCAS GYTCQVLNPF YSQCL |
| SEQ ID NO: 106 | 729649 | Neurospora crassa | MRASLLAFSL AAAVAGGQQA GTLTAKRHPS LTWQKCTRGG CPTLNTTMVL DANWRWTHAT SGSTKCYTGN KWQATLCPDG |
| | | (OR74A) | KSCAANCALD GADYTGTYGI TGSGWSLTLQ FVTDNVGARA YLMADDTQYQ MLELLNQELW FDVDMSNIPC GLNGALYLSA |
| | | | MDADGGMRKY PTNKAGAKYA TGYCDAQCPR DLKYINGIAN VEGWTPSTND ANGIGDHGSC CSEMDIWEAN KVSTAFTPHP |
| | | | CTTIEQHMCE GDSCGGTYSD DRYGVLCDAD GCDFNSYRMG NTTFYGEGKT VDTSSKFTVV TQFIKDSAGD LAEIKAFYVQ |
| | | | NGKVIENSQS NVDGVSGNSI TQSFCKSQKT AFGDIDDFNK KGGLKQMGKA LAQAMVLVMS IWDDHAANML WLDSTYPVPK |
| | | | VPGAYRGSGP TTSGVPAEVD ANAPNSKVAF SNIKFGHLGI SPFSGGSSGT PPSNPSSSAS PTSSTAKPSS TSTASNPSGT |
| | | | GAAHWAQCGG IGFSGPTTCP EPYTCAKDHD IYSQCV |
| SEQ ID NO: 107 | 119472134 | Neosartorya | MLASTFSYRM YKTALILAAL LGSGQAQQVG TSQAEVHPSM TWQSCTAGGS CTTNNGKVVI DANWRWVHKV GDYTNCYTGN |
| | | fischeri NRRL 181 | TWDKTLCPDD ATCASNCALE GANYQSTYGA TTSGDSLRLN FVTTSQQKNI GSRLYMMKDD TTYEMFKLLN QEFTFDVDVS |
| | | | NLPCGLNGAL YFVAMDADGG MSKYPTNKAG AKYGTGYCDS QCPRDLKFIN GQANVEGWQP SSNDANAGTG NHGSCCAEMD |
| | | | IWEANSISTA FTPHPCDTPG QVMCTGDACG GTYSSDRYGG TCDPDGCDFN SFRQGNKTFY GPGMTVDTKS KFTVVTQFIT |
| | | | DDGTASGTLK EIKRFYVQNG KVIPNSESTW SGVGGNSITN DYCTAQKSLF KDQNVFAKHG GMEGMGAALA QGMVLVMSLW |
| | | | DDHAANMLWL DSNYPTTASS STPGVARGTC DISSGVPADV EANHPDASVV YSNIKVGPIG STFNSGGSNP GGGTTTTAKP |
| | | | TTTTTTAGSP GGTGVAQHYG QCGGNGWQGP TTCASPYTCQ KLNDFYSQCL |
| SEQ ID NO: 108 | 117935080 | Chaetomium | MQIKQYLQYL AAALPLVNMA AAQRAGTQQT ETHPRLSWKR CSSGGNCQTV NAEIVIDANW RWLHDSNYQN CYDGNRWTSA |
| | | thermophilum | CSSATDCAQK CYLEGANYGS TYGVSTSGDA LTLKFVTKHE YGTNIGSRVY LMNGSDKYQM FTLMNNEFAF DVDLSKVECG |
| | | | LNSALYFVAM EEDGGMRSYS SNKAGAKYGT GYCDAQCARD LKFVGGKANI EGWRPSTNDA NAGVGPYGAC CAEIDVWESN |
| | | | AYAFAFTPHG CLNNNYHVCE TSNCGGTYSE DRFGGLCDAN GCDYNPYRMG NKDFYGKGKT VDTSRKFTVV TRFEENKLTQ |
| | | | FFIQDGRKID IPPPTWPGLP NSSAITPELC TNLSKVFDDR DRYEETGGFR TINEALRIPM VLVMSIWDGH YASMLWLDSV |
| | | | YPPEKAGQPG AERGPCAPTS GVPAEVEAQF PNAQVIWSNI RFGPIGSTYQ V |
| SEQ ID NO: 109 | 154300584 | Botryotinia | MTSRIALVSL FAAVYGQQVG TYQTETHPSL TWQSCTAKGS CTTNTGSIVL DGNWRWTHGV GTSTNCYTGN TWDATLCPDD |
| | | fuckeliana B05-10 | ATCAQNCALE GADYSGTYGI TTSGNSLRLN FVTQSANKNI GSRVYLMADT THYKTFNLLN QEFTFDVDVS NLPCGLNGAV |
| | | | YFANLPADGG ISSTNTAGAE YGTGYCDSQC PRDMKFIKGQ ANVDGWVPSS NNANTGVGNH GSCCAEMDIW EANSISTAVT |
| | | | PHSCDTVTQT VCTGDDCGGT YSSSRYAGTC DPDGCDFNSY RMGDETFYGP GKTVDTNSVF TVVTQFLTTD GTASGTLNEI |
| | | | KRFYVQDGKV IPNSYSTISG VSGNSITTPF CDAQKTAFGD PTSFSDHGGL ASMSAAFEAG MVLVLSLWDD YYANMLWLDS |
| | | | TYPVGKTSAG GPRGTCDTSS GVPASVEASS PNAYVVYSNI KVGAINSTYG |
| SEQ ID NO: 110 | 15824271 | Pseudotrichonympha | MFVFVLLWLT QSLGTGTNQA ENHPSLSWQN CRSGGSCTQT SGSVVLDSNW RWTHDSSLTN CYDGNEWSSS LCPDPKTCSD |
| | | grassii | NCLIDGADYS GTYGITSSGN SLKLVFVTNG PYSTNIGSRV YLLKDESHYQ IFDLKNKEFT FTVDDSNLDC GLNGALYFVS |
| | | | MDEDGGTSRF SSNKAGAKYG TGYCDAQCPH DIKFINGEAN VENWKPQTND ENAGNGRYGA CCTEMDIWEA NKYATAYTPH |
| | | | ICTVNGEYRC DGSECGDTDS GNRYGGVCDK DGCDFNSYRM GNTSFWGPGL IIDTGKPVTV VTQFVTKDGT DNGQLSEIRR |
| | | | KYVQGGKVIE NTVVNIAGMS SGNSITDDFC NEQKSAFGDT NDFEKKGGLS GLGKAFDYGM VLVLSLWDDH QVNMLWLDSI |
| | | | YPTDQPASQP GVKRGPCATS SGAPSDVESQ HPDSSVTFSD IRFGPIDSTY |
| SEQ ID NO: 111 | 4586345 | Irpex lacteus | MFRKAALLAF SFLAIAHGQQ VGTNQAENHP SLPSQKCTAS GCTTSSTSVV LDANWRWVHT TTGYTNCYTG QTWDASICPD |
| | | | GVTCAKACAL DGADYSGTYG ITTSGNALTL QFVKGTNVGS RVYLLQDASN YQMFQLINQE FTFDVDMSNL PCGLNGAVYL |
| | | | SQMDQDGGVS RFPTNTAGAK YGTGYCDSQC PRDIKFINGE ANVEGWTGSS TDSNSGTGNY GTCCSEMDIW EANSVAAAYT |
| | | | PHPCSVNQQT RCTGADCGQG DDRYDGVCDP DGCDFNSFRM GDQTFLGKGL TVDTSRKFTI VTQFISDDGT TSGNLAEIRR |
| | | | FYVQDGNVIP NSKVSIAGID AVNSITDDFC TQQKTAFGDT NRFAAQGGLK QMGAALKSGM VLALSLWDDH AANMLWLDSD |
| | | | YPTTADASNP GVARGTCPTT SGFPRDVESQ SGSATVTYSN IKWGDLNSTF TGTLTTPSGS SSPSSPASTS GSSTSASSSA |
| | | | SVPTQSGTVA QWAQCGGIGY SGATTCVSPY TCHVVNAYYS QCY |
| SEQ ID NO: 112 | 46241268 | Gibberella | MYRAIATASA LIAAARAQQV CTLTTETKPA LTWSKCTSSG CTDVKGSVGI DANWRWTHQT SSSTNCYTGN KWDTSVCTSG |
| | | avenacea | ETCAQKCCLD GADYAGTYGI TSSGNQLSLG FVTKGSFSTN IGSRTYLMEN ENTYQMFQLL GNEFTFDVDV SNIGCGLNGA |
| | | | LYFVSMDADG GKARYPANKA GAKYGTGYCD AQCPRDVKFI NGKANSDGWK PSDSDINAGI GNMGTCCPEM DIWEANSIST |
| | | | AFTPHPCTKL TQHACTGDSC GGTYSNDRYG GTCDADGCDF NSYRQGNKTF YGRGSDFNVD TTKKVTVVTQ FKKGSNGRLS |
| | | | EITRLYVQNG KVIANSESKI PGNSGSSLTA DFCSKQKSVF GDIDDFSKKG GWSGMSDALE SPPMVLVMSL WHDHHSNMLW |
| | | | LDSTYPTDST KLGAQRGSCA TTSGVPSDLE RDVPNSKVSF SNIKFGPIGS TYSSGTTNPP PSSTDTSTTP TNPPTGGTVG |
| | | | QYGQCGGQTY TGPKDCKSPY TCKKINDFYS QCQ |
| SEQ ID NO: 113 | 6164684 | Aspergillus niger | MSSFQIYRAA LLLSILATAN AQQVGTYTTE THPSLTWQTC TSDGSCTTND GEVVIDANWR WVHSTSSATN CYTGNEWDTS |
| | | | ICTDDVTCAA NCALDGATYE ATYGVTTSGS ELRLNFVTQG SSKNIGSRLY LMSDDSNYEL FKLLGQEFTF DVDVSNLPCG |
| | | | LNGALYFVAM DADGGTSEYS GNKAGAKYGT GYCDSQCPRD LKFINGEANC DGWEPSSNNV NTGVGDHGSC CAEMDVWEAN |
| | | | SISNAFTAHP CDSVSQTMCD GDSCGGTYSA SGDRYSGTCD PDGCDYNPYR LGNTDFYGPG LTVDTNSPFT VVTQFITDDG |
| | | | TSSGTLTEIK RLYVQNGEVI ANGASTYSSV NGSSITSAFC ESEKTLFGDE NVFDKHGGLE GMGEAMAKGM VLVLSLWDDY |
| | | | AADMLWLDSD YPVNSSASTP GVARGTCSTD SGVPATVEAE SPNAYVTYSN IKFGPIGSTY SSGSSSGSGS SSSSSSTTTK |
| | | | ATSTTLKTTS TTSSGSSSTS AAQAYGQCGG QGWTGPTTCV SGYTCTYENA YYSQCL |
| SEQ ID NO: 114 | 6164682 | Aspergillus niger | MHQRALLFSA LLTAVRAQQA GTLTEEVHPS LTWQKCTSEG SCTEQSGSVV IDSNWRWTHS VNDSTNCYTG NTWDATLCPD |
| | | | DETCAANCAL DGADYESTYG VTTDGDSLTL KFVTGSNVGS RLYLMDTSDE GYQTFNLLDA EFTFDVDVSN LPCGLNGALY |
| | | | FTAMDADGGV SKYPANKAGA KYGTGYCDSQ CPRDLKFIDG QANVDGWEPS SNNDNTGIGN HGSCCPEMDI WEANKISTAL |
| | | | TPHPCDSSEQ TMCEGNDCGG TYSDDRYGGT CDPDGCDFNP YRMGNDSFYG PGKTIDTGSK MTVVTQFITD GSGSLSEIKR |
| | | | YYVQNGNVIA NADSNISGVT GNSITTDFCT AQKKAFGDED IFAEHNGLAG ISDAMSSMVL ILSLWDDYYA SMEWLDSDYP |
| | | | ENATATDPGV ARGTCDSESG VPATVEGAHP DSSVTFSNIK FGPINSTFSA SA |
| SEQ ID NO: 115 | 33733371 | Chrysosporium | MYAKFATLAA LVAGAAAQNA CTLTAENHPS LTWSKCTSGG SCTSVQGSIT IDANWRWTHR TDSATNCYEG NKWDTSYCSD |
| | | lucknowense | GPSCASKCCI DGADYSSTYG ITTSGNSLNL KFVTKGQYST NIGSRTYLME SDTKYQMFQL LGNEFTFDVD VSNLGCGLNG |
| | | U.S. Pat. No. 6,573,086-10 | ALYFVSMDAD GGMSKYSGNK AGAKYGTGYC DSQCPRDLKF INGEANVENW QSSTNDANAG TGKYGSCCSE MDVWEANNMA |
| | | | AAFTPHPCXV IGQSRCEGDS CGGTYSTDRY AGICDPDGCD FNSYRQGNKT FYGKGMTVDT TKKITVVTQF LKNSAGELSE |
| | | | IKRFYVQNGK VIPNSESTIP GVEGNSITQD WCDRQKAAFG DVTDXQDKGG MVQMGKALAG PMVLVMSIWD DHAVNMLWLD |
| | | | STWPIDGAGK PGAERGACPT TSGVPAEVEA EAPNSNVIFS NIRFGPIGST VSGLPDGGSG NPNPPVSSST PVPSSSTTSS |
| | | | GSSGPTGGTG VAKHYEQCGG IGFTGPTQCE SPYTCTKLND WYSQCL |
| SEQ ID NO: 116 | 29160311 | Thielavia | MYAKFATLAA LVAGASAQAV CSLTAETHPS LTWQKCTAPG SCTNVAGSIT IDANWRWTHQ TSSATNCYSG SKWDSSICTT |
| | | australiensis | GTDCASKCCI DGAEYSSTYG ITTSGNALNL KFVTKGQYST NIGSRTYLME SDTKYQMFKL LGNEFTFDVD VSNLGCGLNG |
| | | | ALYFVSMDAD GGMSKYSGNK AGAKYGTGYC DAQCPRDLKF INGEANVEGW ESSTNDANAG SGKYGSCCTE MDVWEANNMA |
| | | | TAFTPHPCTT IGQTRCEGDT CGGTYSSDRY AGVCDPDGCD FNSYRQGNKT FYGKGMTVDT TKKITVVTQF LKNSAGELSE |
| | | | IKRFYAQDGK VIPNSESTIA GIPGNSITKA YCDAQKTVFQ NTDDFTAKGG LVQMGKALAG DMVLVMSVWD DHAVNMLWLD |
| | | | STYPTDQVGV AGAERGACPT TSGVPSDVEA NAPNSNVIFS NIRFGPIGST VQGLPSSGGT SSSSSAAPQS TSTKASTTTS |
| | | | AVRTTSTATT KTTSSAPAQG TNTAKHWQQC GGNGWTGPTV CESPYKCTKQ NDWYSQCL |
| SEQ ID NO: 117 | 146197087 | uncultured | MLTLVYFLLS LVVSLEIGTQ QSEDHPKLTW QNGSSSVSGS IVLDSNWRWV HDSGTTNCYD GNLWSKDLCP SSDTCSQKCY |
| | | symbiotic protist | IEGADYSGTY GIQSSGSKLT LKFVTKGSYS TNIGSRVYLL KDENTYESFK LKNKEFTFTV DDSKLNCGLN GALYFVAMDA |
| | | of Reticulitermes | DGGKAKYSSF KPGAKYGMGY CDAQCPHDMK FISGKANVDD WKPQDNDENS GNGKLGTCCS EMDIWEGNMK SQAYTVHACT |
| | | speratus | KSGQYECTGQ QCGDTDSGDR FKGTCDKDGC DYASWRWGDQ SFYGEGKTVD TKQPVTVVTQ FIGDPLTEIR RLYVQGGKTI |
| | | | NNSKTSNLAD TYDSITDKFC DATKEASGDT NDFKAKGAMS GFSTNLNNGQ VLVMSLWDDH TANMLWLDST YPTDSSDSTA |
| | | | QRGPCPTSSG VPKDVESQHG DATVVFSDIK FGAINSTFKY N |
| SEQ ID NO: 118 | 146197237 | uncultured | MLAAALFTFA CSVGVGTKTP ENHPKLNWQN CASKGSCSQV SGEVTMDSNW RWTHDGNGKN CYDGNTWISS LCPDDKTCSD |
| | | symbiotic protist | KCVLDGAEYQ ATYGIQSNGT ALTLKFVTHG SYSTNIGSRL YLLKDKSTYY VFKLNNKEFT FSVDVSKLPC GLNGALYFVE |
| | | of Neotermes | MDADGGKAKY AGAKPGAEYG LGYCDAQCPS DLKFINGEAN SEGWKPQSGD KNAGNGKYGS CCSEMDVWES NSQATALTPH |
| | | koshunensis | VCKTTGQQRC SGKSECGGQD GQDRFAGLCD EDGCDFNNWR MGDKTFFGPG LIVDTKSPFV VVTQFYGSPV TEIRRKYVQN |
| | | | GKVIENSKSN IPGIDATAAI SDHFCEQQKK AFGDTNDFKN KGGFAKLGQV FDRGMVLVLS LWDDHQVAML WLDSTYPTNK |
| | | | DKSQPGVDRG PCPTSSGKPD DVESASADAT VVYGNIKFGA LDSTY |
| SEQ ID NO: 119 | 146197067 | uncultured | MLTLVYFLLS LVVSLEIGTQ QSEDHPKLTW QNGSSSVSGS IVLDSNWRWV HDSGTTNCYD GNLWSKDLCP SSNTCSQKCY |
| | | symbiotic protist | IEGADYSGTY GIQSSGSKLT LKFVTKGSYS TNIGSRVYLL KDENTYESFK LKNKEFTFTV DDSKLNCGLN GALYFVAMDA |
| | | of Reticulitermes | DGGKAKYSSF KPGAKYGMGY CDAQCPHDMK FISGKANVDD WKPQDNDENS GNGKLGTCCS EMDIWEGNMK SQAYTVHACT |
| | | speratus | KSGQYECTGQ QCGDTDSGDR FKGTCDKDGC DYASWRWGDQ SFYGEGKTVD TKQPVTVVTQ FIGDPLTEIR RLYVQGGKTI |
| | | | NNSKTSNLAD TYDSITDKFC DATKEASGDT NDFKAKGAMS GFSTNLNNGQ VLVMSLWDDH TANMLWLDST YPTDSTKTGA |
| | | | SRGPCAVSSG VPKDVESQYG DATVIYSDIK FGAINSTFKW N |
| SEQ ID NO: 120 | 146197407 | uncultured | MILALLSLAK SLGIATNQAE THPKLTWTRY QSKGSGQTVN GEIVLDSNWR WTHHSGTNCY DGNTWSTSLC PDPTTCSNNC |
| | | symbiotic protist | DLDGADYPGT YGISTSGNSL KLGFVTHGSY STNIGSRVYL LRDSKNYEMF KLKNKEFTFT VDDSKLPCGL NGALYFVAMD |
| | | of Cryptocercus | EDGGVSKNSI NKAGAQYGTG YCDAQCPHDM KFINGEANVL DWKPQSNDEN SGNGRYGACC TEMDIWEANS MATAYTPHVC |
| | | punctulatus | TVTGLRRCEG TECGDTDANQ RYNGICDKDG CDFNSYRLGD KTFFGVGKTV DSSKPVTVVT QFVTSNGQDS GTLSEIRRKY |
| | | | VQGGKVIENS KVNIAGITAG NSVTDTFCNE QKKAFGDNND FEKKGGLGAL SKQLDAGMVL VLSLWDDHSV NMLWLDSTYP |
| | | | TNAAAGALGT ERGACATSSG APSDVESQSP DATVTFSDIK FGPIDSTY |
| SEQ ID NO: 121 | 146197157 | uncultured | MLVIALILRG LSVGTGTQQS ETHPSLSWQQ TSKGGSGQSV SGSVVLDSNW RWTHTTDGTT NCYDGNEWSS DLCPDASTCS |
| | | symbiotic protist | SNCVLEGADY SGTYGITGSG SSLKLGFVTK GSYSTNIGSR VYLLGDESHY KLFKLENNEF TFTVDDSNLE CGLNGALYFV |
| | | of Hodotermopsis | AMDEDGGASK YSGAKPGAKY GMGYCDAQCP HDMKFINGDA NVEGWKPSDN DENAGTGKWG ACCTEMDIWE ANKYATAYTP |
| | | sjoestedti | HICTKNGEYR CEGTDCGDTK DNNRYGGVCD KDGCDFNSWR MGNQSFWGPG LIIDTGKPVT VVTQFLADGG SLSEIRRKYV |
| | | | QGGKVIENTV TKISGMDEFD SITDEFCNQQ KKAFRDTNDF EKKGGLKGLG TAVDAGVVLV LSLWDDHDVN MLWLDSIYPT |
| | | | DSGSKAGADR GPCATSSGVP KDVESNYASA SVTFSDIKFG PIDSTY |
| SEQ ID NO: 122 | 146197403 | uncultured | MLLALFAFGK SLGIATNQAE NHPKLTWTRY QSKGSGQTVN GEIVLDSNWR WTHHSGTNCY DGNTWSTSLC PDPTTCSNNC |
| | | symbiotic protist | DLDGADYPGT YGISSSGNSL KLGFVTHGSY STNIGSRVYL LRDSKNYEMF KLKNKEFTFT VDDSKLPCGL NGALYFVAMD |
| | | of Cryptocercus | EDGGVSKNSI NKAGAQYGTG YCDAQCPHDM KFINGEANVL DWKPQSNDEN SGNGRYGACC TEMDIWEANS MATAYTPHVC |
| | | punctutatus | TVTGIRRCEG TECGDTDANQ RYNGICDKDG CDFNSYRLGD KSFFGVGKTV DSSKPVTVVT QFVTSNGQDS GTLSEIRRKY |
| | | | VQGGKVIENS KVNIAGMAAG NSITDTFCNE QKKAFGDNND FEKKGGLGAL SKQLDSGMVL VLSLWDDHSV NMLWLDSTYP |
| | | | TNAAAGALGT ERGACATSSG APSDVESQSP DATVTFSDIK FGPIDSTY |
| SEQ ID NO: 123 | 146197081 | uncultured | MLASVVYLVS LVVSLEIGTQ QSEEHPKLTW QNGSSSVSGS IVLDSNWRWL HDSGTTNCYD GNLWSDDLCP NADTCSSKCY |
| | | symbiotic protist | IEGADYSGTY GITSSGSKVT LKFVTKGSYS TNIGSRIYLL KDENTYETFK LKNKEFTFTV DDSKLDCGLN GALYFVAMDA |
| | | of Reticulitermes | DGGKAKYSSF KPGAKYGMGY CDAQCPHDMK FISGKANVDD WKPQDNDENS GDGKLGTCCS EMDIWEGNAK SQAYTVHACS |
| | | speratus | KSGQYECTGQ QCGDTDSGDR FKGTCDKDGC DYASWRWGDQ SFYGEGKTVD TKSPVTVVTQ FIGDPLTEIR RVYVQGGKTI |
| | | | NNSKTSNLAD TYDSITDKFC DATKDATGDT NDFKAKGAMA GFSTNLNTAQ VLVSVHCGMI IQPICCGLIR RIQRIQQKQV |
| | | | QAVDRVLCRR VFQRMLKASM VMLQSRTRTL SLELSTRPLV GISPAGRLFF F |
| SEQ ID NO: 124 | 146197413 | uncultured | MILALLVLGK SLGIATNQAE THPKLTWTRY QSKGSGSTVN GEIVLDSNWR WTHHSGTNCY DGNTWSTSLC PDPTTCSNNC |
| | | symbiotic protist | DLDGADYPGT YGISTSGNSL KLGFVTHGSY STNIGSRVYL LKDTKSYEMF KLKNKEFTFT VDDSKLPCGL NGALYFVAMD |
| | | of Cryptocercus | EDGGVSKNSI NKAGAQYGTG YCDAQCPHDM KFINGEANVL DWKPQSNDEN SGNGRYGACC TEMDIWEANS MATAYTPHVC |
| | | punctuiatus | TVTGLRRCEG TECGDTDNDQ RYNGICDKDG CDFNSYRLGD KSFFGVGKTV DSSKPVTVVT QFVTSNGQDS GTLSEIRRKY |
| | | | VQGGKVIENS KVNVAGITAG NSVTDTFCNE QKKAFGDNND FEKKGGLGAL SKQLDAGMVL VLSLWDDHSV NMLWLDSTYP |
| | | | TNAAAGALGT ERGACATSSG KPSDVESQSP DATVTFSDIK FGPIDSTY |
| SEQ ID NO: 125 | 146197309 | uncultured | MLCIGLISFV YSLGVGTNTA ETHPKLTWKN GGQTVNGEVT VDSNWRWTHT KGSTKNCYDG NLWSKDLCPD AATCGKNCVL |
| | | symbiotic protist | EGADYSGTYG VTSSGNALTL KFVTHGSYST NVGSRLYLLK DEKTYQMFNL NGKEFTFTVD VSNLPCGLNG ALYHVNMDED |
| | | of Mastotermes | GGTKRYPDNE AGAKYGTGYC DAQCPTDLKF INGIPNSDGW KPQSNDKNSG NGKYGSCCSE MDIWEANSIC SAVTPHVCDN |
| | | darwiniensis | LQQTRCQGTA CGENGGGSRF GSSCDPDGCD FNSWRMGNKT FYGPGLIVDT KSKFTVVTQF VGNPVTEIKR KYVQNGKVIE |
| | | | NSYSNIEGMD KFNSVSDKFC TAQKKAFGDT DSFTKHGGFK QLGSALAKGM VLVLSLWDDH TVNMLWLDSV YPTNSKKAGS |
| | | | DRGPCPTTSG VPADVESKSA DANVIYSDIR FGAIDSTYK |
| SEQ ID NO: 126 | 146197227 | uncultured | MLGALVALAS CIGVGTNTPE KHPDLKWTNG GSSVSGSIVV DSNWRWTHIK GETKNCYDGN LWSDKYCPDA ATCGKNCVLE |
| | | symbiotic protist | GADYSGTYGV TTSGDAATLK FVTHGQYSTN VGSRLYLLKD EKTYQMFNLV GKEFTFTVDV SNLPCGLNGA LYFVQMDSDG |
| | | of Neotermes | GMAKYPDNQA GAKYGTGYCD AQCPTDLKFI NGIPNSDGWK PQKNDKNSGN GKYGSCCSEM DIWEANSMAT AYTPHVCDKL |
| | | koshunensis | EQTRCSGSAC GQNGGGDRFS SSCDPDGCDF NSWRMGNKTF WGPGLIVDTK KPVQVVTQFV GSGGSVTEIK RKYVQGGKVI |
| | | | DNSMTNIAAM SKQYNSVSDE FCQAQKKAFG DNDSFTKHGG FRQLGATLSK GHVLVLSLWD DHDVNMLWLD SVYPTNSNKP |
| | | | GADRGPCKTS SGVPSDVESQ NADSTVKYSD IRFGAIDSTY SK |
| SEQ ID NO: 127 | 146197253 | uncultured | MLAAALFTFA CSVGVGTKTT ETHPKLNWQQ CACKGSCSQV SGEVTMDSNW RWTHDGNGKN CYDGNTWISS LCPDDKTCSD |
| | | symbiotic protist | KCVLDGAEYQ ATYGIQSNGT ALTPKFVTHG SYSTNIGSRL YLLKDKSTYY VFQLNNKEFT FSVDVSKLPC GLNGALYFVE |
| | | of Neotermes | MDADGGKSKY AGAKPGAEYG LGYCDAQCPS DLKFINGEAN SEGWKPQSGD KNAGNGKYGS CCSEMDVWES NSMATALTPH |
| | | koshunensis | VCKTTGQTRC SGKSECGGQD GQDRFAGNCD EDGCDFNNWR MGDKTFFGPG LTVDTKSPFV VVTQFYGSPV TEIRRKYVQN |
| | | | GKVIENAKSN IPGIDATNAI SDTFCEQQKK AFGDTNDFKN KGGFTKLGSV FSRGMVLVLS LWDDHQVAML WLDSTYPTNK |
| | | | DKSVPGVDRG PCPTSSGKPD DVESASGDAT VVYGNIKFGA LDSTY |
| SEQ ID NO: 128 | 146197099 | uncultured | MFGFLLSLFA LQFALEIGTQ TSESHPSITW ELNGARQSGQ IVIDSNWRWL HDSGTTNCYD GNTWSSDLCP DPEKCSQNCY |
| | | symbiotic protist | LEGADYSGTY GISASGSQLT LGFVTKGSYS TNIGSRVYLL KDENTYPMFK LKNKEFTFTV DVSNLPCGLN GALYFVAMPS |
| | | of Reticulitermes | DGGKAKYPLA KPGAKYGMGY CDAQCPHDMK FINGEANVLD WKPQSNDENA GTGRYGTCCT EMDIWEANSQ ATAYTVHACS |
| | | speratus | KNARCEGTEC GDDSASQRYN GICDKDGCDF NSWRWGNKTF FGPGLTVDSS KPVTVVTQFI GDPLTEIRRI WVQGGKVIQN |
| | | | SFTNVSGITS VDSITNTFCD ESKVATGDTN DFKAKGGMSG FSKALDTEVV LVLSLWDDHT ANMLWLDSTY PTDSTAIGAS |
| | | | RGPCATSSGD PKDVESASAN ASVKFSDIKF GALDSTY |
| SEQ ID NO: 129 | 146197409 | uncultured | MLASLLPLSN SLGTASNQAE THPKLTWTQY TGKGAGQTVN GEIVLDSNWR WTHKDGTNCY DGNTWSSSLC PDPTTCSNNC |
| | | symbiotic protist | NLDGADYPGT YGITTSGNQL KLGFVTHGSY STNIGSRVYL LRDSKNYQMF KLKNKEFTFT VDDSKLPCGL NGAVYFVAMD |
| | | of Cryptocercus | EDGGTAKHSI NKAGAQYGTG YCDAQCPHDM KFINGEANVL DWKPQSNDEN SGNGRWGARC TEMDIWEANS RATAYTPHIC |
| | | punctulatus | TKTGLYRCEG TECGDSDTNR YGGVCDKDGC DFNSYRMGDK SFFGQGKTVD SSKPVTVVTQ FITDNNQDSG KLTEIRRKYV |
| | | | QGGKVIDNSK VNIAGITAGN PITDTFCDEA KKAFGDNNDF EKKGGLSALG TQLEAGFVLV LSLWDDHSVN MLWLDSTYPT |
| | | | NASPGALGVE RGDCAITSGV PADVESQSAD ASVTFSDIKF GPIDSTY |
| SEQ ID NO: 130 | 146197315 | uncultured | MLCIGLISFV YSLGVGTNTA ETHPKLTWKN GGQTVNGEVT VDSNWRWTHT KGSTKNCYDG NLWSKDLCPD AATCGKNCVL |
| | | symbiotic protist | EGADYSGTYG VTSSGNALTL KFVTHGSYST NVGSRLYLLK DEKTYQMFNL NGKEFTFTVD VSNLPCGLSG ALYHVNMDED |
| | | of Mastotermes | GGTKRYPDNE AGAKYGTGYC DAQCPTDLKF INGIPNSDGW KPQSNDKNSG NGKYGSCCSE MDIWEANSIC SAVTPHVCDN |
| | | darwiniensis | LQQTRCQGAA CGENGGGSRF GSSCDPDGCD FNSWGMGNKT FYGPGLIVDT KSKFTVVTQF VGNPVTEIKR KYVQNGKVIE |
| | | | NSYSNIEGMD KFNSVSDKFC TAQKKAFGDT DSFTKHGGFK QLGSALAKGM VLVLSLWDDH TVNMLWLDSV YPTNSKKAGS |
| | | | DRGPCPTTSG VPADVESKSA DANVIYSDIR FGAIDSTYK |
| SEQ ID NO: 131 | 146197411 | uncultured | MILALLVLGK SLGIATNQAE THPKLTWTRY QSKGSGSTVN GEIVLDSNWR WTHHSGTNCY DGNTWSTSLC PDPTTCSNNC |
| | | symbiotic protist | DLDGADYPGT YGISTSGNSL KLGFVTHGSY STNIGSRVYL LRDSKNYEMF KLKNKEFTFT VDDSKLPCGL NGALYFVAMD |
| | | of Cryptocercus | EDGGVSKNSI NKAGAQYGTG YCDAQCPHDM KFINGEANVL DWKPQSNDEN SGNGRYGACC TEMDIWEANS MATAYTPHVC |
| | | punctulatus | TVTGLRRCEG TECGDTDNDQ RYNGICDKDG CDFNSYRLGD KSFFGVGKTV DSSKPVTVVT QFVTSNGQDS GILSETRRKY |
| | | | VQGGKVIENS KVNVAGITAG NSVTDTFCNE QKKAFGDNND FEKKGGLGAL SKQLDAGMVL VLSLWDDHSV NMLWLDSTYP |
| | | | TNAAAGALGT ERGACATSSG KPSDVESQSP DATVTFSDIK FGPIDSTY |
| SEQ ID NO: 132 | 146197161 | uncultured | MIGIVLIQTV FGIGVGTQQS ESHPSLSWQQ CSKGGSCTSV SGSIVLDSNW RWTHIPDGTT NCYDGNEWSS DLCPDPTTCS |
| | | symbiotic protist | NNCVLEGADY SGTYGISTSG SSAKLGFVTK GSYSTNIGSR VYLLGDESHY KIFDLKNKEF TFTVDDSNLE CGLNGALYFV |
| | | of Hodotermopsis | AMDEDGGASR FTLAKPGAKY GTGYCDAQCP HDIKFINGEA NVQDWKPSDN DDNAGTGHYG ACCTEMDIWE ANKYATAYTP |
| | | sjoestedti | HICTENGEYR CEGKSCGDSS DDRYGGVCDK DGCDFNSWRL GNQSFWGPGL IIDTGKPVTV VTQFVTKDGT DSGALSEIRR |
| | | | KYVQGGKTIE NTVVKISGID EVDSITDEFC NQQKQAFGDT NDFEKKGGLS GLGKAFDYGV VLVLSLWDDH DVNMLWLDSV |
| | | | YPTNPAGKAG ADRGPCATSS GDPKEVEDKY ASASVTFSDI KFGPIDSTY |
| SEQ ID NO: 133 | 146197323 | uncultured | MLVFGIVSFV YSIGVGTNTA ETHPKLTWKN GGSTTNGEVT VDSNWRWTHT KGSTKNCYDG NLWSKDLCPD AATCGKNCVL |
| | | symbiotic protist | EGADYSGTYG VTSSGDALTL KFVTHGSYST NVGSRLYLLK DEKTYQMFNL NGKEFTFTVD VSQLPCGLNG ALYFVCMDQD |
| | | of Mastotermes | GGMSRYPDNQ AGAKYGTGYC DAQCPTDLKF INGLPNSDGW KPQSNDKNSG NGKYGSCCSE MDIWEANSLA TAVTPHVCDQ |
| | | darwiniensis | VGQTRCEGRA CGENGGGDRF GSICDPDGCD FNSWRMGNKT FWGPGLIIDT KKPVTVVTQF IGSPVTEIKR EYVQGGKVIE |
| | | | NSYTNIEGMD KFNSISDKFC TAQKKAFGDN DSFTKHGGFS KLGQSFTKGQ VLVLSLWDDH TVNMLWLDSV YPTNSKKLGS |
| | | | DRGPCPTSSG VPADVESKNA DSSVKYSDIR FGSIDSTYK |
| SEQ ID NO: 134 | 146197077 | uncultured | MLSFVFLLGF GVSLEIGTQQ SENHPTLSWQ QCTSSGSCTS QSGSIVLDSN WRWVHDSGTT NCYDGNEWSS DLCPDPETCS |
| | | symbiotic protist | KNCYLDGADY SGTYGITSNG SSLKLGFVTE GSYSTNIGSR VYLKKDTNTY QIFKLKNHEF TFTVDVSNLP CGLNGALYFV |
| | | of Reticulitermes | EMEADGGKGK YPLAKPGAQY GMGYCDAQCP HDMKFINGNA NVLDWKPQET DENSGNGRYG TCCTEMDIWE ANSQATAYTP |
| | | speratus | HICTKDGQYQ CEGTECGDSD ANQRYNGVCD KDGCDFNSYR LGNKTFFGPG LIVDSKKPVT VVTQFITSNG QDSGDLTEIR |
| | | | RIYVQGGKTI QNSFTNIAGL TSVDSITEAF CDESKDLFGD TNDFKAKGGF TAMGKSLDTG VVLVLSLWDD HSVNMLWLDS |
| | | | TYPTDAAAGA LGTQRGPCAT SSGAPSDVES QSPDASVTFS DIKFGPLDST Y |
| SEQ ID NO: 135 | 146197089 | uncultured | MLTLVVYLLS LVVSLEIGTQ QSESHPALTW QREGSSASGS IVLDSNWRWV HDSGTTNCYD GNEWSTDLCP SSDTCTQKCY |
| | | symbiotic protist | IEGADYSGTY GITTSGSKLT LKFVTKGSYS TNIGSRVYLL KDENTYETFK LKNKEFTFTV DDSKLDCGLN GALYFVAMDA |
| | | of Reticulitermes | DGGKQKYSSF KPGAKYGMGY CDAQCPHDMK FISGKANVED WKPQDNDENS GNGKLGTCCS EMDIWEGNAK SQAYTVHACT |
| | | speratus | KSGQYECTGT DCGDSDSRYQ GTCDKDGCDY ASYRWGDHSF YGEGKTVDTK QPITVVTQFI GDPLTEIRRL YIQGGKVINN |
| | | | SKTQNLASVY DSITDAFCDA TKAASGDTND FKAKGAMAGF SKNLDTPQVL VLSLWDDHTA NMLWLDSTYP TDSRDATAER |
| | | | GPCATSSGVP KDVESNQADA SVVFSDIKFG AINSTYSYN |
| SEQ ID NO: 136 | 146197091 | uncultured | MFGFLLSLFA LQFALEIGTQ TSESHPSITW ELNGARQSGQ IVIDSNWRWL HDSGTTNCYD GNTWSSDLCP DPEKCSQNCY |
| | | symbiotic protist | LEGADYSGTY GISASGSQLT LGFVTKGSYS TNIGSRVYLL KDENTYQMFK LKNKEFTFTV DVSNLPCGLN GALYFVAMPS |
| | | of Reticulitermes | DGGKAKYPLA KPGAKYGMGY CDAQCPHDMK FINGEANVLD WKPQSNDENA GTGRYGTCCT EMDIWEANSQ ATAYTVHACS |
| | | speratus | KNARCEGTEC GDDSASQRYN GICDKDGCDF NSWRWGNKTF FGPGLTVDSS KPVTVVTQFI GDPLTEIRRI WVQGGKVIQN |
| | | | SFTNVSGITS VDSITNTFCD ESKVATGDTN DFKAKGGMSG FSKALDTEVV LVLSLWDDHT ANMLWLDSTY PSNSTAIGAT |
| | | | RGPCATSSGD PKNVESASAN ASVKFSDIKF GAFDSTY |
| SEQ ID NO: 137 | 146197097 | uncultured | MLALVYFLLS LVVSLEIGTQ QSEDHPKLTW QNGSSSVSGS IVLDSNWRWV HDSGTTNCYD GNLWSTDLCP SSDTCTSKCY |
| | | symbiotic protist | IEGADYSGTY GITSSGSKVT LKFVTKGSYS TNIGSRIYLL KDENTYETFK LKNKEFTFTV DDSQLNCGLN GALYFVAMDA |
| | | of Reticulitermes | DGGKAKYSSF KPGAKYGMGY CDAQCPHDMK FISGKANVDD WKPQDNDENS GNGKLGTCCS EMDIWEGNAK SQAYTVHACT |
| | | speratus | KSGQYECTGQ QCGDTDSGDR FKGTCDKDGC DYASWRWGDQ SFYGEGKTVD TKQPVTVVTQ FIGDPLTEIR RLYVQGGKTI |
| | | | NNSKTSNLAD TYDSITDKFC DATKEASGDT NDFKAKGAMS GFSTNLNTAQ VLVLSLWDDH TANMLWLDST YPTDSTKTGA |
| | | | SRGPCAVTSG VPKDVESQYG SAQVVYSDIK FGAINSTY |
| SEQ ID NO: 138 | 146197095 | uncultured | MLALVYFLLS FVVSLEIGTQ QSEDHPKLTW QNGSSSVSGS IVLDSNWRWV HDSGTTNCYD GNLWSTDLCG SSDTCSSKCY |
| | | symbiotic protist | IEGADYSGTY GISASGSKLT LKFVTKGSYS TNIGSRVYLL KDENTYETFK LKGKEFTFTV DDSKLDCGLN GALYFVAMDA |
| | | of Reticulitermes | DGGKAKYSSF KPGAKYGMGY CDAQCPHDMK FISGKANVDD WKPQDNDENS GNGKLGTCCS EMDIWEGNAK SQAYTVHACT |
| | | speratus | KSGQYECTGQ QCGDTDSGDR FKGTCDKDGC DYASWRWGDQ SFYGEGKTID TKQPVTVVTQ FIGDPLTEIR RVYVQGGKVI |
| | | | NNSKTSNLAN VYDSITDKFC DDTKDATGDT NDFKAKGAMS GFSTNLNTAQ VLVMSLWDDH TANMLWLDST YPTDSTKTGA |
| | | | SRGPCAVLSG VPKNVESQHG DATVIYSDIK FGAINSTFSY N |
| SEQ ID NO: 139 | 146197401 | uncultured | MFLALFVLGK SLGIATNQAE NHPKLTWTRY QSKGSGQTVN GEVVLDSNWR WTHHSGTNCY DGNTWSTSLC PDPQTCSSNC |
| | | symbiotic protist | DLDGADYPGT YGISSSGNSL KLGFVTHGSY STNIGSRVYL LRDSKNYEMF KLKNKEFTFT VDDSKLPCGL NGALYFVAME |
| | | of Cryptocercus | EDGGVAKNSI NKAGAQYGTG YCDAQCPHDM KFINGEANVL DWKPQSNDEN SGNGRYGACC IEMDIWEANS MATAYTPHVC |
| | | punctulatus | TVTGIHRCEG TECGDTDANQ RYNGICDKDG CDFNSYRMGD KSFFGVGKTV DSSKPVTVVT QFVTSNGQDG GTLSEIKRKY |
| | | | VQGGKVIENS KVNIAGITAV NSITDTFCNE QKKAFGDNND FEKKGGLGAL SKQLDLGMVL VLSLWDDHSV NMLWLDSTYP |
| | | | TDAAAGALGT ERGACATSSG KPSDVESQSP DASVTFSDIK FGPIDSTY |
| SEQ ID NO: 140 | 146197225 | uncultured | MLLCLLSIAN SLGVGTNTAE NHPKLSWKNG GSSVSGSVTV DANWRWTHIK GETKNCYDGN LWSDKYCPDA ATCGKNCVIE |
| | | symbiotic protist | GADYQGTYGV SSSGDGLTLT FVTHGQYSTN VGSRLYLMKD EKTYQMFNLN GKEFTFTVDV SNLPCGLNGA LYFVQMDSDG |
| | | of Neotermes | GMAKYPDNQA GAKYGTGYCD AQCPTDLKFI NGIPNSDGWK PQKNDKNSGN GKYGSCCSEM DIWEANSQAT AYTPHVCDKL |
| | | koshunensis | EQTRCSGSSC GHTGGGERFS SSCDPDGCDF NSWRMGNKTF WGPGLIVDTK KPVQVVTQFV GSGNSCTEIK RKYVQGGKVI |
| | | | DNSMSNIAGM SKQYNSVSDD FCQAQKKAFG DNDSFTKHGG FRQLGATLGK GHVLVLSLWD DHDVNMLWLD SVYPTNSNKP |
| | | | GSDRGPCKTS SGIPADVESQ AASSSVKYSD IRFGAIDSTY K |
| SEQ ID NO: 141 | 146197317 | uncultured | MLCIGLISFV YSLGVGTNTA ETHPKLTWKN GGQTVNGEVT VDSNWRWTHT KGSTKNCYDG NLWSKDLCPD AATCGKNCVL |
| | | symbiotic protist | EGADYSGTYG VTSSGNALTL KFVTHGSYST NVGSRLYLMK DEKTYQMFNL NGKEFTFTVD VSNLPCGLNG ALYHVNMDED |
| | | of Mastotermes | GGTKRYPDNE AGAKYGTGYC DAQCPTDLKF INGIPNSDGW KPQSNDKNSG NGKYGSCCSE MDIWEANSIC SAVTPHVCDT |
| | | darwiniensis | LQQTRCQGTA CGENGGGSRF GSSCDPDGCD FNSWRMGNKT FYGPGLIVDT KSKFTVVTQF VGSPVTEIKR KYVQNGKVIE |
| | | | NSFSNIEGMD KFNSISDKFC TAQKKAFGDT DSFTKHGGFK QLGSALAKGM VLVLSLWDDH TVNMLWLDSV YPTNSKKAGS |
| | | | DRGPCPTTSG VPADVESKSA NANVIYSDIR FGAIDSTYK |
| SEQ ID NO: 142 | 146197251 | uncultured | MLLCLLGIAS SLDAGTNTAE NHPQLSWKNG GSSVSGSVTV DANWRWTHIK GETKNCYDGN LWSDKYCPDA ATCGQNCVIE |
| | | symbiotic protist | GADYQGTYGV SASGNALTLT FVTHGQYSTN VGSRLYLLKD EKTYQIFNLI GKEFTFTVDV SNLPCGLNGA LYFVQMDADG |
| | | of Neotermes | GTAKYSDNKA GAKYGTGYCD AQCPTDLKFI NGIPNSDGWK PQKNDKNSGN GRYGSCCSEM DVWEANSLAT AYTPHVCDKL |
| | | koshunensis | EQVRCDGRAC GQNGGGDRFS SSCDPDGCDF NSWRLGNKTF WGPGLIVDTK QPVQVVTQWV GSGTSVTEIK RKYVQGGKVI |
| | | | DNSFTKLDSL TKQYNSVSDE FCVAQKKAFG DNDSFTKHGG FRQLGATLAK GHVLVLSLWD DHDVNMLWLD SVYPTNSNKP |
| | | | GADRGPCKTS SGVPADVESQ AASSSVKYSD IRFGAIDSTY K |
| SEQ ID NO: 143 | 146197319 | uncultured | MLGIGFVCIV YSLGVGTNTA ENHPKLTWKN SGSTTNGEVT VDSNWRWTHT KGTTKNCYDG NLWSKDLCPD AATCGKNCVL |
| | | symbiotic protist | EGADYSGTYG VTSSGDALTL KFVTHGSYST NVGSRLYLLK DEKTYQIFNL NGKEFTFTVD VSNLPCGLNG ALYFVNMDAD |
| | | of Mastotermes | GGTGRYPDNQ AGAKYGTGYC DAQCPTDLKF INGIPNSDGW KPQSNDKNSG NGKYGSCCSE MDIWEANSLA TAVTPHVCDQ |
| | | darwiniensis | VGQTRCEGRA CGENGGGDRF GSSCDPDGCD FNSWRLGNKT FWGPGLIVDT KKPVTVVTQF VGSPVTEIKR KYVQGGKVIE |
| | | | NSYTNIEGLD KFNSISDKFC TAQKKAFGDN DSFIKHGGFR QLGQSFTKGQ VLVLSLWDDH TVNMLWLDSV YPTNSKKPGA |
| | | | DRGPCPTSSG VPADVESKNA GSSVKYSDIR FGSIDSTYK |
| SEQ ID NO: 144 | 146197071 | uncultured | MATLVGILVS LFALEVALEI GTQTSESHPS LSWELNGQRQ TGSIVIDSNW RWLHDSGTTN CYDGNEWSSD LCPDPEKCSQ |
| | | symbiotic protist | NCYLEGADYS GTYGISSSGN SLQLGFVTKG SYSTNIGSRV YLLKDENTYA TFKLKNKEFT FTADVSNLPC GLNGALYFVA |
| | | of Reticulitermes | MPADGGKSKY PLAKPGAKYG MGYCDAQCPH DMKFINGEAN ILDWKPSSND ENAGAGRYGT CCTEMDIWEA NSQATAYTVH |
| | | speratus | ACSKNARCEG TECGDDDGRY NGICDKDGCD FNSWRWGNKT FFGPNLIVDS SKPVTVVTQF IGDPLTEIRR IYVQGGKVIQ |
| | | | NSFTNISGVA SVDSITDAFC NENKVATGDT NDFKAKGGMS GFSKALDTEV VLVLSLWDDH TANMLWLDST YPTDSSALGA |
| | | | SRGPCAITSG EPKDVESASA NASVKFSDIK FGAIDSTY |
| SEQ ID NO: 145 | 146197075 | uncultured | MLTLVYFLLS LVVSLEIGTQ QSESHPQLSW QNGSSSVSGS IVLDSNWRWV HDSGTTNCYD GNLWSTDLCP SSDTCTSKCY |
| | | symbiotic protist | IEGADYSGTY GITSSGSKLT LKFVTKGSYS TNIGSRVYLL KDENTYETFK LKNKEFTFTV DDSKLDCGLN GALYFVAMDA |
| | | of Reticulitermes | DGGKAKYSSF KPGAKYGMGY CDAQCPHDMK FISGKANVDD WKPQDNDENS GNGKLGTCCS EMDIWEGNAK SQAYTVHACT |
| | | speratus | KSGQYECTGQ QCGDTDSGDR FKGTCDKDGC DYASWRWGDQ SFYGEGKTVD TKQPLTVVTQ FVGDPLTEIR RVYVQGGKTI |
| | | | NNSKTSNLAD TYDSITDKFC DATKEASGDT NDFKAKGAMS GFSTNLNTAQ VLVMSLWDDH TANMLWLDST YPTDSTKTGA |
| | | | SRGPCAVSSG VPKDVESQHG DATVIYSDIK FGAINSTFKW N |
| SEQ ID NO: 146 | 146197159 | uncultured | MLSLVSIFLV GLGFSLGVGT QQSESHPSLS WQNCSAKGSC QSVSGSIVLD SNWRWLHDSG TTNCYDGNEW STDLCPDAST |
| | | symbiotic protist | CDKNCYIEGA DYSGTYGITS SGAQLKLGFV TKGSYSTNIG SRVYLLRDES HYQLFKLKNH EFTFTVDDSQ LPCGLNGALY |
| | | of Hodotermopsis | FVEMAEDGGA KPGAQYGMGY CDAQCPHDMK FITGEANVKD WKPQETDENA GNGHYGACCT EMDIWEANSQ ATAYTPHICS |
| | | sjoestedti | KTGIYRCEGT ECGDNDANQR YNGVCDKDGC DFNSYRLGNK TFWGPGLTVD SNKAMIVVTQ FTTSNNQDSG ELSEIRRIYV |
| | | | QGGKTIQNSD TNVQGITTTN KITQAFCDET KVTFGDTNDF KAKGGFSGLS KSLESGAVLV LSLWDDHSVN MLWLDSTYPT |
| | | | DSAGKPGADR GPCAITSGDP KDVESQSPNA SVTFSDIKFG PIDSTY |
| SEQ ID NO: 147 | 146197405 | uncultured | MILALLVLGK SLGIATNQAE THPKLTWTRY QSKGSGSTVN GEIVLDSNWR WTHHSGTNCY DGNTWSTSLC PDPTTCSNNC |
| | | symbiotic protist | DLDGADYPGT YGISTSGNSL KLGFVTHGSY STNIGSRVYL LKDTKSYEMF KLKNKEFTFT VDDSKLPCGL NGALYFVAMD |
| | | of Cryptocercus | EDGGVSKNSI NKAGAQYGTG YCDAQCPHDM KFINGEANVL DWKPQSNDEN SGNGRYGACC TEMDIWEANS MATAYTPHVC |
| | | punctuiatus | TVTGLRRCEG TECGDTDNDQ RYNGICDKDG CDFNSYRLGD KSFFGVGKTV DSSKPVTVVT QFVTSNGQDS GTLSEIRRKY |
| | | | VQGGKVIENS KVNVAGITAG NSVTDTFCNE QKKAFGDNND FEKKGGFGAL SKQLVAGMVL VLSLWDDHSV NMLWLDSTYP |
| | | | TNAAAGALGT ERGACATSSG KPSDVESQSP DATVTFSDIK FGPIDSTY |
| SEQ ID NO: 148 | 146197327 | uncultured | MLCVGLFGLV YSIGVGTNTQ ETHPKLSWKQ CSSGGSCTTQ QGSVVIDSNW RWTHSTKDLT NCYDGNLWDS TLCPDGTTCS |
| | | symbiotic protist | KNCVLEGADY SGTYGITSSG DSLTLKFVTH GSYSTNVGSR LYLLKDDNNY QIFNLAGKEF TFTVDVSNLP CGLNGALYFV |
| | | of Mastotermes | EMDQDGGKGK HKENEAGAKY GTGYCDAQCP TDLKFIDGIA NSDGWKPQDN DENSGNGKYG SCCSEMDIWE ANSLATAYTP |
| | | darwiniensis | HVCDTKGQKR CQGTACGENG GGDRFGSECD PDGCDFNSWR QGNKSFWGPG LIIDTKKSVQ VVTQFIGSGS SVTEIRRKYV |
| | | | QNGKVIENSY STISGTEKYN SISDDYCNAQ KKAFGDTNSF ENHGGFKRFS QHIQDMVLVL SLWDDHTVNM LWLDSVYPTN |
| | | | SNKPGADRGP CETSSGVPAD VESKSASASV KYSDIRFGPI DSTYK |
| SEQ ID NO: 149 | 146197261 | uncultured | MLLCLWSIAY SLGVGTNTAE NHPKLSWKNG GSSVSGSVTV DANWRWTHIK GETKNCYDGN LWSDKYCPDA ATCGKNCVIE |
| | | symbiotic protist | GADYQGTYGV SASGDGLTLT FVTHGQYSTN VGSRLYLMKD EKTYQIFNLN GKEFTFTVDV SNLPCGLNGA LYFVQMDSDG |
| | | of Neotermes | GMAKYPDNQA GAKYGTGYCD AQCPTDLKFI NGIPNSDGWK PQKNDKNSGN GKYGSCCSEM DIWEANSQAT AYTPHVCDKL |
| | | koshunensis | EQTRCSGSAC GHTGGGERFS SSCDPDGCDF NSWRMGNKTF WGPGLIVDTK KPVQVVTQFV GSGNSCTEIK RKYVQGGKVI |
| | | | DNSMSNIAGM TKQYNSVSDD FCQAQKKAFG DNDSFTKHGG FRQLGATLGK GHVLVLSLWD DHDVNMLWLD SVYPTNSNKP |
| | | | GSDRGPCKTS SGIPADVESQ AASSSVKYSD IRFGAIDSTY K |

TABLE 2

| Sequence | Database | | Position | Position |
|---|---|---|---|---|
| Identifier | Accession | | corresponding | corresponding |
| (SEQ ID NO:) | Number | Species of Origin | to position 268 | to position 411 |
| SEQ ID NO: 1 | BD29555* | Unknown | 273 | 422 |
| SEQ ID NO: 2 | 340514556 | Trichoderma reesei | 268 | 411 |
| SEQ ID NO: 3 | 51243029 | Penicillium occitanis | 273 | 422 |
| SEQ ID NO: 4 | 7cel (PDB) & | Trichoderma reesei | 251 | 394 |
| SEQ ID NO: 5 | 67516425 | Aspergillus nidulans FGSC A4 | 274 | 424 |
| SEQ ID NO: 6 | 46107376 | Gibberella zeae PH-1 | 268 | 415 |
| SEQ ID NO: 7 | 70992391 | Aspergillus fumigatus Af293 | 277 | 427 |
| SEQ ID NO: 8 | 121699984 | Aspergillus clavatus NRRL 1 | 277 | 427 |
| SEQ ID NO: 9 | 1906845 | Claviceps purpurea | 269 | 416 |
| SEQ ID NO: 10 | 1gpi (PDB) & | Phanerochaete chrysosporium | 240 | 391 |
| SEQ ID NO: 11 | 119468034 | Neosartorya fischeri NRRL 181 | 265 | 414 |
| SEQ ID NO: 12 | 7804883 | Leptosphaeria maculans | 256 | 401 |
| SEQ ID NO: 13 | 85108032 | Neurospora crassa N150 | 268 | 412 |
| SEQ ID NO: 14 | 169859458 | Coprinopsis cinerea okayama | 270 | 421 |
| SEQ ID NO: 15 | 154292161 | Botryotinia fuckeliana B05-10 | — | 410 |
| SEQ ID NO: 16 | 169615761 # | Phaeosphaeria nodorum SN15 | 246 | 393 |
| SEQ ID NO: 17 | 4883502 | Humicola grisea | 272 | 413 |
| SEQ ID NO: 18 | 950686 | Humicola grisea | 270 | 416 |
| SEQ ID NO: 19 | 124491660 | Chaetomium thermophilum | 272 | 413 |
| SEQ ID NO: 20 | 58045187 | Chaetomium thermophilum | 270 | 416 |
| SEQ ID NO: 21 | 169601100 # | Phaeosphaeria nodorum SN15 | 237 | 383 |
| SEQ ID NO: 22 | 169870197 | Coprinopsis cinerea okayama | 269 | 421 |
| SEQ ID NO: 23 | 3913806 | Agaricus bisporus | 263 | 414 |
| SEQ ID NO: 24 | 169611094 | Phaeosphaeria nodorum SN15 | 270 | 414 |
| SEQ ID NO: 25 | 3131 | Phanerochaete chrysosporium | — | 410 |
| SEQ ID NO: 26 | 70991503 | Aspergillus fumigatus Af293 | 265 | 414 |
| SEQ ID NO: 27 | 294196 | Phanerochaete chrysosporium | 258 | 409 |
| SEQ ID NO: 28 | 18997123 | Thermoascus aurantiacus | 268 | 418 |
| SEQ ID NO: 29 | 4204214 | Humicola grisea var thermoidea | 272 | 413 |
| SEQ ID NO: 30 | 34582632 | Trichoderma viride (also known | 268 | 411 |
| | | as Hypochrea rufa) | | |
| SEQ ID NO: 31 | 156712284 | Thermoascus aurantiacus | 268 | 418 |
| SEQ ID NO: 32 | 39977899 | Magnaporthe grisea (oryzae) 70- | 268 | 414 |
| | | 15 | | |
| SEQ ID NO: 33 | 20986705 | Talaromyces emersonii | 266 | 416 |
| SEQ ID NO: 34 | 22138843 | Aspergillus oryzae | 265 | 414 |
| SEQ ID NO: 35 | 55775695 | Penicillium chrysogenum | 276 | 426 |
| SEQ ID NO: 36 | 171676762 | Podospora anserina | 270 | 417 |
| SEQ ID NO: 37 | 146350520 | Pleurotus sp Florida | 268 | 420 |
| SEQ ID NO: 38 | 37732123 | Gibberella zeae | 268 | 415 |
| SEQ ID NO: 39 | 156055188 | Sclerotinia sclerotiorum 1980 | — | 410 |
| SEQ ID NO: 40 | 453224 | Phanerochaete chrysosporium | 258 | 409 |
| SEQ ID NO: 41 | 50402144 | Trichoderma reesei | 268 | 411 |
| SEQ ID NO: 42 | 115397177 | Aspergillus terreus NIH2624 | 274 | 424 |
| SEQ ID NO: 43 | 154312003 | Botryotinia fuckeliana B05-10 | 266 | 416 |
| SEQ ID NO: 44 | 49333365 | Volvariella volvacea | 268 | 420 |
| SEQ ID NO: 45 | 729650 | Penicillium janthinellum | 274 | 424 |
| SEQ ID NO: 46 | 146424871 | Pleurotus sp Florida | 267 | 418 |
| SEQ ID NO: 47 | 67538012 | Aspergillus nidulans FGSC A4 | 265 | 410 |
| SEQ ID NO: 48 | 62006162 | Fusarium poae | 268 | 415 |
| SEQ ID NO: 49 | 146424873 | Pleurotus sp Florida | 267 | 418 |
| SEQ ID NO: 50 | 295937 | Trichoderma viride | 268 | 411 |
| SEQ ID NO: 51 | 6179889 # | Alternaria alternata | 240 | 386 |
| SEQ ID NO: 52 | 119483864 | Neosartorya fischeri NRRL 181 | 278 | 428 |
| SEQ ID NO: 53 | 85083281 | Neurospora crassa OR74A | 270 | 412 |
| SEQ ID NO: 54 | 3913803 | Cryphonectria parasitica | 269 | 416 |
| SEQ ID NO: 55 | 60729633 | Corticium rolfsii | 265 | 415 |
| SEQ ID NO: 56 | 39971383 | Magnaporthe grisea 70-15 | 268 | 410 |
| SEQ ID NO: 57 | 39973029 | Magnaporthe grisea 70-15 | 269 | 410 |
| SEQ ID NO: 58 | 1170141 | Fusarium oxysporum | 268 | 415 |
| SEQ ID NO: 59 | 121710012 | Aspergillus clavatus NRRL 1 | 265 | 414 |
| SEQ ID NO: 60 | 17902580 | Penicillium funiculosum | 273 | 422 |
| SEQ ID NO: 61 | 1346226 | Humicola grisea var thermoidea | 270 | 416 |
| SEQ ID NO: 62 | 156712282 | Chaetomium thermophilum | 270 | 416 |
| SEQ ID NO: 63 | 169768818 | Aspergillus oryzae RIB40 | 277 | 427 |
| SEQ ID NO: 64 | 46241270 | Gibberella pulicaris | 268 | 415 |
| SEQ ID NO: 65 | 49333363 | Volvariella volvacea | 265 | 418 |
| SEQ ID NO: 66 | 46395332 | Irpex lacteus | 263 | 414 |
| SEQ ID NO: 67 | 50844407 # | Chaetomium thermophilum var | 245 | 391 |
| | | thermophilum | | |
| SEQ ID NO: 68 | 4586347 | Irpex lacteus | 264 | 415 |
| SEQ ID NO: 69 | 3980202 | Phanerochaete chrysosporium | 258 | 410 |
| SEQ ID NO: 70 | 27125837 | Melanocarpus albomyces | 273 | 414 |
| SEQ ID NO: 71 | 171696102 | Podospora anserina | 265 | 415 |
| SEQ ID NO: 72 | 3913802 | Cochliobolus carbonum | 270 | 416 |
| SEQ ID NO: 73 | 50403723 | Trichoderma viride | 268 | 411 |
| SEQ ID NO: 74 | 3913798 | Aspergillus aculeatus | 275 | 425 |
| SEQ ID NO: 75 | 66828465 | Dictyostelium discoideum | 269 | 419 |
| SEQ ID NO: 76 | 156060391 | Sclerotinia sclerotiorum 1980 | 252 | 402 |
| SEQ ID NO: 77 | 116181754 | Chaetomium globosum CBS 148- | 263 | 413 |
| | | 51 | | |
| SEQ ID NO: 78 | 145230535 | Aspergillus niger | 274 | 424 |
| SEQ ID NO: 79 | 46241266 | Nectria haematococca mpVI | 268 | 415 |
| SEQ ID NO: 80 | 1q9h (PDB) # | Talaromyces emersonii | 248 | 398 |
| SEQ ID NO: 81 | 157362170 | Polyporus arcularius | 269 | 420 |
| SEQ ID NO: 82 | 7804885 | Leptosphaeria maculans | 267 | 407 |
| SEQ ID NO: 83 | 121852 | Phanerochaete chrysosporium | 258 | 409 |
| SEQ ID NO: 84 | 126013214 | Penicillium decumbens | 264 | 415 |
| SEQ ID NO: 85 | 156048578 | Sclerotinia sclerotiorum 1980 | 265 | 413 |
| SEQ ID NO: 86 | 156712278 | Acremonium thermophilum | 269 | 414 |
| SEQ ID NO: 87 | 21449327 | Aspergillus nidulans | 265 | 410 |
| SEQ ID NO: 88 | 171683762 | Podospora anserina | 274 | 415 |
| SEQ ID NO: 89 | 56718412 | Thermoascus aurantiacus var | 268 | 418 |
| | | levisporus | | |
| SEQ ID NO: 90 | 15824273 | Pseudotrichonympha grassii | 263 | 414 |
| SEQ ID NO: 91 | 115390801 | Aspergillus terreus NIH2624 | 266 | 411 |
| SEQ ID NO: 92 | 453223 | Phanerochaete chrysosporium | 258 | 409 |
| SEQ ID NO: 93 | 3132 | Phanerochaete chrysosporium | — | 407 |
| SEQ ID NO: 94 | 16304152 | Thermoascus aurantiacus | 268 | 417 |
| SEQ ID NO: 95 | 156712280 | Acremonium thermophilum | 273 | 420 |
| SEQ ID NO: 96 | 5231154 | Volvariella volvacea | 281 | 438 |
| SEQ ID NO: 97 | 116200349 | Chaetomium globosum CBS 148- | 270 | 412 |
| | | 51 | | |
| SEQ ID NO: 98 | 4586343 | Irpex lacteus | 263 | 414 |
| SEQ ID NO: 99 | 15321718 | Lentinula edodes | — | 417 |
| SEQ ID NO: 100 | 146424875 | Pleurotus sp Florida | 267 | 418 |
| SEQ ID NO: 101 | 62006158 | Fusarium venenatum | 268 | 415 |
| SEQ ID NO: 102 | 296027 | Phanerochaete chrysosporium | 258 | 409 |
| SEQ ID NO: 103 | 154449709 | Fusicoccum sp BCC4124 | 272 | 424 |
| SEQ ID NO: 104 | 169859460 | Coprinopsis cinerea okayama | 269 | 421 |
| SEQ ID NO: 105 | 50400675 | Trichoderma harzianum | 264 | 407 |
| SEQ ID NO: 106 | 729649 | Neurospora crassa | 262 | 406 |
| SEQ ID NO: 107 | 119472134 | Neosartorya fischeri NRRL 181 | 277 | 427 |
| SEQ ID NO: 108 | 117935080 | Chaetomium thermophilum | 272 | 413 |
| SEQ ID NO: 109 | 154300584 | Botryotinia fuckeliana B05-10 | 265 | 413 |
| SEQ ID NO: 110 | 15824271 | Pseudotrichonympha grassii | 263 | 414 |
| SEQ ID NO: 111 | 4586345 | Irpex lacteus | 263 | 414 |
| SEQ ID NO: 112 | 46241268 | Gibberella avenacea | 268 | 416 |
| SEQ ID NO: 113 | 6164684 | Aspergillus niger | 274 | 424 |
| SEQ ID NO: 114 | 6164682 | Aspergillus niger | 266 | 412 |
| SEQ ID NO: 115 | 33733371 | Chrysosporium lucknowense | 269 | 415 |
| | | U.S. Pat. No. 6,573,086-10 | | |
| SEQ ID NO: 116 | 29160311 | Thielavia australiensis | 269 | 415 |
| SEQ ID NO: 117 | 146197087 | uncultured symbiotic protist of | 260 | 402 |
| | | Reticulitermes speratus | | |
| SEQ ID NO: 118 | 146197237 | uncultured symbiotic protist of | 264 | 409 |
| | | Neotermes koshunensis | | |
| SEQ ID NO: 119 | 146197067 | uncultured symbiotic protist of | 260 | 402 |
| | | Reticulitermes speratus | | |
| SEQ ID NO: 120 | 146197407 | uncultured symbiotic protist of | 261 | 412 |
| | | Cryptocercus punciulatus | | |
| SEQ ID NO: 121 | 146197157 | uncultured symbiotic protist of | 264 | 410 |
| | | Hodotermopsis sjoestedti | | |
| SEQ ID NO: 122 | 146197403 | uncultured symbiotic protist of | 261 | 412 |
| | | Cryptocercus punctulatus | | |
| SEQ ID NO: 123 | 146197081 | uncultured symbiotic protist of | 260 | 410 |
| | | Reticulitermes speratus | | |
| SEQ ID NO: 124 | 146197413 | uncultured symbiotic protist of | 261 | 412 |
| | | Cryptocercus punctulatus | | |
| SEQ ID NO: 125 | 146197309 | uncultured symbiotic protist of | 259 | 402 |
| | | Mastotermes darwiniensis | | |
| SEQ ID NO: 126 | 146197227 | uncultured symbiotic protist of | 258 | 404 |
| | | Neotermes koshunensis | | |
| SEQ ID NO: 127 | 146197253 | uncultured symbiotic protist of | 264 | 409 |
| | | Neotermes koshunensis | | |
| SEQ ID NO: 128 | 146197099 | uncultured symbiotic protist of | 258 | 401 |
| | | Reticulitermes speratus | | |
| SEQ ID NO: 129 | 146197409 | uncultured symbiotic protist of | 260 | 411 |
| | | Cryptocercus punctulatus | | |
| SEQ ID NO: 130 | 146197315 | uncultured symbiotic protist of | 259 | 402 |
| | | Mastotermes darwiniensis | | |
| SEQ ID NO: 131 | 146197411 | uncultured symbiotic protist of | 261 | 412 |
| | | Cryptocercus punctulatus | | |
| SEQ ID NO: 132 | 146197161 | uncultured symbiotic protist of | 263 | 413 |
| | | Hodotermopsis sjoestedti | | |
| SEQ ID NO: 133 | 146197323 | uncultured symbiotic protist of | 259 | 402 |
| | | Mastotermes darwiniensis | | |
| SEQ ID NO: 134 | 146197077 | uncultured symbiotic protist of | 264 | 415 |
| | | Reticulitermes speratus | | |
| SEQ ID NO: 135 | 146197089 | uncultured symbiotic protist of | 258 | 400 |
| | | Reticulitermes speratus | | |
| SEQ ID NO: 136 | 146197091 | uncultured symbiotic protist of | 258 | 401 |
| | | Reticulitermes speratus | | |
| SEQ ID NO: 137 | 146197097 | uncultured symbiotic protist of | 260 | 402 |
| | | Reticulitermes speratus | | |
| SEQ ID NO: 138 | 146197095 | uncultured symbiotic protist of | 260 | 402 |
| | | Reticulitermes speratus | | |
| SEQ ID NO: 139 | 146197401 | uncultured symbiotic protist of | 261 | 412 |
| | | Cryptocercus punctulatus | | |
| SEQ ID NO: 140 | 146197225 | uncultured symbiotic protist of | 258 | 404 |
| | | Neotermes koshunensis | | |
| SEQ ID NO: 141 | 146197317 | uncultured symbiotic protist of | 259 | 402 |
| | | Mastotermes darwiniensis | | |
| SEQ ID NO: 142 | 146197251 | uncultured symbiotic protist of | 258 | 404 |
| | | Neotermes koshunensis | | |
| SEQ ID NO: 143 | 146197319 | uncultured symbiotic protist of | 259 | 402 |
| | | Mastotermes darwiniensis | | |
| SEQ ID NO: 144 | 146197071 | uncultured symbiotic protist of | 259 | 402 |
| | | Reticulitermes speratus | | |
| SEQ ID NO: 145 | 146197075 | uncultured symbiotic protist of | 260 | 402 |
| | | Reticulitermes speratus | | |
| SEQ ID NO: 146 | 146197159 | uncultured symbiotic protist of | 260 | 410 |
| | | Hodotermopsis sjoestedti | | |
| SEQ ID NO: 147 | 146197405 | uncultured symbiotic protist of | 261 | 412 |
| | | Cryptocercus punctulatus | | |
| SEQ ID NO: 148 | 146197327 | uncultured symbiotic protist of | 264 | 408 |
| | | Mastotermes darwiniensis | | |
| SEQ ID NO: 149 | 146197261 | uncultured symbiotic protist of | 258 | 404 |
| | | Neotermes koshunensis | | |

TABLE 3

| | | | Signal | Catalytic | | Cellulose |
|---|---|---|---|---|---|---|
| | | | sequence | Domain | | Binding |
| | Database | | (SS) start | (CD) start | Linker start | Domain |
| | Accession | | and end | and end | and end | (CBD) start |
| SEQ ID NO: | Number | Species of Origin | position | position | position | and end |
| SEQ ID NO: 1 | BD29555* | Unknown | 1-25 | 26-455 | 456-493 | 494-529 |
| SEQ ID NO: 2 | 340514556 | Trichoderma reesei | 1-17 | 18-444 | 445-479 | 480-514 |
| SEQ ID NO: 3 | 51243029 | Penicillium occitanis | 1-25 | 26-455 | 456-493 | 494-529 |
| SEQ ID NO: 4 | 7cel (PDB) & | Trichoderma reesei | N/A | 1-427 | N/A | N/A |
| SEQ ID NO: 5 | 67516425 | Aspergillus nidulans | 1-23 | 24-457 | 458-490 | 491-526 |
| | | FGSC A4 | | | | |
| SEQ ID NO: 6 | 46107376 | Gibberella zeae PH-1 | 1-17 | 18-448 | 449-476 | 477-512 |
| SEQ ID NO: 7 | 70992391 | Aspergillus | 1-26 | 27-460 | 461-496 | 497-532 |
| | | fumigatus Af293 | | | | |
| SEQ ID NO: 8 | 121699984 | Aspergillus clavatus | 1-27 | 27-460 | 461-503 | 504-539 |
| | | NRRL 1 | | | | |
| SEQ ID NO: 9 | 1906845 | Claviceps purpurea | 1-19 | 20-449 | N/A | N/A |
| SEQ ID NO: 10 | 1gpi (PDB) & | Phanerochaete | N/A | 1-424 | N/A | N/A |
| | | chrysosporium | | | | |
| SEQ ID NO: 11 | 119468034 | Neosartorya fischeri | 1-17 | 18-447 | N/A | N/A |
| | | NRRL 181 | | | | |
| SEQ ID NO: 12 | 7804883 | Leptosphaeria | 1-17 | 18-434 | N/A | N/A |
| | | maculans | | | | |
| SEQ ID NO: 13 | 85108032 | Neurospora crassa | 1-17 | 18-445 | 446-485 | 486-521 |
| | | N150 | | | | |
| SEQ ID NO: 14 | 169859458 | Coprinopsis cinerea | 1-18 | 19-454 | N/A | N/A |
| | | okayama | | | | |
| SEQ ID NO: 15 | 154292161 | Botryotinia | 1-18 | 19-443 | 444-555 | 556-596 |
| | | fuckeliana B05-10 | | | | |
| SEQ ID NO: 16 | 169615761 # | Phaeosphaeria | 1 | 2-426 | N/A | N/A |
| | | nodorum SN15 | | | | |
| SEQ ID NO: 17 | 4883502 | Humicola grisea | 1-22 | 23-446 | N/A | N/A |
| SEQ ID NO: 18 | 950686 | Humicola grisea | 1-18 | 19-449 | 450-489 | 490-525 |
| SEQ ID NO: 19 | 124491660 | Chaetomium | 1-22 | 23-446 | N/A | N/A |
| | | thermophilum | | | | |
| SEQ ID NO: 20 | 58045187 | Chaetomium | 1-18 | 19-449 | 450-494 | 495-530 |
| | | thermophilum | | | | |
| SEQ ID NO: 21 | 169601100 # | Phaeosphaeria | 1 | 2-416 | N/A | N/A |
| | | nodorum SN15 | | | | |
| SEQ ID NO: 22 | 169870197 | Coprinopsis cinerea | 1-18 | 19-454 | N/A | N/A |
| | | okayama | | | | |
| SEQ ID NO: 23 | 3913806 | Agaricus bisporus | 1-18 | 19-447 | 448-470 | 471-506 |
| SEQ ID NO: 24 | 169611094 | Phaeosphaeria | 1-18 | 19-447 | N/A | N/A |
| | | nodorum SN15 | | | | |
| SEQ ID NO: 25 | 3131 | Phanerochaete | 1-19 | 20-443 | N/A | N/A |
| | | chrysosporium | | | | |
| SEQ ID NO: 26 | 70991503 | Aspergillus | 1-17 | 18-447 | N/A | N/A |
| | | fumigatus Af293 | | | | |
| SEQ ID NO: 27 | 294196 | Phanerochaete | 1-18 | 19-442 | 443-480 | 481-516 |
| | | chrysosporium | | | | |
| SEQ ID NO: 28 | 18997123 | Thermoascus | 1-17 | 18-451 | N/A | N/A |
| | | aurantiacus | | | | |
| SEQ ID NO: 29 | 4204214 | Humicola grisea var | 1-22 | 23-446 | N/A | N/A |
| | | thermoidea | | | | |
| SEQ ID NO: 30 | 34582632 | Trichoderma viride | 1-18 | 18-444 | 445-479 | 480-514 |
| | | (also known as | | | | |
| | | Hypochrea rufa) | | | | |
| SEQ ID NO: 31 | 156712284 | Thermoascus | 1-17 | 18-451 | N/A | N/A |
| | | aurantiacus | | | | |
| SEQ ID NO: 32 | 39977899 | Magnaporthe grisea | 1-17 | 18-447 | N/A | N/A |
| | | (oryzae) 70-15 | | | | |
| SEQ ID NO: 33 | 20986705 | Talaromyces | 1-18 | 19-449 | N/A | N/A |
| | | emersonii | | | | |
| SEQ ID NO: 34 | 22138843 | Aspergillus oryzae | 1-17 | 18-447 | N/A | N/A |
| SEQ ID NO: 35 | 55775695 | Penicillium | 1-25 | 26-459 | 460-494 | 495-529 |
| | | chrysogenum | | | | |
| SEQ ID NO: 36 | 171676762 | Podospora anserina | 1-18 | 19-450 | 451-492 | 493-528 |
| SEQ ID NO: 37 | 146350520 | Pleurotus sp Florida | 1-18 | 19-453 | N/A | N/A |
| SEQ ID NO: 38 | 37732123 | Gibberella zeae | 1-17 | 18-448 | 449-476 | 477-512 |
| SEQ ID NO: 39 | 156055188 | Sclerotinia | 1-18 | 19-443 | 444-546 | 547-586 |
| | | sclerotiorum 1980 | | | | |
| SEQ ID NO: 40 | 453224 | Phanerochaete | 1-18 | 19-442 | 443-474 | 475-510 |
| | | chrysosporium | | | | |
| SEQ ID NO: 41 | 50402144 | Trichoderma reesei | 1-17 | 18-444 | 445-478 | 479-513 |
| SEQ ID NO: 42 | 115397177 | Aspergillus terreus | 1-23 | 24-457 | 458-505 | 506-541 |
| | | NIH2624 | | | | |
| SEQ ID NO: 43 | 154312003 | Botryotinia | 1-17 | 18-449 | 450-480 | 481-516 |
| | | fuckeliana B05-10 | | | | |
| SEQ ID NO: 44 | 49333365 | Volvariella volvacea | 1-18 | 19-453 | N/A | N/A |
| SEQ ID NO: 45 | 729650 | Penicillium | 1-25 | 26-456 | 457-502 | 503-537 |
| | | janthinellum | | | | |
| SEQ ID NO: 46 | 146424871 | Pleurotus sp Florida | 1-18 | 19-451 | 452-487 | 488-523 |
| SEQ ID NO: 47 | 67538012 | Aspergillus nidulans | 1-17 | 18-443 | N/A | N/A |
| | | FGSC A4 | | | | |
| SEQ ID NO: 48 | 62006162 | Fusarium poae | 1-17 | 18-448 | 449-475 | 476-511 |
| SEQ ID NO: 49 | 146424873 | Pleurotus sp Florida | 1-18 | 19-451 | 452-487 | 488-523 |
| SEQ ID NO: 50 | 295937 | Trichoderma viride | 1-17 | 18-444 | 445-478 | 479-513 |
| SEQ ID NO: 51 | 6179889 # | Alternaria alternata | 1 | 2-419 | N/A | N/A |
| SEQ ID NO: 52 | 119483864 | Neosartorya fischeri | 1-26 | 27-461 | 462-499 | 500-535 |
| | | NRRL 181 | | | | |
| SEQ ID NO: 53 | 85083281 | Neurospora crassa | 1-20 | 21-445 | N/A | N/A |
| | | OR74A | | | | |
| SEQ ID NO: 54 | 3913803 | Cryphonectria | 1-18 | 19-449 | N/A | N/A |
| | | parasitica | | | | |
| SEQ ID NO: 55 | 60729633 | Corticium rolfsii | 1-18 | 19-448 | 449-492 | 493-528 |
| SEQ ID NO: 56 | 39971383 | Magnaporthe grisea | 1-17 | 18-443 | N/A | N/A |
| | | 70-15 | | | | |
| SEQ ID NO: 57 | 39973029 | Magnaporthe grisea | 1-19 | 20-443 | N/A | N/A |
| | | 70-15 | | | | |
| SEQ ID NO: 58 | 1170141 | Fusarium | 1-17 | 18-448 | 449-478 | 479-514 |
| | | oxysporum | | | | |
| SEQ ID NO: 59 | 121710012 | Aspergillus clavatus | 1-17 | 18-447 | N/A | N/A |
| | | NRRL 1 | | | | |
| SEQ ID NO: 60 | 17902580 | Penicillium | 1-25 | 26-455 | 456-493 | 494-529 |
| | | funiculosum | | | | |
| SEQ ID NO: 61 | 1346226 | Humicola grisea var | 1-18 | 19-449 | 450-489 | 490-525 |
| | | thermoidea | | | | |
| SEQ ID NO: 62 | 156712282 | Chaetomium | 1-18 | 19-449 | 450-496 | 497-532 |
| | | thermophilum | | | | |
| SEQ ID NO: 63 | 169768818 | Aspergillus oryzae | 1-25 | 26-460 | N/A | N/A |
| | | RIB40 | | | | |
| SEQ ID NO: 64 | 46241270 | Gibberella pulicaris | 1-17 | 18-448 | 449-474 | 475-510 |
| SEQ ID NO: 65 | 49333363 | Volvariella volvacea | 1-18 | 19-451 | 452-476 | 477-512 |
| SEQ ID NO: 66 | 46395332 | Irpex lacteus | 1-18 | 19-447 | 448-485 | 486-521 |
| SEQ ID NO: 67 | 50844407 # | Chaetomium | N/A | 1-424 | 425-469 | 470-505 |
| | | thermophilum var | | | | |
| | | thermophilum | | | | |
| SEQ ID NO: 68 | 4586347 | Irpex lacteus | 1-18 | 19-448 | 449-490 | 491-526 |
| SEQ ID NO: 69 | 3980202 | Phanerochaete | 1-18 | 19-443 | 444-475 | 476-511 |
| | | chrysosporium | | | | |
| SEQ ID NO: 70 | 27125837 | Melanocarpus | 1-23 | 23-447 | N/A | N/A |
| | | albomyces | | | | |
| SEQ ID NO: 71 | 171696102 | Podospora anserina | 1-17 | 17-448 | N/A | N/A |
| SEQ ID NO: 72 | 3913802 | Cochliobolus | 1-18 | 19-449 | N/A | N/A |
| | | carbonum | | | | |
| SEQ ID NO: 73 | 50403723 | Trichoderma viride | 1-17 | 18-444 | 445-479 | 480-514 |
| SEQ ID NO: 74 | 3913798 | Aspergillus | 1-22 | 23-458 | 459-505 | 506-540 |
| | | aculeatus | | | | |
| SEQ ID NO: 75 | 66828465 | Dictyostelium | 1-19 | 20-452 | N/A | N/A |
| | | discoideum | | | | |
| SEQ ID NO: 76 | 156060391 | Sclerotinia | 1-17 | 18-435 | 436-470 | 471-504 |
| | | sclerotiorum 1980 | | | | |
| SEQ ID NO: 77 | 116181754 | Chaetomium | 1-17 | 18-446 | N/A | N/A |
| | | globosum CBS 148- | | | | |
| | | 51 | | | | |
| SEQ ID NO: 78 | 145230535 | Aspergillus niger | 1-21 | 22-457 | 458-500 | 501-536 |
| SEQ ID NO: 79 | 46241266 | Nectria | 1-18 | 18-448 | 449-472 | 473-508 |
| | | haematococca mpVI | | | | |
| SEQ ID NO: 80 | 1q9h (PDB) # | Talaromyces | N/A | 1-431 | N/A | N/A |
| | | emersonii | | | | |
| SEQ ID NO: 81 | 157362170 | Polyporus | 1-18 | 19-453 | N/A | N/A |
| | | arcularius | | | | |
| SEQ ID NO: 82 | 7804885 | Leptosphaeria | 1-20 | 21-440 | N/A | N/A |
| | | maculans | | | | |
| SEQ ID NO: 83 | 121852 | Phanerochaete | 1-18 | 19-442 | 443-480 | 481-516 |
| | | chrysosporium | | | | |
| SEQ ID NO: 84 | 126013214 | Penicillium | 1-17 | 18-448 | N/A | N/A |
| | | decumbens | | | | |
| SEQ ID NO: 85 | 156048578 | Sclerotinia | 1-16 | 17-446 | N/A | N/A |
| | | sclerotiorum 1980 | | | | |
| SEQ ID NO: 86 | 156712278 | Acremonium | 1-17 | 18-447 | 448-487 | 488-523 |
| | | thermophilum | | | | |
| SEQ ID NO: 87 | 21449327 | Aspergillus nidulans | 1-17 | 18-443 | N/A | N/A |
| SEQ ID NO: 88 | 171683762 | Podospora anserina | 1-22 | 23-448 | N/A | N/A |
| SEQ ID NO: 89 | 56718412 | Thermoascus | 1-17 | 18-451 | N/A | N/A |
| | | aurantiacus var | | | | |
| | | levisporus | | | | |
| SEQ ID NO: 90 | 15824273 | Pseudotrichonympha | 1-20 | 21-447 | N/A | N/A |
| | | grassii | | | | |
| SEQ ID NO: 91 | 115390801 | Aspergillus terreus | 1-17 | 18-444 | N/A | N/A |
| | | NIH2624 | | | | |
| SEQ ID NO: 92 | 453223 | Phanerochaete | 1-18 | 19-442 | 443-474 | 475-510 |
| | | chrysosporium | | | | |
| SEQ ID NO: 93 | 3132 | Phanerochaete | 1-19 | 20-436 | 437-467 | 468-504 |
| | | chrysosporium | | | | |
| SEQ ID NO: 94 | 16304152 | Thermoascus | 1-17 | 18-450 | N/A | N/A |
| | | aurantiacus | | | | |
| SEQ ID NO: 95 | 156712280 | Acremonium | 1-21 | 22-453 | N/A | N/A |
| | | thermophilum | | | | |
| SEQ ID NO: 96 | 5231154 | Volvariella volvacea | 1-15 | 16-472 | 473-500 | 501-536 |
| SEQ ID NO: 97 | 116200349 | Chaetomium | 1-20 | 21-445 | N/A | N/A |
| | | globosum CBS 148- | | | | |
| | | 51 | | | | |
| SEQ ID NO: 98 | 4586343 | Irpex lacteus | 1-18 | 19-447 | 448-481 | 482-517 |
| SEQ ID NO: 99 | 15321718 | Lentinula edodes | 1-18 | 19-450 | 451-480 | 481-516 |
| SEQ ID NO: 100 | 146424875 | Pleurotus sp Florida | 1-18 | 19-451 | 452-487 | 488-523 |
| SEQ ID NO: 101 | 62006158 | Fusarium venenatum | 1-17 | 18-448 | 449-471 | 472-507 |
| SEQ ID NO: 102 | 296027 | Phanerochaete | 1-18 | 19-442 | 443-480 | 481-516 |
| | | chrysosporium | | | | |
| SEQ ID NO: 103 | 154449709 | Fusicoccum sp | 1-19 | 20-457 | N/A | N/A |
| | | BCC4124 | | | | |
| SEQ ID NO: 104 | 169859460 | Coprinopsis cinerea | 1-18 | 19-454 | N/A | N/A |
| | | okayama | | | | |
| SEQ ID NO: 105 | 50400675 | Trichoderma | 1-17 | 18-440 | 441-470 | 471-505 |
| | | harzianum | | | | |
| SEQ ID NO: 106 | 729649 | Neurospora crassa | 1-17 | 18-439 | 440-480 | 481-516 |
| SEQ ID NO: 107 | 119472134 | Neosartorya fischeri | 1-26 | 27-460 | 461-494 | 495-530 |
| | | NRRL 181 | | | | |
| SEQ ID NO: 108 | 117935080 | Chaetomium | 1-22 | 23-446 | N/A | N/A |
| | | thermophilum | | | | |
| SEQ ID NO: 109 | 154300584 | Botryotinia | 1-16 | 17-446 | N/A | N/A |
| | | fuckeliana B05-10 | | | | |
| SEQ ID NO: 110 | 15824271 | Pseudotrichonympha | 1-20 | 21-447 | N/A | N/A |
| | | grassii | | | | |
| SEQ ID NO: 111 | 4586345 | Irpex lacteus | 1-18 | 19-447 | 448-487 | 488-523 |
| SEQ ID NO: 112 | 46241268 | Gibberella avenacea | 1-17 | 18-449 | 450-478 | 478-513 |
| SEQ ID NO: 113 | 6164684 | Aspergillus niger | 1-21 | 22-457 | 458-500 | 501-536 |
| SEQ ID NO: 114 | 6164682 | Aspergillus niger | 1-17 | 18-445 | N/A | N/A |
| SEQ ID NO: 115 | 33733371 | Chrysosporium | 1-17 | 18-448 | 449-490 | 491-526 |
| | | lucknowense | | | | |
| | | US6573086-10 | | | | |
| SEQ ID NO: 116 | 29160311 | Thielavia | 1-18 | 18-448 | 449-502 | 503-538 |
| | | australiensis | | | | |
| SEQ ID NO: 117 | 146197087 | uncultured symbiotic | 1-22 | 23-435 | N/A | N/A |
| | | protist of | | | | |
| | | Reticulitermes | | | | |
| | | speratus | | | | |
| SEQ ID NO: 118 | 146197237 | uncultured symbiotic | 1-20 | 21-442 | N/A | N/A |
| | | protist of Neotermes | | | | |
| | | koshunensis | | | | |
| SEQ ID NO: 119 | 146197067 | uncultured symbiotic | 1-22 | 23-435 | N/A | N/A |
| | | protist of | | | | |
| | | Reticulitermes | | | | |
| | | speratus | | | | |
| SEQ ID NO: 120 | 146197407 | uncultured symbiotic | 1-19 | 20-445 | N/A | N/A |
| | | protist of | | | | |
| | | Cryptocercus | | | | |
| | | punctulatus | | | | |
| SEQ ID NO: 121 | 146197157 | uncultured symbiotic | 1-20 | 21-443 | N/A | N/A |
| | | protist of | | | | |
| | | Hodotermopsis | | | | |
| | | sjoestedii | | | | |
| SEQ ID NO: 122 | 146197403 | uncultured symbiotic | 1-19 | 20-445 | N/A | N/A |
| | | protist of | | | | |
| | | Cryptocercus | | | | |
| | | punctulatus | | | | |
| SEQ ID NO: 123 | 146197081 | uncultured symbiotic | 1-22 | 23-443 | N/A | N/A |
| | | protist of | | | | |
| | | Reticulitermes | | | | |
| | | speratus | | | | |
| SEQ ID NO: 124 | 146197413 | uncultured symbiotic | 1-19 | 20-445 | N/A | N/A |
| | | protist of | | | | |
| | | Cryptocercus | | | | |
| | | punctulatus | | | | |
| SEQ ID NO: 125 | 146197309 | uncultured symbiotic | 1-20 | 21-435 | N/A | N/A |
| | | protist of | | | | |
| | | Mastotermes | | | | |
| | | darwiniensis | | | | |
| SEQ ID NO: 126 | 146197227 | uncultured symbiotic | 1-19 | 20-437 | N/A | N/A |
| | | protist of Neotermes | | | | |
| | | koshunensis | | | | |
| SEQ ID NO: 127 | 146197253 | uncultured symbiotic | 1-21 | 21-442 | N/A | N/A |
| | | protist of Neotermes | | | | |
| | | koshunensis | | | | |
| SEQ ID NO: 128 | 146197099 | uncultured symbiotic | 1-22 | 23-434 | N/A | N/A |
| | | protist of | | | | |
| | | Reticulitermes | | | | |
| | | speratus | | | | |
| SEQ ID NO: 129 | 146197409 | uncultured symbiotic | 1-19 | 20-444 | N/A | N/A |
| | | protist of | | | | |
| | | Cryptocercus | | | | |
| | | punctulatus | | | | |
| SEQ ID NO: 130 | 146197315 | uncultured symbiotic | 1-20 | 21-435 | N/A | N/A |
| | | protist of | | | | |
| | | Mastotermes | | | | |
| | | darwiniensis | | | | |
| SEQ ID NO: 131 | 146197411 | uncultured symbiotic | 1-19 | 20-445 | N/A | N/A |
| | | protist of | | | | |
| | | Cryptocercus | | | | |
| | | punctulatus | | | | |
| SEQ ID NO: 132 | 146197161 | uncultured symbiotic | 1-20 | 21-446 | N/A | N/A |
| | | protist of | | | | |
| | | Hodotermopsis | | | | |
| | | sjoestedii | | | | |
| SEQ ID NO: 133 | 146197323 | uncultured symbiotic | 1-20 | 21-435 | N/A | N/A |
| | | protist of | | | | |
| | | Mastotermes | | | | |
| | | darwiniensis | | | | |
| SEQ ID NO: 134 | 146197077 | uncultured symbiotic | 1-21 | 22-448 | N/A | N/A |
| | | protist of | | | | |
| | | Reticulitermes | | | | |
| | | speratus | | | | |
| SEQ ID NO: 135 | 146197089 | uncultured symbiotic | 1-22 | 23-433 | N/A | N/A |
| | | protist of | | | | |
| | | Reticulitermes | | | | |
| | | speratus | | | | |
| SEQ ID NO: 136 | 146197091 | uncultured symbiotic | 1-22 | 23-434 | N/A | N/A |
| | | protist of | | | | |
| | | Reticulitermes | | | | |
| | | speratus | | | | |
| SEQ ID NO: 137 | 146197097 | uncultured symbiotic | 1-22 | 23-435 | N/A | N/A |
| | | protist of | | | | |
| | | Reticulitermes | | | | |
| | | speratus | | | | |
| SEQ ID NO: 138 | 146197095 | uncultured symbiotic | 1-22 | 23-435 | N/A | N/A |
| | | protist of | | | | |
| | | Reticulitermes | | | | |
| | | speratus | | | | |
| SEQ ID NO: 139 | 146197401 | uncultured symbiotic | 1-19 | 20-445 | N/A | N/A |
| | | protist of | | | | |
| | | Cryptocercus | | | | |
| | | punctulatus | | | | |
| SEQ ID NO: 140 | 146197225 | uncultured symbiotic | 1-19 | 20-437 | N/A | N/A |
| | | protist of Neotermes | | | | |
| | | koshunensis | | | | |
| SEQ ID NO: 141 | 146197317 | uncultured symbiotic | 1-20 | 21-435 | N/A | N/A |
| | | protist of | | | | |
| | | Mastotermes | | | | |
| | | darwiniensis | | | | |
| SEQ ID NO: 142 | 146197251 | uncultured symbiotic | 1-19 | 20-437 | N/A | N/A |
| | | protist of Neotermes | | | | |
| | | koshunensis | | | | |
| SEQ ID NO: 143 | 146197319 | uncultured symbiotic | 1-20 | 21-435 | N/A | N/A |
| | | protist of | | | | |
| | | Mastotermes | | | | |
| | | darwiniensis | | | | |
| SEQ ID NO: 144 | 146197071 | unculturcd symbiotic | 1-25 | 26-435 | N/A | N/A |
| | | protist of | | | | |
| | | Reticulitermes | | | | |
| | | speratus | | | | |
| SEQ ID NO: 145 | 146197075 | uncultured symbiotic | 1-22 | 23-435 | N/A | N/A |
| | | protist of | | | | |
| | | Reticulitermes | | | | |
| | | speratus | | | | |
| SEQ ID NO: 146 | 146197159 | uncultured symbiotic | 1-23 | 24-443 | N/A | N/A |
| | | protist of | | | | |
| | | Hodotermopsis | | | | |
| | | sjoestedti | | | | |
| SEQ ID NO: 147 | 146197405 | uncultured symbiotic | 1-19 | 20-445 | N/A | N/A |
| | | protist of | | | | |
| | | Cryptocercus | | | | |
| | | punctulatus | | | | |
| SEQ ID NO: 148 | 146197327 | uncultured symbiotic | 1-20 | 21-441 | N/A | N/A |
| | | protist of | | | | |
| | | Mastotermes | | | | |
| | | darwiniensis | | | | |
| SEQ ID NO: 149 | 146197261 | uncultured symbiotic | 1-19 | 20-437 | N/A | N/A |
| | | protist of Neotermes | | | | |
| | | koshunensis | | | | |

TABLE 4

| | | | | Amino acid | Amino acid | |
|---|---|---|---|---|---|---|
| | | | | positions of | positions of | |
| Sequence | Database | | Amino acid sequence of | fragment in | active site loop | Position of catalytic |
| Identifier | Accession | Species of | fragment of catalytic domain | sequence | in sequence | residues in sequence |
| (SEQ ID NO:) | Number | Origin | including loop and catalytic residue | identifer | identifer | identifier |
| SEQ ID NO: 150 | BD29555* | Unknown | NVEGWTPSSNNANTGLGNHGACCAELDIWEANS | 210-242 | 214-226 | 234, 239 |
| SEQ ID NO: 151 | 340514556 | Trichoderma | NVEGWEPSSNNANTGIGGHGSCCSEMDIWEANS | 205-237 | 209-221 | 229, 234 |
| | | reesei | | | | |
| SEQ ID NO: 152 | 51243029 | Penicillium | NVEGWTPSANNANTGIGNHGACCAELDIWEANS | 210-242 | 214-226 | 234, 239 |
| | | occitanis | | | | |
| SEQ ID NO: 153 | 7cel (PDB) & | Trichoderma | NVEGWEPSSNNANTGIGGHGSCCSEMDIWQANS | 188-220 | 192-204 | 212, 217 |
| | | reesei | | | | |
| SEQ ID NO: 154 | 67516425 | Aspergillus | NVEGWESSDTNPNGGVGNHGSCCAEMDIWEANS | 211-243 | 215-227 | 235, 240 |
| | | nidulans FGSC | | | | |
| | | A4 | | | | |
| SEQ ID NO: 155 | 46107376 | Gibberella zeae | NSDGWQPSDSDVNGGIGNLGTCCPEMDIWEANS | 205-237 | 209-221 | 229, 234 |
| | | PH-1 | | | | |
| SEQ ID NO: 156 | 70992391 | Aspergillus | NVEGWQPSSNDANAGTGNHGSCCAEMDIWEANS | 214-246 | 218-230 | 238, 243 |
| | | fumigatus Af293 | | | | |
| SEQ ID NO: 157 | 121699984 | Aspergillus | NVEGWTPSSSDANAGNGGHGSCCAEMDIWEANS | 214-246 | 218-230 | 238, 243 |
| | | clavatus NRRL 1 | | | | |
| SEQ ID NO: 158 | 1906845 | Claviceps | NSKDWIPSKSDANAGIGSLGACCREMDIWEANN | 206-238 | 210-222 | 230, 235 |
| | | purpurca | | | | |
| SEQ ID NO: 159 | 1gpi (PDB) & | Phanerochaete | NVGNWTETG SNTGTGSYGTCCSEMDIWEANN | 185-215 | 189-199 | 207, 212 |
| | | chrysosporium | | | | |
| SEQ ID NO: 160 | 119468034 | Neosartorya | NVEGWKPSSNDKNAGVGGHGSCCPEMDIWEANS | 202-234 | 206-218 | 226, 231 |
| | | fischeri NRRL | | | | |
| | | 181 | | | | |
| SEQ ID NO: 161 | 7804883 | Leptosphaeria | NVEGWQPSKNDQNAGVGGHGSCCAEMDIWEANS | 193-225 | 197-209 | 217, 222 |
| | | maculans | | | | |
| SEQ ID NO: 162 | 85108032 | Neurospora | NVEGWTPSTNDANAGIGDHGTCCSEMDIWEANK | 205-237 | 209-221 | 229, 234 |
| | | crassa N150 | | | | |
| | | (OR74A) | | | | |
| SEQ ID NO: 163 | 169859458 | Coprinopsis | NSADWTPSETDPNAGRGRYGICCAEMDIWEANS | 207-239 | 211-223 | 231, 236 |
| | | cinerea okayama | | | | |
| SEQ ID NO: 164 | 154292161 | Botryotinia | NVEGWVPDSNSANSGTGNIGSCCSEFDVWEANS | 203-235 | 207-219 | 227, 232 |
| | | fuckeliana B05- | | | | |
| | | 10 | | | | |
| SEQ ID NO: 165 | 169615761 # | Phaeosphaeria | NADGWQASTSDPNAGVGKKGACCAEMDVWEANS | 183-215 | 187-199 | 207, 212 |
| | | nodorum SN15 | | | | |
| SEQ ID NO: 166 | 4883502 | Humicola grisea | NIEGWRPSTNDPNAGVGPMGACCAEIDVWESNA | 208-240 | 212-224 | 232, 237 |
| SEQ ID NO: 167 | 950686 | Humicola grisea | NIEGWTGSTNDPNAGAGRYGTCCSEMDIWEANN | 207-239 | 211-223 | 231, 236 |
| SEQ ID NO: 168 | 124491660 | Chaetomium | NIEGWRPSTNDANAGVGPYGACCAEIDVWESNA | 209-241 | 213-225 | 233, 238 |
| | | thermophilum | | | | |
| SEQ ID NO: 169 | 58045187 | Chaetomium | NIENWTPSTNDANAGFGRYGSCCSEMDIWEANN | 207-239 | 211-223 | 231, 236 |
| | | thermophilum | | | | |
| SEQ ID NO: 170 | 169601100 # | Phaeosphaeria | NVEGWKPSDNDANAGVGGHGSCCAEMDIWEANS | 174-206 | 178-190 | 198, 203 |
| | | nodorum SN15 | | | | |
| SEQ ID NO: 171 | 169870197 | Coprinopsis | NSVGWEPSETDSNAGRGRYGICCAEMDIWEANS | 207-239 | 211-223 | 231, 236 |
| | | cinerea okayama | | | | |
| SEQ ID NO: 172 | 3913806 | Agaricus | NSEGWEGSPNDVNAGTGNFGACCGEMDIWEANS | 203-235 | 207-219 | 227, 232 |
| | | bisporus | | | | |
| SEQ ID NO: 173 | 169611094 | Phaeosphaeria | NVEGWNPSDADPNAGSGKIGACCPEMDIWEANS | 208-240 | 212-224 | 232, 237 |
| | | nodorum SN15 | | | | |
| SEQ ID NO: 174 | 3131 | Phanerochaete | NVQGWNATS--ATTGTGSYGSCCTELDIWEANS | 204-234 | 208-218 | 226, 231 |
| | | chrysosporium | | | | |
| SEQ ID NO: 175 | 70991503 | Aspergillus | NVEGWEPSSSDKNAGVGGHGSCCPEMDIWEANS | 202-234 | 206-218 | 226, 231 |
| | | fumigatus Af293 | | | | |
| SEQ ID NO: 176 | 294196 | Phanerochaete | NVEGWNATS--ANAGTGNYGTCCTEMDIWEANN | 203-233 | 207-217 | 225, 230 |
| | | chrysosporium | | | | |
| SEQ ID NO: 177 | 18997123 | Thermoascus | NVEGWQPSANDPNAGVGNHGSSCAEMDVWEANS | 205-237 | 209-221 | 229, 234 |
| | | aurantiacus | | | | |
| SEQ ID NO: 178 | 4204214 | Humicola grisea | NIEGWRPSTNDPNAGVGPMGACCAEIDVWESNA | 208-240 | 212-224 | 232, 237 |
| | | var thermoidea | | | | |
| SEQ ID NO: 179 | 34582632 | Trichoderma | NVEGWEPSSNNANTGIGGHGSCCSEMDIWEANS | 205-237 | 209-221 | 229, 234 |
| | | viride (also | | | | |
| | | known as | | | | |
| | | Hypochrea rufa) | | | | |
| SEQ ID NO: 180 | 156712284 | Thermoascus | NVEGWQPSANDPNAGVGNHGSCCAEMDVWEANS | 205-237 | 209-221 | 229, 234 |
| | | aurantiacus | | | | |
| SEQ ID NO: 181 | 39977899 | Magnaporthe | NVEGWQPSSGDANSGVGNMGSCCAEMDIWEANS | 205-237 | 209-221 | 229, 234 |
| | | grisea (oryzae) | | | | |
| | | 70-15 | | | | |
| SEQ ID NO: 182 | 20986705 | Talaromyces | NVEGWQPSSNNANTGIGDHGSCCAEMDVWEANS | 203-235 | 207-219 | 227, 232 |
| | | emersonii | | | | |
| SEQ ID NO: 183 | 22138843 | Aspergillus | R-KGWEPSDSDKNAGVGGHGSCCPQMDIWEANS | 203-234 | 206-218 | 226, 231 |
| | | oryzae | | | | |
| SEQ ID NO: 184 | 55775695 | Penicillium | NVEGWEPSSSDVNGGTGNYGSCCAEMDIWEANS | 213-245 | 217-229 | 237, 242 |
| | | chrysogenum | | | | |
| SEQ ID NO: 185 | 171676762 | Podospora | NIEGWNPSTNDVNAGAGRYGTCCSEMDIWEANN | 207-239 | 211-223 | 231, 236 |
| | | anserina | | | | |
| SEQ ID NO: 186 | 146350520 | Pleurotus sp | NVQGWQPSPNDSNAGKGQYGSCCAEMDIWEANS | 207-239 | 211-223 | 231, 236 |
| | | Florida | | | | |
| SEQ ID NO: 187 | 37732123 | Gibberella zeae | NSDGWQPSDSDVNGGIGNLGTCCPEMDIWEANS | 205-237 | 209-221 | 229, 234 |
| SEQ ID NO: 188 | 156055188 | Sclerotinia | NNEGWVPDSNSANSGTGNIGSCCSEFDVWEANS | 203-235 | 207-219 | 227, 232 |
| | | sclerotiorum | | | | |
| | | 1980 | | | | |
| SEQ ID NO: 189 | 453224 | Phanerochaete | NVGNWTETG--SNTGTGSYGTCCSEMDIWEANN | 203-233 | 207-217 | 225, 230 |
| | | chrysosporium | | | | |
| SEQ ID NO: 190 | 50402144 | Trichoderma | NVEGWEPSSNNANTGIGGHGSCCSEMDIWEANS | 205-237 | 209-221 | 229, 234 |
| | | reesei | | | | |
| SEQ ID NO: 191 | 115397177 | Aspergillus | NVEGWEPSANDANAGTGNHGSCCAEMDIWEANS | 211-243 | 215-227 | 235, 240 |
| | | terreus NIH2624 | | | | |
| SEQ ID NO: 192 | 154312003 | Botryotinia | NSVGWTPSSNDVNAGAGQYGSCCSEMDIWEANK | 206-238 | 210-222 | 230, 235 |
| | | fuckeliana B05- | | | | |
| | | 10 | | | | |
| SEQ ID NO: 193 | 49333365 | Volvariella | NVQGWQPSPNDTNAGTGNYGACCNEMDVWEANS | 207-239 | 211-223 | 231, 236 |
| | | volvacea | | | | |
| SEQ ID NO: 194 | 729650 | Penicillium | NVDGWTPSKNDVNSGIGNHGSCCAEMDIWEANS | 211-243 | 215-227 | 235, 240 |
| | | janthinellum | | | | |
| SEQ ID NO: 195 | 146424871 | Pleurotus sp | NILDWSASATDANAGNGRYGACCAEMDIWEANS | 206-238 | 210-222 | 230, 235 |
| | | Florida | | | | |
| SEQ ID NO: 196 | 67538012 | Aspergillus | NVEGWEPSDSDANAGVGGMGTCCPEMDIWEANS | 202-234 | 206-218 | 226, 231 |
| | | nidulans FGSC | | | | |
| | | A4 | | | | |
| SEQ ID NO: 197 | 62006162 | Fusarium poae | NSDGWEPSKSDVNGGIGNLGTCCPEMDIWEANS | 205-237 | 209-221 | 229, 234 |
| SEQ ID NO: 198 | 146424873 | Pleurotus sp | NILDWSGSATDPNAGNGRYGACCAEMDIWEANS | 206-238 | 210-222 | 230, 235 |
| | | Florida | | | | |
| SEQ ID NO: 199 | 295937 | Trichoderma | NVEGWEPSSNNANTGIGGHGSCCSEMDIWEANS | 205-237 | 209-221 | 229, 234 |
| | | viride | | | | |
| SEQ ID NO: 200 | 6179889 # | Alternaria | NVEGWKPSSNDANAGVGGHGSCCAEMDIWEANS | 177-209 | 181-193 | 201, 206 |
| | | alternata | | | | |
| SEQ ID NO: 201 | 119483864 | Neosartorya | NVEGWTPSSNNENTGLGNYGSCCAELDIWESNS | 215-247 | 219-231 | 239, 244 |
| | | fischeri NRRL | | | | |
| | | 181 | | | | |
| SEQ ID NO: 202 | 85083281 | Neurospora | NIEGWTPSTNDANAGVGPYGGCCAEIDVWESNA | 207-239 | 211-223 | 231, 236 |
| | | crassa OR74A | | | | |
| SEQ ID NO: 203 | 3913803 | Cryphonectria | NVEGWTPSTNDANAGVGGLGSCCSEMDVWEANS | 206-238 | 210-222 | 230, 235 |
| | | parasitica | | | | |
| SEQ ID NO: 204 | 60729633 | Corticium rolfsii | NLLDWNATS--ANSGTGSYGSCCPEMDIWEANK | 206-236 | 210-220 | 228, 233 |
| SEQ ID NO: 205 | 39971383 | Magnaporthe | NIEGWQPSSTDSSAGIGAQGACCAEIDIWESNK | 205-237 | 209-221 | 229, 234 |
| | | grisea 70-15 | | | | |
| SEQ ID NO: 206 | 39973029 | Magnaporthe | NIEGWKPSSNDANAGVGPYGACCAEIDVWESNA | 206-238 | 210-222 | 230, 235 |
| | | grisea 70-15 | | | | |
| SEQ ID NO: 207 | 1170141 | Fusarium | NSEGWKPSDSDVNAGVGNLGTCCPEMDIWEANS | 205-237 | 209-221 | 229, 234 |
| | | oxysporum | | | | |
| SEQ ID NO: 208 | 121710012 | Aspergillus | NVEGWKPSDNDKNAGVGGYGSCCPEMDIWEANS | 202-234 | 206-218 | 226, 231 |
| | | clavatus NRRL 1 | | | | |
| SEQ ID NO: 209 | 17902580 | Penicillium | NVEGWTPSTNNSNTGIGNHGSCCAELDIWEANS | 210-242 | 214-226 | 234, 239 |
| | | funiculosum | | | | |
| SEQ ID NO: 210 | 1346226 | Humicola grisea | NIEGWTGSTNDPNAGAGRYGTCCSEMDIWEANN | 207-239 | 211-223 | 231, 236 |
| | | var thermoidea | | | | |
| SEQ ID NO: 211 | 156712282 | Chaetomium | NVGNWTPSTNDANAGFGRYGSCCSEMDVWEANN | 207-239 | 211-223 | 231, 236 |
| | | thermophilum | | | | |
| SEQ ID NO: 212 | 169768818 | Aspergillus | NVEGWVSSTNNANTGTGNHGSCCAELDIWESNS | 214-246 | 218-230 | 238, 243 |
| | | oryzae RIB40 | | | | |
| SEQ ID NO: 213 | 46241270 | Gibberella | NSDGWQPSKSDVNAGIGNMGTCCPEMDIWEANS | 205-237 | 209-221 | 229, 234 |
| | | pulicaris | | | | |
| SEQ ID NO: 214 | 49333363 | Volvariella | NVAGWNGSPNDTNAGTGNWGACCNEMDIWEANS | 205-237 | 209-221 | 229, 234 |
| | | volvacea | | | | |
| SEQ ID NO: 215 | 46395332 | Irpex lacteus | NVAGWTGSSSDPNSGTGNYGTCCSEMDIWEANS | 202-234 | 206-218 | 226, 231 |
| SEQ ID NO: 216 | 50844407 # | Chaetomium | NIENWTPSTNDANAGFGRYGSCCSEMDIWEANN | 182-214 | 186-198 | 206, 211 |
| | | thermophilum var | | | | |
| | | thermophilum | | | | |
| SEQ ID NO: 217 | 4586347 | Irpex lacteus | NIVDWTASAGDANSGTGSFGTCCQEMDIWEANS | 203-235 | 207-219 | 227, 232 |
| SEQ ID NO: 218 | 3980202 | Phanerochaete | NVGNWTETG--SNTGTGSYGTCCSEMDIWEANN | 203-233 | 207-217 | 225, 230 |
| | | chrysosporium | | | | |
| SEQ ID NO: 219 | 27125837 | Melanocarpus | NIEGWKSSTSDPNAGVGPYGSCCAEIDVWESNA | 210-242 | 214-226 | 234, 239 |
| | | albomyces | | | | |
| SEQ ID NO: 220 | 171696102 | Podospora | NVEGWGGAD--GNSGTGKYGICCAEMDIWEANS | 206-236 | 210-220 | 228, 233 |
| | | anserina | | | | |
| SEQ ID NO: 221 | 3913802 | Cochliobolus | NVEGWNPSDADPNGGAGKIGACCPEMDIWEANS | 208-240 | 212-224 | 232, 237 |
| | | carbonum | | | | |
| SEQ ID NO: 222 | 50403723 | Trichoderma | NVEGWEPSSNNANTGIGGHGSCCSEMDIWEANS | 205-237 | 209-221 | 229, 234 |
| | | viride | | | | |
| SEQ ID NO: 223 | 3913798 | Aspergillus | NIEGWEPSSTDVNAGTGNHGSCCPEMDIWEANS | 210-242 | 214-226 | 234, 239 |
| | | aculeatus | | | | |
| SEQ ID NO: 224 | 66828465 | Dictyostelium | NVDGWIPSTNNPNTGYGNLGSCCAEMDLWEANN | 206-238 | 210-222 | 230, 235 |
| | | discoideum | | | | |
| SEQ ID NO: 225 | 156060391 | Sclerotinia | NSVGWTPSSNDVNTGTGQYGSCCSEMDIWEANK | 192-224 | 196-208 | 216, 221 |
| | | sclerotiorum | | | | |
| | | 1980 | | | | |
| SEQ ID NO: 226 | 116181754 | Chaetomium | NSEGWGGED--GNSGTGKYGTCCAEMDIWEANL | 203-233 | 207-217 | 225, 230 |
| | | globosum CBS | | | | |
| | | 148-51 | | | | |
| SEQ ID NO: 227 | 145230535 | Aspergillus niger | NCDGWEPSSNNVNTGVGDHGSCCAEMDVWEANS | 209-241 | 213-225 | 233, 238 |
| SEQ ID NO: 228 | 46241266 | Nectria | NSDEWKPSDSDKNAGVGKYGTCCPEMDIWEANK | 205-237 | 209-221 | 229, 234 |
| | | haematococca | | | | |
| | | mpVI | | | | |
| SEQ ID NO: 229 | 1q9h (PDB) # | Talaromyces | NVEGWQPSSNNANTGIGDHGSCCAEMDVWEANS | 185-217 | 189-201 | 209, 214 |
| | | emersonii | | | | |
| SEQ ID NO: 230 | 157362170 | Polyporus | NVLDWAGSSNDPNAGTGHYGTCCNEMDIWEANS | 208-240 | 212-224 | 232, 237 |
| | | arcularius | | | | |
| SEQ ID NO: 231 | 7804885 | Leptosphaeria | NAEGWTKSASDPNSGVGKKGACCAQMDVWEANS | 204-236 | 208-220 | 228, 233 |
| | | maculans | | | | |
| SEQ ID NO: 232 | 121852 | Phanerochaete | NVEGWNATS--ANAGTGNYGTCCTEMDIWEANN | 203-233 | 207-217 | 225, 230 |
| | | chrysosporium | | | | |
| SEQ ID NO: 233 | 126013214 | Penicillium | NVEGWKPSANDKNAGVGPHGSCCAEMDIWEANS | 201-233 | 205-217 | 225, 230 |
| | | decumbens | | | | |
| SEQ ID NO: 234 | 156048578 | Sclerotinia | NVDGWVPSSNNPNTGVGNYGSCCAEMDIWEANS | 202-234 | 206-218 | 226, 231 |
| | | sclerotiorum | | | | |
| | | 1980 | | | | |
| SEQ ID NO: 235 | 156712278 | Acremonium | NIDGWQPSSNDANAGLGNHGSCCSEMDIWEANK | 206-238 | 210-222 | 230, 235 |
| | | thermophilum | | | | |
| SEQ ID NO: 236 | 21449327 | Aspergillus | NVEGWEPSDSDANAGVGGMGTCCPEMDIWEANS | 202-234 | 206-218 | 226, 231 |
| | | nidulans (also | | | | |
| | | known as | | | | |
| | | Emericella | | | | |
| | | nidulans) | | | | |
| SEQ ID NO: 237 | 171683762 | Podospora | NIEGWRESSNDENAGVGPYGGCCAEIDVWESNA | 211-243 | 215-227 | 235, 240 |
| | | anserine (S | | | | |
| | | mat+) | | | | |
| SEQ ID NO: 238 | 56718412 | Thermoascus | NVEGWQPSANDPNAGVGNHGSCCAEMDVWEANS | 205-237 | 209-221 | 229, 234 |
| | | aurantiacus var | | | | |
| | | levisporus | | | | |
| SEQ ID NO: 239 | 15824273 | Pseudotrichonympha | NVENWKPQTNDENAGNGRYGACCTEMDIWEANK | 200-232 | 204-216 | 224, 229 |
| | | grassii | | | | |
| SEQ ID NO: 240 | 115390801 | Aspergillus | NVEGWTPSDNDKNAGVGGHGSCCPELDIWEANS | 203-235 | 207-219 | 227, 232 |
| | | terreus NIH2624 | | | | |
| SEQ ID NO: 241 | 453223 | Phanerochaete | NVGNWTETG--SNTGTGSYGTCCSEMDIWEANN | 203-233 | 207-217 | 225, 230 |
| | | chrysosporium | | | | |
| SEQ ID NO: 242 | 3132 | Phanerochaete | NVEGWLGTT--ATTGTGFFGSCCTDIALWEAND | 202-232 | 206-216 | 224, 229 |
| | | chrysosporium | | | | |
| SEQ ID NO: 243 | 16304152 | Thermoascus | NVEGWQPSANDPNAGVGNHGSSCAEMDVWEANS | 205-237 | 209-221 | 229, 234 |
| | | aurantiacus | | | | |
| SEQ ID NO: 244 | 156712280 | Acremonium | NSASWQPSSNDQNAGVGGMGSCCAEMDIWEANS | 210-242 | 214-226 | 234, 239 |
| | | thermophilum | | | | |
| SEQ ID NO: 245 | 5231154 | Volvariella | NVQGWQPSPNDTNAGTGNYGACCNKMDVWEANS | 220-252 | 224-236 | 244, 249 |
| | | volvacea | | | | |
| SEQ ID NO: 246 | 116200349 | Chaetomium | NYDGWTPSSNDANAGVGALGGCCAEIDVWESNA | 207-239 | 211-223 | 231, 236 |
| | | globosum CBS | | | | |
| | | 148-51 | | | | |
| SEQ ID NO: 247 | 4586343 | Irpex lacteus | NVAGWAGSASDPNAGSGTLGTCCSEMDIWEANN | 202-234 | 206-218 | 226, 231 |
| SEQ ID NO: 248 | 15321718 | Lentinula edodes | NVEGWTPSSTSPNAGTGGTGICCNEMDIWEANS | 208-240 | 212-224 | 232, 237 |
| SEQ ID NO: 249 | 146424875 | Pleurotus sp | NVLDWSASATDDNAGNGRYGACCAEMDIWEANS | 206-238 | 210-222 | 230, 235 |
| | | Florida | | | | |
| SEQ ID NO: 250 | 62006158 | Fusarium | NSDGWQPSKSDVNGGIGNLGTCCPEMDIWEANS | 205-237 | 209-221 | 229, 234 |
| | | venenatum | | | | |
| SEQ ID NO: 251 | 296027 | Phanerochaete | NVEGWNATS--ANAGTGNYGTCCTEMDIWEANN | 203-233 | 207-217 | 225, 230 |
| | | chrysosporium | | | | |
| SEQ ID NO: 252 | 154449709 | Fusicoccum sp | NVQNWTASSTDKNAGTGHYGSCCNEMDIWEANS | 209-241 | 213-225 | 233, 238 |
| | | BCC4124 | | | | |
| SEQ ID NO: 253 | 169859460 | Coprinopsis | NSVGWEPSETDPNAGKGQYGICCAEMDIWEANS | 207-239 | 211-223 | 231, 236 |
| | | cinerea okayama | | | | |
| SEQ ID NO: 254 | 50400675 | Trichoderma | NVEGWEPSSNNANTGVGGHGSCCSEMDIWEANS | 201-233 | 205-217 | 225, 230 |
| | | harzianum | | | | |
| | | (anamorph of | | | | |
| | | Hypocrea lixii) | | | | |
| SEQ ID NO: 255 | 729649 | Neurospora | NVEGWTPSTNDAN-GIGDHGSCCSEMDIWEANK | 200-231 | 204-215 | 223, 228 |
| | | crassa (OR74A) | | | | |
| SEQ ID NO: 256 | 119472134 | Neosartorya | NVEGWQPSSNDANAGTGNHGSCCAEMDIWEANS | 214-246 | 218-230 | 238, 243 |
| | | fischeri NRRL | | | | |
| | | 181 | | | | |
| SEQ ID NO: 257 | 117935080 | Chaetomium | NIEGWRPSTNDANAGVGPYGACCAEIDVWESNA | 209-241 | 213-225 | 233, 238 |
| | | thermophilum | | | | |
| SEQ ID NO: 258 | 154300584 | Botryotinia | NVDGWVPSSNNANTGVGNHGSCCAEMDIWEANS | 202-234 | 206-218 | 226, 231 |
| | | fuckeliana B05- | | | | |
| | | 10 | | | | |
| SEQ ID NO: 259 | 15824271 | Pseudotrichonympha | NVENWKPQTNDENAGNGRYGACCTEMDIWEANK | 200-232 | 204-216 | 224, 229 |
| | | grassii | | | | |
| SEQ ID NO: 260 | 4586345 | Irpex lacteus | NVEGWTGSSTDSNSGTGNYGTCCSEMDIWEANS | 202-234 | 206-218 | 226, 231 |
| SEQ ID NO: 261 | 46241268 | Gibberella | NSDGWKPSDSDINAGIGNMGTCCPEMDIWEANS | 205-237 | 209-221 | 229, 234 |
| | | avenacea | | | | |
| SEQ ID NO: 262 | 6164684 | Aspergillus niger | NCDGWEPSSNNVNTGVGDHGSCCAEMDVWEANS | 209-241 | 213-225 | 233, 238 |
| SEQ ID NO: 263 | 6164682 | Aspergillus niger | NVDGWEPSSNNDNTGIGNHGSCCPEMDIWEANK | 203-235 | 207-219 | 227, 232 |
| SEQ ID NO: 264 | 33733371 | Chrysosporium | NVENWQSSTNDANAGTGKYGSCCSEMDVWEANN | 206-238 | 210-222 | 230, 235 |
| | | luckowense | | | | |
| | | U.S. Pat. No. 6,573,086-10 | | | | |
| SEQ ID NO: 265 | 29160311 | Thielavia | NVEGWESSTNDANAGSGKYGSCCTEMDVWEANN | 206-238 | 210-222 | 230, 235 |
| | | australiensis | | | | |
| SEQ ID NO: 266 | 146197087 | uncultured | NVDDWKPQDNDENSGNGKLGTCCSEMDIWEGNM | 197-229 | 201-213 | 221, 226 |
| | | symbiotic protist | | | | |
| | | of Reticulitermes, | | | | |
| | | speratus | | | | |
| SEQ ID NO: 267 | 146197237 | uncultured | NSEGWKPQSGDKNAGNGKYGSCCSEMDVWESNS | 200-232 | 204-216 | 224, 229 |
| | | symbiotic protist | | | | |
| | | of Neotermes | | | | |
| | | koshunensis | | | | |
| SEQ ID NO: 268 | 146197067 | uncultured | NVDDWKPQDNDENSGNGKLGTCCSEMDIWEGNM | 197-229 | 201-213 | 221, 226 |
| | | symbiotic protist | | | | |
| | | of Reticulitermes | | | | |
| | | speratus | | | | |
| SEQ ID NO: 269 | 146197407 | uncultured | NVLDWKPQSNDENSGNGRYGACCTEMDIWEANS | 198-230 | 202-214 | 222, 227 |
| | | symbiotic protist | | | | |
| | | of Cryptocercus | | | | |
| | | punctulatus | | | | |
| SEQ ID NO: 270 | 146197157 | uncultured | NVEGWKPSDNDENAGTGKWGACCTEMDIWEANK | 201-233 | 205-217 | 225, 230 |
| | | symbiotic protist | | | | |
| | | of Hodotermopsis | | | | |
| | | sjoestedti | | | | |
| SEQ ID NO: 271 | 146197403 | uncultured | NVLDWKPQSNDENSGNGRYGACCTEMDIWEANS | 198-230 | 202-214 | 222, 227 |
| | | symbiotic protist | | | | |
| | | of Cryptocercus | | | | |
| | | punctulatus | | | | |
| SEQ ID NO: 272 | 146197081 | uncultured | NVDDWKPQDNDENSGDGKLGTCCSEMDIWEGNA | 197-229 | 201-213 | 221, 226 |
| | | symbiotic protist | | | | |
| | | of Reticulitermes | | | | |
| | | speratus | | | | |
| SEQ ID NO: 273 | 146197413 | uncultured | NVLDWKPQSNDENSGNGRYGACCTEMDIWEANS | 198-230 | 202-214 | 222, 227 |
| | | symbiotic protist | | | | |
| | | of Cryptocercus | | | | |
| | | punctulatus | | | | |
| SEQ ID NO: 274 | 146197309 | uncultured | NSDGWKPQSNDKNSGNGKYGSCCSEMDIWEANS | 196-228 | 200-212 | 220, 225 |
| | | symbiotic protist | | | | |
| | | of Mastotermes | | | | |
| | | darwiniensis | | | | |
| SEQ ID NO: 275 | 146197227 | uncultured | NSDGWKPQKNDKNSGNGKYGSCCSEMDIWEANS | 195-227 | 199-211 | 219, 224 |
| | | symbiotic protist | | | | |
| | | of Neotermes | | | | |
| | | koshunensis | | | | |
| SEQ ID NO: 276 | 146197253 | uncultured | NSEGWKPQSGDKNAGNGKYGSCCSEMDVWESNS | 200-232 | 204-216 | 224, 229 |
| | | symbiotic protist | | | | |
| | | of Neotermes | | | | |
| | | koshunensis | | | | |
| SEQ ID NO: 277 | 146197099 | uncultured | NVLDWKPQSNDENAGTGRYGTCCTEMDIWEANS | 197-229 | 201-213 | 221, 226 |
| | | symbiotic protist | | | | |
| | | of Reticulitermes | | | | |
| | | speratus | | | | |
| SEQ ID NO: 278 | 146197409 | uncultured | NVLDWKPQSNDENSGNGRWGARCTEMDIWEANS | 198-230 | 202-214 | 222, 227 |
| | | symbiotic protist | | | | |
| | | of Cryptocercus | | | | |
| | | punctulatus | | | | |
| SEQ ID NO: 279 | 146197315 | uncultured | NSDGWKPQSNDKNSGNGKYGSCCSEMDIWEANS | 196-228 | 200-212 | 220, 225 |
| | | symbiotic protist | | | | |
| | | of Mastotermes | | | | |
| | | darwiniensis | | | | |
| SEQ ID NO: 280 | 146197411 | uncultured | NVLDWKPQSNDENSGNGRYGACCTEMDIWEANS | 198-230 | 202-214 | 222, 227 |
| | | symbiotic protist | | | | |
| | | of Cryptocercus | | | | |
| | | punctulatus | | | | |
| SEQ ID NO: 281 | 146197161 | uncultured | NVQDWKPSDNDDNAGTGHYGACCTEMDIWEANK | 201-233 | 205-217 | 225, 230 |
| | | symbiotic protist | | | | |
| | | of Hodotermopsis | | | | |
| | | sjoestedti | | | | |
| SEQ ID NO: 282 | 146197323 | uncultured | NSDGWKPQSNDKNSGNGKYGSCCSEMDIWEANS | 196-228 | 200-212 | 220, 225 |
| | | symbiotic protist | | | | |
| | | of Mastotermes | | | | |
| | | darwiniensis | | | | |
| SEQ ID NO: 283 | 146197077 | uncultured | NVLDWKPQETDENSGNGRYGTCCTEMDIWEANS | 201-233 | 205-217 | 225, 230 |
| | | symbiotic protist | | | | |
| | | of Reticulitermes | | | | |
| | | speratus | | | | |
| SEQ ID NO: 284 | 146197089 | uncultured | NVEDWKPQDNDENSGNGKLGTCCSEMDIWEGNA | 197-229 | 201-213 | 221, 226 |
| | | symbiotic protist | | | | |
| | | of Reticulitermes | | | | |
| | | speratus | | | | |
| SEQ ID NO: 285 | 146197091 | uncultured | NVLDWKPQSNDENAGTGRYGTCCTEMDIWEANS | 197-229 | 201-213 | 221, 226 |
| | | symbiotic protist | | | | |
| | | of Reticulitermes | | | | |
| | | speratus | | | | |
| SEQ ID NO: 286 | 146197097 | uncultured | NVDDWKPQDNDENSGNGKLGTCCSEMDIWEGNA | 197-229 | 201-213 | 221, 226 |
| | | symbiotic protist | | | | |
| | | of Reticulitermes | | | | |
| | | speratus | | | | |
| SEQ ID NO: 287 | 146197095 | uncultured | NVDDWKPQDNDENSGNGKLGTCCSEMDIWEGNA | 197-229 | 201-213 | 221, 226 |
| | | symbiotic protist | | | | |
| | | of Reticulitermes | | | | |
| | | speratus | | | | |
| SEQ ID NO: 288 | 146197401 | uncultured | NVLDWKPQSNDENSGNGRYGACCIEMDIWEANS | 198-230 | 202-214 | 222, 227 |
| | | symbiotic protist | | | | |
| | | of Cryptocercus | | | | |
| | | punctulatus | | | | |
| SEQ ID NO: 289 | 146197225 | uncultured | NSDGWKPQKNDKNSGNGKYGSCCSEMDIWEANS | 195-227 | 199-211 | 219, 224 |
| | | symbiotic protist | | | | |
| | | of Neotermes | | | | |
| | | koshunensis | | | | |
| SEQ ID NO: 290 | 146197317 | uncultured | NSDGWKPQSNDKNSGNGKYGSCCSEMDIWEANS | 196-228 | 200-212 | 220, 225 |
| | | symbiotic protist | | | | |
| | | of Mastotermes | | | | |
| | | darwiniensis | | | | |
| SEQ ID NO: 291 | 146197251 | uncultured | NSDGWKPQKNDKNSGNGRYGSCCSEMDVWEANS | 195-227 | 199-211 | 219, 224 |
| | | symbiotic protist | | | | |
| | | of Neotermes | | | | |
| | | koshunensis | | | | |
| SEQ ID NO: 292 | 146197319 | uncultured | NSDGWKPQSNDKNSGNGKYGSCCSEMDIWEANS | 196-228 | 200-212 | 220, 225 |
| | | symbiotic protist | | | | |
| | | of Mastotermes | | | | |
| | | darwiniensis | | | | |
| SEQ ID NO: 293 | 146197071 | uncultured | NILDWKPSSNDENAGAGRYGTCCTEMDIWEANS | 200-232 | 204-216 | 224, 229 |
| | | symbiotic protist | | | | |
| | | of Reticulitermes | | | | |
| | | speratus | | | | |
| SEQ ID NO: 294 | 146197075 | uncultured | NVDDWKPQDNDENSGNGKLGTCCSEMDIWEGNA | 197-229 | 201-213 | 221, 226 |
| | | symbiotic protist | | | | |
| | | of Reticulitermes | | | | |
| | | speratus | | | | |
| SEQ ID NO: 295 | 146197159 | uncultured | NVKDWKPQETDENAGNGHYGACCTEMDIWEANS | 197-229 | 201-213 | 221, 226 |
| | | symbiotic protist | | | | |
| | | of Hodotermopsis | | | | |
| | | sjoestedti | | | | |
| SEQ ID NO: 296 | 146197405 | uncultured | NVLDWKPQSNDENSGNGRYGACCTEMDIWEANS | 198-230 | 202-214 | 222, 227 |
| | | symbiotic protist | | | | |
| | | of Cryptocercus | | | | |
| | | punctulatus | | | | |
| SEQ ID NO: 297 | 146197327 | uncultured | NSDGWKPQDNDENSGNGKYGSCCSEMDIWEANS | 201-233 | 205-217 | 225, 230 |
| | | symbiotic protist | | | | |
| | | of Mastotermes | | | | |
| | | darwiniensis | | | | |
| SEQ ID NO: 298 | 146197261 | uncultured | NSDGWKPQKNDKNSGNGKYGSCCSEMDIWEANS | 195-227 | 199-211 | 219, 224 |
| | | symbiotic protist | | | | |
| | | of Neotermes | | | | |
| | | koshunensis | | | | |

TABLE 5

| | Tolerance to | Tolerance to |
|---|---|---|
| | 250 mg/L cellobiose | cellobiose accumulation |
| | % Activity in 4- | % Activity in |
| | MUL Assay | Bagasse Assay |
| Substitution(s) | (+/−Cellobiose)* | (+/−BG)^{¥} |
| None | 25% | 60% |
| R273K/R422K | 95% | 84% |
| R273K/Y274Q/D281K/ | 78% | ND |
| Y410H/P411G/R422K | | |

TABLE 6

| | Tolerance to | |
|---|---|---|
| | 250 mg/L cellobiose | Tolerance to |
| | % Activity in 4- | cellobiose accumulation |
| | MUL Assay | % Activity in |
| Substitution(s) | (+/−Cellobiose)* | Bagasse Assay (+/−BG)^{¥} |
| None | 23% | 74% |
| R268K/R411K | 92% | 94% |
| R268A/R411A | 92% | 95% |
| R268A/R411K | 97% | 94% |
| R268K/R411A | 97% | 102% |
| R268K | ND | 92% |
| R268A | ND | 86% |
| R411K | ND | 89% |
| R411A | ND | 94% |

TABLE 7

| SEQ ID NO. | Amino acid sequence |
|---|---|
| SEQ ID NO: 1 | MSALNSFNMY KSALILGSLL ATAGAQQIGT YTAETHPSLS WSTCKSGGSC TTNSGAITLD ANWRWVHGVN TSTNCYTGNT WNTAICDTDA SCAQDCALDG ADYSGTYGIT |
| | TSGNSLRLNF VTGSNVGSRT YLMADNTHYQ IFDLLNQEFT FTVDVSHLPC GLNGALYFVT MDADGGVSKY PNNKAGAQYG VGYCDSQCPR DLKFIAGQAN VEGWTPSSNN |
| | ANTGLGNHGA CCAELDIWEA NSISEALTPH PCDTPGLSVC TTDACGGTYS SDRYAGTCDP DGCDFNPYRL GVTDFYGSGK TVDTTKPITV VTQFVTDDGT STGTLSEIRR |
| | YYVQNGVVIP QPSSKISGVS GNVINSDFCD AEISTFGETA SFSKHGGLAK MGAGMEAGMV LVMSLWDDYS VNMLWLDSTY PTNATGTPGA ARGSCPTTSG DPKTVESQSG |
| | SSYVTFSDIR VGPFNSTFSG GSSTGGSSTT TASGTTTTKA SSTSTSSTST GTGVAAHWGQ CGGQGWTGPT TCASGTTCTV VNPYYSQCL |
| SEQ ID NO: 2 | MYRKLAVISA FLATARAQSA CTLQSETHPP LTWQKCSSGG TCTQQTGSVV IDANWRWTHA TNSSTNCYDG NTWSSTLCPD NETCAKNCCL DGAAYASTYG VTTSGNSLSI |
| | GFVTQSAQKN VGARLYLMAS DTTYQEFTLL GNEFSFDVDV SQLPCGLNGA LYFVSMDADG GVSKYPTNTA GAKYGTGYCD SQCPRDLKFI NGQANVEGWE PSSNNANTGI |
| | GGHGSCCSEM DIWEANSISE ALTPHPCTTV GQEICEGDGC GGTYSDNRYG GTCDPDGCDW NPYRLGNTSF YGPGSSFTLD TTKKLTVVTQ FETSGAINRY YVQNGVTFQQ |
| | PNAELGSYSG NELNDDYCTA EEAEFGGSSF SDKGGLTQFK KATSGGMVLV MSLWDDYYAN MLWLDSTYPT NETSSTPGAV RGSCSTSSGV PAQVESQSPN AKVTFSNIKF |
| | GPIGSTGNPS GGNPPGGNPP GTTTTRRPAT TTGSSPGPTQ SHYGQCGGIG YSGPTVCASG TTCQVLNPYY SQCL |
| SEQ ID NO: 3 | MSALNSFNMY KSALILGSLL ATAGAQQIGT YTAETHPSLS WSTCKSGGSC TTNSGAITLD ANWRWVHGVN TSTNCYTGNT WNSAICDTDA SCAQDCALDG ADYSGTYGIT |
| | TSGNSLRLNF VTGSNVGSRT YLMADNTHYQ IFDLLNQEFT FTVDVSHLPC GLNGALYFVT MDADGGVSKY PNNKAGAQYG VGYCDSQCPR DLKFIAGQAN VEGWTPSANN |
| | ANTGIGNHGA CCAELDIWEA NSISEALTPH PCDTPGLSVC TTDACGGTYS SDRYAGTCDP DGCDFNPYRL GVTDFYGSGK TVDTTKPFTV VTQFVTNDGT STGSLSEIRR |
| | YYVQNGVVIP QPSSKISGIS GNVINSDYCA AEISTFGGTA SFNKHGGLTN MAAGMEAGMV LVMSLWDDYA VNMLWLDSTY PTNATGTPGA ARGTCATTSG DPKTVESQSG |
| | SSYVTFSDIR VGPFNSTFSG GSSTGGSTTT TASRTTTTSA SSTSTSSTST GTGVAGHWGQ CGGQGWTGPT TCVSGTTCTV VNPYYSQCL |
| SEQ ID NO: 4 | ESACTLQSET HPPLTWQKCS SGGTCTQQTG SVVIDANWRW THATNSSTNC YDGNTWSSTL CPDNETCAKN CCLDGAAYAS TYGVTTSGNS LSIDFVTQSA QKNVGARLYL |
| | MASDTTYQEF TLLGNEFSFD VDVSQLPCGL NGALYFVSMD ADGGVSKYPT NTAGAKYGTG YCDSQCPRDL KFINGQANVE GWEPSSNNAN TGIGGHGSCC SEMDIWQANS |
| | ISEALTPHPC TTVGQEICEG DGCGGTYSDN RYGGTCDPDG CDWNPYRLGN TSFYGPGSSF TLDTTKKLTV VTQFETSGAI NRYYVQNGVT FQQPNAELGS YSGNELNDDY |
| | CTAEEAEFGG SSFSDKGGLT QFKKATSGGM VLVMSLWDDY YANMLWLDST YPTNETSSTP GAVRGSCSTS SGVPAQVESQ SPNAKVTFSN IKFGPIGSTG NPSG |
| SEQ ID NO: 5 | MASSFQLYKA LLFFSSLLSA VQAQKVGTQQ AEVHPGLTWQ TCTSSGSCTT VNGEVTIDAN WRWLHTVNGY TNCYTGNEWD TSICTSNEVC AEQCAVDGAN YASTYGITTS |
| | GSSLRLNFVT QSQQKNIGSR VYLMDDEDTY TMFYLLNKEF TFDVDVSELP CGLNGAVYFV SMDADGGKSR YATNEAGAKY GTGYCDSQCP RDLKFINGVA NVEGWESSDT |
| | NPNGGVGNHG SCCAEMDIWE ANSISTAFTP HPCDTPGQTL CTGDSCGGTY SNDRYGGTCD PDGCDFNSYR QGNKTFYGPG LTVDTNSPVT VVTQFLTDDN TDTGTLSEIK |
| | RFYVQNGVVI PNSESTYPAN PGNSITTEFC ESQKELFGDV DVFSAHGGMA GMGAALEQGM VLVLSLWDDN YSNMLWLDSN YPTDADPTQP GIARGTCPTD SGVPSEVEAQ |
| | YPNAYVVYSN IKFGPIGSTF GNGGGSGPTT TVTTSTATST TSSATSTATG QAQHWEQCGG NGWTGPTVCA SPWACTVVNS WYSQCL |
| SEQ ID NO: 6 | MYRAIATASA LIAAVRAQQV CSLTQESKPS LNWSKCTSSG CSNVKGSVTI DANWRWTHQV SGSTNCYTGN KWDTSVCTSG KVCAEKCCLD GADYASTYGI TSSGDQLSLS |
| | FVTKGPYSTN IGSRTYLMED ENTYQMFQLL GNEFTFDVDV SNIGCGLNGA LYFVSMDADG GKAKYPGNKA GAKYGTGYCD AQCPRDVKFI NGQANSDGWQ PSDSDVNGGI |
| | GNLGTCCPEM DIWEANSIST AYTPHPCTKL TQHSCTGDSC GGTYSNDRYG GTCDADGCDF NSYRQGNKTF YGPGSGFNVD TTKKVTVVTQ FHKGSNGRLS EITRLYVQNG |
| | KVIANSESKI AGVPGNSLTA DFCTKQKKVF NDPDDFTKKG AWSGMSDALE APMVLVMSLW HDHHSNMLWL DSTYPTDSTK LGSQRGSCST SSGVPADLEK NVPNSKVAFS |
| | NIKFGPIGST YKSDGTTPTN PTNPSEPSNT ANPNPGTVDQ WGQCGGSNYS GPTACKSGFT CKKINDFYSQ CQ |
| SEQ ID NO: 7 | MLASTFSYRM YKTALILAAL LGSGQAQQVG TSQAEVHPSM TWQSCTAGGS CTTNNGKVVI DANWRWVHKV GDYTNCYTGN TWDTTICPDD ATCASNCALE GANYESTYGV |
| | TASGNSLRLN FVTTSQQKNI GSRLYMMKDD STYEMFKLLN QEFTFDVDVS NLPCGLNGAL YFVAMDADGG MSKYPTNKAG AKYGTGYCDS QCPRDLKFIN GQANVEGWQP |
| | SSNDANAGTG NHGSCCAEMD IWEANSISTA FTPHPCDTPG QVMCTGDACG GTYSSDRYGG TCDPDGCDFN SFRQGNKTFY GPGMTVDTKS KFTVVTQFIT DDGTSSGTLK |
| | EIKRFYVQNG KVIPNSESTW TGVSGNSITT EYCTAQKSLF QDQNVFEKHG GLEGMGAALA QGMVLVMSLW DDHSANMLWL DSNYPTTASS TTPGVARGTC DISSGVPADV |
| | EANHPDAYVV YSNIKVGPIG STFNSGGSNP GGGTTTTTTT QPTTTTTTAG NPGGTGVAQH YGQCGGIGWT GPTTCASPYT CQKLNDYYSQ CL |
| SEQ ID NO: 8 | MLPSTISYRI YKNALFFAAL FGAVQAQKVG TSKAEVHPSM AWQTCAADGT CTTKNGKVVI DANWRWVHDV KGYTNCYTGN TWNAELCPDN ESCAENCALE GADYAATYGA |
| | TTSGNALSLK FVTQSQQKNI GSRLYMMKDD NTYETFKLLN QEFTFDVDVS NLPCGLNGAL YFVSMDADGG LSRYTGNEAG AKYGTGYCDS QCPRDLKFIN GLANVEGWTP |
| | SSSDANAGNG GHGSCCAEMD IWEANSISTA YTPHPCDTPG QAMCNGDSCG GTYSSDRYGG TCDPDGCDFN SYRQGNKSFY GPGMTVDTKK KMTVVTQFLT NDGTATGTLS |
| | EIKRFYVQDG KVIANSESTW PNLGGNSLTN DFCKAQKTVF GDMDTFSKHG GMEGMGAALA EGMVLVMSLW DDHNSNMLWL DSNSPTTGTS TTPGVARGSC DISSGDPKDL |
| | EANHPDASVV YSNIKVGPIG STFNSGGSNP GGSTTTTKPA TSTTTTKATT TATTNTTGPT GTGVAQPWAQ CGGIGYSGPT QCAAPYTCTK QNDYYSQCL |
| SEQ ID NO: 9 | MHPSLQTILL SALFTTAHAQ QACSSKPETH PPLSWSRCSR SGCRSVQGAV TVDANWLWTT VDGSQNCYTG NRWDTSICSS EKTCSESCCI DGADYAGTYG VTTTGDALSL |
| | KFVQQGPYSK NVGSRLYLMK DESRYEMFTL LGNEFTFDVD VSKLGCGLNG ALYFVSMDED GGMKRFPMNK AGAKFGTGYC DSQCPRDVKF INGMANSKDW IPSKSDANAG |
| | IGSLGACCRE MDIWEANNIA SAFTPHPCKN SAYHSCTGDG CGGTYSKNRY SGDCDPDGCD FNSYRLGNTT FYGPGPKFTI DTTRKISVVT QFLKGRDGSL REIKRFYVQN |
| | GKVIPNSVSR VRGVPGNSIT QGFCNAQKKM FGAHESFNAK GGMKGMSAAV SKPMVLVMSL WDDHNSNMLW LDSTYPTNSR QRGSKRGSCP ASSGRPTDVE SSAPDSTVVF |
| | SNIKFGPIGS TFSRGK |
| SEQ ID NO: 10 | EQAGTNTAEN HPQLQSQQCT TSGGCKPLST KVVLDSNWRW VHSTSGYTNC YTGNEWDTSL CPDGKTCAAN CALDGADYSG TYGITSTGTA LTLKFVTGSN VGSRVYLMAD |
| | DTHYQLLKLL NQEFTFDVDM SNLPCGLNGA LYLSAMDADG GMSKYPGNKA GAKYGTGYCD SQCPKDIKFI NGEANVGNWT ETGSNTGTGS YGTCCSEMDI WEANNDAAAF |
| | TPHPCTTTGQ TRCSGDDCAR NTGLCDGDGC DFNSFRMGDK TFLGKGMTVD TSKPFTVVTQ FLTNDNTSTG TLSEIRRIYI QNGKVIQNSV ANIPGVDPVN SITDNFCAQQ |
| | KTAFGDTNWF AQKGGLKQMG EALGNGMVLA LSIWDDHAAN MLWLDSDYPT DKDPSAPGVA RGTCATTSGV PSDVESQVPN SQVVFSNIKF GDIGSTFSGT S |
| SEQ ID NO: 11 | MHQRALLFSA LAVAANAQQV GTQKPETHPP LTWQKCTAAG SCSQQSGSVV IDANWRWLHS TKDTTNCYTG NTWNTELCPD NESCAQNCAV DGADYAGTYG VTTSGSELKL |
| | SFVTGANVGS RLYLMQDDET YQHFNLLNNE FTFDVDVSNL PCGLNGALYF VAMDADGGMS KYPSNKAGAK YGTGYCDSQC PRDLKFINGM ANVEGWKPSS NDKNAGVGGH |
| | GSCCPEMDIW EANSISTAVT PHPCDDVSQT MCSGDACGGT YSATRYAGTC DPDGCDFNPF RMGNESFYGP GKIVDTKSEM TVVTQFITAD GTDTGALSEI KRLYVQNGKV |
| | IANSVSNVAD VSGNSISSDF CTAQKKAFGD EDIFAKHGGL SGMGKALSEM VLIMSIWDDH HSSMMWLDST YPTDADPSKP GVARGTCEHG AGDPEKVESQ HPDASVTFSN |
| | IKFGPIGSTY KA |
| SEQ ID NO: 12 | MYRSLIFATS LLSLAKGQLV GNLYCKGSCT AKNGKVVIDA NWRWLHVKGG YTNCYTGNEW NATACPDNKS CATNCAIDGA DYRRLRHYCE RQLLGTEVHH QGLYSTNIGS |
| | RTYLMQDDST YQLFKFTGSQ EFTFDVDLSN LPCGLNGALY FVSMDADGGL KKYPTNKAGA KYGTGYCDAQ CPRDLKFING EGNVEGWQPS KNDQNAGVGG HGSCCAEMDI |
| | WEANSVSTAV TPHSCSTIEQ SRCDGDGCGG TYSADRYAGV CDPDGCDFNS YRMGVKDFYG KGKTVDTSKK FTVVTQFIGS GDAMEIKRFY VQNGKTIPQP DSTIPGVTGN |
| | SITTFFCDAQ KKAFGDKYTF KDKGGMANMP STCNGMVLVM SLWDDHYSNM LWLDSTYPTD KNPDTDAGSG RGECAITSGV PADVESQHPD ASVIYSNIKF GPINTTFG |
| SEQ ID NO: 13 | MLAKFAALAA LVASANAQAV CSLTAETHPS LNWSKCTSSG CTNVAGSITV DANWRWTHIT SGSTNCYSGN EWDTSLCSTN TDCATKCCVD GAEYSSTYGI QTSGNSLSLQ |
| | FVTKGSYSTN IGSRTYLMNG ADAYQGFELL GNEFTFDVDV SGTGCGLNGA LYFVSMDLDG GKAKYTNNKA GAKYGTGYCD AQCPRDLKYI NGIANVEGWT PSTNDANAGI |
| | GDHGTCCSEM DIWEANKVST AFTPHPCTTI EQHMCEGDSC GGTYSDDRYG GTCDADGCDF NSYRMGNTTF YGEGKTVDTS SKFTVVTQFI KDSAGDLAEI KRFYVQNGKV |
| | IENSQSNVDG VSGNSITQSF CNAQKTAFGD IDDFNKKGGL KQMGKALAKP MVLVMSIWDD HAANMLWLDS TYPVEGGPGA YRGECPTTSG VPAEVEANAP NSKVIFSNIK |
| | FGPIGSTFSG GSSGTPPSNP SSSVKPVTST AKPSSTSTAS NPSGTGAAHW AQCGGIGFSG PTTCQSPYTC QKINDYYSQC V |
| SEQ ID NO: 14 | MFKKVALTAL CFLAVAQAQQ VGREVAENHP RLPWQRCTRN GGCQTVSNGQ VVLDANWRWL HVTDGYTNCY TGNSWNSTVC SDPTTCAQRC ALEGANYQQT YGITTNGDAL |
| | TIKFLTRSQQ TNVGARVYLM ENENRYQMFN LLNKEFTFDV DVSKVPCGIN GALYFIQMDA DGGMSKQPNN RAGAKYGTGY CDSQCPRDIK FIDGVANSAD WTPSETDPNA |
| | GRGRYGICCA EMDIWEANSI SNAYTPHPCR TQNDGGYQRC EGRDCNQPRY EGLCDPDGCD YNPFRMGNKD FYGPGKTVDT NRKMTVVTQF ITHDNTDTGT LVDIRRLYVQ |
| | DGRVIANPPT NFPGLMPAHD SITEQFCTDQ KNLFGDYSSF ARDGGLAHMG RSLAKGHVLA LSIWNDHGAH MLWLDSNYPT DADPNKPGIA RGTCPTTGGT PRETEQNHPD |
| | AQVIFSNIKF GDIGSTFSGY |
| SEQ ID NO: 15 | MYSAAVLATF SFLLGAGAQQ VGTSTAETHP ALTVQKCAAG GTCTDESDSI VLDANWRWLH STSGSTNCYT GNTWDTTLCP DAATCTTNCA LDGADYEGTY GITTSGDSLK |
| | LSFVTGSNVG SRTYLMDSET TYKEFALLGN EFTFTVDVSK LPCGLNGALY FVPMDADGGM SKYPTNKAGA KYGTGYCDAQ CPQDMKFVNG TANVEGWVPD SNSANSGTGN |
| | IGSCCSEFDV WEANSMSQAL TPHVCTVDSQ TACTGDDCAS NTGVCDGDGC DFNPYRMGNT TFYGSGMTID TSKPFSVVTQ FITDDGTETG TLTEIKRFYV QDDVVYEQPS |
| | SDISGVSGNS ITDDFCAAQK TAFGDTDYFT QNGGMAAMGK KMADGMVLVL SIWDDYNVNM LWLDSDYPTT KDASTPGVSR GSCATDSGVP ATVEAASGSA YVTFSSIKYG |
| | PIGSTFNAPA DSSSSVSASS SPAPIASSSS SASIAPVSSV VAAIVSSSAQ AISSAAPVVS SSAQAISSAA PVVSSVVSSA APVATSSTKS KCSKVSSTLK TSVAAPATSA |
| | TSAAVVATSS AASSTGSVPL YGNCTGGKTC SEGTCVVQND YYSQCVASS |
| SEQ ID NO: 16 | MTWQRCTGTG GSSCTNVNGE IVIDANWRWI HATGGYTNCF DGNEWNKTAC PSNAACTKNC AIEGSDYRGT YGITTSGNSL TLKFITKGQY STNVGSRTYL MKDTNNYEMF |
| | NLIGNEFTFD VDLSQLPCGL NGALYFVSMP EKGQGTPGAK YGTGKLSQCS VHISKTLTDA CARDLKFVGG EANADGWQAS TSDPNAGVGK KGACCAEMDV WEANSMSTAL |
| | TPHSCQPEGY AVCEESNCGG TYSLDRYAGT CDANGCDFNP YRVGNKDFYG KGKTVDTSKK MTVVTQFLGT GSDLTELKRF YVQDGKVISN PEPTIPGMTG NSITQKWCDT |
| | QKEVFKEEVY PFNQWGGMAS MGKGMAQGMV LVMSLWDDHY SNMLWLDSTY PTDRDPESPG AARGECAITS GAPAEVEANN PDASVMFSNI KFGPIGSTFQ QPA |
| SEQ ID NO: 17 | MQIKSYIQYL AAALPLLSSV AAQQAGTITA ENHPRMTWKR CSGPGNCQTV QGEVVIDANW RWLHNNGQNC YEGNKWTSQC SSATDCAQRC ALDGANYQST YGASTSGDSL |
| | TLKFVTKHEY GTNIGSRFYL MANQNKYQMF TLMNNEFAFD VDLSKVECGI NSALYFVAME EDGGMASYPS NRAGAKYGTG YCDAQCARDL KFIGGKANIE GWRPSTNDPN |
| | AGVGPMGACC AEIDVWESNA YAYAFTPHAC GSKNRYHICE TNNCGGTYSD DRFAGYCDAN GCDYNPYRMG NKDFYGKGKT VDTNRKFTVV SRFERNRLSQ FFVQDGRKIE |
| | VPPPTWPGLP NSADITPELC DAQFRVFDDR NRFAETGGFD ALNEALTIPM VLVMSIWDDH HSNMLWLDSS YPPEKAGLPG GDRGPCPTTS GVPAEVEAQY PNAQVVWSNI |
| | RFGPIGSTVN V |
| SEQ ID NO: 18 | MRTAKFATLA ALVASAAAQQ ACSLTTERHP SLSWKKCTAG GQCQTVQASI TLDSNWRWTH QVSGSTNCYT GNKWDTSICT DAKSCAQNCC VDGADYTSTY GITTNGDSLS |
| | LKFVTKGQYS TNVGSRTYLM DGEDKYQTFE LLGNEFTFDV DVSNIGCGLN GALYFVSMDA DGGLSRYPGN KAGAKYGTGY CDAQCPRDIK FINGEANIEG WTGSTNDPNA |
| | GAGRYGTCCS EMDIWEANNM ATAFTPHPCT IIGQSRCEGD SCGGTYSNER YAGVCDPDGC DFNSYRQGNK TFYGKGMTVD TTKKITVVTQ FLKDANGDLG EIKRFYVQDG |
| | KIIPNSESTI PGVEGNSITQ DWCDRQKVAF GDIDDFNRKG GMKQMGKALA GPMVLVMSIW DDHASNMLWL DSTFPVDAAG KPGAERGACP TTSGVPAEVE AEAPNSNVVF |
| | SNIRFGPIGS TVAGLPGAGN GGNNGGNPPP PTTTTSSAPA TTTTASAGPK AGRWQQCGGI GFTGPTQCEE PYTCTKLNDW YSQCL |
| SEQ ID NO: 19 | MQIKQYLQYL AAALPLVNMA AAQRAGTQQT ETHPRLSWKR CSSGGNCQTV NAEIVIDANW RWLHDSNYQN CYDGNRWTSA CSSATDCAQK CYLEGANYGS TYGVSTSGDA |
| | LTLKFVTKHE YGTNIGSRVY LMNGSDKYQM FTLMNNEFAF DVDLSKVECG LNSALYFVAM EEDGGMRSYS SNKAGAKYGT GYCDAQCARD LKFVGGKANI EGWRPSTNDA |
| | NAGVGPYGAC CAEIDVWESN AYAFAFTPHG CLNNNYHVCE TSNCGGTYSE DRFGGLCDAN GCDYNPYRMG NKDFYGKGKT VDTSRKFTVV TRFEENKLTQ FFIQDGRKID |
| | IPPPTWPGLP NSSAITPELC TNLSKVFDDR DRYEETGGFR TINEALRIPM VLVMSIWDGH YANMLWLDSV YPPEKAGQPG AERGPCAPTS GVPAEVEAQF PNAQVIWSNI |
| | RFGPIGSTYQ V |
| SEQ ID NO: 20 | MMYKKFAALA ALVAGAAAQQ ACSLTTETHP RLTWKRCTSG GNCSTVNGAV TIDANWRWTH TVSGSTNCYT GNEWDTSICS DGKSCAQTCC VDGADYSSTY GITTSGDSLN |
| | LKFVTKHQHG TNVGSRVYLM ENDTKYQMFE LLGNEFTFDV DVSNLGCGLN GALYFVSMDA DGGMSKYSGN KAGAKYGTGY CDAQCPRDLK FINGEANIEN WTPSTNDANA |
| | GFGRYGSCCS EMDIWDANNM ATAFTPHPCT IIGQSRCEGN SCGGTYSSER YAGVCDPDGC DFNAYRQGDK TFYGKGMTVD TTKKMTVVTQ FHKNSAGVLS EIKRFYVQDG |
| | KIIANAESKI PGNPGNSITQ EWCDAQKVAF GDIDDFNRKG GMAQMSKALE GPMVLVMSVW DDHYANMLWL DSTYPIDKAG TPGAERGACP TTSGVPAEIE AQVPNSNVIF |
| | SNIRFGPIGS TVPGLDGSTP SNPTATVAPP TSTTTSVRSS TTQISTPTSQ PGGCTTQKWG QCGGIGYTGC TNCVAGTTCT ELNPWYSQCL |
| SEQ ID NO: 21 | MYRNFLYAAS LLSVARSQLV GTQTTETHPG MTWQSCTAKG SCTTCSDNKA CASNCAVDGA DYKGTYGITA SGNSLQLKFI TKGSYSTNIG SRTYLMASDT AYQMFKFDGN |
| | KEFTFDVDLS GLPCGFNGAL YFVSMDEDGG LKKYSGNKAG AKYGTGYCDA QCPRDLKFIN GEGNVEGWKP SDNDANAGVG GHGSCCAEMD IWEANSISTA VTPHACSTIE |
| | QTRCDGDGCG GTYSADRYAG VCDPDGCDFN AYRMGVKNFY GKGMTVDTSK KFTVVTQFIG TGDAMEIKRF YVQGGKTIEQ PASTIPGVEG NSITTKFCDQ QKQVFGDRYT |
| | YKEKGGTANM AKALAQGMVL VMSLWDDHYS NMLWLDSTYP TDKNPDTDLG SGRGSCDVKS GAPADVESKS PDATVIYSNI KFGPLNSTY |
| SEQ ID NO: 22 | MLGKIAIASL SFLAIAKGQQ VGREVAENHP RLPWQRCTRN GGCQTVSNGQ VVLDANWRWL HVTDGYTNCY TGNSWNSSVC SDGTTCAQRC ALEGANYQQT YGITTSGNSL |
| | TMKFLTRSQG TNVGGRVYLM ENENRYQMFN LLNKEFTFDV DVSKVPCGIN GALYFIQMDA DGGMSSQPNN RAGAKYGTGY CDSQCPRDIK FIDGVANSVG WEPSETDSNA |
| | GRGRYGICCA EMDIWEANSI SNAYTPHPCR TQNDGGYQRC EGRDCNQPRY EGLCDPDGCD YNPFRMGNKD FYGPGKTIDT NRKMTVVTQF ITHDNTDTGT LVDIRRLYVQ |
| | DGRVIANPPT NFPGLMPAHD SITEQFCTDQ KNLFGDYSSF ARDGGLAHMG RSLAKGHVLA LSIWNDHGAH MLWLDSNYPT DADPNKPGIA RGTCPTTGGT PRETEQNHPD |
| | AQVIFSNIKF GDIGSTFSGY |
| SEQ ID NO: 23 | MFPRSILLAL SLTAVALGQQ VGTNMAENHP SLTWQRCTSS GCQNVNGKVT LDANWRWTHR INDFTNCYTG NEWDTSICPD GVTCAENCAL DGADYAGTYG VTSSGTALTL |
| | KFVTESQQKN IGSRLYLMAD DSNYEIFNLL NKEFTFDVDV SKLPCGLNGA LYFSEMAADG GMSSTNTAGA KYGTGYCDSQ CPRDIKFIDG EANSEGWEGS PNDVNAGTGN |
| | FGACCGEMDI WEANSISSAY TPHPCREPGL QRCEGNTCSV NDRYATECDP DGCDFNSFRM GDKSFYGPGM TVDTNQPITV VTQFITDNGS DNGNLQEIRR IYVQNGQVIQ |
| | NSNVNIPGID SGNSISAEFC DQAKEAFGDE RSFQDRGGLS GMGSALDRGM VLVLSIWDDH AVNMLWLDSD YPLDASPSQP GISRGTCSRD SGKPEDVEAN AGGVQVVYSN |
| | IKFGDINSTF NNNGGGGGNP SPTTTRPNSP AQTMWGQCGG QGWTGPTACQ SPSTCHVIND FYSQCF |
| SEQ ID NO: 24 | MYRNLALASL SLFGAARAQQ AGTVTTETHP SLSWKTCTGT GGTSCTTKAG KITLDANWRW THVTTGYTNC YDGNSWNTTA CPDGATCTKN CAVDGADYSG TYGITTSSNS |
| | LSIKFVTKGS NSANIGSRTY LMESDTKYQM FNLIGQEFTF DVDVSKLPCG LNGALYFVEM AADGGIGKGN NKAGAKYGTG YCDSQCPHDI KFINGKANVE GWNPSDADPN |
| | AGSGKIGACC PEMDIWEANS ISTAYTPHPC KGTGLQECTD DVSCGDGSNR YSGLCDKDGC DFNSYRMGVK DFYGPGATLD TTKKMTVVTQ FLGSGSTLSE IKRFYVQNGK |
| | VFKNSDSAIE GVTGNSITES FCAAQKTAFG DTNSFKTLGG LNEMGASLAR GHVLVMSLWD DHAVNMLWLD STYPTNSTKL GAQRGTCAID SGKPEDVEKN HPDATVVFSD |
| | IKFGPIGSTF QQPS |
| SEQ ID NO: 25 | MVDIQIATFL LLGVVGVAAQ QVGTYIPENH PLLATQSCTA SGGCTTSSSK IVLDANRRWI HSTLGTTSCL TANGWDPTLC PDGITCANYC ALDGVSYSST YGITTSGSAL |
| | RLQFVTGTNI GSRVFLMADD THYRTFQLLN QELAFDVDVS KLPCGLNGAL YFVAMDADGG KSKYPGNRAG AKYGTGYCDS QCPRDVQFIN GQANVQGWNA TSATTGTGSY |
| | GSCCTELDIW EANSNAAALT PHTCTNNAQT RCSGSNCTSN TGFCDADGCD FNSFRLGNTT FLGAGMSVDT TKTFTVVTQF ITSDNTSTGN LTEIRRFYVQ NGNVIPNSVV |
| | NVTGIGAVNS ITDPFCSQQK KAFIETNYFA QHGGLAQLGQ ALRTGMVLAF SISDDPANHM LWLDSNFPPS ANPAVPGVAR GMCSITSGNP ADVGILNPSP YVSFLNIKFG |
| | SIGTTFRPA |
| SEQ ID NO: 26 | MHQRALLFSA LAVAANAQQV GTQTPETHPP LTWQKCTAAG SCSQQSGSVV IDANWRWLHS TKDTTNCYTG NTWNTELCPD NESCAQNCAL DGADYAGTYG VTTSGSELKL |
| | SFVTGANVGS RLYLMQDDET YQHFNLLNHE FTFDVDVSNL PCGLNGALYF VAMDADGGMS KYPSNKAGAK YGTGYCDSQC PRDLKFINGM ANVEGWEPSS SDKNAGVGGH |
| | GSCCPEMDIW EANSISTAVT PHPCDDVSQT MCSGDACGGT YSESRYAGTC DPDGCDFNPF RMGNESFYGP GKIVDTKSKM TVVTQFITAD GTDSGALSEI KRLYVQNGKV |
| | IANSVSNVAG VSGNSITSDF CTAQKKAFGD EDIFAKHGGL SGMGKALSEM VLIMSIWDDH HSSMMWLDST YPTDADPSKP GVARGTCEHG AGDPENVESQ HPDASVTFSN |
| | IKFGPIGSTY EG |
| SEQ ID NO: 27 | MFRTATLLAF TMAAMVFGQQ VGTNTAENHR TLTSQKCTKS GGCSNLNTKI VLDANWRWLH STSGYTNCYT GNQWDATLCP DGKTCAANCA LDGADYTGTY GITASGSSLK |
| | LQFVTGSNVG SRVYLMADDT HYQMFQLLNQ EFTFDVDMSN LPCGLNGALY LSAMDADGGM AKYPTNKAGA KYGTGYCDSQ CPRDIKFING EANVEGWNAT SANAGTGNYG |
| | TCCTEMDIWE ANNDAAAYTP HPCTTNAQTR CSGSDCTRDT GLCDADGCDF NSFRMGDQTF LGKGLTVDTS KPFTVVTQFI TNDGTSAGTL TEIRRLYVQN GKVIQNSSVK |
| | IPGIDPVNSI TDNFCSQQKT AFGDTNYFAQ HGGLKQVGEA LRTGMVLALS IWDDYAANML WLDSNYPTNK DPSTPGVARG TCATTSGVPA QIEAQSPNAY VVFSNIKFGD |
| | LNTTYTGTVS SSSVSSSHSS TSTSSSHSSS STPPTQPTGV TVPQWGQCGG IGYTGSTTCA SPYTCHVLNP YYSQCY |
| SEQ ID NO: 28 | MYQRALLFSF FLAAARAHEA GTVTAENHPS LTWQQCSSGG SCTTQNGKVV IDANWRWVHT TSGYTNCYTG NTWDTSICPD DVTCAQNCAL DGADYSGTYG VTTSGNALRL |
| | NFVTQSSGKN IGSRLYLLQD DTTYQIFKLL GQEFTFDVDV SNLPCGLNGA LYFVAMDADG NLSKYPGNKA GAKYGTGYCD SQCPRDLKFI NGQANVEGWQ PSANDPNAGV |
| | GNHGSSCAEM DVWEANSIST AVTPHPCDTP GQTMCQGDDC GGTYSSTRYA GTCDPDGCDF NPYQPGNHSF YGPGKIVDTS SKFTVVTQFI TDDGTPSGTL TEIKRFYVQN |
| | GKVIPQSEST ISGVTGNSIT TEYCTAQKAA FGDNTGFFTH GGLQKISQAL AQGMVLVMSL WDDHAANMLW LDSTYPTDAD PDTPGVARGT CPTTSGVPAD VESQNPNSYV |
| | IYSNIKVGPI NSTFTAN |
| SEQ ID NO: 29 | MQIKSYIQYL AAALPLLSSV AAQQAGTITA ENHPRMTWKR CSGPGNCQTV QGEVVIDANW RWLHNNGQNC YEGNKWTSQC SSATDCAQRC ALDGANYQST YGASTSGDSL |
| | TLKFVTKHEY GTNIGSRFYL MANQNKYQMF TLMNNEFAFD VDLSKVECGI NSALYFVAME EDGGMASYPS NRAGAKYGTG YCDAQCARDL KFIGGKANIE GWRPSTNDPN |
| | AGVGPMGACC AEIDVWESNA YAYAFTPHAC GSKNRYHICE TNNCGGTYSD DRFAGYCDAN GCDYNPYRMG NKDFYGKGKT VDTNRKFTVV SRFERNRLSQ FFVQDGRKIE |
| | VPPPTWPGLP NSADITPELC DAQFRVFDDR NRFAETGGFD ALNEALTIPM VLVMSIWDDH HSNMLWLDSS YPPEKAGLPG GDRGPCPTTS GVPAEVEAQY PDAQVVWSNI |
| | RFGPIGSTVN V |
| SEQ ID NO: 30 | MYRKLAVISA FLATARAQSA CTLQSETHPP LTWQKCSSGG TCTQQTGSVV IDANWRWTHA TNSSTNCYDG NTWSSTLCPD NETCAKNCCL DGAAYASTYG VTTSGNSLSI |
| | GFVTQSAQKN VGARLYLMAS DTTYQEFTLL GNEFSFDVDV SQLPCGLNGA LYFVSMDADG GVSKYPTNTA GAKYGTGYCD SQCPRDLKFI NGQANVEGWE PSSNNANTGI |
| | GGHGSCCSEM DIWEANSISE ALTPHPCTTV GQEICEGDGC GGTYSDNRYG GTCDPDGCDW DPYRLGNTSF YGPGSSFTLD TTKKLTVVTQ FETSGAINRY YVQNGVTFQQ |
| | PNAELGSYSG NGLNDDYCTA EEAEFGGSSF SDKGGLTQFK KATSGGMVLV MSLWDDYYAN MLWLDSTYPT NETSSTPGAV RGSCSTSSGV PAQVESQSPN AKVTFSNIKF |
| | GPIGSTGDPS GGNPPGGNPP GTTTTRRPAT TTGSSPGPTQ SHYGQCGGIG YSGPTVCASG TTCQVLNPYY SQCL |
| SEQ ID NO: 31 | MYQRALLFSF FLAAARAQQA GTVTAENHPS LTWQQCSSGG SCTTQNGKVV IDANWRWVHT TSGYTNCYTG NTWDTSICPD DVTCAQNCAL DGADYSGTYG VTTSGNALRL |
| | NFVTQSSGKN IGSRLYLLQD DTTYQIFKLL GQEFTFDVDV SNLPCGLNGA LYFVAMDADG GLSKYPGNKA GAKYGTGYCD SQCPRDLKFI NGQANVEGWQ PSANDPNAGV |
| | GNHGSCCAEM DVWEANSIST AVTPHPCDTP GQTMCQGDDC GGTYSSTRYA GTCDPDGCDF NPYRQGNHSF YGPGQIVDTS SKFTVVTQFI TDDGTPSGTL TEIKRFYVQN |
| | GKVIPQSEST ISGVTGNSIT TEYCTAQKAA FGDNTGFFTH GGLQKISQAL AQGMVLVMSL WDDHAANMLW LDSTYPTDAD PDTPGVARGT CPTTSGVPAD VESQYPNSYV |
| | IYSNIKVGPI NSTFTAN |
| SEQ ID NO: 32 | MIRKITTLAA LVGVVRGQAA CSLTAETHPS LTWQKCSSGG SCTNVAGSVT IDANWRWTHT TSGYTNCYTG NKWDTSICST NADCASKCCV DGANYQQTYG ASTSGNALSL |
| | QYVTQSSGKN VGSRLYLLES ENKYQMFNLL GNEFTFDVDA SKLGCGLNGA VYFVSMDADG GQSKYSGNKA GAKYGTGYCD SQCPRDLKYI NGAANVEGWQ PSSGDANSGV |
| | GNMGSCCAEM DIWEANSIST AYTPHPCSNN AQHSCKGDDC GGTYSSVRYA GDCDPDGCDF NSYRQGNRTF YGPGSNFNVD SSKKVTVVTQ FISSGGQLTD IKRFYVQNGK |
| | VIPNSQSTIT GVTGNSVTQD YCDKQKTAFG DQNVFNQRGG LRQMGDALAK GMVLVMSVWD DHHSQMLWLD STYPTTSTAP GAARGSCSTS SGKPSDVQSQ TPGATVVYSN |
| | IKFGPIGSTF KSS |
| SEQ ID NO: 33 | MLRRALLLSS SAILAVKAQQ AGTATAENHP PLTWQECTAP GSCTTQNGAV VLDANWRWVH DVNGYTNCYT GNTWDPTYCP DDETCAQNCA LDGADYEGTY GVTSSGSSLK |
| | LNFVTGSNVG SRLYLLQDDS TYQIFKLLNR EFSFDVDVSN LPCGLNGALY FVAMDADGGV SKYPNNKAGA KYGTGYCDSQ CPRDLKFIDG EANVEGWQPS SNNANTGIGD |
| | HGSCCAEMDV WEANSISNAV TPHPCDTPGQ TMCSGDDCGG TYSNDRYAGT CDPDGCDFNP YRMGNTSFYG PGKIIDTTKP FTVVTQFLTD DGTDTGTLSE IKRFYIQNSN |
| | VIPQPNSDIS GVTGNSITTE FCTAQKQAFG DTDDFSQHGG LAKMGAAMQQ GMVLVMSLWD DYAAQMLWLD SDYPTDADPT TPGIARGTCP TDSGVPSDVE SQSPNSYVTY |
| | SNIKFGPINS TFTAS |
| SEQ ID NO: 34 | MHQRALLFSA FWTAVQAQQA GTLTAETHPS LTWQKCAAGG TCTEQKGSVV LDSNWRWLHS VDGSTNCYTG NTWDATLCPD NESCASNCAL DGADYEGTYG VTTSGDALTL |
| | QFVTGANIGS RLYLMADDDE SYQTFNLLNN EFTFDVDASK LPCGLNGAVY FVSMDADGGV AKYSTNKAGA KYGTGYCDSQ CPRDLKFING QVRKGWEPSD SDKNAGVGGH |
| | GSCCPQMDIW EANSISTAYT PHPCDDTAQT MCEGDTCGGT YSSERYAGTC DPDGCDFNAY RMGNESFYGP SKLVDSSSPV TVVTQFITAD GTDSGALSEI KRFYVQGGKV |
| | IANAASNVDG VTGNSITADF CTAQKKAFGD DDIFAQHGGL QGMGNALSSM VLTLSIWDDH HSSMMWLDSS YPEDADATAP GVARGTCEPH AGDPEKVESQ SGSATVTYSN |
| | IKYGPIGSTF DAPA |
| SEQ ID NO: 35 | MASTLSFKIY KNALLLAAFL GAAQAQQVGT STAEVHPSLT WQKCTAGGSC TSQSGKVVID SNWRWVHNTG GYTNCYTGND WDRTLCPDDV TCATNCALDG ADYKGTYGVT |
| | ASGSSLRLNF VTQASQKNIG SRLYLMADDS KYEMFQLLNQ EFTFDVDVSN LPCGLNGALY FVAMDEDGGM ARYPTNKAGA KYGTGYCDAQ CPRDLKFING QANVEGWEPS |
| | SSDVNGGTGN YGSCCAEMDI WEANSISTAF TPHPCDDPAQ TRCTGDSCGG TYSSDRYGGT CDPDGCDFNP YRMGNQSFYG PSKIVDTESP FTVVTQFITN DGTSTGTLSE |
| | IKRFYVQNGK VIPQSVSTIS AVTGNSITDS FCSAQKTAFK DTDVFAKHGG MAGMGAGLAE GMVLVMSLWD DHAANMLWLD STYPTSASST TPGAARGSCD ISSGEPSDVE |
| | ANHSNAYVVY SNIKVGPLGS TFGSTDSGSG TTTTKVTTTT ATKTTTTTGP STTGAAHYAQ CGGQNWTGPT TCASPYTCQR QGDYYSQCL |
| SEQ ID NO: 36 | MVSAKFAALA ALVASASAQQ VCSLTPESHP PLTWQRCSAG GSCTNVAGSV TLDSNWRWTH TLQGSTNCYS GNEWDTSICT TGTKCAQNCC VEGAEYAATY GITTSGNQLN |
| | LKFVTEGKYS TNVGSRTYLM ENATKYQGFN LLGNEFTFDV DVSNIGCGLN GALYFVSMDL DGGLAKYSGN KAGAKYGTGY CDAQCPRDIK FINGEANIEG WNPSTNDVNA |
| | GAGRYGTCCS EMDIWEANNM ATAYTPHSCT ILDQSRCEGE SCGGTYSSDR YGGVCDPDGC DFNSYRMGNK EFYGKGKTVD TTKKMTVVTQ FLKNAAGELS EIKRFYVQNG |
| | VVIPNSVSSI PGVPNQNSIT QDWCDAQKIA FGDPDDNTAK GGLRQMGLAL DKPMVLVMSI WNDHAAHMLW LDSTYPVDAA GRPGAERGAC PTTSGVPSEV EAEAPNSNVA |
| | FSNIKFGPIG STFNSGSTNP NPISSSTATT PTSTRVSSTS TAAQTPTSAP GGTVPRWGQC GGQGYTGPTQ CVAPYTCVVS NQWYSQCL |
| SEQ ID NO: 37 | MFPYIALVSF SFLSVVLAQQ VGTLTAETHP QLTVQQCTRG GSCTTQQRSV VLDGNWRWLH STSGSNNCYT GNTWDTSLCP DAATCSRNCA LDGADYSGTY GITSSGNALT |
| | LKFVTHGPYS TNIGSRVYLL ADDSHYQMFN LKNKEFTFDV DVSQLPCGLN GALYFSQMDA DGGTGRFPNN KAGAKYGTGY CDSQCPHDIK FINGEANVQG WQPSPNDSNA |
| | GKGQYGSCCA EMDIWEANSM ASAYTPHPCT VTTPTRCQGN DCGDGDNRYG GVCDKDGCDF NSFRMGDKNF LGPGKTVNTN SKFTVVTQFL TSDNTTSGTL SEIRRLYVQN |
| | GRVIQNSKVN IPGMASTLDS ITESFCSTQK TVFGDTNSFA SKGGLRAMGN AFDKGMVLVL SIWDDHEAKM LWLDSNYPLD KSASAPGVAR GTCATTSGEP KDVESQSPNA |
| | QVIFSNIKYG DIGSTYSN |
| SEQ ID NO: 38 | MYRAIATASA LIAAVRAQQV CSLTQESKPS LNWSKCTSSG CSNVKGSVTI DANWRWTHQV SGSTNCYTGN KWDTSVCTSG KVCAERCCLD GADYASTYGI TSSGDQLSLS |
| | FVTKGPYSTN IGSRTYLMED ENTYQMFQLL GNEFTFDVDV SNIGCGLNGA LYFVSMDADG GKAKYPGNKA GAKYGTGYCD AQCPRDVKFI NGQANSDGWQ PSDSDVNGGI |
| | GNLGTCCPEM DIWEANSIST AYTPHPCTKL TQHSCTGDSC GGTYSNDRYG GTCDADGCDF NSYRQGNKTF YGPGSGFNVD TTKKVTVVTQ FHKGSNGRLS EITRLYVQNG |
| | KVIANSESKI AGVPGNSLTA DFCTKQKKVF NDPDDFTKKG AWSGMSDALE APMVLVMSLW HDHHSNMLWL DSTYPTDSTK LGSQRGSCST SSGVPADLEK NVPNSKVAFS |
| | NIKFGPIGST YKSDGTTPTN PTNPSEPSNT ANPNPGTVDQ WGQCGGSNYS GPTACKSGFT CKKINDFYSQ CQ |
| SEQ ID NO: 39 | MYSAAVLATF SFLLGAGAQQ VGTLKTESHP PLTIQKCAAG GTCTDEADSV VLDANWRWLH STSGSTNCYT GNTWDTTLCP DAATCTANCA FDGADYEGTY GITSSGDSLK |
| | LSFVTGSNVG SRTYLMDSET TYKEFALLGN EFTFTVDVSK LPCGLNGALY FVPMDADGGM SKYPTNKAGA KYGTGYCDAQ CPQDMKFVSG GANNEGWVPD SNSANSGTGN |
| | IGSCCSEFDV WEANSMSQAL TPHTCTVDGQ TACTGDDCAG NTGVCDADGC DFNPYRMGNT TFYGSGKTID TTKPFSVVTQ FITDDGTETG TLTEIKRFYV QDDVVYEQPN |
| | SDISGVSGNS ITDDFCTAQK TAFGDTDYFS QKGGMAAMGK KMADGMVLVL SIWDDYNVNM LWLDSDYPTT KDASTPGVSR GSCATTSGVP ATVEAASGSA YVTFSSIKYG |
| | PIGSTFKAPA DSSSPVVASS SPAAVAAVVS TSSAQAVPSH PAVSSSQAAV STPEAVSSAP EVPASSSAAQ SVAPTSTKPK CSKVSQSSTL ATSVAAPATT ATSAAVAATS |
| | AASSSGSVPL YGNCTGGKTC SEGTCVVQNP WYSQCVASS |
| SEQ ID NO: 40 | MFRAAALLAF TCLAMVSGQQ AGTNTAENHP QLQSQQCTTS GGCKPLSTKV VLDSNWRWVH STSGYTNCYT GNEWDTSLCP DGKTCAANCA LDGADYSGTY GITSTGTALT |
| | LKFVTGSNVG SRVYLMADDT HYQLLKLLNQ EFTFDVDMSN LPCGLNGALY LSAMDADGGM SKYPGNKAGA KYGTGYCDSQ CPKDIKFING EANVGNWTET GSNTGTGSYG |
| | TCCSEMDIWE ANNDAAAFTP HPCTTTGQTR CSGDDCARNT GLCDGDGCDF NSFRMGDKTF LGKGMTVDTS KPFTVVTQFL TNDNTSTGTL SEIRRIYIQN GKVIQNSVAN |
| | IPGVDPVNSI TDNFCAQQKT AFGDTNWFAQ KGGLKQMGEA LGNGMVLALS IWDDHAANML WLDSDYPTDK DPSAPGVARG TCATTSGVPS DVESQVPNSQ VVFSNIKFGD |
| | IGSTFSGTSS PNPPGGSTTS SPVTTSPTPP PTGPTVPQWG QCGGIGYSGS TTCASPYTCH VLNPYYSQCY |
| SEQ ID NO: 41 | MYRKLAVISA FLATARAQSA CTLQSETHPP LTWQKCSSGG TCTQQTGSVV IDANWRWTHA TNSSTNCYDG NTWSSTLCPD NETCAKNCCL DGAAYASTYG VTTSGNSLSI |
| | GFVTQSAQKN VGARLYLMAS DTTYQEFTLL GNEFSFDVDV SQLPCGLNGA LYFVSMDADG GVSKYPTNTA GAKYGTGYCD SQCPRDLKFI NGQANVEGWE PSSNNANTGI |
| | GGHGSCCSEM DIWEANSISE ALTPHPCTTV GQEICEGDGC GGTYSDNRYG GTCDPDGCDW NPYRLGNTSF YGPGSSFTLD TTKKLTVVTQ FETSGAINRY YVQNGVTFQQ |
| | PNAELGSYSG NELNDDYCTA EEAEFGGSSF SDKGGLTQFK KATSGGMVLV MSLWDDYYAN MLWLDSTYPT NETSSTPGAV RGSCSTSSGV PAQVESQSPN AKVTFSNIKF |
| | GPIGSTGNPS GGNPPGGNRG TTTTRRPATT TGSSPGPTQS HYGQCGGIGY SGPTVCASGT TCQVLNPYYS QCL |
| SEQ ID NO: 42 | MPSTYDIYKK LLLLASFLSA SQAQQVGTSK AEVHPSLTWQ TCTSGGSCTT VNGKVVVDAN WRWVHNVDGY NNCYTGNTWD TTLCPDDETC ASNCALEGAD YSGTYGVTTS |
| | GNSLRLNFVT QASQKNIGSR LYLMEDDSTY KMFKLLNQEF TFDVDVSNLP CGLNGAVYFV SMDADGGMAK YPANKAGAKY GTGYCDSQCP RDLKFINGMA NVEGWEPSAN |
| | DANAGTGNHG SCCAEMDIWE ANSISTAYTP HPCDTPGQVM CTGDSCGGTY SSDRYGGTCD PDGCDFNSYR QGNKTFYGPG MTVDTKSKIT VVTQFLTNDG TASGTLSEIK |
| | RFYVQNGKVI PNSESTWSGV SGNSITTAYC NAQKTLFGDT DVFTKHGGME GMGAALAEGM VLVLSLWDDH NSNMLWLDSN YPTDKPSTTP GVARGSCDIS SGDPKDVEAN |
| | DANAYVVYSN IKVGPIGSTF SGSTGGGSSS STTATSKTTT TSATKTTTTT TKTTTTTSAS STSTGGAQHW AQCGGIGWTG PTTCVAPYTC QKQNDYYSQC L |
| SEQ ID NO: 43 | MISKVLAFTS LLAAARAQQA GTLTTETHPP LSVSQCTASG CTTSAQSIVV DANWRWLHST TGSTNCYTGN TWDKTLCPDG ATCAANCALD GADYSGVYGI TTSGNSIKLN |
| | FVTKGANTNV GSRTYLMAAG STTQYQMLKL LNQEFTFDVD VSNLPCGLNG ALYFAAMDAD GGLSRFPTNK AGAKYGTGYC DAQCPQDIKF INGVANSVGW TPSSNDVNAG |
| | AGQYGSCCSE MDIWEANKIS AAYTPHPCSV DTQTRCTGTD CGIGARYSSL CDADGCDFNS YRQGNTSFYG AGLTVNTNKV FTVVTQFITN DGTASGTLKE IRRFYVQNGV |
| | VIPNSQSTIA GVPGNSITDS FCAAQKTAFG DTNEFATKGG LATMSKALAK GMVLVMSIWD DHTANMLWLD APYPATKSPS APGVTRGSCS ATSGNPVDVE ANSPGSSVTF |
| | SNIKWGPINS TYTGSGAAPS VPGTTTVSSA PASTATSGAG GVAKYAQCGG SGYSGATACV SGSTCVALNP YYSQCQ |
| SEQ ID NO: 44 | MFPAATLFAF SLFAAVYGQQ VGTQLAETHP RLTWQKCTRS GGCQTQSNGA IVLDANWRWV HNVGGYTNCY TGNTWNTSLC PDGATCAKNC ALDGANYQST YGITTSGNAL |
| | TLKFVTQSEQ KNIGSRVYLL ESDTKYQLFN PLNQEFTFDV DVSQLPCGLN GAVYFSAMDA DGGMSKFPNN AAGAKYGTGY CDSQCPRDIK FINGEANVQG WQPSPNDTNA |
| | GTGNYGACCN EMDVWEANSI STAYTPHPCT QQGLVRCSGT ACGGGSNRYG SICDPDGCDF NSFRMGDKSF YGPGLTVNTQ QKFTVVTQFL TNNNSSSGTL REIRRLYVQN |
| | GRVIQNSKVN IPGMPSTMDS VTTEFCNAQK TAFNDTFSFQ QKGGMANMSE ALRRGMVLVL SIWDDHAANM LWLDSNYPTD RPASQPGVAR GTCPTSSGKP SDVENSTANS |
| | QVIYSNIKFG DIGSTYSA |
| SEQ ID NO: 45 | MKGSISYQIY KGALLLSALL NSVSAQQVGT LTAETHPALT WSKCTAGXCS QVSGSVVIDA NWPXVHSTSG STNCYTGNTW DATLCPDDVT CAANCAVDGA RRQHLRVTTS |
| | GNSLRINFVT TASQKNIGSR LYLLENDTTY QKFNLLNQEF TFDVDVSNLP CGLNGALYFV DMDADGGMAK YPTNKAGAKY GTGYCDSQCP RDLKFINGQA NVDGWTPSKN |
| | DVNSGIGNHG SCCAEMDIWE ANSISNAVTP HPCDTPSQTM CTGQRCGGTY STDRYGGTCD PDGCDFNPYR MGVTNFYGPG ETIDTKSPFT VVTQFLTNDG TSTGTLSEIK |
| | RFYVQGGKVI GNPQSTIVGV SGNSITDSWC NAQKSAFGDT NEFSKHGGMA GMGAGLADGM VLVMSLWDDH ASDMLWLDST YPTNATSTTP GAKRGTCDIS RRPNTVESTY |
| | PNAYVIYSNI KTGPLNSTFT GGTTSSSSTT TTTSKSTSTS SSSKTTTTVT TTTTSSGSSG TGARDWAQCG GNGWTGPTTC VSPYTCTKQN DWYSQCL |
| SEQ ID NO: 46 | MFRTAALTAF TLAAVVLGQQ VGTLTAENHP ALSIQQCTAS GCTTQQKSVV LDSNWRWTHS LPVHTNCYTG NAWDASLCPD PTTCATNCAI DGADYSGTYG ITTSGNALTL |
| | RFVTNGPYSK NIGSRVYLLD DADHYKMFDL KNQEFTFDVD MSGLPCGLNG ALYFSEMPAD GGKAAHTSNK AGAKYGTGYC DAQCPHDIKW INGEANILDW SASATDANAG |
| | NGRYGACCAE MDIWEANSEA TAYTPHVCRD EGLYRCSGTE CGDGDNRYGG VCDKDGCDFN SYRMGDKNFL GRGKTIDTTK KITVVTQFIT DDNTSSGNLV EIRRVYVQDG |
| | VTYQNSFSTF PSLSQYNSIS DDFCVAQKTL FGDNQYYNTH GGTEKMGDAM ANGMVLIMSL WSDHAAHMLW LDSDYPLDKS PSEPGVSRGA CATTTGDPDD VVANHPNASV |
| | TFSNIKYGPI GSTYGGSTPP VSSGNTSAPP VTSTTSSGPT TPTGPTGTVP KWGQCGGNGY SGPTTCVAGS TCTYSNDWYS QCL |
| SEQ ID NO: 47 | MYQRALLFSA LLSVSRAQQA GTAQEEVHPS LTWQRCEASG SCTEVAGSVV LDSNWRWTHS VDGYTNCYTG NEWDATLCPD NESCAQNCAV DGADYEATYG ITSNGDSLTL |
| | KFVTGSNVGS RVYLMEDDET YQMFDLLNNE FTFDVDVSNL PCGLNGALYF TSMDADGGLS KYEGNTAGAK YGTGYCDSQC PRDIKFINGL GNVEGWEPSD SDANAGVGGM |
| | GTCCPEMDIW EANSISTAYT PHPCDSVEQT MCEGDSCGGT YSDDRYGGTC DPDGCDFNSY RMGNTSFYGP GAIIDTSSKF TVVTQFIADG GSLSEIKRFY VQNGEVIPNS |
| | ESNISGVEGN SITSEFCTAQ KTAFGDEDIF AQHGGLSAMG DAASAMVLIL SIWDDHHSSM MWLDSSYPTD ADPSQPGVAR GTCEQGAGDP DVVESEHADA SVTFSNIKFG |
| | PIGSTF |
| SEQ ID NO: 48 | MYRAIATASA LIAAVRAQQV CSLTTETKPA LTWSKCTSSG CSNVQGSVTI DANWRWTHQV SGSTNCHTGN KWDTSVCTSG KVCAEKCCVD GADYASTYGI TSSGNQLSLS |
| | FVTKGSYGTN IGSRTYLMED ENTYQMFQLL GNEFTFDVDV SNIGCGLNGA LYFVSMDADG GKAKYPGNKA GAKYGTGYCD AQCPRDVKFI NGQANSDGWE PSKSDVNGGI |
| | GNLGTCCPEM DIWEANSIST AYTPHPCTKL TQHACTGDSC GGTYSNDRYG GTCDADGCDF NAYRQGNKTF YGPGSGFNVD TTKKVTVVTQ FHKGSNGRLS EITRLYVQNG |
| | KVIANSESKI AGNPGSSLTS DFCTTQKKVF GDIDDFAKKG AWNGMSDALE APMVLVMSLW HDHHSNMLWL DSTYPTDSTA LGSQRGSCST SSGVPADLEK NVPNSKVAFS |
| | NIKFGPIGST YNKEGTQPQP TNPTNPNPTN PTNPGTVDQW GQCGGTNYSG PTACKSPFTC KKINDFYSQC Q |
| SEQ ID NO: 49 | MFRTAALTAF TLAAVVLGQQ VGTLAAENHP ALSIQQCTAS GCTTQQKSVV LDSNWRWTHS TAGATNCYTG NAWDSSLCPN PTTCATNCAI DGADYSGTYG ITTSGNSLTL |
| | RFVTNGQYSE NIGSRVYLLD DADHYKLFNL KNQEFTFDVD MSGLPCGLNG ALYFSEMAAD GGKAAHTGNN AGAKYGTGYC DAQCPHDIKW INGEANILDW SGSATDPNAG |
| | NGRYGACCAE MDIWEANSEA TAYTPHVCRD EGLYRCSGTE CGDGDNRYGG VCDKDGCDFN SYRMGDKNFL GRGKTIDTTK KITVVTQFIT DDNTPTGNLV EIRRVYVQDG |
| | VTYQNSFSTF PSLSQYNSIS DDFCVAQKTL FGDNQYYNTH GGTEKMGDSL ANGMVLIMSL WSDHAAHMLW LDSDYPLDKS PSEPGVSRGA CATTTGDPDD VVANHPNASV |
| | TFSNIKYGPI GSTYGGSTPP VSSGNTSVPP VTSTTSSGPT TPTGPTGTVP KWGQCGGIGY SGPTSCVAGS TCTYSNEWYS QCL |
| SEQ ID NO: 50 | MYQKLALISA FLATARAQSA CTLQAETHPP LTWQKCSSGG TCTQQTGSVV IDANWRWTHA TNSSTNCYDG NTWSSTLCPD NETCAKNCCL DGAAYASTYG VTTSADSLSI |
| | GFVTQSAQKN VGARLYLMAS DTTYQEFTLL GNEFSFDVDV SQLPCGLNGA LYFVSMDADG GVTKYPTNTA GAKYGTGYCD SQCPRDLKFI NGQANVEGWE PSSNNANTGI |
| | GGHGSCCSEM DIWEANSISE ALTPHPCTTV GQEICEGDSC GGTYSGDRYG GTCDPDGCDW NPYRLGNTSF YGPGSSFTLD TTKKLTVVTQ FETSGAINRY YVQNGVTFQQ |
| | PNAELGDYSG NSLDDDYCAA EEAEFGGSSF SDKGGLTQFK KATSGGMVLV MSLWDDYYAN MLWLDSTYPT DETSSTPGAV RGSSSTSSGV PAQLESNSPN AKVVYSNIKF |
| | GPIGSTGNPS GGNPPGGNPP GTTTPRPATS TGSSPGPTQT HYGQCGGIGY IGPTVCASGS TCQVLNPYYS QCL |
| SEQ ID NO: 51 | MTWQSCTAKG SCTNKNGKIV IDANWRWLHK KEGYDNCYTG NEWDATACPD NKACAANCAV DGADYSGTYG ITAGSNSLKL KFITKGSYST NIGSRTYLMK DDTTYEMFKF |
| | TGNQEFTFDV DVSNLPCGFN GALYFVSMDA DGGLKKYSTN KAGAKYGTGY CDAQCPRDLK FINGEGNVEG WKPSSNDANA GVGGHGSCCA EMDIWEANSV STAVTPHSCS |
| | TIEQSRCDGD GCGGTYSADR YAGVCDPDGC DFNSYRMGVK DFYGKGKTVD TSKKFTVVTQ FIGTGDAMEI KRFYVQNGKT IAQPASAVPG VEGNSITTKF CDQQKAVFGD |
| | TYTFKDKGGM ANMAKALANG MVLVMSLWDD HYSNMLWLDS TYPTDKNPDT DLGTGRGECE TSSGVPADVE SQHADATVVY SNIKFGPLNS TFG |
| SEQ ID NO: 52 | MASAISFQVY RSALILSAFL PSITQAQQIG TYTTETHPSM TWETCTSGGS CATNQGSVVM DANWRWVHQV GSTTNCYTGN TWDTSICDTD ETCATECAVD GADYESTYGV |
| | TTSGSQIRLN FVTQNSNGAN VGSRLYMMAD NTHYQMFKLL NQEFTFDVDV SNLPCGLNGA LYFVTMDEDG GVSKYPNNKA GAQYGVGYCD SQCPRDLKFI QGQANVEGWT |
| | PSSNNENTGL GNYGSCCAEL DIWESNSISQ ALTPHPCDTA TNTMCTGDAC GGTYSSDRYA GTCDPDGCDF NPYRMGNTTF YGPGKTIDTN SPFTVVTQFI TDDGTDTGTL |
| | SEIRRYYVQN GVTYAQPDSD ISGITGNAIN ADYCTAENTV FDGPGTFAKH GGFSAMSEAM STGMVLVMSL WDDYYADMLW LDSTYPTNAS SSTPGAVRGS CSTDSGVPAT |
| | IESESPDSYV TYSNIKVGPI GSTFSSGSGS GSSGSGSSGS ASTSTTSTKT TAATSTSTAV AQHYSQCGGQ DWTGPTTCVS PYTCQVQNAY YSQCL |
| SEQ ID NO: 53 | MKAYFEYLVA ALPLLGLATA QQVGKQTTET HPKLSWKKCT GKANCNTVNA EVVIDSNWRW LHDSSGKNCY DGNKWTSACS SATDCASKCQ LDGANYGTTY GASTSGDALT |
| | LKFVTKHEYG TNIGSRFYLM NGASKYQMFT LMNNEFAFDV DLSTVECGLN AALYFVAMEE DGGMASYSSN KAGAKYGTGY CDAQCARDLK FVGGKANIEG WTPSTNDANA |
| | GVGPYGGCCA EIDVWESNAH SFAFTPHACK TNKYHVCERD NCGGTYSEDR FAGLCDANGC DYNPYRMGNT DFYGKGKTVD TSKKFTVVSR FEENKLTQFF VQNGQKIEIP |
| | GPKWDGIPSD NANITPEFCS AQFQAFGDRD RFAEVGGFAQ LNSALRMPMV LVMSIWDDHY ANMLWLDSVY PPEKEGQPGA ARGDCPQSSG VPAEVESQYA NSKVVYSNIR |
| | FGPVGSTVNV |
| SEQ ID NO: 54 | MFSKFALTGS LLAGAVNAQG VGTQQTETHP QMTWQSCTSP SSCTTNQGEV VIDSNWRWVH DKDGYVNCYT GNTWNTTLCP DDKTCAANCV LDGADYSSTY GITTSGNALS |
| | LQFVTQSSGK NIGSRTYLME SSTKYHLFDL IGNEFAFDVD LSKLPCGLNG ALYFVTMDAD GGMAKYSTNT AGAEYGTGYC DSQCPRDLKF INGQGNVEGW TPSTNDANAG |
| | VGGLGSCCSE MDVWEANSMD MAYTPHPCET AAQHSCNADE CGGTYSSSRY AGDCDPDGCD WNPFRMGNKD FYGSGDTVDT SQKFTVVTQF HGSGSSLTEI SQYYIQGGTK |
| | IQQPNSTWPT LTGYNSITDD FCKAQKVEFN DTDVFSEKGG LAQMGAGMAD GMVLVMSLWD DHYANMLWLD STYPVDADAS SPGKQRGTCA TTSGVPADVE SSDASATVIY |
| | SNIKFGPIGA TY |
| SEQ ID NO: 55 | MFPAAALLSF TLLAVASAQQ IGTNTAEVHP SLTVSQCTTS GGCTSSTQSI VLDANWRWLH STSGYTNCYT GNQWNSDLCP DPDTCATNCA LDGASYESTY GISTDGNAVT |
| | LNFVTQGSQT NVGSRVYLLS DDTHYQTFSL LNKEFSFDVD ASNIGCGING AVYFVQMDAD GGLSKYSSNK AGAQYGTGYC DSQCPQDIKF INGEANLLDW NATSANSGTG |
| | SYGSCCPEMD IWEANKYAAA YTPHPCSVSG QTRCTGTSCG AGSERYDGYC DKDGCDFNSW RMGNETFLGP GMTIDTNKKF TIVTQFITDD NTANGTLSEI RRLYVQGGTV |
| | IQNSVANQPN IPKVNSITDS FCTAQKTEFG DQDYFGTIGG LSQMGKAMSD MVLVMSIWDD YDAEMLWLDS NYPTSGSAST PGISRGPCSA TSGLPATVES QQASASVTYS |
| | NIKWGDIGST YSGSGSSGSS SSSSSSAASA STSTHTSAAA TATSSAAAAT GSPVPAYGQC GGQSYTGSTT CASPYVCKVS NAYYSQCLPA |
| SEQ ID NO: 56 | MKRALCASLS LLAAAVAQQV GTNEPEVHPK MTWKKCSSGG SCSTVNGEVV IDGNWRWIHN IGGYENCYSG NKWTSVCSTN ADCATKCAME GAKYQETYGV STSGDALTLK |
| | FVQQNSSGKN VGSRMYLMNG ANKYQMFTLK NNEFAFDVDL SSVECGMNSA LYFVPMKEDG GMSTEPNNKA GAKYGTGYCD AQCARDLKFI GGKGNIEGWQ PSSTDSSAGI |
| | GAQGACCAEI DIWESNKNAF AFTPHPCENN EYHVCTEPNC GGTYADDRYG GGCDANGCDY NPYRMGNPDF YGPGKTIDTN RKFTVISRFE NNRNYQILMQ DGVAHRIPGP |
| | KFDGLEGETG ELNEQFCTDQ FTVFDERNRF NEVGGWSKLN AAYEIPMVLV MSIWSDHFAN MLWLDSTYPP EKAGQPGSAR GPCPADGGDP NGVVNQYPNA KVIWSNVRFG |
| | PIGSTYQVD |
| SEQ ID NO: 57 | MQLTKAGVFL GALMGGAAAQ QVGTQTAENH PKMTWKKCTG KASCTTVNGE VVIDANWRWL HDASSKNCYD GNRWTDSCRT ASDCAAKCSL EGADYAKTYG ASTSGDALSL |
| | KFVTRHDYGT NIGSRFYLMN GASKYQMFSL LGNEFAFDVD LSTIECGLNS ALYFVAMEED GGMKSYSSNK AGAKYGTGYC DAQCARDLKF VGGKANIEGW KPSSNDANAG |
| | VGPYGACCAE IDVWESNAHA FAFTPHPCTD NKYHVCQDSN CGGTYSDDRF AGKCDANGCD INPYRLGNTD FYGKGKTVDT SKKFTVVTRF ERDALTQFFV QNNKRIDMPS |
| | PALEGLPATG AITAEYCTNV FNVFGDRNRF DEVGGWSQLQ QALSLPMVLV MSIWDDHYSN MLWLDSVYPP DKEGSPGAAR GDCPQDSGVP SEVESQIPGA TVVWSNIRFG |
| | PVGSTVNV |
| SEQ ID NO: 58 | MYRIVATASA LIAAARAQQV CSLNTETKPA LTWSKCTSSG CSDVKGSVVI DANWRWTHQT SGSTNCYTGN KWDTSICTDG KTCAEKCCLD GADYSGTYGI TSSGNQLSLG |
| | FVTNGPYSKN IGSRTYLMEN ENTYQMFQLL GNEFTFDVDV SGIGCGLNGA PHFVSMDEDG GKAKYSGNKA GAKYGTGYCD AQCPRDVKFI NGVANSEGWK PSDSDVNAGV |
| | GNLGTCCPEM DIWEANSIST AFTPHPCTKL TQHSCTGDSC GGTYSSDRYG GTCDADGCDF NAYRQGNKTF YGPGSNFNID TTKKMTVVTQ FHKGSNGRLS EITRLYVQNG |
| | KVIANSESKI AGNPGSSLTS DFCSKQKSVF GDIDDFSKKG GWNGMSDALS APMVLVMSLW HDHHSNMLWL DSTYPTDSTK VGSQRGSCAT TSGKPSDLER DVPNSKVSFS |
| | NIKFGPIGST YKSDGTTPNP PASSSTTGSS TPTNPPAGSV DQWGQCGGQN YSGPTTCKSP FTCKKINDFY SQCQ |
| SEQ ID NO: 59 | MYQRALLFSA LATAVSAQQV GTQKAEVHPA LTWQKCTAAG SCTDQKGSVV IDANWRWLHS TEDTTNCYTG NEWNAELCPD NEACAKNCAL DGADYSGTYG VTADGSSLKL |
| | NFVTSANVGS RLYLMEDDET YQMFNLLNNE FTFDVDVSNL PCGLNGALYF VSMDADGGLS KYPGNKAGAK YGTGYCDSQC PRDLKFINGE ANVEGWKPSD NDKNAGVGGY |
| | GSCCPEMDIW EANSISTAYT PHPCDGMEQT RCDGNDCGGT YSSTRYAGTC DPDGCDFNSF RMGNESFYGP GGLVDTKSPI TVVTQFVTAG GTDSGALKEI RRVYVQGGKV |
| | IGNSASNVAG VEGDSITSDF CTAQKKAFGD EDIFSKHGGL EGMGKALNKM ALIVSIWDDH ASSMMWLDST YPVDADASTP GVARGTCEHG LGDPETVESQ HPDASVTFSN |
| | IKFGPIGSTY KSV |
| SEQ ID NO: 60 | MSALNSFNMY KSALILGSLL ATAGAQQIGT YTAETHPSLS WSTCKSGGSC TTNSGAITLD ANWRWVHGVN TSTNCYTGNT WNTAICDTDA SCAQDCALDG ADYSGTYGIT |
| | TSGNSLRLNF VTGSNVGSRT YLMADNTHYQ IFDLLNQEFT FTVDVSNLPC GLNGALYFVT MDADGGVSKY PNNKAGAQYG VGYCDSQCPR DLKFIAGQAN VEGWTPSTNN |
| | SNTGIGNHGS CCAELDIWEA NSISEALTPH PCDTPGLTVC TADDCGGTYS SNRYAGTCDP DGCDFNPYRL GVTDFYGSGK TVDTTKPFTV VTQFVTDDGT SSGSLSEIRR |
| | YYVQNGVVIP QPSSKISGIS GNVINSDFCA AELSAFGETA SFTNHGGLKN MGSALEAGMV LVMSLWDDYS VNMLWLDSTY PANETGTPGA ARGSCPTTSG NPKTVESQSG |
| | SSYVVFSDIK VGPFNSTFSG GTSTGGSTTT TASGTTSTKA STTSTSSTST GTGVAAHWGQ CGGQGWTGPT TCASGTTCTV VNPYYSQCL |
| SEQ ID NO: 61 | MRTAKFATLA ALVASAAAQQ ACSLTTERHP SLSWNKCTAG GQCQTVQASI TLDSNWRWTH QVSGSTNCYT GNKWDTSICT DAKSCAQNCC VDGADYTSTY GITTNGDSLS |
| | LKFVTKGQHS TNVGSRTYLM DGEDKYQTFE LLGNEFTFDV DVSNIGCGLN GALYFVSMDA DGGLSRYPGN KAGAKYGTGY CDAQCPRDIK FINGEANIEG WTGSTNDPNA |
| | GAGRYGTCCS EMDIWEANNM ATAFTPHPCT IIGQSRCEGD SCGGTYSNER YAGVCDPDGC DFNSYRQGNK TFYGKGMTVD TTKKITVVTQ FLKDANGDLG EIKRFYVQDG |
| | KIIPNSESTI PGVEGNSITQ DWCDRQKVAF GDIDDFNRKG GMKQMGKALA GPMVLVMSIW DDHASNMLWL DSTFPVDAAG KPGAERGACP TTSGVPAEVE AEAPNSNVVF |
| | SNIRFGPIGS TVAGLPGAGN GGNNGGNPPP PTTTTSSAPA TTTTASAGPK AGRWQQCGGI GFTGPTQCEE PYICTKLNDW YSQCL |
| SEQ ID NO: 62 | MMYKKFAALA ALVAGASAQQ ACSLTAENHP SLTWKRCTSG GSCSTVNGAV TIDANWRWTH TVSGSTNCYT GNQWDTSLCT DGKSCAQTCC VDGADYSSTY GITTSGDSLN |
| | LKFVTKHQYG TNVGSRVYLM ENDTKYQMFE LLGNEFTFDV DVSNLGCGLN GALYFVSMDA DGGMSKYSGN KAGAKYGTGY CDAQCPRDLK FINGEANVGN WTPSTNDANA |
| | GFGRYGSCCS EMDVWEANNM ATAFTPHPCT TVGQSRCEAD TCGGTYSSDR YAGVCDPDGC DFNAYRQGDK TFYGKGMTVD TNKKMTVVTQ FHKNSAGVLS EIKRFYVQDG |
| | KIIANAESKI PGNPGNSITQ EYCDAQKVAF SNTDDFNRKG GMAQMSKALA GPMVLVMSVW DDHYANMLWL DSTYPIDQAG APGAERGACP TTSGVPAEIE AQVPNSNVIF |
| | SNIRFGPIGS TVPGLDGSNP GNPTTTVVPP ASTSTSRPTS STSSPVSTPT GQPGGCTTQK WGQCGGIGYT GCTNCVAGTT CTQLNPWYSQ CL |
| SEQ ID NO: 63 | MASLSLSKIC RNALILSSVL STAQGQQVGT YQTETHPSMT WQTCGNGGSC STNQGSVVLD ANWRWVHQTG SSSNCYTGNK WDTSYCSTND ACAQKCALDG ADYSNTYGIT |
| | TSGSEVRLNF VTSNSNGKNV GSRVYMMADD THYEVYKLLN QEFTFDVDVS KLPCGLNGAL YFVVMDADGG VSKYPNNKAG AKYGTGYCDS QCPRDLKFIQ GQANVEGWVS |
| | STNNANTGTG NHGSCCAELD IWESNSISQA LTPHPCDTPT NTLCTGDACG GTYSSDRYSG TCDPDGCDFN PYRVGNTTFY GPGKTIDTNK PITVVTQFIT DDGTSSGTLS |
| | EIKRFYVQDG VTYPQPSADV SGLSGNTINS EYCTAENTLF EGSGSFAKHG GLAGMGEAMS TGMVLVMSLW DDYYANMLWL DSNYPTNEST SKPGVARGTC STSSGVPSEV |
| | EASNPSAYVA YSNIKVGPIG STFKS |
| SEQ ID NO: 64 | MYRAIATASA LIAAVRAQQV CSLTPETKPA LSWSKCTSSG CSNVQGSVTI DANWRWTHQL SGSTNCYTGN KWDTSICTSG KVCAEKCCID GAEYASTYGI TSSGNQLSLS |
| | FVTKGAYGTN IGSRTYLMED ENTYQMFQLL GNEFTFDVDV SNIGCGLNGA LYFVSMDADG GKAKYPGNKA GAKYGTGYCD AQCPRDVKFI NGQANSDGWQ PSKSDVNAGI |
| | GNMGTCCPEM DIWEANSIST AYTPHPCTKL TQHSCTGDSC GGTYSNDRYG GTCDADGCDF NAYRQGNKTF YGPGSGFNVD TTKKVTVVTQ FHKGSNGRLS EITRLYVQNG |
| | KVIANSESKI AGVPGSSLTP EFCTAQKKVF GDTDDFAKKG AWSGMSDALE APMVLVMSLW HDHHSNMLWL DSTYPTDSTK LGAQRGSCST SSGVPADLEK NVPNSKVAFS |
| | NIKFGPIGST YKEGVPEPTN PTNPTNPTNP TNPGTVDQWA QCGGTNYSGP TACKSPFTCK KINDFYSQCQ |
| SEQ ID NO: 65 | MFPKSSLLVL SFLATAYAQQ VGTQTAEVHP SLNWARCTSS GCTNVAGSVT LDANWRWLHT TSGYTNCYTG NSWNTTLCPD GATCAQNCAL DGANYQSTCG ITTSGNALTL |
| | KFVTQGEQKN IGSRVYLMAS ESRYEMFGLL NKEFTFDVDV SNLPCGLNGA LYFSSMDADG GMAKNPGNKA GAKYGTGYCD SQCPRDIKFI NGEANVAGWN GSPNDTNAGT |
| | GNWGACCNEM DIWEANSISA AYTPHPCTVQ GLSRCSGTAC GTNDRYGTVC DPDGCDFNSY RMGDKTYYGP GGTGVDTRSK FTVVTQFLTN NNSSSGTLSE IRRLYVQNGR |
| | VVQNSKVNIP GMSNTLDSIT TGFCDSQKTA FGDTRSFQNK GGMSAMGQAL GAGMVLVLSV WDDHAANMLW LDSNYPVDAD PSKPGIARGT CSTTSGKPTD VEQSAANSSV |
| | TFSNIKFGDI GTTYTGGSVT TTPGNPGTTT STAPGAVQTK WGQCGGQGWT GPTRCESGST CTVVNQWYSQ CI |
| SEQ ID NO: 66 | MFRKAALLAF SFLAIAHGQQ VGTNQAENHP SLPSQHCTAS GCTTSSTSVV LDANWRWVHT TTGYTNCYTG QTWDASICPD GVTCAKACAL DGADYSGTYG ITTSGNALTL |
| | QFVKGTNVGS RVYLLQDASN YQLFKLINQE FTFDVDMSNL PCGLNGAVYL SQMDQDGGVS RFPTNTAGAK YGTGYCDSQC PRDIKFINGE ANVAGWTGSS SDPNSGTGNY |
| | GTCCSEMDIW EANSVAAAYT PHPCSVNQQT RCTGADCGQD ANRYKGVCDP DGCDFNSFRM GDQTFLGKGL TVDTSRKFTI VTQFISDDGT SSGNLAEIRR FYVQDGKVIP |
| | NSKVNIAGCD AVNSITDKFC TQQKTAFGDT NRFADQGGLK QMGAALKSGM VLALSLWDDH AANMLWLDSD YPTTADASKP GVARGTCPNT SGVPKDVESQ SGSATVTYSN |
| | IKWGDLNSTF SGTASNPTGP SSSPSGPSSS SSSTAGSQPT QPSSGSVAQW GQCGGIGYSG ATGCVSPYTC HVVNPYYSQC Y |
| SEQ ID NO: 67 | TETHPRLTWK RCTSGGNCST VNGAVTIDAN WRWTHTVSGS TNCYTGNEWD TSICSDGKSC AQTCCVDGAD YSSTYGITTS GDSLNLKFVT KHQHGTNVGS RVYLMENDTK |
| | YQMFELLGNE FTFDVDVSNL GCGLNGALYF VSMDADGGMS KYSGNKAGAK YGTGYCDAQC PRDLKFINGE ANIENWTPST NDANAGFGRY GSCCSEMDIW EANNMATAFT |
| | PHPCTIIGQS RCEGNSCGGT YSSERYAGVC DPDGCDFNAY RQGDKTFYGK GMTVDTTKKM TVVTQFHKNS AGVLSEIKRF YVQDGKIIAN AESKIPGNPG NSITQEWCDA |
| | QKVAFGDIDD FNRKGGMAQM SKALEGPMVL VMSVWDDHYA NMLWLDSTYP IDKAGTPGAE RGACPTTSGV PAEIEAQVPN SNVIFSNIRF GPIGSTVPGL DGSTPSNPTA |
| | TVAPPTSTTT SVRSSTTQIS TPTSQPGGCT TQKWGQCGGI GYTGCTNCVA GTTCTELNPW YSQCL |
| SEQ ID NO: 68 | MFHKAVLVAF SLVTIVHGQQ AGTQTAENHP QLSSQKCTAG GSCTSASTSV VLDSNWRWVH TTSGYTNCYT GNTWDASICS DPVSCAQNCA LDGADYAGTY GITTSGDALT |
| | LKFVTGSNVG SRVYLMEDET NYQMFKLMNQ EFTFDVDVSN LPCGLNGAVY FVQMDQDGGT SKFPNNKAGA KFGTGYCDSQ CPQDIKFING EANIVDWTAS AGDANSGTGS |
| | FGTCCQEMDI WEANSISAAY TPHPCTVTEQ TRCSGSDCGQ GSDRFNGICD PDGCDFNSFR MGNTEFYGKG LTVDTSQKFT IVTQFISDDG TADGNLAEIR RFYVQNGKVI |
| | PNSVVQITGI DPVNSITEDF CTQQKTVFGD TNNFAAKGGL KQMGEAVKNG MVLALSLWDD YAAQMLWLDS DYPTTADPSQ PGVARGTCPT TSGVPSQVEG QEGSSSVIYS |
| | NIKFGDLNST FTGTLTNPSS PAGPPVTSSP SEPSQSTQPS QPAQPTQPAG TAAQWAQCGG MGFTGPTVCA SPFTCHVLNP YYSQCY |
| SEQ ID NO: 69 | MFRAAALLAF TCLAMVSGQQ AGTNTAENHP QLQSQQCTTS GGCKPLSTKV VLDSNWRWVH STSGYTNCYT GNEWNTSLCP DGKTCAANCA LDGADYSGTY GITSTGTALT |
| | LKFVTGSNVG SRVYLMADDT HYQLLKLLNQ EFTFDVDMSN LPCGLNGALY LSAMDADGGM SKYPGNKAGA KYGTGYCDSQ CPKDIKFING EANVGNWTET GSNTGTGSYG |
| | TCCSEMDIWE ANNDAAAFTP HPCTTTGQTR CSGDDCARNT GLCDHGDGCD FNSFRMGDKT FLGKGMTVDT SKPFTDVTQF LTNDNTSTGT LSEIRRIYIQ NGKVIQNSVA |
| | NIPGVDPVNS ITDNFCAQQK TAFGDTNWFA QKGGLKQMGE ALGNGMVLAL SIWDDHAANM LWLDSDYPTD KDPSAPGVAR GTCATTSGVP SDVESQVPNS QVVFSNIKFG |
| | DIGSTFSGTS SPNPPGGSTT SSPVTTSPTP PPTGPTVPQW GQCGGIGYSG STTCASPYTC HVLNPYYSQC Y |
| SEQ ID NO: 70 | MMMKQYLQYL AAALPLVGLA AGQRAGNETP ENHPPLTWQR CTAPGNCQTV NAEVVIDANW RWLHDDNMQN CYDGNQWTNA CSTATDCAEK CMIEGAGDYL GTYGASTSGD |
| | ALTLKFVTKH EYGTNVGSRF YLMNGPDKYQ MFNLMGNELA FDVDLSTVEC GINSALYFVA MEEDGGMASY PSNQAGARYG TGYCDAQCAR DLKFVGGKAN IEGWKSSTSD |
| | PNAGVGPYGS CCAEIDVWES NAYAFAFTPH ACTTNEYHVC ETTNCGGTYS EDRFAGKCDA NGCDYNPYRM GNPDFYGKGK TLDTSRKFTV VSRFEENKLS QYFIQDGRKI |
| | EIPPPTWEGM PNSSEITPEL CSTMFDVFND RNRFEEVGGF EQLNNALRVP MVLVMSIWDD HYANMLWLDS IYPPEKEGQP GAARGDCPTD SGVPAEVEAQ FPDAQVVWSN |
| | IRFGPIGSTY DF |
| SEQ ID NO: 71 | MYRSATFLTF ASLVLGQQVG TYTAERHPSM PIQVCTAPGQ CTRESTEVVL DANWRWTHIT NGYTNCYTGN EWNATACPDG ATCAKNCAVD GADYSGTYGI TTPSSGALRL |
| | QFVKKNDNGQ NVGSRVYLMA SSDKYKLFNL LNKEFTFDVD VSKLPCGLNG AVYFSEMLED GGLKSFSGNK AGAKYGTGYC DSQCPQDIKF INGEANVEGW GGADGNSGTG |
| | KYGICCAEMD IWEANSDATA YTPHVCSVNE QTRCEGVDCG AGSDRYNSIC DKDGCDFNSY RLGNREFYGP GKTVDTTRPF TIVTQFVTDD GTDSGNLKSI HRYYVQDGNV |
| | IPNSVTEVAG VDQTNFISEG FCEQQKSAFG DNNYFGQLGG MRAMGESLKK MVLVLSIWDD HAVNMNWLDS IFPNDADPEQ PGVARGRCDP ADGVPATIEA AHPDAYVIYS |
| | NIKFGAINST FTAN |
| SEQ ID NO: 72 | MYRTLAFASL SLYGAARAQQ VGTSTAENHP KLTWQTCTGT GGTNCSNKSG SVVLDSNWRW AHNVGGYTNC YTGNSWSTQY CPDGDSCTKN CAIDGADYSG TYGITTSNNA |
| | LSLKFVTKGS FSSNIGSRTY LMETDTKYQM FNLINKEFTF DVDVSKLPCG LNGALYFVEM AADGGIGKGN NKAGAKYGTG YCDSQCPHDI KFINGKANVE GWNPSDADPN |
| | GGAGKIGACC PEMDIWEANS ISTAYTPHPC RGVGLQECSD AASCGDGSNR YDGQCDKDGC DFNSYRMGVK DFYGPGATLD TTKKMTVITQ FLGSGSSLSE IKRFYVQNGK |
| | VYKNSQSAVA GVTGNSITES FCTAQKKAFG DTSSFAALGG LNEMGASLAR GHVLIMSLWG DHAVNMLWLD STYPTDADPS KPGAARGTCP TTSGKPEDVE KNSPDATVVF |
| | SNIKFGPIGS TFAQPA |
| SEQ ID NO: 73 | MYQKLALISA FLATARAQSA CTLQAETHPP LTWQKCSSGG TCTQQTGSVV IDANWRWTHA TNSSTNCYDG NTWSSTLCPD NETCAKNCCL DGAAYASTYG VTTSADSLSI |
| | GFVTQSAQKN VGARLYLMAS DTTYQEFTLL GNEFSFDVDV SQLPCGLNGA LYFVSMDADG GVSKYPTNTA GAKYGTGYCD SQCPRDLKFI NGQANVEGWE PSSNNANTGI |
| | GGHGSCCSEM DIWEANSISE ALTPHPCTTV GQEICDGDSC GGTYSGDRYG GTCDPDGCDW NPYRLGNTSF YGPGSSFTLD TTKKLTVVTQ FETSGAINRY YVQNGVTFQQ |
| | PNAELGDYSG NSLDDDYCAA EEAEFGGSSF SDKGGLTQFK KATSGGMVLV MSLWDDYYAN MLWLDSTYPT NETSSTPGAV RGSCSTSSGV PAQLESNSPN AKVVYSNIKF |
| | GPIGSTGNSS GGNPPGGNPP GTTTTRRPAT STGSSPGPTQ THYGQCGGIG YSGPTVCASG STCQVLNPYY SQCL |
| SEQ ID NO: 74 | MVDSFSIYKT ALLLSMLATS NAQQVGTYTA ETHPSLTWQT CSGSGSCTTT SGSVVIDANW RWVHEVGGYT NCYSGNTWDS SICSTDTTCA SECALEGATY ESTYGVTTSG |
| | SSLRLNFVTT ASQKNIGSRL YLLADDSTYE TFKLFNREFT FDVDVSNLPC GLNGALYFVS MDADGGVSRF PTNKAGAKYG TGYCDSQCPR DLKFIDGQAN IEGWEPSSTD |
| | VNAGTGNHGS CCPEMDIWEA NSISSAFTAH PCDSVQQTMC TGDTCGGTYS DTTDRYSGTC DPDGCDFNPY RFGNTNFYGP GKTVDNSKPF TVVTQFITHD GTDTGTLTEI |
| | RRLYVQNGVV IGNGPSTYTA ASGNSITESF CKAEKTLFGD TNVFETHGGL SAMGDALGDG MVLVLSLWDD HAADMLWLDS DYPTTSCASS PGVARGTCPT TTGNATYVEA |
| | NYPNSYVTYS NIKFGTLNST YSGTSSGGSS SSSTTLTTKA STSTTSSKTT TTTSKTSTTS SSSTNVAQLY GQCGGQGWTG PTTCASGTCTKQNDYYSQCL |
| SEQ ID NO: 75 | MYRILKSFIL LSLVNMSLSQ KIGKLTPEVH PPMTFQKCSE GGSCETIQGE VVVDANWRWV HSAQGQNCYT GNTWNPTICP DDETCAENCY LDGANYESVY GVTTSEDSVR |
| | LNFVTQSQGK NIGSRLFLMS NESNYQLFHV LGQEFTFDVD VSNLDCGLNG ALYLVSMDSD GGSARFPTNE AGAKYGTGYC DAQCPRDLKF ISGSANVDGW IPSTNNPNTG |
| | YGNLGSCCAE MDLWEANNMA TAVTPHPCDT SSQSVCKSDS CGGAASSNRY GGICDPDGCD YNPYRMGNTS FFGPNKMIDT NSVITVVTQF ITDDGSSDGK LTSIKRLYVQ |
| | DGNVISQSVS TIDGVEGNEV NEEFCTNQKK VFGDEDSFTK HGGLAKMGEA LKDGMVLVLS LWDDYQANML WLDSSYPTTS SPTDPGVARG SCPTTSGVPS KVEQNYPNAY |
| | VVYSNIKVGP IDSTYKK |
| SEQ ID NO: 76 | MISRVLAISS LLAAARAQQI GTNTAEVHPA LTSIVIDANW RWLHTTSGYT NCYTGNSWDA TLCPDAVTCA ANCALDGADY SGTYGITTSG NSLKLNFVTK GANTNVGSRT |
| | YLMAAGSKTQ YQLLKLLGQE FTFDVDVSNL PCGLNGALYF AEMDADGGVS RFPTNKAGAQ YGTGYCDAQC PQDIKFINGQ ANSVGWTPSS NDVNTGTGQY GSCCSEMDIW |
| | EANKISAAYT PHPCSVDGQT RCTGTDCGIG ARYSSLCDAD GCDFNSYRMG DTGFYGAGLT VDTSKVFTVV TQFITNDGTT SGTLSEIRRF YVQNGKVIPN SQSKVTGVSG |
| | NSITDSFCAA QKTAFGDTNE FATKGGLATM SKALAKGMVL VMSIWDDHSA NMLWLDAPYP ASKSPSAAGV SRGSCSASSG VPADVEANSP GASVTYSNIK WGPINSTYSA |
| | GTGSNTGSGS GSTTTLVSSV PSSTPTSTTG VPKYGQCGGS GYTGPTNCIG STCVSMGQYY SQCQ |
| SEQ ID NO: 77 | MYRQVATALS FASLVLGQQV GTLTAETHPS LPIEVCTAPG SCTKEDTTVV LDANWRWTHV TDGYTNCYTG NAWNETACPD GKTCAANCAI DGAEYEKTYG ITTPEEGALR |
| | LNFVTESNVG SRVYLMAGED KYRLFNLLNK EFTMDVDVSN LPCGLNGAVY FSEMDEDGGM SRFEGNKAGA KYGTGYCDSQ CPRDIKFING EANSEGWGGE DGNSGTGKYG |
| | TCCAEMDIWE ANLDATAYTP HPCKVTEQTR CEDDTECGAG DARYEGLCDR DGCDFNSFRL GNKEFYGPEK TVDTSKPFTL VTQFVTADGT DTGALQSIRR FYVQDGTVIP |
| | NSETVVEGVD PTNEITDDFC AQQKTAFGDN NHFKTIGGLP AMGKSLEKMV LVLSIWDDHA VYMNWLDSNY PTDADPTKPG VARGRCDPEA GVPETVEAAH PDAYVIYSNI |
| | KIGALNSTFA AA |
| SEQ ID NO: 78 | MSSFQVYRAA LLLSILATAN AQQVGTYTTE THPSLTWQTC TSDGSCTTND GEVVIDANWR WVHSTSSATN CYTGNEWDTS ICTDDVTCAA NCALDGATYE ATYGVTTSGS |
| | ELRLNFVTQG SSKNIGSRLY LMSDDSNYEL FKLLGQEFTF DVDVSNLPCG LNGALYFVAM DADGGTSEYS GNKAGAKYGT GYCDSQCPRD LKFINGEANC DGWEPSSNNV |
| | NTGVGDHGSC CAEMDVWEAN SISNAFTAHP CDSVSQTMCD GDSCGGTYSA SGDRYSGTCD PDGCDYNPYR LGNTDFYGPG LTVDTNSPFT VVTQFITDDG TSSGTLTEIK |
| | RLYVQNGEVI ANGASTYSSV NGSSITSAFC ESEKTLFGDE NVFDKHGGLE GMGEAMAKGM VLVLSLWDDY AADMLWLDSD YPVNSSASTP GVARGTCSTD SGVPATVEAE |
| | SPNAYVTYSN IKFGPIGSTY SSGSSSGSGS SSSSSSTTTK ATSTTLKTTS TTSSGSSSTS AAQAYGQCGG QGWTGPTTCV SGYTCTYENA YYSQCL |
| SEQ ID NO: 79 | MYRAIATASA LLATARAQQV CTLNTENKPA LTWAKCTSSG CSNVRGSVVV DANWRWAHST SSSTNCYTGN TWDKTLCPDG KTCADKCCLD GADYSGTYGV TSSGNQLNLK |
| | FVTVGPYSTN VGSRLYLMED ENNYQMFDLL GNEFTFDVDV NNIGCGLNGA LYFVSMDKDG GKSRFSTNKA GAKYGTGYCD AQCPRDVKFI NGVANSDEWK PSDSDKNAGV |
| | GKYGTCCPEM DIWEANKIST AYTPHPCKSL TQQSCEGDAC GGTYSATRYA GTCDPDGCDF NPYRQGNKTF YGPGSGFNVD TTKKVTVVTQ FIKGSDGKLS EIKRLYVQNG |
| | KVIGNPQSEI ANNPGSSVTD SFCKAQKVAF NDPDDFNKKG GWSGMSDALA KPMVLVMSLW HDHYANMLWL DSTYPKGSKT PGSARGSCPE DSGDPDTLEK EVPNSGVSFS |
| | NIKFGPIGST YTGTGGSNPD PEEPEEPEEP VGTVPQYGQC GGINYSGPTA CVSPYKCNKI NDFYSQCQ |
| SEQ ID NO: 80 | EQAGTATAEN HPPLTWQECT APGSCTTQNG AVVLDANWRW VHDVNGYTNC YTGNTWDPTY CPDDETCAQN CALDGADYEG TYGVTSSGSS LKLNFVTGSN VGSRLYLLQD |
| | DSTYQIFKLL NREFSFDVDV SNLPCGLNGA LYFVAMDADG GVSKYPNNKA GAKYGTGYCD SQCPRDLKFI DGEANVEGWQ PSSNNANTGI GDHGSCCAEM DVWEANSISN |
| | AVTPHPCDTP GQTMCSGDDC GGTYSNDRYA GTCDPDGCDF NPYRMGNTSF YGPGKIIDTT KPFTVVTQFL TDDGTDTGTL SEIKRFYIQN SNVIPQPNSD ISGVTGNSIT |
| | TEFCTAQKQA FGDTDDFSQH GGLAKMGAAM QQGMVLVMSL WDDYAAQMLW LDSDYPTDAD PTTPGIARGT CPTDSGVPSD VESQSPNSYV TYSNIKFGPI NSTFTAS |
| SEQ ID NO: 81 | MFPTLALVSL SFLAIAYGQQ VGTLTAETHP KLSVSQCTAG GSCTTVQRSV VLDSNWRWLH DVGGSTNCYT GNTWDDSLCP DPTTCAANCA LDGADYSGTY GITTSGNALS |
| | LKFVTQGPYS TNIGSRVYLL SEDDSTYEMF NLKNQEFTFD VDMSALPCGL NGALYFVEMD KDGGSGRFPT NKAGSKYGTG YCDTQCPHDI KFINGEANVL DWAGSSNDPN |
| | AGTGHYGTCC NEMDIWEANS MGAAVTPHVC TVQGQTRCEG TDCGDGDERY DGICDKDGCD FNSWRMGDQT FLGPGKTVDT SSKFTVVTQF ITADNTTSGD LSEIRRLYVQ |
| | NGKVIANSKT QIAGMDAYDS ITDDFCNAQK TTFGDTNTFE QMGGLATMGD AFETGMVLVM SIWDDHEAKM LWLDSDYPTD ADASAPGVSR GPCPTTSGDP TDVESQSPGA |
| | TVIFSNIKTG PIGSTFTS |
| SEQ ID NO: 82 | MLSASKAAAI LAFCAHTASA WVVGDQQTET HPKLNWQRCT GKGRSSCTNV NGEVVIDANW RWLAHRSGYT NCYTGSEWNQ SACPNNEACT KNCAIEGSDY AGTYGITTSG |
| | NQMNIKFITK RPYSTNIGAR TYLMKDEQNY EMFQLIGNEF TFDVDLSQRC GMNGALYFVS MPQKGQGAPG AKYGTGYCDA QCARDLKFVR GSANAEGWTK SASDPNSGVG |
| | KKGACCAQMD VWEANSAATA LTPHSCQPAG YSVCEDTNCG GTYSEDRYAG TCDANGCDFN PFRVGVKDFY GKGKTVDTTK KMTVVTQFVG SGNQLSEIKR FYVQDGKVIA |
| | NPEPTIPGME WCNTQKKVFQ EEAYPFNEFG GMASMSEGMS QGMVLVMSLW DDHYANMLWL DSNWPREADP AKPGVARRDC PTSGGKPSEV EAANPNAQVM FSNIKFGPIG |
| | STFAHAA |
| SEQ ID NO: 83 | MFRTATLLAF TMAAMVFGQQ VGTNTARSHP ALTSQKCTKS GGCSNLNTKI VLDANWRWLH STSGYTNCYT GNQWDATLCP DGKTCAANCA LDGADYTGTY GITASGSSLK |
| | LQFVTGSNVG SRVYLMADDT HYQMFQLLNQ EFTFDVDMSN LPCGLNGALY LSAMDADGGM AKYPTNKAGA KYGTGYCDSQ CPRDIKFING EANVEGWNAT SANAGTGNYG |
| | TCCTEMDIWE ANNDAAAYTP HPCTTNAQTR CSGSDCTRDT GLCDADGCDF NSFRMGDQTF LGKGLTVDTS KPFTVVTQFI TNDGTSAGTL TEIRRLYVQN GKVIQNSSVK |
| | IPGIDPVNSI TDNFCSQQKT AFGDTNYFAQ HGGLKQVGEA LRTGMVLALS IWDDYAANML WLDSNYPTNK DPSTPGVARG TCATTSGVPA QIEAQSPNAY VVFSNIKFGD |
| | LNTTYTGTVS SSSVSSSHSS TSTSSSHSSS STPPTQPTGV TVPQWGQCGG IGYTGSTTCA SPYTCHVLNP YYSQCY |
| SEQ ID NO: 84 | MYQRALLFSA LMAGVSAQQV GTQKPETHPP LAWKECTSSG CTSKDGSVVI DANWRWVHSV DGYKNCYTGN EWDSTLCPDD ATCATNCAVD GADYAGTYGA TTEGDSLSIN |
| | FVTGSNIGSR FYLMEDENKY QMFKLLNKEF TFDVDVSTLP CGLNGALYFV SMDADGGMSK YETNKAGAKY GTGYCDSQCP RDLKFINGKG NVEGWKPSAN DKNAGVGPHG |
| | SCCAEMDIWE ANSISTALTP HPCDTNGQTI CEGDSCGGTY STTRYAGTCD PDGCDFNPFR MGNESFYGPG KMVDTKSKMT VVTQFITSDG TDTGSLKEIK RVYVQNGKVI |
| | ANSASDVSGI TGNSITSDFC TAQKKTFGDE DVFNKHGGLS GMGDALGEGM VLVMSLWDDH NSNMLWLDGE KYPTDAAASK AGVSRGTCST DSGKPSTVES ESGSAKVVFS |
| | NIKVGSIGST FSA |
| SEQ ID NO: 85 | MTSKIALASL FAAAYGQQIG TYTTETHPSL TWQSCTAKGS CTTQSGSIVL DGNWRWTHST TSSTNCYTGN TWDATLCPDD ATCAQNCALD GADYSGTYGI TTSGDSLRLN |
| | FVTQTANKNV GSRVYLLADN THYKTFNLLN QEFTFDVDVS NLPCGLNGAV YFANLPADGG ISSTNKAGAQ YGTGYCDSQC PRDGKFINGK ANVDGWVPSS NNPNTGVGNY |
| | GSCCAEMDIW EANSISTAVT PHSCDTVTQT VCTGDNCGGT YSTTRYAGTC DPDGCDFNPY RQGNESFYGP GKTVDTNSVF TIVTQFLTTD GTSSGTLNEI KRFYVQNGKV |
| | IPNSESTISG VTGNSITTPF CTAQKTAFGD PTSFSDHGGL ASMSAAFEAG MVLVLSLWDD YYANMLWLDS TYPTTKTGAG GPRGTCSTSS GVPASVEASS PNAYVVYSNI |
| | KVGAINSTFG |
| SEQ ID NO: 86 | MYTKFAALAA LVATVRGQAA CSLTAETHPS LQWQKCTAPG SCTTVSGQVT IDANWRWLHQ TNSSTNCYTG NEWDTSICSS DTDCATKCCL DGADYTGTYG VTASGNSLNL |
| | KFVTQGPYSK NIGSRMYLME SESKYQGFTL LGQEFTFDVD VSNLGCGLNG ALYFVSMDLD GGVSKYTTNK AGAKYGTGYC DSQCPRDLKF INGQANIDGW QPSSNDANAG |
| | LGNHGSCCSE MDIWEANKVS AAYTPHPCTT IGQTMCTGDD CGGTYSSDRY AGICDPDGCD FNSYRMGDTS FYGPGKTVDT GSKFTVVTQF LTGSDGNLSE IKRFYVQNGK |
| | VIPNSESKIA GVSGNSITTD FCTAQKTAFG DTNVFEERGG LAQMGKALAE PMVLVLSVWD DHAVNMLWLD STYPTDSTKP GAARGDCPIT SGVPADVESQ APNSNVIYSN |
| | IRFGPINSTY TGTPSGGNPP GGGTTTTTTT TTSKPSGPTT TTNPSGPQQT HWGQCGGQGW TGPTVCQSPY TCKYSNDWYS QCL |
| SEQ ID NO: 87 | MYQRALLFSA LLSVSRAQQA GTAQEEVHPS LTWQRCEASG SCTEVAGSVV LDSNWRWTHS VDGYTNCYTG NEWDATLCPD NESCAQNCAV DGADYEATYG ITSNGDSLTL |
| | KFVTGSNVGS RVYLMEDDET YQMFDLLNNE FTFDVDVSNF PCGLNGALYF TSMDADGGLS KYEGNTAGAK YGTGYCDSQC PRDIKFINGL GNVEGWEPSD SDANAGVGGM |
| | GTCCPEMDIW EANSISTAYT PHPCDSVEQT MCEGDSCGGT YSDDRYGGTC DPDGCDFNSY RMGNTRFYGP GAIIDTSSKF TVVTQFIADG GSLSEIKRFY VQNGEVIPNS |
| | ESNISGVEGN SITSEFCTAQ KTAFGDEDIF AQHGGLSAMG DAASAMVLIL SIWDDHHSSM MWLDSSYPTD ADPSQPGVAR GTCEQGAGDP DVVESEHADA SVTFSNIKFG |
| | PIGSTF |
| SEQ ID NO: 88 | MMMKQYLQYL AAGSLMTGLV AGQGVGTQQT ETHPRITWKR CTGKANCTTV QAEVVIDSNW RWIHTSGGTN CYDGNAWNTA ACSTATDCAS KCLMEGAGNY QQTYGASTSG |
| | DSLTLKFVTK HEYGTNVGSR FYLMNGASKY QMFTLMNNEF TFDVDLSTVE CGLNSALYFV AMEEDGGMRS YPTNKAGAKY GTGYCDAQCA RDLKFVGGKA NIEGWRESSN |
| | DENAGVGPYG GCCAEIDVWE SNAHAYAFTP HACENNNYHV CERDTCGGTY SEDRFAGGCD ANGCDYNPYR MGNPDFYGKG KTVDTTKKFT VVTRFQDDNL EQFFVQNGQK |
| | ILAPAPTFDG IPASPNLTPE FCSTQFDVFT DRNRFREVGD FPQLNAALRI PMVLVMSIWA DHYANMLWLD SVYPPEKEGE PGAARGPCAQ DSGVPSEVKA NYPNAKVVWS |
| | NIRFGPIGST VNV |
| SEQ ID NO: 89 | MYQRALLFSF FLAAARAQQA GTVTAENHPS LTWQQCSSGG SCTTQNGKVV IDANWRWVHT TSGYTNCYTG NTWDTSICPD DVTCAQNCAL DGADYSGTYG VTTSGNALRL |
| | NFVTQSSGKN IGSRLYLLQD DTTYQIFKLL GQEFTFDVDV SNLPCGLNGA LYFVAMDADG GLSKYPGNKA GAKYGTGYCD SQCPRDLKFI NGQANVEGWQ PSANDPNAGV |
| | GNHGSCCAEM DVWEANSIST AVTPHPCDTP GQTMCQGDDC GGTYSSTRYA GTCDPDGCDF NPYRQGNHSF YGPGKIVDTS SKFTVVTQFI TDDGTPSGTL TEIKRFYVQN |
| | GKVIPQSEST ISGVTGNSIT TEYCTAQKAA FGDNTGFFTH GGLQKISQAL AQGMVLVMSL WDDHAANMLW LDSTYPTDAD PDTPGVARGT CPTTSGVPAD VESQNPNSYV |
| | IYSNIKVGPI NSTFTAN |
| SEQ ID NO: 90 | MFAIVLLGLT RSLGTGTNQA ENHPSLSWQN CRSGGSCTQT SGSVVLDSNW RWTHDSSLTN CYDGNEWSSS LCPDPKTCSD NCLIDGADYS GTYGITSSGN SLKLVFVTNG |
| | PYSTNIGSRV YLLKDESHYQ IFDLKNKEFT FTVDDSNLDC GLNGALYFVS MDEDGGTSRF SSNKAGAKYG TGYCDAQCPH DIKFINGEAN VENWKPQTND ENAGNGRYGA |
| | CCTEMDIWEA NKYATAYTPH ICTVNGEYRC DGSECGDTDS GNRYGGVCDK DGCDFNSYRM GNTSFWGPGL IIDTGKPVTV VTQFVTKDGT DNGQLSEIRR KYVQGGKVIE |
| | NTVVNIAGMS SGNSITDDFC NEQKSAFGDT NDFEKKGGLS GLGKAFDYGM VLVLSLWDDH QVNMLWLDSI YPTDQPASQP GVKRGPCATS SGAPSDVESQ HPDSSVTFSD |
| | IRFGPIDSTY |
| SEQ ID NO: 91 | MHQRALLFSA LVGAVRAQQA GTLTEEVHPP LTWQKCTADG SCTEQSGSVV IDSNWRWLHS TNGSTNCYTG NTWDESLCPD NEACAANCAL DGADYESTYG ITTSGDALTL |
| | TFVTGENVGS RVYLMAEDDE SYQTFDLVGN EFTFDVDVSN LPCGLNGALY FTSMDADGGV SKYPANKAGA KYGTGYCDSQ CPRDLKFING MANVEGWTPS DNDKNAGVGG |
| | HGSCCPELDI WEANSISSAF TPHPCDDLGQ TMCSGDDCGG TYSETRYAGT CDPDGCDFNA YRMGNTSYYG PDKIVDTNSV MTVVTQFIGD GGSLSEIKRL YVQNGKVIAN |
| | AQSNVDGVTG NSITSDFCTA QKTAFGDQDI FSKHGGLSGM GDAMSAMVLI LSIWDDHNSS MMWLDSTYPE DADASEPGVA RGTCEHGVGD PETVESQHPG ATVTFSKIKF |
| | GPIGSTYSSN STA |
| SEQ ID NO: 92 | MFRAAALLAF TCLAMVSGQQ AGTNTAENHP QLQSQQCTTS GGCKPLSTKV VLDSNWRWVH STSGYTNCYT GNEWDTSLCP DGKTCAANCA LDGADYSGTY GITSTGTALT |
| | LKFVTGSNVG SRVYLMADDT HYQLLKLLNQ EFTFDVDMSN LPCGLNGALY LSAMDADGGM SKYPGNKAGA KYGTGYCDSQ CPKDIKFING EANVGNWTET GSNTGTGSYG |
| | TCCSEMDIWE ANNDAAAFTP HPCTTTGQTR CSGDDCARNT GLCDGDGCDF NSFRMGDKTF LGKGMTVDTS KPFTVVTQFL TNDNTSTGTL SEIRRIYIQN GKVIQNSVAN |
| | IPGVDPVNSI TDNFCAQQKT AFGDTNWFAQ KGGLKQMGEA LGNGMVLALS IWDDHAANML WLDSDYPTDK DPSAPGVARG TCATTSGVPS DVESQVPNSQ VVFSNIKFGD |
| | IGSTFSGTSS PNPPGGSTTS SPVTTSPTPP PTGPTVPQWG QCGGIGYSGS TTCASPYTCH VLNPCESILS LQRSSNADQY LQTTRSATKR RLDTALQPRK |
| SEQ ID NO: 93 | MRTALALILA LAAFSAVSAQ QAGTITAETH PTLTIQQCTQ SGGCAPLTTK VVLDVNWRWI HSTTGYTNCY SGNTWDAILC PDPVTCAANC ALDGADYTGT FGILPSGTSV |
| | TLRPVDGLGL RLFLLADDSH YQMFQLLNKE FTFDVEMPNM RCGSSGAIHL TAMDADGGLA KYPGNQAGAK YGTGFCSAQC PKGVKFINGQ ANVEGWLGTT ATTGTGFFGS |
| | CCTDIALWEA NDNSASFAPH PCTTNSQTRC SGSDCTADSG LCDADGCNFN SFRMGNTTFF GAGMSVDTTK LFTVVTQFIT SDNTSMGALV EIHRLYIQNG QVIQNSVVNI |
| | PGINPATSIT DDLCAQENAA FGGTSSFAQH GGLAQVGEAL RSGMVLALSI VNSAADTLWL DSNYPADADP SAPGVARGTC PQDSASIPEA PTPSVVFSNI KLGDIGTTFG |
| | AGSALFSGRS PPGPVPGSAP ASSATATAPP FGSQCGGLGY AGPTGVCPSP YTCQALNIYY SQCI |
| SEQ ID NO: 94 | MYQRALLFSF FLAAARAHEA GTVTAENHPS LTWQQCSSGG SCTTQNGKVV IDANWRWVHT TSGYTNCYTG NTWDTSICPD DVTCAQNCAL DGADYSGTYG VTTSGNALRL |
| | NFVTQSSGKN IGSRLYLLQD DTTYQIFKLL GQEFTFDVDV SNLPCGLNGA LYFVAMDADG NLSKYPGNKA GAKYGTGYCD SQCPRDLKFI NGQANVEGWQ PSANDPNAGV |
| | GNHGSSCAEM DVWEANSIST AVTPHPCDTP GQTMCQGDDC GGTYSSTRYA GTCDTDGCDF NPYQPGNHSF YGPGKIVDTS SKFTVVTQFI TDDGTPSGTL TEIKRFYVQN |
| | GKVIPQSEST ISGVTGNSIT TEYCTAQKAA FDNTGFFTHG GLQKISQALA QGMVLVMSLW DDHAANMLWL DSTYPTDADP DTPGVARGTC PTTSGVPADV ESQNPNSYVI |
| | YSNIKVGPIN STFTAN |
| SEQ ID NO: 95 | MHKRAATLSA LVVAAAGFAR GQGVGTQQTE THPKLTFQKC SAAGSCTTQN GEVVIDANWR WVHDKNGYTN CYTGNEWNTT ICADAASCAS NCVVDGADYQ GTYGASTSGN |
| | ALTLKFVTKG SYATNIGSRM YLMASPTKYA MFTLLGHEFA FDVDLSKLPC GLNGAVYFVS MDEDGGTSKY PSNKAGAKYG TGYCDSQCPR DLKFIDGKAN SASWQPSSND |
| | QNAGVGGMGS CCAEMDIWEA NSVSAAYTPH PCQNYQQHSC SGDDCGGTYS ATRFAGDCDP DGCDWNAYRM GVHDFYGNGK TVDTGKKFSI VTQFKGSGST LTEIKQFYVQ |
| | DGRKIENPNA TWPGLEPFNS ITPDFCKAQK QVFGDPDRFN DMGGFTNMAK ALANPMVLVL SLWDDHYSNM LWLDSTYPTD ADPSAPGKGR GTCDTSSGVP SDVESKNGDA |
| | TVIYSNIKFG PLDSTYTAS |
| SEQ ID NO: 96 | MRASLLAFSL NSAAGQQAGT LQTKNHPSLT SQKCRQGGCP QVNTTIVLDA NWRWTHSTSG STNCYTGNTW QATLCPDGKT CAANCALDGA DYTGTYGVTT SGNSLTLQFV |
| | TQSNVGARLG YLMADDTTYQ MFNLLNQEFW FDVDMSNLPC GLNGALYFSA MARTAAWMPM VVCASTPLIS TRRSTARLLR LPVPPRSRYG RGICDSQCPR DIKFINGEAN |
| | VQGWQPSPND TNAGTGNYGA CCNKMDVWEA NSISTAYTPH PCTQRGLVRC SGTACGGGSN RYGSICDHDG LGFQNLFGMG RTRVRARVGR VKQFNRSSRV VEPISWTKQT |
| | TLHLGNLPWK SADCNVQNGR VIQNSKVNIP GMPSTMDSVT TEFCNAQKTA FNDTFSFQQK GGMANMSEAL RRGMVLVLSI WDDHAANMLW LDSITSAAAC RSTPSEVHAT |
| | PLRESQIRSS HSRQTRYVTF TNIKFGPFNS TGTTYTTGSV PTTSTSTGTT GSSTPPQPTG VTVPQGQCGG IGYTGPTTCA SPTTCHVLNP YYSQCY |
| SEQ ID NO: 97 | MKQYLQYLAA ALPLMSLVSA QGVGTSTSET HPKITWKKCS SGGSCSTVNA EVVIDANWRW LHNADSKNCY DGNEWTDACT SSDDCTSKCV LEGAEYGKTY GASTSGDSLS |
| | LKFLTKHEYG TNIGSRFYLM NGASKYQMFT LMNNEFAFDV DLSTVECGLN SALYFVAMEE DGGMASYSTN KAGAKYGTGY CDAQCARDLK FVGGKANYDG WTPSSNDANA |
| | GVGALGGCCA EIDVWESNAH AFAFTPHACE NNNYHVCEDT TCGGTYSEDR FAGDCDANGC DYNPYRVGNT DFYGKGMTVD TSKKFTVVSQ FQENKLTQFF VQNGKKIEIP |
| | GPKHEGLPTE SSDITPELCS AMPEVFGDRD RFAEVGGFDA LNKALAVPMV LVMSIWDDHY ANMLWLDSSY PPEKAGTPGG DRGPCAQDSG VPSEVESQYP DATVVWSNIR |
| | FGPIGSTVQV |
| SEQ ID NO: 98 | MFPKASLIAL SFIAAVYGQQ VGTQMAEVHP KLPSQLCTKS GCTNQNTAVV LDANWRWLHT TSGYTNCYTG NSWDATLCPD ATTCAQNCAV DGADYSGTYG ITTSGNALTL |
| | KFKTGTNVGS RVYLMQTDTA YQMFQLLNQE FTFDVDMSNL PCGLNGALYL SQMDQDGGLS KFPTNKAGAK YGTGYCDSQC PHDIKFINGM ANVAGWAGSA SDPNAGSGTL |
| | GTCCSEMDIW EANNDAAAFT PHPCSVDGQT QCSGTQCGDD DERYSGLCDK DGCDFNSFRM GDKSFLGKGM TVDTSRKFTV VTQFVTTDGT TNGDLHEIRR LYVQDGKVIQ |
| | NSVVSIPGID AVDSITDNFC AQQKSVFGDT NYFATLGGLK KMGAALKSGM VLAMSVWDDH AASMQWLDSN YPADGDATKP GVARGTCSAD SGLPTNVESQ SASASVTFSN |
| | IKWGDINTTF TGTGSTSPSS PAGPVSSSTS VASQPTQPAQ GTVAQWGQCG GTGFTGPTVC ASPFTCHVVN PYYSQCY |
| SEQ ID NO: 99 | MFRTAALLSF AYLAVVYGQQ AGTSTAETHP PLTWEQCTSG GSCTTQSSSV VLDSNWRWTH VVGGYTNCYT GNEWNTTVCP DGTTCAANCA LDGADYEGTY GISTSGNALT |
| | LKFVTASAQT NVGSRVYLMA PGSETEYQMF NPLNQEFTFD VDVSALPCGL NGALYFSEMD ADGGLSEYPT NKAGAKYGTG YCDSQCPRDI KFIEGKANVE GWTPSSTSPN |
| | AGTGGTGICC NEMDIWEANS ISEALTPHPC TAQGGTACTG DSCSSPNSTA GICDQAGCDF NSFRMGDTSF YGPGLTVDTT SKITVVTQFI TSDNTTTGDL TAIRRIYVQN |
| | GQVIQNSMSN IAGVTPTNEI TTDFCDQQKT AFGDTNTFSE KGGLTGMGAA FSRGMVLVLS IWDDDAAEML WLDSTYPVGK TGPGAARGTC ATTSGQPDQV ETQSPNAQVV |
| | FSNIKFGAIG STFSSTGTGT GTGTGTGTGT GTTTSSAPAA TQTKYGQCGG QGWTGATVCA SGSTCTSSGP YYSQCL |
| SEQ ID NO: 100 | MFRTAALTAF TFAAVVLGQQ VGTLTTENHP ALSIQQCTAT GCTTQQKSVV LDSNWRWTHS TAGATNCYTG NAWDPALCPD PATCATNCAI DGADYSGTYG ITTSGNALTL |
| | RFVTNGQYSQ NIGSRVYLLD DADHYKLFDL KNQEFTFDVD MSGLPCGLNG ALYFSEMAAD GGKAAHAGNN AGAKYGTGYC DAQCPHDIKW INGEANVLDW SASATDDNAG |
| | NGRYGACCAE MDIWEANSEA TAYTPHVCRD EGLYRCSGTE CGDGNNRYGG VCDKDGCDFN SYRMGDKNFL GRGKTIDTTK KVTVVTQFIT DNNTPTGNLV EIRRVYVQNG |
| | VVYQNSFSTF PSLSQYNSIS DEFCVAQKTL FGDNQYYNTH GGTTKMGDAF DNGMVLIMSL WSDHAAHMLW LDSDYPLDKS PSEPGVSRGA CPTSSGDPDD VVANHPNASV |
| | TFSNIKYGPI GSTFGGSTPP VSSGGSSVPP VTSTTSSGTT TPTGPTGTVP KWGQCGGIGY SGPTACVAGS TCTYSNDWYS QCL |
| SEQ ID NO: 101 | MYRAIATASA LIAAVRAQQV CSLTPETKPA LSWSKCTSSG CSNVQGSVTI DANWRWTHQL SGSTNCYTGN KWDTSICTSG KVCAEKCCID GAEYASTYGI TSSGNQLSLS |
| | FVTKGTYGTN IGSRTYLMED ENTYQMFQLL GNEFTFDVDV SNIGCGLNGA LYFVSMDADG GKAKYPGNKA GAKYGTGYCD AQCPRDVKFI NGQANSDGWQ PSKSDVNGGI |
| | GNLGTCCPEM DIWEANSIST AHTPHPCTKL TQHSCTGDSC GGTYSEDRYG GTCDADGCDF NAYRQGNKTF YGPGSGFNVD TTKKVTVVTQ FHKGSNGRLS EITRLYVQNG |
| | KVIANSESKI AGVPGSSLTP EFCTAQKKVF GDIDDFEKKG AWGGMSDALE APMVLVMSLW HDHHSNMLWL DSTYPTDSTK LGAQRGSCST SSGVPADLEK NVPNSKVAFS |
| | NIKFGPIGST YKEGQPEPTN PTNPNPTTPG GTVDQWGQCG GTNYSGPTAC KSPFTCKKIN DFYSQCQ |
| SEQ ID NO: 102 | MFRTATLLAF TMAAMVFGQQ VGTNTAENHR TLTSQKCTKS GGCSNLNTKI VLDANWRWLH STSGYTNCYT GNQWDATLCP DGKTCAANCA LDGADYTGTY GITASGSSLK |
| | LQFVTGSNVG SRVYLMADDT HYQMFQLLNQ EFTFDVDMSN LPCGLNGALY LSAMDADGGM AKYPTNKAGA KYGTGYCDSQ CPRDIKFING EANVEGWNAT SANAGTGNYG |
| | TCCTEMDIWE ANNDAAAYTP HPCTTNAQTR CSGSDCTRDT GLCDADGCDF NSFRMGDQTF LGKGLTVDTS KPFTVVTQFI TNDGTSAGTL TEIRRLYVQN GKVIQNSSVK |
| | IPGIDLVNSI TDNFCSQQKT AFGDTNYFAQ HGGLKQVGEA LRTGMVLALS IWDDYAANML WLDSNYPTNK DPSTPGVARG TCATTSGVPA QIEAQSPNAY VVFSNIKFGD |
| | LNTTYTGTVS SSSVSSSHSS TSTSSSHSSS STPPTQPTGV TVPQWGQCGG IGYTGSTTCA SPYTCHVLNP YYSQCY |
| SEQ ID NO: 103 | MYQTSLLASL SFLLATSQAQ QVGTQTAETH PKLTTQKCTT AGGCTDQSTS IVLDANWRWL HTVDGYTNCY TGQEWDTSIC TDGKTCAEKC ALDGADYEST YGISTSGNAL |
| | TMNFVTKSSQ TNIGGRVYLL AADSDDTYEL FKLKNQEFTF DVDVSNLPCG LNGALYFSEM DSDGGLSKYT TNKAGAKYGT GYCDTQCPHD IKFINGEANV QNWTASSTDK |
| | NAGTGHYGSC CNEMDIWEAN SQATAFTPHV CEAKVEGQYR CEGTECGDGD NRYGGVCDKD GCDFNSYRMG NETFYGSNGS TIDTTKKFTV VTQFITADNT ATGALTEIRR |
| | KYVQNDVVIE NSYADYETLS KFNSITDDFC AAQKTLSGDT NDFKTKGGIA RMGESFERGM VLVMSVWDDH AANALWLDSS YPTDADASKP GVKRGPCSTS SGVPSDVEAN |
| | DADSSVIYSN IRYGDIGSTF NKTA |
| SEQ ID NO: 104 | MFSKVALTAL CFLAVAQAQQ VGREVAENHP RLPWQRCTRN GGCQTVSNGQ VVLDANWRWL HVTDGYTNCY TGNAWNSSVC SDGATCAQRC ALEGANYQQT YGITTSGDAL |
| | TIKFLTRSEQ TNIGARVYLM ENEDRYQMFN LLNKEFTFDV DVSKVPCGIN GALYFIQMDA DGGLSSQPNN RAGAKYGTGY CDSQCPRDIK FINGEANSVG WEPSETDPNA |
| | GKGQYGICCA EMDIWEANSI SNAYTPHPCQ TVNDGGYQRC QGRDCNQPRY EGLCDPDGCD YNPFRMGNKD FYGPGKTVDT NRKMTVVTQF ITHDNTDTGT LVDIRRLYVQ |
| | DGRVIANPPT NFPGLMPAHD SITQEFCDDA KRAFEDNDSF GRNGGLAHMG RSLAKGHVLA LSIWNDHTAH MLWLDSNYPT DADPNKPGIA RGTCPTTGGS PRDTEQNHPD |
| | AQVIFSNIKF GDIGSTFSGN |
| SEQ ID NO: 105 | MYRKLAVISA FLAAARAQQV CTQQAETHPP LTWQKCTASG CTPQQGSVVL DANWRWTHDT KSTTNCYDGN TWSSTLCPDD ATCAKNCCLD GANYSGTYGV TTSGDALTLQ |
| | FVTASNVGSR LYLMANDSTY QEFTLSGNEF SFDVDVSQLP CGLNGALYFV SMDADGGQSK YPGNAAGAKY GTGYCDSQCP RDLKFINGQA NVEGWEPSSN NANTGVGGHG |
| | SCCSEMDIWE ANSISEALTP HPCETVGQTM CSGDSCGGTY SNDRYGGTCD PDGCDWNPYR LGNTSFYGPG SSFALDTTKK LTVVTQFATD GSISRYYVQN GVKFQQPNAQ |
| | VGSYSGNTIN TDYCAAEQTA FGGTSFTDKG GLAQINKAFQ GGMVLVMSLW DDYAVNMLWL DSTYPTNATA STPGAKRGSC STSSGVPAQV EAQSPNSKVI YSNIRFGPIG |
| | STGGNTGSNP PGTSTTRAPP SSTGSSPTAT QTHYGQCGGT GWTGPTRCAS GYTCQVLNPF YSQCL |
| SEQ ID NO: 106 | MRASLLAFSL AAAVAGGQQA GTLTAKRHPS LTWQKCTRGG CPTLNTTMVL DANWRWTHAT SGSTKCYTGN KWQATLCPDG KSCAANCALD GADYTGTYGI TGSGWSLTLQ |
| | FVTDNVGARA YLMADDTQYQ MLELLNQELW FDVDMSNIPC GLNGALYLSA MDADGGMRKY PTNKAGAKYA TGYCDAQCPR DLKYINGIAN VEGWTPSTND ANGIGDHGSC |
| | CSEMDIWEAN KVSTAFTPHP CTTIEQHMCE GDSCGGTYSD DRYGVLCDAD GCDFNSYRMG NTTFYGEGKT VDTSSKFTVV TQFIKDSAGD LAEIKAFYVQ NGKVIENSQS |
| | NVDGVSGNSI TQSFCKSQKT AFGDIDDFNK KGGLKQMGKA LAQAMVLVMS IWDDHAANML WLDSTYPVPK VPGAYRGSGP TTSGVPAEVD ANAPNSKVAF SNIKFGHLGI |
| | SPFSGGSSGT PPSNPSSSAS PTSSTAKPSS TSTASNPSGT GAAHWAQCGG IGFSGPTTCP EPYTCAKDHD IYSQCV |
| SEQ ID NO: 107 | MLASTFSYRM YKTALILAAL LGSGQAQQVG TSQAEVHPSM TWQSCTAGGS CTTNNGKVVI DANWRWVHKV GDYTNCYTGN TWDKTLCPDD ATCASNCALE GANYQSTYGA |
| | TTSGDSLRLN FVTTSQQKNI GSRLYMMKDD TTYEMFKLLN QEFTFDVDVS NLPCGLNGAL YFVAMDADGG MSKYPTNKAG AKYGTGYCDS QCPRDLKFIN GQANVEGWQP |
| | SSNDANAGTG NHGSCCAEMD IWEANSISTA FTPHPCDTPG QVMCTGDACG GTYSSDRYGG TCDPDGCDFN SFRQGNKTFY GPGMTVDTKS KFTVVTQFIT DDGTASGTLK |
| | EIKRFYVQNG KVIPNSESTW SGVGGNSITN DYCTAQKSLF KDQNVFAKHG GMEGMGAALA QGMVLVMSLW DDHAANMLWL DSNYPTTASS STPGVARGTC DISSGVPADV |
| | EANHPDASVV YSNIKVGPIG STFNSGGSNP GGGTTTTAKP TTTTTTAGSP GGTGVAQHYG QCGGNGWQGP TTCASPYTCQ KLNDFYSQCL |
| SEQ ID NO: 108 | MQIKQYLQYL AAALPLVNMA AAQRAGTQQT ETHPRLSWKR CSSGGNCQTV NAEIVIDANW RWLHDSNYQN CYDGNRWTSA CSSATDCAQK CYLEGANYGS TYGVSTSGDA |
| | LTLKFVTKHE YGTNIGSRVY LMNGSDKYQM FTLMNNEFAF DVDLSKVECG LNSALYFVAM EEDGGMRSYS SNKAGAKYGT GYCDAQCARD LKFVGGKANI EGWRPSTNDA |
| | NAGVGPYGAC CAEIDVWESN AYAFAFTPHG CLNNNYHVCE TSNCGGTYSE DRFGGLCDAN GCDYNPYRMG NKDFYGKGKT VDTSRKFTVV TRFEENKLTQ FFIQDGRKID |
| | IPPPTWPGLP NSSAITPELC TNLSKVFDDR DRYEETGGFR TINEALRIPM VLVMSIWDGH YASMLWLDSV YPPEKAGQPG AERGPCAPTS GVPAEVEAQF PNAQVIWSNI |
| | RFGPIGSTYQ V |
| SEQ ID NO: 109 | MTSRIALVSL FAAVYGQQVG TYQTETHPSL TWQSCTAKGS CTTNTGSIVL DGNWRWTHGV GTSTNCYTGN TWDATLCPDD ATCAQNCALE GADYSGTYGI TTSGNSLRLN |
| | FVTQSANKNI GSRVYLMADT THYKTFNLLN QEFTFDVDVS NLPCGLNGAV YFANLPADGG ISSTNTAGAE YGTGYCDSQC PRDMKFIKGQ ANVDGWVPSS NNANTGVGNH |
| | GSCCAEMDIW EANSISTAVT PHSCDTVTQT VCTGDDCGGT YSSSRYAGTC DPDGCDFNSY RMGDETFYGP GKTVDTNSVF TVVTQFLTTD GTASGTLNEI KRFYVQDGKV |
| | IPNSYSTISG VSGNSITTPF CDAQKTAFGD PTSFSDHGGL ASMSAAFEAG MVLVLSLWDD YYANMLWLDS TYPVGKTSAG GPRGTCDTSS GVPASVEASS PNAYVVYSNI |
| | KVGAINSTYG |
| SEQ ID NO: 110 | MFVFVLLWLT QSLGTGTNQA ENHPSLSWQN CRSGGSCTQT SGSVVLDSNW RWTHDSSLTN CYDGNEWSSS LCPDPKTCSD NCLIDGADYS GTYGITSSGN SLKLVFVTNG |
| | PYSTNIGSRV YLLKDESHYQ IFDLKNKEFT FTVDDSNLDC GLNGALYFVS MDEDGGTSRF SSNKAGAKYG TGYCDAQCPH DIKFINGEAN VENWKPQTND ENAGNGRYGA |
| | CCTEMDIWEA NKYATAYTPH ICTVNGEYRC DGSECGDTDS GNRYGGVCDK DGCDFNSYRM GNTSFWGPGL IIDTGKPVTV VTQFVTKDGT DNGQLSEIRR KYVQGGKVIE |
| | NTVVNIAGMS SGNSITDDFC NEQKSAFGDT NDFEKKGGLS GLGKAFDYGM VLVLSLWDDH QVNMLWLDSI YPTDQPASQP GVKRGPCATS SGAPSDVESQ HPDSSVTFSD |
| | IRFGPIDSTY |
| SEQ ID NO: 111 | MFRKAALLAF SFLAIAHGQQ VGTNQAENHP SLPSQKCTAS GCTTSSTSVV LDANWRWVHT TTGYTNCYTG QTWDASICPD GVTCAKACAL DGADYSGTYG ITTSGNALTL |
| | QFVKGTNVGS RVYLLQDASN YQMFQLINQE FTFDVDMSNL PCGLNGAVYL SQMDQDGGVS RFPTNTAGAK YGTGYCDSQC PRDIKFINGE ANVEGWTGSS TDSNSGTGNY |
| | GTCCSEMDIW EANSVAAAYT PHPCSVNQQT RCTGADCGQG DDRYDGVCDP DGCDFNSFRM GDQTFLGKGL TVDTSRKFTI VTQFISDDGT TSGNLAEIRR FYVQDGNVIP |
| | NSKVSIAGID AVNSITDDFC TQQKTAFGDT NRFAAQGGLK QMGAALKSGM VLALSLWDDH AANMLWLDSD YPTTADASNP GVARGTCPTT SGFPRDVESQ SGSATVTYSN |
| | IKWGDLNSTF TGTLTTPSGS SSPSSPASTS GSSTSASSSA SVPTQSGTVA QWAQCGGIGY SGATTCVSPY TCHVVNAYYS QCY |
| SEQ ID NO: 112 | MYRAIATASA LIAAARAQQV CTLTTETKPA LTWSKCTSSG CTDVKGSVGI DANWRWTHQT SSSTNCYTGN KWDTSVCTSG ETCAQKCCLD GADYAGTYGI TSSGNQLSLG |
| | FVTKGSFSTN IGSRTYLMEN ENTYQMFQLL GNEFTFDVDV SNIGCGLNGA LYFVSMDADG GKARYPANKA GAKYGTGYCD AQCPRDVKFI NGKANSDGWK PSDSDINAGI |
| | GNMGTCCPEM DIWEANSIST AFTPHPCTKL TQHACTGDSC GGTYSNDRYG GTCDADGCDF NSYRQGNKTF YGRGSDFNVD TTKKVTVVTQ FKKGSNGRLS EITRLYVQNG |
| | KVIANSESKI PGNSGSSLTA DFCSKQKSVF GDIDDFSKKG GWSGMSDALE SPPMVLVMSL WHDHHSNMLW LDSTYPTDST KLGAQRGSCA TTSGVPSDLE RDVPNSKVSF |
| | SNIKFGPIGS TYSSGTTNPP PSSTDTSTTP TNPPTGGTVG QYGQCGGQTY TGPKDCKSPY TCKKINDFYS QCQ |
| SEQ ID NO: 113 | MSSFQIYRAA LLLSILATAN AQQVGTYTTE THPSLTWQTC TSDGSCTTND GEVVIDANWR WVHSTSSATN CYTGNEWDTS ICTDDVTCAA NCALDGATYE ATYGVTTSGS |
| | ELRLNFVTQG SSKNIGSRLY LMSDDSNYEL FKLLGQEFTF DVDVSNLPCG LNGALYFVAM DADGGTSEYS GNKAGAKYGT GYCDSQCPRD LKFINGEANC DGWEPSSNNV |
| | NTGVGDHGSC CAEMDVWEAN SISNAFTAHP CDSVSQTMCD GDSCGGTYSA SGDRYSGTCD PDGCDYNPYR LGNTDFYGPG LTVDTNSPFT VVTQFITDDG TSSGTLTEIK |
| | RLYVQNGEVI ANGASTYSSV NGSSITSAFC ESEKTLFGDE NVFDKHGGLE GMGEAMAKGM VLVLSLWDDY AADMLWLDSD YPVNSSASTP GVARGTCSTD SGVPATVEAE |
| | SPNAYVTYSN IKFGPIGSTY SSGSSSGSGS SSSSSSTTTK ATSTTLKTTS TTSSGSSSTS AAQAYGQCGG QGWTGPTTCV SGYTCTYENA YYSQCL |
| SEQ ID NO: 114 | MHQRALLFSA LLTAVRAQQA GTLTEEVHPS LTWQKCTSEG SCTEQSGSVV IDSNWRWTHS VNDSTNCYTG NTWDATLCPD DETCAANCAL DGADYESTYG VTTDGDSLTL |
| | KFVTGSNVGS RLYLMDTSDE GYQTFNLLDA EFTFDVDVSN LPCGLNGALY FTAMDADGGV SKYPANKAGA KYGTGYCDSQ CPRDLKFIDG QANVDGWEPS SNNDNTGIGN |
| | HGSCCPEMDI WEANKISTAL TPHPCDSSEQ TMCEGNDCGG TYSDDRYGGT CDPDGCDFNP YRMGNDSFYG PGKTIDTGSK MTVVTQFITD GSGSLSEIKR YYVQNGNVIA |
| | NADSNISGVT GNSITTDFCT AQKKAFGDED IFAEHNGLAG ISDAMSSMVL ILSLWDDYYA SMEWLDSDYP ENATATDPGV ARGTCDSESG VPATVEGAHP DSSVTFSNIK |
| | FGPINSTFSA SA |
| SEQ ID NO: 115 | MYAKFATLAA LVAGAAAQNA CTLTAENHPS LTWSKCTSGG SCTSVQGSIT IDANWRWTHR TDSATNCYEG NKWDTSYCSD GPSCASKCCI DGADYSSTYG ITTSGNSLNL |
| | KFVTKGQYST NIGSRTYLME SDTKYQMFQL LGNEFTFDVD VSNLGCGLNG ALYFVSMDAD GGMSKYSGNK AGAKYGTGYC DSQCPRDLKF INGEANVENW QSSTNDANAG |
| | TGKYGSCCSE MDVWEANNMA AAFTPHPCXV IGQSRCEGDS CGGTYSTDRY AGICDPDGCD FNSYRQGNKT FYGKGMTVDT TKKITVVTQF LKNSAGELSE IKRFYVQNGK |
| | VIPNSESTIP GVEGNSITQD WCDRQKAAFG DVTDXQDKGG MVQMGKALAG PMVLVMSIWD DHAVNMLWLD STWPIDGAGK PGAERGACPT TSGVPAEVEA EAPNSNVIFS |
| | NIRFGPIGST VSGLPDGGSG NPNPPVSSST PVPSSSTTSS GSSGPTGGTG VAKHYEQCGG IGFTGPTQCE SPYTCTKLND WYSQCL |
| SEQ ID NO: 116 | MYAKFATLAA LVAGASAQAV CSLTAETHPS LTWQKCTAPG SCTNVAGSIT IDANWRWTHQ TSSATNCYSG SKWDSSICTT GTDCASKCCI DGAEYSSTYG ITTSGNALNL |
| | KFVTKGQYST NIGSRTYLME SDTKYQMFKL LGNEFTFDVD VSNLGCGLNG ALYFVSMDAD GGMSKYSGNK AGAKYGTGYC DAQCPRDLKF INGEANVEGW ESSTNDANAG |
| | SGKYGSCCTE MDVWEANNMA TAFTPHPCTT IGQTRCEGDT CGGTYSSDRY AGVCDPDGCD FNSYRQGNKT FYGKGMTVDT TKKITVVTQF LKNSAGELSE IKRFYAQDGK |
| | VIPNSESTIA GIPGNSITKA YCDAQKTVFQ NTDDFTAKGG LVQMGKALAG DMVLVMSVWD DHAVNMLWLD STYPTDQVGV AGAERGACPT TSGVPSDVEA NAPNSNVIFS |
| | NIRFGPIGST VQGLPSSGGT SSSSSAAPQS TSTKASTTTS AVRTTSTATT KTTSSAPAQG TNTAKHWQQC GGNGWTGPTV CESPYKCTKQ NDWYSQCL |
| SEQ ID NO: 117 | MLTLVYFLLS LVVSLEIGTQ QSEDHPKLTW QNGSSSVSGS IVLDSNWRWV HDSGTTNCYD GNLWSKDLCP SSDTCSQKCY IEGADYSGTY GIQSSGSKLT LKFVTKGSYS |
| | TNIGSRVYLL KDENTYESFK LKNKEFTFTV DDSKLNCGLN GALYFVAMDA DGGKAKYSSF KPGAKYGMGY CDAQCPHDMK FISGKANVDD WKPQDNDENS GNGKLGTCCS |
| | EMDIWEGNMK SQAYTVHACT KSGQYECTGQ QCGDTDSGDR FKGTCDKDGC DYASWRWGDQ SFYGEGKTVD TKQPVTVVTQ FIGDPLTEIR RLYVQGGKTI NNSKTSNLAD |
| | TYDSITDKFC DATKEASGDT NDFKAKGAMS GFSTNLNNGQ VLVMSLWDDH TANMLWLDST YPTDSSDSTA QRGPCPTSSG VPKDVESQHG DATVVFSDIK FGAINSTFKY |
| | N |
| SEQ ID NO: 118 | MLAAALFTFA CSVGVGTKTP ENHPKLNWQN CASKGSCSQV SGEVTMDSNW RWTHDGNGKN CYDGNTWISS LCPDDKTCSD KCVLDGAEYQ ATYGIQSNGT ALTLKFVTHG |
| | SYSTNIGSRL YLLKDKSTYY VFKLNNKEFT FSVDVSKLPC GLNGALYFVE MDADGGKAKY AGAKPGAEYG LGYCDAQCPS DLKFINGEAN SEGWKPQSGD KNAGNGKYGS |
| | CCSEMDVWES NSQATALTPH VCKTTGQQRC SGKSECGGQD GQDRFAGLCD EDGCDFNNWR MGDKTFFGPG LIVDTKSPFV VVTQFYGSPV TEIRRKYVQN GKVIENSKSN |
| | IPGIDATAAI SDHFCEQQKK AFGDTNDFKN KGGFAKLGQV FDRGMVLVLS LWDDHQVAML WLDSTYPTNK DKSQPGVDRG PCPTSSGKPD DVESASADAT VVYGNIKFGA |
| | LDSTY |
| SEQ ID NO: 119 | MLTLVYFLLS LVVSLEIGTQ QSEDHPKLTW QNGSSSVSGS IVLDSNWRWV HDSGTTNCYD GNLWSKDLCP SSNTCSQKCY IEGADYSGTY GIQSSGSKLT LKFVTKGSYS |
| | TNIGSRVYLL KDENTYESFK LKNKEFTFTV DDSKLNCGLN GALYFVAMDA DGGKAKYSSF KPGAKYGMGY CDAQCPHDMK FISGKANVDD WKPQDNDENS GNGKLGTCCS |
| | EMDIWEGNMK SQAYTVHACT KSGQYECTGQ QCGDTDSGDR FKGTCDKDGC DYASWRWGDQ SFYGEGKTVD TKQPVTVVTQ FIGDPLTEIR RLYVQGGKTI NNSKTSNLAD |
| | TYDSITDKFC DATKEASGDT NDFKAKGAMS GFSTNLNNGQ VLVMSLWDDH TANMLWLDST YPTDSTKTGA SRGPCAVSSG VPKDVESQYG DATVIYSDIK FGAINSTFKW |
| | N |
| SEQ ID NO: 120 | MILALLSLAK SLGIATNQAE THPKLTWTRY QSKGSGQTVN GEIVLDSNWR WTHHSGTNCY DGNTWSTSLC PDPTTCSNNC DLDGADYPGT YGISTSGNSL KLGFVTHGSY |
| | STNIGSRVYL LRDSKNYEMF KLKNKEFTFT VDDSKLPCGL NGALYFVAMD EDGGVSKNSI NKAGAQYGTG YCDAQCPHDM KFINGEANVL DWKPQSNDEN SGNGRYGACC |
| | TEMDIWEANS MATAYTPHVC TVTGLRRCEG TECGDTDANQ RYNGICDKDG CDFNSYRLGD KTFFGVGKTV DSSKPVTVVT QFVTSNGQDS GTLSEIRRKY VQGGKVIENS |
| | KVNIAGITAG NSVTDTFCNE QKKAFGDNND FEKKGGLGAL SKQLDAGMVL VLSLWDDHSV NMLWLDSTYP TNAAAGALGT ERGACATSSG APSDVESQSP DATVTFSDIK |
| | FGPIDSTY |
| SEQ ID NO: 121 | MLVIALILRG LSVGTGTQQS ETHPSLSWQQ TSKGGSGQSV SGSVVLDSNW RWTHTTDGTT NCYDGNEWSS DLCPDASTCS SNCVLEGADY SGTYGITGSG SSLKLGFVTK |
| | GSYSTNIGSR VYLLGDESHY KLFKLENNEF TFTVDDSNLE CGLNGALYFV AMDEDGGASK YSGAKPGAKY GMGYCDAQCP HDMKFINGDA NVEGWKPSDN DENAGTGKWG |
| | ACCTEMDIWE ANKYATAYTP HICTKNGEYR CEGTDCGDTK DNNRYGGVCD KDGCDFNSWR MGNQSFWGPG LIIDTGKPVT VVTQFLADGG SLSEIRRKYV QGGKVIENTV |
| | TKISGMDEFD SITDEFCNQQ KKAFRDTNDF EKKGGLKGLG TAVDAGVVLV LSLWDDHDVN MLWLDSIYPT DSGSKAGADR GPCATSSGVP KDVESNYASA SVTFSDIKFG |
| | PIDSTY |
| SEQ ID NO: 122 | MLLALFAFGK SLGIATNQAE NHPKLTWTRY QSKGSGQTVN GEIVLDSNWR WTHHSGTNCY DGNTWSTSLC PDPTTCSNNC DLDGADYPGT YGISSSGNSL KLGFVTHGSY |
| | STNIGSRVYL LRDSKNYEMF KLKNKEFTFT VDDSKLPCGL NGALYFVAMD EDGGVSKNSI NKAGAQYGTG YCDAQCPHDM KFINGEANVL DWKPQSNDEN SGNGRYGACC |
| | TEMDIWEANS MATAYTPHVC TVTGIRRCEG TECGDTDANQ RYNGICDKDG CDFNSYRLGD KSFFGVGKTV DSSKPVTVVT QFVTSNGQDS GTLSEIRRKY VQGGKVIENS |
| | KVNIAGMAAG NSITDTFCNE QKKAFGDNND FEKKGGLGAL SKQLDSGMVL VLSLWDDHSV NMLWLDSTYP TNAAAGALGT ERGACATSSG APSDVESQSP DATVTFSDIK |
| | FGPIDSTY |
| SEQ ID NO: 123 | MLASVVYLVS LVVSLEIGTQ QSEEHPKLTW QNGSSSVSGS IVLDSNWRWL HDSGTTNCYD GNLWSDDLCP NADTCSSKCY IEGADYSGTY GITSSGSKVT LKFVTKGSYS |
| | TNIGSRIYLL KDENTYETFK LKNKEFTFTV DDSKLDCGLN GALYFVAMDA DGGKAKYSSF KPGAKYGMGY CDAQCPHDMK FISGKANVDD WKPQDNDENS GDGKLGTCCS |
| | EMDIWEGNAK SQAYTVHACS KSGQYECTGQ QCGDTDSGDR FKGTCDKDGC DYASWRWGDQ SFYGEGKTVD TKSPVTVVTQ FIGDPLTEIR RVYVQGGKTI NNSKTSNLAD |
| | TYDSITDKFC DATKDATGDT NDFKAKGAMA GFSTNLNTAQ VLVSVHCGMI IQPICCGLIR RIQRIQQKQV QAVDRVLCRR VFQRMLKASM VMLQSRTRTL SLELSTRPLV |
| | GISPAGRLFF F |
| SEQ ID NO: 124 | MILALLVLGK SLGIATNQAE THPKLTWTRY QSKGSGSTVN GEIVLDSNWR WTHHSGTNCY DGNTWSTSLC PDPTTCSNNC DLDGADYPGT YGISTSGNSL KLGFVTHGSY |
| | STNIGSRVYL LKDTKSYEMF KLKNKEFTFT VDDSKLPCGL NGALYFVAMD EDGGVSKNSI NKAGAQYGTG YCDAQCPHDM KFINGEANVL DWKPQSNDEN SGNGRYGACC |
| | TEMDIWEANS MATAYTPHVC TVTGLRRCEG TECGDTDNDQ RYNGICDKDG CDFNSYRLGD KSFFGVGKTV DSSKPVTVVT QFVTSNGQDS GTLSEIRRKY VQGGKVIENS |
| | KVNVAGITAG NSVTDTFCNE QKKAFGDNND FEKKGGLGAL SKQLDAGMVL VLSLWDDHSV NMLWLDSTYP TNAAAGALGT ERGACATSSG KPSDVESQSP DATVTFSDIK |
| | FGPIDSTY |
| SEQ ID NO: 125 | MLCIGLISFV YSLGVGTNTA ETHPKLTWKN GGQTVNGEVT VDSNWRWTHT KGSTKNCYDG NLWSKDLCPD AATCGKNCVL EGADYSGTYG VTSSGNALTL KFVTHGSYST |
| | NVGSRLYLLK DEKTYQMFNL NGKEFTFTVD VSNLPCGLNG ALYHVNMDED GGTKRYPDNE AGAKYGTGYC DAQCPTDLKF INGIPNSDGW KPQSNDKNSG NGKYGSCCSE |
| | MDIWEANSIC SAVTPHVCDN LQQTRCQGTA CGENGGGSRF GSSCDPDGCD FNSWRMGNKT FYGPGLIVDT KSKFTVVTQF VGNPVTEIKR KYVQNGKVIE NSYSNIEGMD |
| | KFNSVSDKFC TAQKKAFGDT DSFTKHGGFK QLGSALAKGM VLVLSLWDDH TVNMLWLDSV YPTNSKKAGS DRGPCPTTSG VPADVESKSA DANVIYSDIR FGAIDSTYK |
| SEQ ID NO: 126 | MLGALVALAS CIGVGTNTPE KHPDLKWTNG GSSVSGSIVV DSNWRWTHIK GETKNCYDGN LWSDKYCPDA ATCGKNCVLE GADYSGTYGV TTSGDAATLK FVTHGQYSTN |
| | VGSRLYLLKD EKTYQMFNLV GKEFTFTVDV SNLPCGLNGA LYFVQMDSDG GMAKYPDNQA GAKYGTGYCD AQCPTDLKFI NGIPNSDGWK PQKNDKNSGN GKYGSCCSEM |
| | DIWEANSMAT AYTPHVCDKL EQTRCSGSAC GQNGGGDRFS SSCDPDGCDF NSWRMGNKTF WGPGLIVDTK KPVQVVTQFV GSGGSVTEIK RKYVQGGKVI DNSMTNIAAM |
| | SKQYNSVSDE FCQAQKKAFG DNDSFTKHGG FRQLGATLSK GHVLVLSLWD DHDVNMLWLD SVYPTNSNKP GADRGPCKTS SGVPSDVESQ NADSTVKYSD IRFGAIDSTY |
| | SK |
| SEQ ID NO: 127 | MLAAALFTFA CSVGVGTKTT ETHPKLNWQQ CACKGSCSQV SGEVTMDSNW RWTHDGNGKN CYDGNTWISS LCPDDKTCSD KCVLDGAEYQ ATYGIQSNGT ALTPKFVTHG |
| | SYSTNIGSRL YLLKDKSTYY VFQLNNKEFT FSVDVSKLPC GLNGALYFVE MDADGGKSKY AGAKPGAEYG LGYCDAQCPS DLKFINGEAN SEGWKPQSGD KNAGNGKYGS |
| | CCSEMDVWES NSMATALTPH VCKTTGQTRC SGKSECGGQD GQDRFAGNCD EDGCDFNNWR MGDKTFFGPG LTVDTKSPFV VVTQFYGSPV TEIRRKYVQN GKVIENAKSN |
| | IPGIDATNAI SDTFCEQQKK AFGDTNDFKN KGGFTKLGSV FSRGMVLVLS LWDDHQVAML WLDSTYPTNK DKSVPGVDRG PCPTSSGKPD DVESASGDAT VVYGNIKFGA |
| | LDSTY |
| SEQ ID NO: 128 | MFGFLLSLFA LQFALEIGTQ TSESHPSITW ELNGARQSGQ IVIDSNWRWL HDSGTTNCYD GNTWSSDLCP DPEKCSQNCY LEGADYSGTY GISASGSQLT LGFVTKGSYS |
| | TNIGSRVYLL KDENTYPMFK LKNKEFTFTV DVSNLPCGLN GALYFVAMPS DGGKAKYPLA KPGAKYGMGY CDAQCPHDMK FINGEANVLD WKPQSNDENA GTGRYGTCCT |
| | EMDIWEANSQ ATAYTVHACS KNARCEGTEC GDDSASQRYN GICDKDGCDF NSWRWGNKTF FGPGLTVDSS KPVTVVTQFI GDPLTEIRRI WVQGGKVIQN SFTNVSGITS |
| | VDSITNTFCD ESKVATGDTN DFKAKGGMSG FSKALDTEVV LVLSLWDDHT ANMLWLDSTY PTDSTAIGAS RGPCATSSGD PKDVESASAN ASVKFSDIKF GALDSTY |
| SEQ ID NO: 129 | MLASLLPLSN SLGTASNQAE THPKLTWTQY TGKGAGQTVN GEIVLDSNWR WTHKDGTNCY DGNTWSSSLC PDPTTCSNNC NLDGADYPGT YGITTSGNQL KLGFVTHGSY |
| | STNIGSRVYL LRDSKNYQMF KLKNKEFTFT VDDSKLPCGL NGAVYFVAMD EDGGTAKHSI NKAGAQYGTG YCDAQCPHDM KFINGEANVL DWKPQSNDEN SGNGRWGARC |
| | TEMDIWEANS RATAYTPHIC TKTGLYRCEG TECGDSDTNR YGGVCDKDGC DFNSYRMGDK SFFGQGKTVD SSKPVTVVTQ FITDNNQDSG KLTEIRRKYV QGGKVIDNSK |
| | VNIAGITAGN PITDTFCDEA KKAFGDNNDF EKKGGLSALG TQLEAGFVLV LSLWDDHSVN MLWLDSTYPT NASPGALGVE RGDCAITSGV PADVESQSAD ASVTFSDIKF |
| | GPIDSTY |
| SEQ ID NO: 130 | MLCIGLISFV YSLGVGTNTA ETHPKLTWKN GGQTVNGEVT VDSNWRWTHT KGSTKNCYDG NLWSKDLCPD AATCGKNCVL EGADYSGTYG VTSSGNALTL KFVTHGSYST |
| | NVGSRLYLLK DEKTYQMFNL NGKEFTFTVD VSNLPCGLSG ALYHVNMDED GGTKRYPDNE AGAKYGTGYC DAQCPTDLKF INGIPNSDGW KPQSNDKNSG NGKYGSCCSE |
| | MDIWEANSIC SAVTPHVCDN LQQTRCQGAA CGENGGGSRF GSSCDPDGCD FNSWGMGNKT FYGPGLIVDT KSKFTVVTQF VGNPVTEIKR KYVQNGKVIE NSYSNIEGMD |
| | KFNSVSDKFC TAQKKAFGDT DSFTKHGGFK QLGSALAKGM VLVLSLWDDH TVNMLWLDSV YPTNSKKAGS DRGPCPTTSG VPADVESKSA DANVIYSDIR FGAIDSTYK |
| SEQ ID NO: 131 | MILALLVLGK SLGIATNQAE THPKLTWTRY QSKGSGSTVN GEIVLDSNWR WTHHSGTNCY DGNTWSTSLC PDPTTCSNNC DLDGADYPGT YGISTSGNSL KLGFVTHGSY |
| | STNIGSRVYL LRDSKNYEMF KLKNKEFTFT VDDSKLPCGL NGALYFVAMD EDGGVSKNSI NKAGAQYGTG YCDAQCPHDM KFINGEANVL DWKPQSNDEN SGNGRYGACC |
| | TEMDIWEANS MATAYTPHVC TVTGLRRCEG TECGDTDNDQ RYNGICDKDG CDFNSYRLGD KSFFGVGKTV DSSKPVTVVT QFVTSNGQDS GILSETRRKY VQGGKVIENS |
| | KVNVAGITAG NSVTDTFCNE QKKAFGDNND FEKKGGLGAL SKQLDAGMVL VLSLWDDHSV NMLWLDSTYP TNAAAGALGT ERGACATSSG KPSDVESQSP DATVTFSDIK |
| | FGPIDSTY |
| SEQ ID NO: 132 | MIGIVLIQTV FGIGVGTQQS ESHPSLSWQQ CSKGGSCTSV SGSIVLDSNW RWTHIPDGTT NCYDGNEWSS DLCPDPTTCS NNCVLEGADY SGTYGISTSG SSAKLGFVTK |
| | GSYSTNIGSR VYLLGDESHY KIFDLKNKEF TFTVDDSNLE CGLNGALYFV AMDEDGGASR FTLAKPGAKY GTGYCDAQCP HDIKFINGEA NVQDWKPSDN DDNAGTGHYG |
| | ACCTEMDIWE ANKYATAYTP HICTENGEYR CEGKSCGDSS DDRYGGVCDK DGCDFNSWRL GNQSFWGPGL IIDTGKPVTV VTQFVTKDGT DSGALSEIRR KYVQGGKTIE |
| | NTVVKISGID EVDSITDEFC NQQKQAFGDT NDFEKKGGLS GLGKAFDYGV VLVLSLWDDH DVNMLWLDSV YPTNPAGKAG ADRGPCATSS GDPKEVEDKY ASASVTFSDI |
| | KFGPIDSTY |
| SEQ ID NO: 133 | MLVFGIVSFV YSIGVGTNTA ETHPKLTWKN GGSTTNGEVT VDSNWRWTHT KGSTKNCYDG NLWSKDLCPD AATCGKNCVL EGADYSGTYG VTSSGDALTL KFVTHGSYST |
| | NVGSRLYLLK DEKTYQMFNL NGKEFTFTVD VSQLPCGLNG ALYFVCMDQD GGMSRYPDNQ AGAKYGTGYC DAQCPTDLKF INGLPNSDGW KPQSNDKNSG NGKYGSCCSE |
| | MDIWEANSLA TAVTPHVCDQ VGQTRCEGRA CGENGGGDRF GSICDPDGCD FNSWRMGNKT FWGPGLIIDT KKPVTVVTQF IGSPVTEIKR EYVQGGKVIE NSYTNIEGMD |
| | KFNSISDKFC TAQKKAFGDN DSFTKHGGFS KLGQSFTKGQ VLVLSLWDDH TVNMLWLDSV YPTNSKKLGS DRGPCPTSSG VPADVESKNA DSSVKYSDIR FGSIDSTYK |
| SEQ ID NO: 134 | MLSFVFLLGF GVSLEIGTQQ SENHPTLSWQ QCTSSGSCTS QSGSIVLDSN WRWVHDSGTT NCYDGNEWSS DLCPDPETCS KNCYLDGADY SGTYGITSNG SSLKLGFVTE |
| | GSYSTNIGSR VYLKKDTNTY QIFKLKNHEF TFTVDVSNLP CGLNGALYFV EMEADGGKGK YPLAKPGAQY GMGYCDAQCP HDMKFINGNA NVLDWKPQET DENSGNGRYG |
| | TCCTEMDIWE ANSQATAYTP HICTKDGQYQ CEGTECGDSD ANQRYNGVCD KDGCDFNSYR LGNKTFFGPG LIVDSKKPVT VVTQFITSNG QDSGDLTEIR RIYVQGGKTI |
| | QNSFTNIAGL TSVDSITEAF CDESKDLFGD TNDFKAKGGF TAMGKSLDTG VVLVLSLWDD HSVNMLWLDS TYPTDAAAGA LGTQRGPCAT SSGAPSDVES QSPDASVTFS |
| | DIKFGPLDST Y |
| SEQ ID NO: 135 | MLTLVVYLLS LVVSLEIGTQ QSESHPALTW QREGSSASGS IVLDSNWRWV HDSGTTNCYD GNEWSTDLCP SSDTCTQKCY IEGADYSGTY GITTSGSKLT LKFVTKGSYS |
| | TNIGSRVYLL KDENTYETFK LKNKEFTFTV DDSKLDCGLN GALYFVAMDA DGGKQKYSSF KPGAKYGMGY CDAQCPHDMK FISGKANVED WKPQDNDENS GNGKLGTCCS |
| | EMDIWEGNAK SQAYTVHACT KSGQYECTGT DCGDSDSRYQ GTCDKDGCDY ASYRWGDHSF YGEGKTVDTK QPITVVTQFI GDPLTEIRRL YIQGGKVINN SKTQNLASVY |
| | DSITDAFCDA TKAASGDTND FKAKGAMAGF SKNLDTPQVL VLSLWDDHTA NMLWLDSTYP TDSRDATAER GPCATSSGVP KDVESNQADA SVVFSDIKFG AINSTYSYN |
| SEQ ID NO: 136 | MFGFLLSLFA LQFALEIGTQ TSESHPSITW ELNGARQSGQ IVIDSNWRWL HDSGTTNCYD GNTWSSDLCP DPEKCSQNCY LEGADYSGTY GISASGSQLT LGFVTKGSYS |
| | TNIGSRVYLL KDENTYQMFK LKNKEFTFTV DVSNLPCGLN GALYFVAMPS DGGKAKYPLA KPGAKYGMGY CDAQCPHDMK FINGEANVLD WKPQSNDENA GTGRYGTCCT |
| | EMDIWEANSQ ATAYTVHACS KNARCEGTEC GDDSASQRYN GICDKDGCDF NSWRWGNKTF FGPGLTVDSS KPVTVVTQFI GDPLTEIRRI WVQGGKVIQN SFTNVSGITS |
| | VDSITNTFCD ESKVATGDTN DFKAKGGMSG FSKALDTEVV LVLSLWDDHT ANMLWLDSTY PSNSTAIGAT RGPCATSSGD PKNVESASAN ASVKFSDIKF GAFDSTY |
| SEQ ID NO: 137 | MLALVYFLLS LVVSLEIGTQ QSEDHPKLTW QNGSSSVSGS IVLDSNWRWV HDSGTTNCYD GNLWSTDLCP SSDTCTSKCY IEGADYSGTY GITSSGSKVT LKFVTKGSYS |
| | TNIGSRIYLL KDENTYETFK LKNKEFTFTV DDSQLNCGLN GALYFVAMDA DGGKAKYSSF KPGAKYGMGY CDAQCPHDMK FISGKANVDD WKPQDNDENS GNGKLGTCCS |
| | EMDIWEGNAK SQAYTVHACT KSGQYECTGQ QCGDTDSGDR FKGTCDKDGC DYASWRWGDQ SFYGEGKTVD TKQPVTVVTQ FIGDPLTEIR RLYVQGGKTI NNSKTSNLAD |
| | TYDSITDKFC DATKEASGDT NDFKAKGAMS GFSTNLNTAQ VLVLSLWDDH TANMLWLDST YPTDSTKTGA SRGPCAVTSG VPKDVESQYG SAQVVYSDIK FGAINSTY |
| SEQ ID NO: 138 | MLALVYFLLS FVVSLEIGTQ QSEDHPKLTW QNGSSSVSGS IVLDSNWRWV HDSGTTNCYD GNLWSTDLCG SSDTCSSKCY IEGADYSGTY GISASGSKLT LKFVTKGSYS |
| | TNIGSRVYLL KDENTYETFK LKGKEFTFTV DDSKLDCGLN GALYFVAMDA DGGKAKYSSF KPGAKYGMGY CDAQCPHDMK FISGKANVDD WKPQDNDENS GNGKLGTCCS |
| | EMDIWEGNAK SQAYTVHACT KSGQYECTGQ QCGDTDSGDR FKGTCDKDGC DYASWRWGDQ SFYGEGKTID TKQPVTVVTQ FIGDPLTEIR RVYVQGGKVI NNSKTSNLAN |
| | VYDSITDKFC DDTKDATGDT NDFKAKGAMS GFSTNLNTAQ VLVMSLWDDH TANMLWLDST YPTDSTKTGA SRGPCAVLSG VPKNVESQHG DATVIYSDIK FGAINSTFSY |
| | N |
| SEQ ID NO: 139 | MFLALFVLGK SLGIATNQAE NHPKLTWTRY QSKGSGQTVN GEVVLDSNWR WTHHSGTNCY DGNTWSTSLC PDPQTCSSNC DLDGADYPGT YGISSSGNSL KLGFVTHGSY |
| | STNIGSRVYL LRDSKNYEMF KLKNKEFTFT VDDSKLPCGL NGALYFVAME EDGGVAKNSI NKAGAQYGTG YCDAQCPHDM KFINGEANVL DWKPQSNDEN SGNGRYGACC |
| | IEMDIWEANS MATAYTPHVC TVTGIHRCEG TECGDTDANQ RYNGICDKDG CDFNSYRMGD KSFFGVGKTV DSSKPVTVVT QFVTSNGQDG GTLSEIKRKY VQGGKVIENS |
| | KVNIAGITAV NSITDTFCNE QKKAFGDNND FEKKGGLGAL SKQLDLGMVL VLSLWDDHSV NMLWLDSTYP TDAAAGALGT ERGACATSSG KPSDVESQSP DASVTFSDIK |
| | FGPIDSTY |
| SEQ ID NO: 140 | MLLCLLSIAN SLGVGTNTAE NHPKLSWKNG GSSVSGSVTV DANWRWTHIK GETKNCYDGN LWSDKYCPDA ATCGKNCVIE GADYQGTYGV SSSGDGLTLT FVTHGQYSTN |
| | VGSRLYLMKD EKTYQMFNLN GKEFTFTVDV SNLPCGLNGA LYFVQMDSDG GMAKYPDNQA GAKYGTGYCD AQCPTDLKFI NGIPNSDGWK PQKNDKNSGN GKYGSCCSEM |
| | DIWEANSQAT AYTPHVCDKL EQTRCSGSSC GHTGGGERFS SSCDPDGCDF NSWRMGNKTF WGPGLIVDTK KPVQVVTQFV GSGNSCTEIK RKYVQGGKVI DNSMSNIAGM |
| | SKQYNSVSDD FCQAQKKAFG DNDSFTKHGG FRQLGATLGK GHVLVLSLWD DHDVNMLWLD SVYPTNSNKP GSDRGPCKTS SGIPADVESQ AASSSVKYSD IRFGAIDSTY |
| | K |
| SEQ ID NO: 141 | MLCIGLISFV YSLGVGTNTA ETHPKLTWKN GGQTVNGEVT VDSNWRWTHT KGSTKNCYDG NLWSKDLCPD AATCGKNCVL EGADYSGTYG VTSSGNALTL KFVTHGSYST |
| | NVGSRLYLMK DEKTYQMFNL NGKEFTFTVD VSNLPCGLNG ALYHVNMDED GGTKRYPDNE AGAKYGTGYC DAQCPTDLKF INGIPNSDGW KPQSNDKNSG NGKYGSCCSE |
| | MDIWEANSIC SAVTPHVCDT LQQTRCQGTA CGENGGGSRF GSSCDPDGCD FNSWRMGNKT FYGPGLIVDT KSKFTVVTQF VGSPVTEIKR KYVQNGKVIE NSFSNIEGMD |
| | KFNSISDKFC TAQKKAFGDT DSFTKHGGFK QLGSALAKGM VLVLSLWDDH TVNMLWLDSV YPTNSKKAGS DRGPCPTTSG VPADVESKSA NANVIYSDIR FGAIDSTYK |
| SEQ ID NO: 142 | MLLCLLGIAS SLDAGTNTAE NHPQLSWKNG GSSVSGSVTV DANWRWTHIK GETKNCYDGN LWSDKYCPDA ATCGQNCVIE GADYQGTYGV SASGNALTLT FVTHGQYSTN |
| | VGSRLYLLKD EKTYQIFNLI GKEFTFTVDV SNLPCGLNGA LYFVQMDADG GTAKYSDNKA GAKYGTGYCD AQCPTDLKFI NGIPNSDGWK PQKNDKNSGN GRYGSCCSEM |
| | DVWEANSLAT AYTPHVCDKL EQVRCDGRAC GQNGGGDRFS SSCDPDGCDF NSWRLGNKTF WGPGLIVDTK QPVQVVTQWV GSGTSVTEIK RKYVQGGKVI DNSFTKLDSL |
| | TKQYNSVSDE FCVAQKKAFG DNDSFTKHGG FRQLGATLAK GHVLVLSLWD DHDVNMLWLD SVYPTNSNKP GADRGPCKTS SGVPADVESQ AASSSVKYSD IRFGAIDSTY |
| | K |
| SEQ ID NO: 143 | MLGIGFVCIV YSLGVGTNTA ENHPKLTWKN SGSTTNGEVT VDSNWRWTHT KGTTKNCYDG NLWSKDLCPD AATCGKNCVL EGADYSGTYG VTSSGDALTL KFVTHGSYST |
| | NVGSRLYLLK DEKTYQIFNL NGKEFTFTVD VSNLPCGLNG ALYFVNMDAD GGTGRYPDNQ AGAKYGTGYC DAQCPTDLKF INGIPNSDGW KPQSNDKNSG NGKYGSCCSE |
| | MDIWEANSLA TAVTPHVCDQ VGQTRCEGRA CGENGGGDRF GSSCDPDGCD FNSWRLGNKT FWGPGLIVDT KKPVTVVTQF VGSPVTEIKR KYVQGGKVIE NSYTNIEGLD |
| | KFNSISDKFC TAQKKAFGDN DSFIKHGGFR QLGQSFTKGQ VLVLSLWDDH TVNMLWLDSV YPTNSKKPGA DRGPCPTSSG VPADVESKNA GSSVKYSDIR FGSIDSTYK |
| SEQ ID NO: 144 | MATLVGILVS LFALEVALEI GTQTSESHPS LSWELNGQRQ TGSIVIDSNW RWLHDSGTTN CYDGNEWSSD LCPDPEKCSQ NCYLEGADYS GTYGISSSGN SLQLGFVTKG |
| | SYSTNIGSRV YLLKDENTYA TFKLKNKEFT FTADVSNLPC GLNGALYFVA MPADGGKSKY PLAKPGAKYG MGYCDAQCPH DMKFINGEAN ILDWKPSSND ENAGAGRYGT |
| | CCTEMDIWEA NSQATAYTVH ACSKNARCEG TECGDDDGRY NGICDKDGCD FNSWRWGNKT FFGPNLIVDS SKPVTVVTQF IGDPLTEIRR IYVQGGKVIQ NSFTNISGVA |
| | SVDSITDAFC NENKVATGDT NDFKAKGGMS GFSKALDTEV VLVLSLWDDH TANMLWLDST YPTDSSALGA SRGPCAITSG EPKDVESASA NASVKFSDIK FGAIDSTY |
| SEQ ID NO: 145 | MLTLVYFLLS LVVSLEIGTQ QSESHPQLSW QNGSSSVSGS IVLDSNWRWV HDSGTTNCYD GNLWSTDLCP SSDTCTSKCY IEGADYSGTY GITSSGSKLT LKFVTKGSYS |
| | TNIGSRVYLL KDENTYETFK LKNKEFTFTV DDSKLDCGLN GALYFVAMDA DGGKAKYSSF KPGAKYGMGY CDAQCPHDMK FISGKANVDD WKPQDNDENS GNGKLGTCCS |
| | EMDIWEGNAK SQAYTVHACT KSGQYECTGQ QCGDTDSGDR FKGTCDKDGC DYASWRWGDQ SFYGEGKTVD TKQPLTVVTQ FVGDPLTEIR RVYVQGGKTI NNSKTSNLAD |
| | TYDSITDKFC DATKEASGDT NDFKAKGAMS GFSTNLNTAQ VLVMSLWDDH TANMLWLDST YPTDSTKTGA SRGPCAVSSG VPKDVESQHG DATVIYSDIK FGAINSTFKW |
| | N |
| SEQ ID NO: 146 | MLSLVSIFLV GLGFSLGVGT QQSESHPSLS WQNCSAKGSC QSVSGSIVLD SNWRWLHDSG TTNCYDGNEW STDLCPDAST CDKNCYIEGA DYSGTYGITS SGAQLKLGFV |
| | TKGSYSTNIG SRVYLLRDES HYQLFKLKNH EFTFTVDDSQ LPCGLNGALY FVEMAEDGGA KPGAQYGMGY CDAQCPHDMK FITGEANVKD WKPQETDENA GNGHYGACCT |
| | EMDIWEANSQ ATAYTPHICS KTGIYRCEGT ECGDNDANQR YNGVCDKDGC DFNSYRLGNK TFWGPGLTVD SNKAMIVVTQ FTTSNNQDSG ELSEIRRIYV QGGKTIQNSD |
| | TNVQGITTTN KITQAFCDET KVTFGDTNDF KAKGGFSGLS KSLESGAVLV LSLWDDHSVN MLWLDSTYPT DSAGKPGADR GPCAITSGDP KDVESQSPNA SVTFSDIKFG |
| | PIDSTY |
| SEQ ID NO: 147 | MILALLVLGK SLGIATNQAE THPKLTWTRY QSKGSGSTVN GEIVLDSNWR WTHHSGTNCY DGNTWSTSLC PDPTTCSNNC DLDGADYPGT YGISTSGNSL KLGFVTHGSY |
| | STNIGSRVYL LKDTKSYEMF KLKNKEFTFT VDDSKLPCGL NGALYFVAMD EDGGVSKNSI NKAGAQYGTG YCDAQCPHDM KFINGEANVL DWKPQSNDEN SGNGRYGACC |
| | TEMDIWEANS MATAYTPHVC TVTGLRRCEG TECGDTDNDQ RYNGICDKDG CDFNSYRLGD KSFFGVGKTV DSSKPVTVVT QFVTSNGQDS GTLSEIRRKY VQGGKVIENS |
| | KVNVAGITAG NSVTDTFCNE QKKAFGDNND FEKKGGFGAL SKQLVAGMVL VLSLWDDHSV NMLWLDSTYP TNAAAGALGT ERGACATSSG KPSDVESQSP DATVTFSDIK |
| | FGPIDSTY |
| SEQ ID NO: 148 | MLCVGLFGLV YSIGVGTNTQ ETHPKLSWKQ CSSGGSCTTQ QGSVVIDSNW RWTHSTKDLT NCYDGNLWDS TLCPDGTTCS KNCVLEGADY SGTYGITSSG DSLTLKFVTH |
| | GSYSTNVGSR LYLLKDDNNY QIFNLAGKEF TFTVDVSNLP CGLNGALYFV EMDQDGGKGK HKENEAGAKY GTGYCDAQCP TDLKFIDGIA NSDGWKPQDN DENSGNGKYG |
| | SCCSEMDIWE ANSLATAYTP HVCDTKGQKR CQGTACGENG GGDRFGSECD PDGCDFNSWR QGNKSFWGPG LIIDTKKSVQ VVTQFIGSGS SVTEIRRKYV QNGKVIENSY |
| | STISGTEKYN SISDDYCNAQ KKAFGDTNSF ENHGGFKRFS QHIQDMVLVL SLWDDHTVNM LWLDSVYPTN SNKPGADRGP CETSSGVPAD VESKSASASV KYSDIRFGPI |
| | DSTYK |
| SEQ ID NO: 149 | MLLCLWSIAY SLGVGTNTAE NHPKLSWKNG GSSVSGSVTV DANWRWTHIK GETKNCYDGN LWSDKYCPDA ATCGKNCVIE GADYQGTYGV SASGDGLTLT FVTHGQYSTN |
| | VGSRLYLMKD EKTYQIFNLN GKEFTFTVDV SNLPCGLNGA LYFVQMDSDG GMAKYPDNQA GAKYGTGYCD AQCPTDLKFI NGIPNSDGWK PQKNDKNSGN GKYGSCCSEM |
| | DIWEANSQAT AYTPHVCDKL EQTRCSGSAC GHTGGGERFS SSCDPDGCDF NSWRMGNKTF WGPGLIVDTK KPVQVVTQFV GSGNSCTEIK RKYVQGGKVI DNSMSNIAGM |
| | TKQYNSVSDD FCQAQKKAFG DNDSFTKHGG FRQLGATLGK GHVLVLSLWD DHDVNMLWLD SVYPTNSNKP GSDRGPCKTS SGIPADVESQ AASSSVKYSD IRFGAIDSTY |
| | K |
| SEQ ID NO: 299 | QSACTLQSET HPPLTWQKCS SGGTCTQQTG SVVIDANWRW THATNSSTNC YDGNTWSSTL CPDNETCAKN CCLDGAAYAS TYGVTTSGNS LSIGFVTQSA QKNVGARLYL |
| | MASDTTYQEF TLLGNEFSFD VDVSQLPCGL NGALYFVSMD ADGGVSKYPT NTAGAKYGTG YCDSQCPRDL KFINGQANVE GWEPSSNNAN TGIGGHGSCC SEMDIWEANS |
| | ISEALTPHPC TTVGQEICEG DGCGGTYSDN AYGGTCDPDG CDWNPYRLGN TSFYGPGSSF TLDTTKKLTV VTQFETSGAI NRYYVQNGVT FQQPNAELGS YSGNELNDDY |
| | CTAEEAEFGG SSFSDKGGLT QFKKATSGGM VLVMSLWDDY YANMLWLDST YPTNETSSTP GAVRGSCSTS SGVPAQVESQ SPNAKVTFSN IKFGPIGSTG NPSGGNPPGG |
| | NPPGTTTTRR PATTTGSSPG PTQSHYGQCG GIGYSGPTVC ASGTTCQVLN PYYSQCL |
| SEQ ID NO: 300 | QSACTLQSET HPPLTWQKCS SGGTCTQQTG SVVIDANWRW THATNSSTNC YDGNTWSSTL CPDNETCAKN CCLDGAAYAS TYGVTTSGNS LSIGFVTQSA QKNVGARLYL |
| | MASDTTYQEF TLLGNEFSFD VDVSQLPCGL NGALYFVSMD ADGGVSKYPT NTAGAKYGTG YCDSQCPRDL KFINGQANVE GWEPSSNNAN TGIGGHGSCC SEMDIWEANS |
| | ISEALTPHPC TTVGQEICEG DGCGGTYSDN RYGGTCDPDG CDWNPYRLGN TSFYGPGSSF TLDTTKKLTV VTQFETSGAI NRYYVQNGVT FQQPNAELGS YSGNELNDDY |
| | CTAEEAEFGG SSFSDKGGLT QFKKATSGGM VLVMSLWDDY YANMLWLDST YPTNETSSTP GAVAGSCSTS SGVPAQVESQ SPNAKVTFSN IKFGPIGSTG NPSGGNPPGG |
| | NPPGTTTTRR PATTTGSSPG PTQSHYGQCG GIGYSGPTVC ASGTTCQVLN PYYSQCL |
| SEQ ID NO: 301 | MSALNSFNMY KSALILGSLL ATAGAQQIGT YTAETHPSLS WSTCKSGGSC TTNSGAITLD ANWRWVHGVN TSTNCYTGNT WNTAICDTDA SCAQDCALDG ADYSGTYGIT |
| | TSGNSLRLNF VTGSNVGSRT YLMADNTHYQ IFDLLNQEFT FTVDVSHLPC GLNGALYFVT MDADGGVSKY PNNKAGAQYG VGYCDSQCPR DLKFIAGQAN VEGWTPSSNN |
| | ANTGLGNHGA CCAELDIWEA NSISEALTPH PCDTPGLSVC TTDACGGTYS SDKYAGTCDP DGCDFNPYRL GVTDFYGSGK TVDTTKPITV VTQFVTDDGT STGTLSEIRR |
| | YYVQNGVVIP QPSSKISGVS GNVINSDFCD AEISTFGETA SFSKHGGLAK MGAGMEAGMV LVMSLWDDYS VNMLWLDSTY PTNATGTPGA AKGSCPTTSG DPKTVESQSG |
| | SSYVTFSDIR VGPFNSTFSG GSSTGGSSTT TASGTTTTKA SSTSTSSTST GTGVAAHWGQ CGGQGWTGPT TCASGTTCTV VNPYYSQCL |
| SEQ ID NO: 302 | QQIGTYTAET HPSLSWSTCK SGGSCTTNSG AITLDANWRW VHGVNTSTNC YTGNTWNTAI CDTDASCAQD CALDGADYSG TYGITTSGNS LRLNFVTGSN VGSRTYLMAD |
| | NTHYQIFDLL NQEFTFTVDV SHLPCGLNGA LYFVTMDADG GVSKYPNNKA GAQYGVGYCD SQCPRDLKFI AGQANVEGWT PSSNNANTGL GNHGACCAEL DIWEANSISE |
| | ALTPHPCDTP GLSVCTTDAC GGTYSSDKYA GTCDPDGCDF NPYRLGVTDF YGSGKTVDTT KPITVVTQFV TDDGTSTGTL SEIRRYYVQN GVVIPQPSSK ISGVSGNVIN |
| | SDFCDAEIST FGETASFSKH GGLAKMGAGM EAGMVLVMSL WDDYSVNMLW LDSTYPTNAT GTPGAAKGSC PTTSGDPKTV ESQSGSSYVT FSDIRVGPFN STFSGGSSTG |
| | GSSTTTASGT TTTKASSTST SSTSTGTGVA AHWGQCGGQG WTGPTTCASG TTCTVVNPYY SQCL |

TABLE 8

| | | | MUL Data | | Saccharification | | |
|---|---|---|---|---|---|---|---|
| | | | | Tolerance | % | % | RPLC |
| | | | SA (μmol | at 1 mM | Conversion | Conversion | Quantification |
| Group | variant | IC₅₀ | 4 MU/min/mg) | CB | (measured) | (measured) | (μg/mL) |
| WT | control | 0.05 | 0.60 | 6% | 9.8% | 5.9% | 21.6 |
| 268_{—} | Ala | 0.87 | 1.42 | 48% | 4.8% | 4.7% | 16.9 |
| 268_{—} | Ile | 0.61 | 1.61 | 40% | 4.4% | 3.4% | 11.5 |
| 268_{—} | Leu | 0.58 | 11.27 | 36% | 5.1% | 0.9% | 1.7 |
| 268_{—} | Val | 0.56 | 1.39 | 37% | 3.1% | 2.6% | 8.6 |
| 268_{—} | Phe | 0.40 | 0.70 | 21% | 1.8% | 1.1% | 2.5 |
| 268_{—} | Trp | 0.61 | 1.31 | 42% | 2.1% | 2.3% | 7.0 |
| 268_{—} | Tyr | 0.45 | 0.65 | 35% | 2.6% | 2.9% | 9.8 |
| 268_{—} | Asp | 0.90 | 0.67 | 44% | 3.0% | 2.5% | 7.8 |
| 268_{—} | Glu | 0.87 | 0.88 | 52% | 2.4% | 2.1% | 6.3 |
| 268_{—} | Arg | 0.03 | 0.52 | 3% | 8.3% | 5.7% | 20.8 |
| 268_{—} | His | 0.25 | 1.21 | 20% | 5.2% | 4.9% | 17.5 |
| 268_{—} | Lys | 0.15 | 1.28 | 12% | 5.8% | 6.5% | 24.2 |
| 268_{—} | Asn | 0.67 | 13.97 | 41% | 2.6% | 0.6% | 0.5 |
| 268_{—} | Gln | ND | | | | | |
| 268_{—} | Ser | 0.74 | 1.00 | 45% | 2.7% | 2.6% | 8.3 |
| 268_{—} | Thr | 0.60 | 0.97 | 42% | 2.1% | 1.9% | 5.5 |
| 268_{—} | Cys | 0.52 | 0.86 | 35% | 2.4% | 2.2% | 6.7 |
| 268_{—} | Gly | 0.64 | 0.93 | 43% | 3.6% | 3.3% | 11.1 |
| 268_{—} | Met | | | | | | |
| 268_{—} | Pro | 0.62 | 1.70 | 40% | 2.7% | 2.7% | 8.9 |
| 268_+411A | Ala | 1.33 | 0.52 | 65% | 4.7% | 4.1% | 14.4 |
| 268_+411A | Ile | 10.38 | 0.89 | 90% | 2.8% | 3.1% | 10.3 |
| 268_+411A | Leu | 7.05 | 0.82 | 88% | 2.7% | 3.5% | 12.1 |
| 268_+411A | Val | 7.48 | 1.33 | 93% | 3.7% | 3.2% | 10.6 |
| 268_+411A | Phe | | | — | 0.7% | 0.5% | |
| 268_+411A | Trp | 7.01 | 0.81 | 84% | 2.7% | 3.4% | 11.8 |
| 268_+411A | Tyr | | | | | | |
| 268_+411A | Asp | 11.26 | 0.22 | 85% | 1.2% | 1.3% | 3.4 |
| 268_+411A | Glu | | | | | | |
| 268_+411A | Arg | 1.60 | 0.38 | 72% | 2.5% | 2.3% | 7.0 |
| 268_+411A | His | 4.84 | 0.98 | 95% | 3.5% | 3.6% | 12.5 |
| 268_+411A | Lys | 6.32 | 1.03 | 93% | 1.4% | 1.0% | 2.1 |
| 268_+411A | Asn | | | | | | |
| 268_+411A | Gln | | −0.45 | — | 0.6% | 0.7% | 0.9 |
| 268_+411A | Ser | 6.31 | 1.62 | 96% | 2.9% | 2.4% | 7.8 |
| 268_+411A | Thr | | | | | | |
| 268_+411A | Cys | 17.68 | 0.28 | — | 0.9% | 0.8% | 1.4 |
| 268_+411A | Gly | 9.53 | 0.80 | 99% | 2.9% | 3.5% | 12.0 |
| 268_+411A | Met | 8.66 | 0.83 | 95% | 2.6% | 3.1% | 10.4 |
| 268_+411A | Pro | 7.31 | 1.80 | 80% | 2.8% | 3.3% | 11.2 |
| 268A+411_{—} | Ala | 5.56 | 1.19 | 83% | 3.3% | 4.8% | 17.0 |
| 268A+411_{—} | Ile | 28.03 | 0.58 | 107% | 1.2% | 1.2% | 2.6 |
| 268A+411_{—} | Leu | 25.06 | 1.72 | 99% | 1.4% | 0.9% | 1.6 |
| 268A+411_{—} | Val | 15.07 | 1.39 | 102% | 1.7% | 2.2% | 6.6 |
| 268A+411_{—} | Phe | 19.07 | 0.97 | 100% | 1.8% | 3.0% | 10.1 |
| 268A+411_{—} | Trp | 28.40 | 3.07 | 97% | 1.5% | 1.1% | 2.5 |
| 268A+411_{—} | Tyr | | | | | | |
| 268A+411_{—} | Asp | 10.25 | 2.12 | 93% | 1.9% | 1.9% | 5.4 |
| 268A+411_{—} | Glu | 16.89 | 0.74 | 95% | 1.9% | 1.8% | 5.3 |
| 268A+411_{—} | Arg | 0.61 | 1.56 | 39% | 4.6% | 5.2% | 18.6 |
| 268A+411_{—} | His | 29.34 | 0.38 | — | 0.9% | 0.8% | 1.0 |
| 268A+411_{—} | Lys | 7.36 | 1.08 | 88% | 1.8% | 2.8% | 9.1 |
| 268A+411_{—} | Asn | | | | | | |
| 268A+411_{—} | Gln | 15.11 | 1.33 | 99% | 2.0% | 2.2% | 6.7 |
| 268A+411_{—} | Ser | 5.69 | 3.19 | 91% | 3.3% | 2.1% | 6.3 |
| 268A+411_{—} | Thr | 10.12 | 1.39 | 91% | 1.8% | 2.6% | 8.3 |
| 268A+411_{—} | Cys | 7.66 | 1.58 | 85% | 2.7% | 3.9% | 13.7 |
| 268A+411_{—} | Gly | 12.07 | 0.88 | 91% | 2.3% | 2.4% | 7.7 |
| 268A+411_{—} | Met | 11.51 | 0.87 | 97% | 2.1% | 3.4% | 11.5 |
| 268A+411_{—} | Pro | 17.92 | 0.18 | — | 1.1% | 0.8% | 1.3 |
| 411_{—} | Ala | 1.79 | 0.35 | 65% | 2.5% | 1.9% | 5.5 |
| 411_{—} | Ile | | | | | | |
| 411_{—} | Leu | 6.86 | 0.25 | — | 1.6% | 0.9% | 1.7 |
| 411_{—} | Val | 3.35 | 0.51 | 82% | 4.2% | 3.3% | 11.2 |
| 411_{—} | Phe | 6.26 | 0.43 | 89% | 3.0% | 3.7% | 12.7 |
| 411_{—} | Trp | 10.91 | 2.19 | 100% | 2.1% | 0.9% | 1.6 |
| 411_{—} | Tyr | 5.40 | 0.67 | 85% | 3.5% | 3.9% | 13.4 |
| 411_{—} | Asp | 2.08 | 0.23 | 106% | 1.7% | 1.2% | 2.6 |
| 411_{—} | Glu | 2.95 | 0.38 | 76% | 2.5% | 2.0% | 6.1 |
| 411_{—} | Arg | 0.09 | 0.60 | — | 4.0% | 2.6% | 8.3 |
| 411_{—} | His | 3.66 | 0.52 | 84% | 4.7% | 5.1% | 18.4 |
| 411_{—} | Lys | 3.13 | 0.46 | 82% | 4.7% | 4.6% | 16.2 |
| 411_{—} | Asn | 5.16 | 0.20 | 75% | 2.7% | 2.4% | 7.5 |
| 411_{—} | Gln | | −0.85 | — | 0.8% | 0.6% | 0.4 |
| 411_{—} | Ser | 1.05 | 0.51 | 60% | 4.2% | 3.2% | 10.6 |
| 411_{—} | Thr | 1.78 | 0.49 | 65% | 3.9% | 3.6% | 12.2 |
| 411_{—} | Cys | 1.60 | 0.52 | 71% | 4.7% | 5.1% | 18.4 |
| 411_{—} | Gly | 2.01 | 0.48 | 72% | 4.4% | 3.5% | 12.1 |
| 411_{—} | Met | 3.88 | 0.45 | 84% | 3.3% | 3.1% | 10.3 |
| 411_{—} | Pro | 1.13 | 0.58 | 61% | 3.6% | 2.0% | 5.8 |

TABLE 9

| Sample Name | Average IC₅₀ | StDev IC₅₀ |
|---|---|---|
| 268A+411A | 8.550 | 0.150 |
| 268A+411V | 15.982 | 0.839 |
| 268A+411F | 23.082 | 2.644 |
| 268A+411D | 11.846 | 0.587 |
| 268A+411R | 0.414 | 0.076 |
| 268A+411K | 9.234 | 0.101 |
| 268A+411Q | 14.057 | 0.512 |
| 268A+411S | 8.280 | 0.260 |
| 268A+411T | 13.457 | 0.654 |
| 268A+411C | 12.552 | 0.267 |
| 268A+411G | 17.298 | 1.035 |
| 268A+411M | 12.192 | 0.038 |
| 268A+411A | 0.933 | 0.095 |
| 268I+411A | 13.958 | 0.142 |
| 268L+411A | 13.906 | 1.055 |
| 268V+411A | 10.879 | 0.763 |
| 268F+411A | 9.648 | 0.155 |
| 268W+411A | 11.486 | 0.437 |
| 268R+411A | 0.994 | 0.089 |
| 268H+411A | 5.319 | 0.411 |
| 268Q+411A | 9.731 | 1.985 |
| 268S+411A | 11.430 | 0.126 |
| 268G+411A | 9.823 | 0.503 |
| 268M+411A | 13.355 | 1.405 |
| 268P+411A | 8.945 | 0.560 |
| R268A | 0.423 | 0.002 |
| R268I | 0.320 | 0.008 |
| R268L | 0.373 | 0.020 |
| R268V | 0.335 | 0.000 |
| R268W | 0.475 | 0.017 |
| R268Y | 0.344 | 0.015 |
| R268D | 0.431 | 0.067 |
| R268E | 0.540 | 0.068 |
| R268R | 0.046 | 0.004 |
| R268H | 0.209 | 0.007 |
| R268K | 0.093 | 0.024 |
| R268N | 0.405 | 0.064 |
| R268S | 0.406 | 0.021 |
| R268T | 0.360 | 0.041 |
| R268C | 0.335 | 0.025 |
| R268G | 0.358 | 0.016 |
| R268P | 0.440 | 0.039 |
| R411A | 0.918 | 0.002 |
| R411V | 3.193 | 0.379 |
| R411F | 5.386 | |
| R411Y | 4.954 | 0.068 |
| R411R | 0.035 | 0.008 |
| R411H | 2.429 | 0.426 |
| R411K | 2.080 | 0.329 |
| R411N | 6.722 | |
| R411S | 0.762 | 0.024 |
| R411C | 0.886 | 0.023 |
| R411G | 1.470 | 0.386 |
| R411M | 2.597 | 0.428 |
| R411P | 1.048 | 0.145 |
| WT | 0.029 | 0.002 |
| WT | 0.034 | 0.005 |
| WT | 0.030 | 0.000 |
| WT | 0.047 | 0.002 |
| WT | 0.038 | 0.003 |
| WT | 0.038 | 0.001 |
| WT | 0.042 | 0.005 |

TABLE 10

| Variant | IC₅₀ | StDev | n |
|---|---|---|---|
| 268A+411A | 6.855 | | 1 |
| 268A+411V | 12.311 | | 1 |
| 268A+411F | 15.108 | | 1 |
| 268A+411W | 42.065 | 4.169 | 3 |
| 268A+411D | 11.675 | 3.164 | 2 |
| 268A+411R | 0.453 | | 1 |
| 268A+411K | 7.784 | | 1 |
| 268A+411Q | 12.145 | | 1 |
| 268A+411S | 8.366 | 2.211 | 2 |
| 268A+411T | 9.647 | | 1 |
| 268A+411C | 9.054 | 3.663 | 2 |
| 268A+411G | 13.492 | | 1 |
| 268A+411M | 10.734 | | 1 |
| 268A+411P | 9.310 | 0.656 | 3 |
| 268A+411A | 1.030 | | 1 |
| 268I+411A | 11.502 | | 1 |
| 268L+411A | 11.422 | | 1 |
| 268V+411A | 8.721 | | 1 |
| 268F+411A | 9.795 | | 1 |
| 268W+411A | 9.902 | | 1 |
| 268Y+411A | 10.917 | 2.034 | 3 |
| 268D+411A | 14.351 | 1.620 | 2 |
| 268E+411A | 16.694 | 0.479 | 3 |
| 268R+411A | 1.296 | | 1 |
| 268H+411A | 5.581 | | 1 |
| 268N+411A | 13.277 | 0.914 | 3 |
| 268Q+411A | 7.931 | | 1 |
| 268S+411A | 9.122 | | 1 |
| 268G+411A | 8.997 | | 1 |
| 268M+411A | 12.050 | | 1 |
| 268P+411A | 9.085 | | 1 |
| R268A | 0.574 | | 1 |
| R268I | 0.484 | | 1 |
| R268L | 0.484 | | 1 |
| R268V | 0.383 | | 1 |
| R268W | 0.497 | | 1 |
| R268Y | 0.434 | | 1 |
| R268D | 0.467 | | 1 |
| R268E | 0.555 | | 1 |
| R268R | 0.052 | | 1 |
| R268H | 0.283 | | 1 |
| R268K | 0.134 | | 1 |
| R268N | 0.482 | | 1 |
| R268S | 0.452 | | 1 |
| R268T | 0.349 | | 1 |
| R268C | 0.351 | | 1 |
| R268G | 0.455 | | 1 |
| R268P | 0.591 | | 1 |
| R411A | 1.063 | | 1 |
| R411V | 2.903 | | 1 |
| R411F | 7.577 | | 1 |
| R411Y | 5.252 | | 1 |
| R411D | 1.578 | 0.139 | 2 |
| R411R | 0.055 | | 1 |
| R411H | 3.223 | | 1 |
| R411K | 3.055 | | 1 |
| R411S | 0.895 | | 1 |
| R411T | 1.999 | 0.092 | 3 |
| R411C | 1.314 | | 1 |
| R411G | 2.307 | | 1 |
| R411M | 4.263 | | 1 |
| R411P | 1.270 | | 1 |
| WT | 0.070 | 0.003 | 7 |

TABLE 11

| Variant | IC₅₀ | StDev | n |
|---|---|---|---|
| 268A+411A | 12.089 | | 1 |
| 268A+411I | 35.003 | 4.911 | 2 |
| 268A+411L | 21.530 | 1.050 | 2 |
| 268A+411W^{¥} | 32.376 | | 1 |
| 268A+411E | 13.144 | 4.574 | 2 |
| 268A+411H | 21.293 | 4.387 | 2 |
| 268A+411Q | 13.304 | | |
| 268A+411P^{¥} | 14.485 | | 1 |
| 268D+411A | 17.680 | | 1 |
| 268K+411A | 6.084 | 1.054 | 3 |
| 268C+411A | 24.892 | 4.393 | 2 |
| R268F | 0.515 | 0.028 | 2 |
| R411L | 6.387 | 0.136 | 2 |
| R411W | 7.739 | 0.260 | 2 |
| R411D | 1.636 | 0.279 | 2 |
| R411E | 3.381 | 0.649 | 2 |
| R411N^{¥} | 7.896 | | 1 |
| R411Q | 2.513 | | 1 |
| R411T | 2.025 | 0.280 | 2 |
| WT | 0.056 | 0.020 | 2 |
| WT | 0.066 | 0.011 | 2 |

^{¥}poor fit; R² < 0.95

TABLE 12

| | | 268_{—} | | 411_{—} | | 268A+411_{—} | | |
|---|---|---|---|---|---|---|---|---|
| AA class | Variant | Measured | ↑ in IC₅₀ | Measured | ↑ in IC₅₀* | Measured | ↑ in IC₅₀* | Expected IC₅₀ |
| Aliphatic | Ala | 0.57 | 12.5 | 1.17 | 26 | 8.32 | 181 | 1.75 |
| Aliphatic | Ile | 0.43 | 9.4 | ND | 0 | 32.68 | 712 | ND |
| Aliphatic | Leu | 0.45 | 9.8 | 6.54 | 143 | 22.71 | 495 | 7.12 |
| Aliphatic | Val | 0.40 | 8.8 | 3.16 | 69 | 14.83 | 323 | 3.73 |
| Aromatic | Phe | 0.48 | 10.4 | 6.41 | 140 | 20.09 | 437 | 6.98 |
| Aromatic | Trp | 0.51 | 11.2 | 8.80 | 192 | 37.39 | 814 | 9.37 |
| Aromatic | Tyr | 0.39 | 8.5 | 5.14 | 112 | ND | | |
| Charged-Acidic | Asp | 0.56 | 12.1 | 1.70 | 37 | 11.46 | 250 | 2.28 |
| Charged-Acidic | Glu | 0.63 | 13.6 | 3.24 | 70 | 14.39 | 313 | 3.81 |
| Charged-Basic | Arg | 0.04 | 1.0 | 0.05 | 1 | 0.47 [symbol] | 10 | 0.63 |
| Charged-Basic | His | 0.24 | 5.2 | 2.93 | 64 | 23.97 | 522 | 3.51 |
| Charged-Basic | Lys | 0.12 | 2.5 | 2.59 | 56 | 8.40 | 183 | 3.16 |
| Polar | Asn | 0.49 | 10.7 | 6.59 | 144 | ND | | |
| Polar | Gln | ND | — | 2.51 [symbol] | 55 | 13.73 | 299 | 3.09 |
| Polar | Ser | 0.50 | 10.9 | 0.87 | 19 | 7.80 | 170 | 1.44 |
| Polar | Thr | 0.42 | 9.1 | 1.97 | 43 | 11.67 | 254 | 2.54 |
| Special | Cys | 0.38 | 8.4 | 1.17 | 25 | 10.17 | 222 | 1.74 |
| Special | Gly | 0.45 | 9.9 | 1.81 | 40 | 15.04 | 328 | 2.39 |
| Special | Met | ND | | 3.34 | 73 | 11.66 | 254 | 3.91 |
| Special | Pro | 0.52 | 11.4 | 1.13 | 25 | 12.07 | 263 | 1.70 |

| | | | | | | | |
|---|---|---|---|---|---|---|---|
| average | 0.42 | 8.3 | 3.22 | 67 | 15.38 | 335 | 3.48 |

| | | 268A+411_{—} | 268_+411A | | | |
|---|---|---|---|---|---|---|
| AA class | Variant | Synergistic**↑ | Measured | ↑ in IC₅₀* | Expected IC₅₀ | Synergistic**↑ |
| Aliphatic | Ala | 4.8 | 8.70 | 189 | 1.75 | 5.0 |
| Aliphatic | Ile | — | 12.45 | 271 | 1.61 | 7.8 |
| Aliphatic | Leu | 3.2 | 11.57 | 252 | 1.63 | 7.1 |
| Aliphatic | Val | 4.0 | 9.49 | 207 | 1.57 | 6.0 |
| Aromatic | Phe | 2.9 | 9.70 | 211 | 1.65 | 5.9 |
| Aromatic | Trp | 4.0 | 9.97 | 217 | 1.69 | 5.9 |
| Aromatic | Tyr | | 10.92 | 238 | 1.56 | 7.0 |
| Charged-Acidic | Asp | 5.0 | 14.41 | 314 | 1.73 | 8.3 |
| Charged-Acidic | Glu | 3.8 | 16.69 | 364 | 1.80 | 9.3 |
| Charged-Basic | Arg | 0.8 | 1.22 | 27 | 1.22 | 1.0 |
| Charged-Basic | His | 6.8 | 5.27 | 115 | 1.41 | 3.7 |
| Charged-Basic | Lys | 2.7 | 6.14 | 134 | 1.29 | 4.8 |
| Polar | Asn | | 13.28 | 289 | 1.66 | 8.0 |
| Polar | Gln | 4.5 | 9.13 | 199 | ND | |
| Polar | Ser | 5.4 | 9.57 | 209 | 1.67 | 5.7 |
| Polar | Thr | 4.6 | ND | | | |
| Special | Cys | 5.8 | 22.4 [symbol] | 490 | 1.56 | 14.4 |
| Special | Gly | 6.3 | 9.54 | 208 | 1.63 | 5.9 |
| Special | Met | 3.0 | 11.85 | 258 | | |
| Special | Pro | 7.1 | 8.57 | 187 | 1.70 | 5.1 |

| | | | | | |
|---|---|---|---|---|---|
| average | 4.4 | 10.58 | 230 | 1.59 | 6.5 |

[symbol]

indicates data missing or illegible when filed

TABLE 13

| | | 268_{—} | | | 411_{—} | | |
|---|---|---|---|---|---|---|---|
| AA class | Variant | Measured | Δ SA* | Std. Dev.* | Measured | Δ SA* | Std. Dev.* |
| Aliphatic | Ala | 2.97 | 3.9 | 0.29 | 0.52 | 0.7 | 0.12 |
| Aliphatic | Ile | 1.98 | 2.6 | 0.25 | ND | — | — |
| Aliphatic | Leu | 1.83 | 2.4 | 0.25 | 0.18 [symbol] | 0.2 | 0.11 [symbol] |
| Aliphatic | Val | 2.36 | 3.1 | 0.10 | 0.65 | 0.9 | 0.10 |
| Aromatic | Phe | 0.37 [symbol] | 0.5 | 0.32 [symbol] | 0.65 | 0.9 | 0.19 |
| Aromatic | Trp | 2.51 | 3.3 | 0.02 | 1.35 [symbol] | 1.8 | 1.19 [symbol] |
| Aromatic | Tyr | 1.25 | 1.6 | 0.03 | 0.82 | 1.1 | 0.10 |
| Charged-Acidic | Asp | 1.46 | 1.9 | 0.04 | 0.47 | 0.6 | 0.21 |
| Charged-Acidic | Glu | 1.84 | 2.4 | 0.17 | 0.28 [symbol] | 0.4 | 0.14 [symbol] |
| Charged-Basic | Arg | 0.97 | 1.3 | 0.04 | 0.83 | 1.1 | 0.05 |
| Charged-Basic | His | 1.72 | 2.3 | 0.63 | 0.80 | 1.0 | 0.18 |
| Charged-Basic | Lys | 3.34 | 4.4 | 0.42 | 0.80 | 1.0 | 0.03 |
| Polar | Asn | 1.56 | 2.1 | 0.07 | 0.15 [symbol] | 0.2 | 0.07 [symbol] |
| Polar | Gln | ND | — | — | −0.45 [symbol] | −0.6 | 0.57 [symbol] |
| Polar | Ser | 1.85 | 2.4 | 0.11 | 0.60 | 0.8 | 0.07 |
| Polar | Thr | 1.63 | 2.1 | 0.45 | 0.48 | 0.6 | 0.06 |
| Special | Cys | 1.81 | 2.4 | 0.13 | 0.92 | 1.2 | 0.04 |
| Special | Gly | 1.43 | 1.9 | 0.33 | 0.60 | 0.8 | 0.09 |
| Special | Met | ND | — | — | 0.57 | 0.7 | 0.09 |
| Special | Pro | 3.38 | 4.4 | 0.09 | 0.70 | 0.9 | 0.09 |
| Average | | 1.90 | 2.5 | | 0.6 | 0.8 | |
| | | 268A+411_{—} | | | 268_{—} +411 A | | |
| AA class | Variant | Measured | Δ SA* | Std. Dev.* | Measured | Δ SA* | Std. Dev.* |
| Aliphatic | Ala | 1.82 | 2.4 | 0.46 | 0.85 | 1.1 | 0.253 |
| Aliphatic | Ile | 0.35 [symbol] | 0.5 | 0.33 [symbol] | 1.15 | 1.5 | 0.18 |
| Aliphatic | Leu | 0.96 [symbol] | 1.3 | 0.67 [symbol] | 0.99 | 1.3 | 0.12 |
| Aliphatic | Val | 1.78 | 2.3 | 0.27 | 1.43 | 1.9 | 0.07 |
| Aromatic | Phe | 1.85 | 2.4 | 0.10 | 2.07 | 2.7 | 0.16 |
| Aromatic | Trp | 0.72 | 1.0 | 0.06 | 1.37 | 1.8 | 0.17 |
| Aromatic | Tyr | ND | — | — | 0.74 | 1.0 | 0.03 |
| Charged-Acidic | Asp | 2.21 | 2.9 | 0.32 | 0.52 [symbol] | 0.7 | 0.26 [symbol] |
| Charged-Acidic | Glu | 0.49 [symbol] | 0.6 | 0.35 [symbol] | 0.58 | 0.8 | 0.12 |
| Charged-Basic | Arg | 2.32 | 3.0 | 0.52 | 0.53 | 0.7 | 0.10 |
| Charged-Basic | His | 0.77 [symbol] | 1.0 | 0.68 [symbol] | 1.35 | 1.8 | 0.26 |
| Charged-Basic | Lys | 1.18 [symbol] | 1.5 | 0.44 [symbol] | 0.56 [symbol] | 0.7 | 0.67 [symbol] |
| Polar | Asn | ND | — | — | 0.84 | 1.1 | 0.20 |
| Polar | Gln | 1.22 | 1.6 | 0.31 | 2.13 | 2.8 | 0.18 |
| Polar | Ser | 2.19 | 2.9 | 0.60 | 1.59 | 2.1 | 0.03 |
| Polar | Thr | 1.78 | 2.3 | 0.27 | ND | — | — |
| Special | Cys | 1.85 | 2.4 | 0.20 | 0.16 [symbol] | 0.2 | 0.17 [symbol] |
| Special | Gly | 0.99 | 1.3 | 0.08 | 2.11 | 2.8 | 0.13 |
| Special | Met | 1.78 | 2.3 | 0.45 | 1.13 | 1.5 | 0.20 |
| Special | Pro | 1.90 | 2.5 | 0.07 | 2.16 | 2.8 | 0.24 |

| | | | | |
|---|---|---|---|---|
| Average | 1.5 | 1.7 | 1.2 | 1.5 |

[symbol]

indicates data missing or illegible when filed

TABLE 14

| Variant No. | R268 Substituent | R411 Substituent |
|---|---|---|
| 1. | A | A |
| 2. | C | A |
| 3. | D | A |
| 4. | E | A |
| 5. | F | A |
| 6. | G | A |
| 7. | H | A |
| 8. | I | A |
| 9. | K | A |
| 10. | L | A |
| 11. | M | A |
| 12. | N | A |
| 13. | P | A |
| 14. | Q | A |
| 15. | — | A |
| 16. | S | A |
| 17. | T | A |
| 18. | V | A |
| 19. | W | A |
| 20. | Y | A |
| 21. | A | C |
| 22. | C | C |
| 23. | D | C |
| 24. | E | C |
| 25. | F | C |
| 26. | G | C |
| 27. | H | C |
| 28. | I | C |
| 29. | K | C |
| 30. | L | C |
| 31. | M | C |
| 32. | N | C |
| 33. | P | C |
| 34. | Q | C |
| 35. | — | C |
| 36. | S | C |
| 37. | T | C |
| 38. | V | C |
| 39. | W | C |
| 40. | Y | C |
| 41. | A | D |
| 42. | C | D |
| 43. | D | D |
| 44. | E | D |
| 45. | F | D |
| 46. | G | D |
| 47. | H | D |
| 48. | I | D |
| 49. | K | D |
| 50. | L | D |
| 51. | M | D |
| 52. | N | D |
| 53. | P | D |
| 54. | Q | D |
| 55. | — | D |
| 56. | S | D |
| 57. | T | D |
| 58. | V | D |
| 59. | W | D |
| 60. | Y | D |
| 61. | A | E |
| 62. | C | E |
| 63. | D | E |
| 64. | E | E |
| 65. | F | E |
| 66. | G | E |
| 67. | H | E |
| 68. | I | E |
| 69. | K | E |
| 70. | L | E |
| 71. | M | E |
| 72. | N | E |
| 73. | P | E |
| 74. | Q | E |
| 75. | — | E |
| 76. | S | E |
| 77. | T | F |
| 78. | V | E |
| 79. | W | E |
| 80. | Y | E |
| 81. | A | F |
| 82. | C | F |
| 83. | D | F |
| 84. | E | F |
| 85. | F | F |
| 86. | G | F |
| 87. | H | F |
| 88. | I | F |
| 89. | K | F |
| 90. | L | F |
| 91. | M | F |
| 92. | N | F |
| 93. | P | F |
| 94. | Q | F |
| 95. | — | F |
| 96. | S | F |
| 97. | T | F |
| 98. | V | F |
| 99. | W | F |
| 100. | Y | F |
| 101. | A | G |
| 102. | C | G |
| 103. | D | G |
| 104. | E | G |
| 105. | F | G |
| 106. | G | G |
| 107. | H | G |
| 108. | I | G |
| 109. | K | G |
| 110. | L | G |
| 111. | M | G |
| 112. | N | G |
| 113. | P | G |
| 114. | Q | G |
| 115. | — | G |
| 116. | S | G |
| 117. | T | G |
| 118. | V | G |
| 119. | W | G |
| 120. | Y | G |
| 121. | A | H |
| 122. | C | H |
| 123. | D | H |
| 124. | E | H |
| 125. | F | H |
| 126. | G | H |
| 127. | H | H |
| 128. | I | H |
| 129. | K | H |
| 130. | L | H |
| 131. | M | H |
| 132. | N | H |
| 133. | P | H |
| 134. | Q | H |
| 135. | — | H |
| 136. | S | H |
| 137. | T | H |
| 138. | V | H |
| 139. | W | H |
| 140. | Y | H |
| 141. | A | I |
| 142. | C | I |
| 143. | D | I |
| 144. | E | I |
| 145. | F | I |
| 146. | G | I |
| 147. | H | I |
| 148. | I | I |
| 149. | K | I |
| 150. | L | I |
| 151. | M | I |
| 152. | N | I |
| 153. | P | I |
| 154. | Q | I |
| 155. | — | I |
| 156. | S | I |
| 157. | T | I |
| 158. | V | I |
| 159. | W | I |
| 160. | Y | I |
| 161. | A | K |
| 162. | C | K |
| 163. | D | K |
| 164. | E | K |
| 165. | F | K |
| 166. | G | K |
| 167. | H | K |
| 168. | I | K |
| 169. | K | K |
| 170. | L | K |
| 171. | M | K |
| 172. | N | K |
| 173. | P | K |
| 174. | Q | K |
| 175. | — | K |
| 176. | S | K |
| 177. | T | K |
| 178. | V | K |
| 179. | W | K |
| 180. | Y | K |
| 181. | A | L |
| 182. | C | L |
| 183. | D | L |
| 184. | E | L |
| 185. | F | L |
| 186. | G | L |
| 187. | H | L |
| 188. | I | L |
| 189. | K | L |
| 190. | L | L |
| 191. | M | L |
| 192. | N | L |
| 193. | P | L |
| 194. | Q | L |
| 195. | — | L |
| 196. | S | L |
| 197. | T | L |
| 198. | V | L |
| 199. | W | L |
| 200. | Y | L |
| 201. | A | M |
| 202. | C | M |
| 203. | D | M |
| 204. | E | M |
| 205. | F | M |
| 206. | G | M |
| 207. | H | M |
| 208. | I | M |
| 209. | K | M |
| 210. | L | M |
| 211. | M | M |
| 212. | N | M |
| 213. | P | M |
| 214. | Q | M |
| 215. | — | M |
| 216. | S | M |
| 217. | T | M |
| 218. | V | M |
| 219. | W | M |
| 220. | Y | M |
| 221. | A | N |
| 222. | C | N |
| 223. | D | N |
| 224. | E | N |
| 225. | F | N |
| 226. | G | N |
| 227. | H | N |
| 228. | I | N |
| 229. | K | N |
| 230. | L | N |
| 231. | M | N |
| 232. | N | N |
| 233. | P | N |
| 234. | Q | N |
| 235. | — | N |
| 236. | S | N |
| 237. | T | N |
| 238. | V | N |
| 239. | W | N |
| 240. | Y | N |
| 241. | A | P |
| 242. | C | P |
| 243. | D | P |
| 244. | E | P |
| 245. | F | P |
| 246. | G | P |
| 247. | H | P |
| 248. | I | P |
| 249. | K | P |
| 250. | L | P |
| 251. | M | P |
| 252. | N | P |
| 253. | P | P |
| 254. | Q | P |
| 255. | — | P |
| 256. | S | P |
| 257. | T | P |
| 258. | V | P |
| 259. | W | P |
| 260. | Y | P |
| 261. | A | Q |
| 262. | C | Q |
| 263. | D | Q |
| 264. | E | Q |
| 265. | F | Q |
| 266. | G | Q |
| 267. | H | Q |
| 268. | I | Q |
| 269. | K | Q |
| 270. | L | Q |
| 271. | M | Q |
| 272. | N | Q |
| 273. | P | Q |
| 274. | Q | Q |
| 275. | — | Q |
| 276. | S | Q |
| 277. | T | Q |
| 278. | V | Q |
| 279. | W | Q |
| 280. | Y | Q |
| 281. | A | — |
| 282. | C | — |
| 283. | D | — |
| 284. | E | — |
| 285. | F | — |
| 286. | G | — |
| 287. | H | — |
| 288. | I | — |
| 289. | K | — |
| 290. | L | — |
| 291. | M | — |
| 292. | N | — |
| 293. | P | — |
| 294. | Q | — |
| Wild Type | — | — |
| 295. | S | — |
| 296. | T | — |
| 297. | V | — |
| 298. | W | — |
| 299. | Y | — |
| 300. | A | S |
| 301. | C | S |
| 302. | D | S |
| 303. | E | S |
| 304. | F | S |
| 305. | G | S |
| 306. | H | S |
| 307. | I | S |
| 308. | K | S |
| 309. | L | S |
| 310. | M | S |
| 311. | N | S |
| 312. | P | S |
| 313. | Q | S |
| 314. | — | S |
| 315. | S | S |
| 316. | T | S |
| 317. | V | S |
| 318. | W | S |
| 319. | Y | S |
| 320. | A | T |
| 321. | C | T |
| 322. | D | T |
| 323. | E | T |
| 324. | F | T |
| 325. | G | T |
| 326. | H | T |
| 327. | I | T |
| 328. | K | T |
| 329. | L | T |
| 330. | M | T |
| 331. | N | T |
| 332. | P | T |
| 333. | Q | T |
| 334. | — | T |
| 335. | S | T |
| 336. | T | T |
| 337. | V | T |
| 338. | W | T |
| 339. | Y | T |
| 340. | A | V |
| 341. | C | V |
| 342. | D | V |
| 343. | E | V |
| 344. | F | V |
| 345. | G | V |
| 346. | H | V |
| 347. | I | V |
| 348. | K | V |
| 349. | L | V |
| 350. | M | V |
| 351. | N | V |
| 352. | P | V |
| 353. | Q | V |
| 354. | — | V |
| 355. | S | V |
| 356. | T | V |
| 357. | V | V |
| 358. | W | V |
| 359. | Y | V |
| 360. | A | W |
| 361. | C | W |
| 362. | D | W |
| 363. | E | W |
| 364. | F | W |
| 365. | G | W |
| 366. | H | W |
| 367. | I | W |
| 368. | K | W |
| 369. | L | W |
| 370. | M | W |
| 371. | N | W |
| 372. | P | W |
| 373. | Q | W |
| 374. | — | W |
| 375. | S | W |
| 376. | T | W |
| 377. | V | W |
| 378. | W | W |
| 379. | Y | W |
| 380. | A | Y |
| 381. | C | Y |
| 382. | D | Y |
| 383. | E | Y |
| 384. | F | Y |
| 385. | G | Y |
| 386. | H | Y |
| 387. | I | Y |
| 388. | K | Y |
| 389. | L | Y |
| 390. | M | Y |
| 391. | N | Y |
| 392. | P | Y |
| 393. | Q | Y |
| 394. | — | Y |
| 395. | S | Y |
| 396. | T | Y |
| 397. | V | Y |
| 398. | W | Y |
| 399. | Y | Y |

What is claimed is:

1. A polypeptide comprising a variant cellobiohydrolase I (“CBH I”) catalytic domain as compared to a reference CBH I catalytic domain, comprising:
(a) a substitution at the amino acid position corresponding to R268 of T. reesei CBH I (“R268 substitution”);
(b) a substitution at the amino acid position corresponding to R411 of T. reesei CBH I (“R411 substitution”); or
(c) both an R268 substitution and an R411 substitution,
wherein substitution (a), (b) or (c) decreases product inhibition as compared to the reference CBH I catalytic domain.

2. The polypeptide of claim 1, which has a single (R268 or R411) or double (R268 and R411) substitution selected from Table 14.

3. The polypeptide of claim 2, which does not have the same substitutions as one or more of variants 1, 9, 15, 161, 169, 175, 281 and/or 289 of Table 14.

4. The polypeptide of claim 1, towards which the IC₅₀ of cellobiose is at least 2-fold, at least 5-fold, at least 10-fold, at least 15-fold, at least 20-fold, at least 25-fold, at least 50-fold, at least 100-fold, at least 150-fold, at least 200-fold, at least 250-fold, at least 500-fold or at least 700-fold the IC₅₀ of cellobiose towards a reference CBH I which does not have a substitution at the amino acid corresponding to R268 or the amino acid position corresponding to R411.

5. The polypeptide of claim 1, towards which the IC₅₀ of cellobiose is up to 750-fold or up to 1,000-fold the IC₅₀ of cellobiose towards a reference CBH I which does not have a substitution at the amino acid corresponding to R268 or the amino acid position corresponding to R411.

6. The polypeptide of claim 1, towards which the IC₅₀ of cellobiose is at least 0.1 mM, at least 0.5 mM, at least 1 mM, at least 2 mM, at least 3 mM, at least 5 mM, at least 7 mM, at least 10 mM, at least 12 mM, at least 15 mM, at least 20 mM, at least 25 mM or at least 30 mM.

7. The polypeptide of claim 1, which comprises an R268 substitution.

8. The polypeptide of claim 7, wherein the R268 substituent is a histidine or lysine.

9. The polypeptide of claim 7, wherein the R268 substituent is an isoleucine, leucine, valine, phenylalanine, tyrosine, asparagine, serine, threonine, cysteine, or glycine.

10. The polypeptide of claim 7, wherein the R268 substituent is an alanine, tryptophan, aspartate, glutamate, or proline.

11. The polypeptide of claim 7, wherein the R268 substituent is a glutamine or methionine.

12. The polypeptide of claim 7, wherein said R268 substitution results in an IC₅₀ of cellobiose that is at least 2-fold, at least 5-fold, at least 7.5-fold or at least 10-fold the IC₅₀ of cellobiose towards a reference CBH I which does not have said R268 substitution.

13. The polypeptide of claim 7, wherein said R268 substitution results in an IC₅₀ of at least 0.1 mM, at least 0.25 mM, or at least 0.5 mM.

14. The polypeptide of claim 1, which comprises an R411 substitution.

15. The polypeptide of claim 14, wherein the R411 substituent is an alanine, aspartate, serine, cysteine, threonine, glycine or proline.

16. The polypeptide of claim 14, wherein the R411 substituent is a valine, glutamate, histidine, lysine, glutamine, or methionine.

17. The polypeptide of claim 16, wherein the R411 substituent is a valine, histidine, lysine, glutamate, threonine, glycine or methionine.

18. The polypeptide of claim 14, wherein the R411 substituent is a leucine, phenylalanine, tryptophan, tyrosine, or asparagine.

19. The polypeptide of claim 14, wherein the R411 substituent is an isoleucine.

20. The polypeptide of claim 14, wherein said R411 substitution results in an IC₅₀ of cellobiose that is at least 10-fold, at least 15-fold, at least 20-fold, at least 25-fold, at least 50-fold, at least 100-fold or at least 140-fold the IC₅₀ of cellobiose on a reference CBH I which does not have said R411 substitution.

21. The polypeptide of claim 14, wherein said R411 substitution results in an IC₅₀ of at least 1 mM, at least 2 mM, at least 3 mM, at least 4 mM, at least 5 mM, at least 6 mM, at least 7 mM or at least 8 mM.

22. The polypeptide of claim 1, which has R268A substitution and an R411 substitution.

23. The polypeptide of claim 22, wherein the R411 substituent is an alanine, valine, phenylalanine, aspartate, glutamate, lysine, glutamine, serine, threonine, cysteine, glycine, methionine, isoleucine, leucine, tryptophan, histidine, or proline.

24. The polypeptide of claim 22, wherein the R411 substituent is a tyrosine or an asparagine.

25. The polypeptide of claim 1, which has R268 substitution and an R411A substitution.

26. The polypeptide of claim 25, wherein the R268 substituent is an alanine, isoleucine, leucine, valine, phenylalanine, tryptophan, histidine, lysine, glutamine, serine, glycine, methionine, proline, cysteine, aspartate, tyrosine, glutamate, asparagine or threonine.

27. The polypeptide of claim 1, which has at least 0.7-fold the specific activity of a reference CBH I without said R268 or said R411 substitutions.

28. The polypeptide of claim 27, which has up to 4.5-fold the specificity activity of a reference CBH I without said R268 or said R411 substitutions.

29. The polypeptide of claim 28, which has at least 1-fold the specific activity of a reference CBH I without said R268 or said R411 substitutions.

30. The polypeptide of claim 28, which has at least 2-fold the specific activity of a reference CBH I without said R268 or said R411 substitutions.

31. The polypeptide of claim 1, wherein the variant CBH I catalytic domain comprises an amino acid sequence having at least 90% sequence identity to amino acids 18-444 of SEQ ID NO:2.

32. The polypeptide of claim 31, wherein the variant CBH I catalytic domain comprises an amino acid sequence having at least 95% sequence identity to amino acids 18-444 of SEQ ID NO:2.

33. The polypeptide of claim 32, wherein, other than said R268 and/or R411 substitutions, the variant CBH I catalytic domain comprises the sequence of amino acids 18-444 of SEQ ID NO:2.

34. The polypeptide of claim 1, wherein the variant CBH I catalytic domain does not comprise a R268A substitution.

35. The polypeptide of claim 34 whose amino acid sequence does not comprise SEQ ID NO:299.

36. The polypeptide of claim 34 whose amino acid sequence does not consist of SEQ ID NO:299.

37. The polypeptide of claim 1, wherein the variant CBH I catalytic domain does not comprise a R411A substitution.

38. The polypeptide of claim 37 whose amino acid sequence does not comprise SEQ ID NO:301 or SEQ ID NO:300.

39. The polypeptide of claim 37 whose amino acid sequence does not consist of SEQ ID NO:301 or SEQ ID NO:300.

40. A polypeptide comprising an amino acid sequence having at least 95% sequence identity to the amino acid sequence corresponding to positions 18-444 of SEQ ID NO:2, which has an R268K substitution and an R411A substitution as compared to a protein of SEQ ID NO:2.

41. The polypeptide of claim 40 in which said amino acid sequence has at least 97% sequence identity to the amino acid sequence corresponding to positions 18-444 of SEQ ID NO:2.

42. The polypeptide of claim 1, wherein the variant CBH I catalytic domain comprises an amino acid sequence having at least 90% sequence identity to amino acids 26-455 of SEQ ID NO:1.

43. The polypeptide of claim 42, wherein the variant CBH I catalytic domain comprises an amino acid sequence having at least 95% sequence identity to amino acids 26-455 of SEQ ID NO:1.

44. The polypeptide of claim 43, wherein, other than said R268 and/or R411 substitutions, the variant CBH I catalytic domain comprises the sequence of amino acids 26-455 of SEQ ID NO:1.

45. The polypeptide of claim 42, wherein the variant CBH I catalytic domain comprises one of the following amino acid substitutions or pairs of amino acid substitutions as compared to a protein of SEQ ID NO:1:
(a) R273K and R422K;
(b) R273K and R422A;
(c) R273A and R422K;
(d) R273A and R422A;
(e) R273A;
(f) R273K;
(g) R422A; and
(h) R422K.

46. The polypeptide of claim 42, wherein the variant CBH I catalytic domain comprises the amino acid substitutions R273K and R422K as compared to a protein of SEQ ID NO:1.

47. The polypeptide of claim 42, wherein the variant CBH I catalytic domain does not comprise both R273K and R422K substitutions as compared to a protein of SEQ ID NO:1.

48. The polypeptide of claim 47 whose amino acid sequence does not comprise SEQ ID NO:301 or SEQ ID NO:302.

49. The polypeptide of claim 47 whose amino acid sequence does not consist of SEQ ID NO:301 or SEQ ID NO:302.

50. The polypeptide of claim 1, wherein the variant CBH I catalytic domain comprises an amino acid sequence having at least 90%, at least 95% or at least 97% sequence identity of the amino acid sequence of the catalytic domain of any one of SEQ ID NOs:1-149.

51. The polypeptide of claim 1 in which the variant CBH I catalytic domain is operably linked to a cellulose binding domain.

52. The polypeptide of claim 51 in which the catalytic domain is operably linked to a cellulose binding domain via a linker.

53. The polypeptide of claim 51 in which the cellulose binding domain is C-terminal to the catalytic domain.

54. The polypeptide of claim 51 in which the cellulose binding domain is N-terminal to the catalytic domain.

55. The polypeptide of claim 1 which is a mature polypeptide.

56. The polypeptide of claim 55, wherein the mature polypeptide comprises an amino acid sequence having at least 90%, at least 95% or at least 97% sequence identity of mature portion of a polypeptide according to any one of SEQ ID NOs:1-149.

57. The polypeptide of claim 1 which further comprises a signal sequence.

58. The polypeptide of claim 56, which upon expression produces comprises a mature polypeptide comprising an amino acid sequence having at least 90%, at least 95% or at least 97% sequence identity of mature portion of a polypeptide according to any one of SEQ ID NOs:1-149.

59. The polypeptide of claim 1 towards which cellobiose has an IC₅₀ that is at least 2-fold the IC₅₀ of a reference CBH I lacking said R268 substitution and/or R411 substitution.

60. The polypeptide of claim 1 which CBH I activity that is at least 50% the CBH I activity of a reference CBH I lacking said R268 substitution and/or R411 substitution.

61. A composition comprising a polypeptide according to claim 1.

62. The composition of claim 61 in which said polypeptide represents at least 1% of all polypeptides in said composition.

63. The composition of claim 62 in which said polypeptide represents at least 5% of all polypeptide in said composition.

64. The composition of claim 63 in which said polypeptide represents at least 25% of all polypeptide in said composition.

65. The composition of claim 61 which is a whole cellulase.

66. The composition of claim 65, wherein the whole cellulase is produced by a host cell that recombinantly expresses said polypeptide.

67. The composition of claim 61 which is filamentous fungal whole cellulase.

68. A fermentation broth comprising a polypeptide according to claim 1.

69. The fermentation broth of claim 68, which is a filamentous fungal fermentation broth.

70. The fermentation broth of claim 68 which is a cell-free fermentation broth.

71. A method for saccharifying biomass, comprising: treating biomass with a composition according to claim 61 or with a fermentation broth according to claim 68.

72. The method of claim 71, further comprising recovering fermentable sugars.

73. The method of claim 72, wherein the fermentable sugars comprise disaccharides.

74. The method of claim 72, wherein the fermentable sugars comprise monosaccharides.

75. The method of claim 74, wherein monosaccharides are produced by a β-glucosidase in said composition or said fermentation broth.

76. A method for producing a fermentation product, comprising:
(a) treating biomass with a composition according to claim 61 or with a fermentation broth according to claim 68, thereby producing fermentable sugars; and
(b) culturing a fermenting microorganism in the presence of the fermentable sugars produced in step (a) under fermentation conditions, thereby producing a fermentation product.

77. The method of claim 76, wherein said fermentable sugars comprise disaccharides.

78. The method of claim 76, wherein the fermentable sugars comprise monosaccharides.

79. The method of claim 78, wherein monosaccharides are produced by a β-glucosidase in said composition or said fermentation broth.

80. The method of claim 76, wherein the fermentation product is ethanol.

81. The method of claim 76, further comprising, prior to step (a), pretreating the biomass.

82. The method of claim 76, wherein said fermenting microorganism is a bacterium or a yeast.

83. The method of claim 82, wherein said fermenting microorganism is a bacterium selected from Zymomonas mobilis, Escherichia coli and Klebsiella oxytoca.

84. The method of claim 82, wherein said fermenting microorganism is a yeast selected from Saccharomyces cerevisiae, Saccharomyces uvarum, Kluyveromyces fragilis, Kluyveromyces lactis, Candida pseudotropicalis, and Pachysolen tannophilus.

85. The method of claim 76, wherein said biomass is corn stover, bagasses, sorghum, giant reed, elephant grass, miscanthus, Japanese cedar, wheat straw, switchgrass, hardwood pulp, softwood pulp, crushed sugar cane, energy cane, or Napier grass.

86. A nucleic acid comprising a nucleotide sequence encoding the polypeptide of claim 1.

87. A vector comprising the nucleic acid of claim 86.

88. The vector of claim 87 which further comprises an origin of replication.

89. The vector of claim 87 which further comprises a promoter sequence operably linked to said nucleotide sequence.

90. The vector of claim 89, wherein the promoter sequence is operable in yeast.

91. The vector of claim 89, wherein the promoter sequence is operable in filamentous fungi.

92. A recombinant cell engineered to express the nucleic acid of claim 86.

93. The recombinant cell of claim 92 which is a eukaryotic cell.

94. The recombinant cell of claim 93 which is a filamentous fungal cell.

95. The recombinant cell of claim 94, wherein the filamentous fungal cell is of the genus Aspergillus, Penicillium, Rhizopus, Chrysosporium, Myceliophthora, Trichoderma, Humicola, Acremonium or Fusarium.

96. The recombinant cell of claim 94, wherein the filamentous fungal cell is of the species Aspergillus niger, Aspergillus oryzae, Trichoderma reesei, Penicillium chrysogenum, Myceliophthora thermophila, or Rhizopus oryzae.

97. The recombinant cell of claim 93 which is a yeast cell.

98. The recombinant cell of claim 97 which is a yeast cell of the genus Saccharomyces, Kluyveromyces, Candida, Pichia, Schizosaccharomyces, Hansenula, Klockera, Schwanniomyces or Yarrowia.

99. The recombinant cell of claim 98, wherein the yeast cell is of the species S. cerevisiae, S. bulderi, S. barnetti, S. exiguus, S. uvarum, S. diastaticus, K. lactis, K. marxianus or K. fragilis.

100. The recombinant cell of claim 99, which is a S. cerevisiae cell.

101. A host cell transformed with the vector of claim 87.

102. The host cell of claim 101 which is a prokaryotic cell.

103. The host cell of claim 102 which is a bacterial cell.

104. The host cell of claim 101 which is a eukaryotic cell.

105. A method of producing a polypeptide according to claim 1, comprising culturing a recombinant cell engineered to express said polypeptide under conditions in which the polypeptide is expressed.

106. The method of claim 105, wherein the polypeptide comprises a signal sequence and wherein the recombinant cell is cultured under conditions in which the polypeptide is secreted from the recombinant cell.

107. The method of claim 106, further comprising recovering the polypeptide from the cell culture.

108. The method of claim 107, wherein recovering the polypeptide comprises a step of centrifuging away cells and/or cellular debris.

109. The method of claim 107, wherein recovering the polypeptide comprises a step of filtering away cells and/or cellular debris.

110. A method for generating a product tolerant variant CBH I polypeptide, comprising
(a) modifying the nucleotide sequence of a CBH I-encoding nucleic acid so that the nucleic acid encodes a variant CBH I polypeptide, wherein said variant CBH I polypeptide comprises:
(i) an R268 substitution;
(ii) an R411 substitution; or
(iii) both an R268 substitution and an R411 substitution; and
(b) expressing said variant CBH I polypeptide,
thereby generating a product tolerant variant CBH I polypeptide.

111. A method for generating a nucleic acid that encodes a product tolerant variant CBH I polypeptide, comprising modifying the nucleotide sequence of a CBH I-encoding nucleic acid so that the nucleic acid encodes a variant CBH I polypeptide, wherein said variant CBH I polypeptide comprises:
(i) an R268 substitution;
(ii) an R411 substitution; or
(iii) both an R268 substitution and an R411 substitution,
thereby generating a nucleic acid that encodes a product tolerant variant CBH I polypeptide.

112. The method of claim 110 or claim 111, wherein the modification is by site directed mutagenesis.

113. The method of claim 110 or claim 111, wherein variant CBH I polypeptide comprises an R268 substitution.

114. The method of claim 113, wherein the R268 substituent is not an alanine.

115. The method of claim 113, wherein the R268 substituent is a lysine.

116. The method of claim 113, wherein the R268 substituent is an alanine.

117. The method of claim 110 or claim 111, which comprises an R411 substitution.

118. The method of claim 117, wherein the R411 substituent is not an alanine

119. The method of claim 117, wherein the R411 substituent is a lysine.

120. The method of claim 117, wherein the R411 substituent is an alanine.
